(19) United States
(12) Patent Application Publication
(10) Pub. No.: US 2025/0066335 A1
(43) Pub. Date: Feb. 27, 2025

(54) POLYSUBSTITUTED 4-HYDROXYPYRIDINE AND 4-HYDROXYQUINOLINE DERIVATIVES AS GPR84 ANTAGONISTS

(71) Applicant: UNIVERSITY OF COPENHAGEN, Copenhagen K (DK)

(72) Inventors: Trond Ulven, Copenhagen K (DK); Loukas Ieremias, Copenhagen K (DK); Asmita Manandhar, Copenhagen K (DK)

(21) Appl. No.: 18/570,133

(22) PCT Filed: Jun. 17, 2022
(86) PCT No.: PCT/EP2022/066636

§ 371 Date: Dec. 14, 2023

(30) Foreign Application Priority Data
Jun. 18, 2021 (GR) 20210100404
Jun. 25, 2021 (EP) 21181769.7

(51) Int. Cl. C07D 405/12; A61K 31/4433; A61K 31/4439; A61K 31/444; A61K 31/4709; C07D 405/14; C07D 413/14

(52) U.S. Cl. CPC C07D 405/12; A61K 31/4433; A61K 31/4439; A61K 31/444; A61K 31/4709; C07D 405/14; C07D 413/14

(57) ABSTRACT

The invention concerns a compound of formula (I), a pharmaceutical formulation comprising the compound of formula (I) and use of the pharmaceutical composition for treating fibrotic, inflammatory, diabetic or cognitive disease.

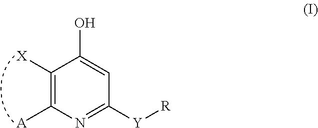

### FIELD OF THE INVENTION

The invention concerns a compound of formula I, a phamaceutical compostion comprising the compound of formula I and use of the pharmaceutical composition for treating fibrotic, inflammatory, diabetic or cognitive disease.

### BACKGROUND OF THE INVENTION

GPR84 is a G protein-coupled receptor (GPCR) that is activated by a relatively high concentration of decanoic acid and some related compounds, however its natural activator is still unknown and the receptor is thus considered to be “orphan” GPCR. To date, three classes of ligands are suggested to bind to three distinct sites on orphan GPR84, with the medium chain fatty acids agonist ligands (in particular decanoic acid) binding to one site; a second class of allosteric agonists represented by 3,3′-diindolylmethane (DIM), while a third class of allosteric antagonists represented by G9543 may bind to a third site (Mahmud et al. 2017).

GPCRs are cell-surface receptors that are known to be highly “druggable”, with ⅓ of current drugs acting through members of this class. GPR84 is expressed on immune cells, especially neutrophils and eosinophils, and is found in organs such as bone marrow, liver, lung, intestines and brain. The receptor has a pro-inflammatory effect and is induced in monocytes and macrophages by lipopolysaccharide (LPS) and activation of GPR84 induces secretion of pro-inflammatory cytokines such as IL-4, IL-8, IL-12B, CXCL1 and TNF-α. Several scientific groups have suggested that inhibition of GPR84 could represent a useful treatment of inflammatory diseases, including asthma, atopic dermatitis, cancer, diabetes, fibrosis and Inflammatory bowel disease (IBD) (Milligan et al. 2018; Miyamoto et al. 2017; Lynch & Wang 2016; Suckow & Briscoe 2017; Gagnon et al. 2018).

Recently, a low-potency dual GPR84 antagonist/GPR40 agonist was demonstrated to have efficacy against fibrosis in both mice and humans (Gagnon et al. 2018). This strongly suggests that GPR84 antagonism has potential for treatment of fibrosis. Besides this low-potency compound, only one series of GPR84 antagonists, discovered by Galapagos Pharmaceuticals, is known (WO2013/092791A1, WO2014/095798A1, WO2016/169911A1).

Liu et al. (ACS Med. Chem. Lett. 2016, 7:579-583) describes a series of alkylpyrimidine-4,6-diol derivatives which were designed and synthesized as novel GRP84 agonists based on a high-throughput screening (HTS) hit. 6-Nonylpyridine-2,4-diol was identified as the most potent agonist of GPR84 reported so far, with an EC50 of 0.189 nM. Yang Liu et al. concludes that these novel GPR84 agonists will provide valuable tools for the study of the physiological functions of GPR84.

Nan et al (WO2017/076264A1) have invented dihydroxypyrimidines and similar compounds as GPR84 agonists for treatment of septicemia.

Pillalyar et al. (2017) have reported optimized DIM GPR84 agonists, whereas Pillalyar et al. (2018) and Köse et al. (2019) have reported potent uracil-derived GPR84 agonists.

International patent application published under number WO2019/096944 (Galapagos N.V.) discloses compounds useful in the prophylaxis and/or treatment of one or more fibrotic diseases. In particular, the compounds antagonize GPR84, a G-protein-coupled receptor. The document also discloses pharmaceutical compositions comprising the compounds for use and methods for the prophylaxis and/or treatment of one or more fibrotic diseases by administering said compound.

### SUMMARY OF THE INVENTION

The present invention pertains to the following summarized embodiments:

In one preferred embodiment, the invention provides a compound of the formula I:

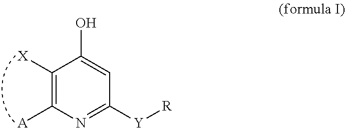

wherein

A is -(A1)ⱼ—(B1)ₖ-(A2)ₗ—(B2)ₘ—H, wherein

A1 is C₁₋₇ alkylene, C₂₋₇ alkenylene, C₂₋₇ alkynylene, or C₂₋₇ heteroalkylene, optionally substituted with one or two of independently selected U1;

A2 is C₁₋₁₄ alkylene, C₂₋₁₄ alkenylene, C₂₋₁₄ alkynylene, or C₂₋₁₄ heteroalkylene, optionally substituted with one, two, three or four of independently selected U1;

B1 and B2 are independently a C₃₋₇ aliphatic ring, a mono- or bicyclic aromatic ring or a fused ring, optionally substituted with one, two, three or four of independently selected U2; H is hydrogen;

j, k, l, and m are independently 0 or 1, wherein at least one of j, k, l and m is 1;

X is C₁₋₇ alkyl, C₂₋₇ heteroalkyl, C₂₋₇ alkenyl, C₂₋₇ heteroalkenyl, C₂₋₇ alkynyl, aryl, arylalkyl, aromatic ring, halogen, —CN, —NO₂, CF₃, —OH or —NH₂, optionally substituted with one, two or three independently selected U3;

------ defines an optional bond between X and A which is formed by substitution of one or two hydrogen radicals at a position on each of A and X such that a single or double bond is formed that results in a 5- or 6-membered aliphatic or aromatic ring fused with the pyridine;

Y is —OCH₂— or —N(R′)CH₂—, where R′ is hydrogen or C₁-C₃ alkyl; and

R is an aliphatic ring or a fused ring, optionally substituted with one, two or three of independently selected U4;

U1, U2, U3 and U4 are independently hydrogen, C₁₋₃ alkyl, C₁₋₃ haloalkyl, C₂₋₄ heteroalkyl, halogen, hydroxyl, ═O, =NR′═N—OR′—NR′R″, —SR′, —CN, —CO₂H, —SO₃H, —CO₂R′, —CONR′, C₂₋₅ alkynyl, C₂₋₅ alkenyl, C₃₋₅ cycloalkyl, C₃₋₅ heterocycloalkyl or —NO₂, where R′, and R″ independently refer to hydrogen, unsubstituted C₁₋₃ alkyl and C₂₋₃ heteroalkyl that can be connected by bonds to form heterocycloalkyl;

or a pharmaceutically acceptable salt thereof, or a solvate thereof, or a solvate of the pharmaceutically acceptable salt thereof.

In a further preferred embodiment, the present invention pertains to the compound of the formula I:

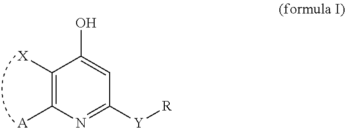

wherein

A is -(A1)ⱼ—(B1)ₖ-(A2)ₗ—(B2)ₘ—H, wherein

A1 and A2 independently are C₁₋₇ alkylene, C₂₋₇ alkenylene, C₂₋₇ alkynylene, or C₁₋₇ heteroalkylene, optionally substituted with one or two of independently selected U1;

B1 and B2 are independently an aliphatic ring, an aromatic ring or a fused ring, optionally substituted with one or two of independently selected U2; H is hydrogen;

j, k, l, and m are independently 0 or 1;

X is C₁₋₇ alkyl, C₂₋₇ heteroalkyl, C₂₋₇ alkenyl, C₂₋₇ heteroalkenyl, C₂₋₇ alkynyl, halogen, —CN, —NO₂, CF₃, —OH or —NH₂, optionally substituted with one, two or three independently selected U3;

------ defines an optional bond between X and A which is formed by substitution of one or two hydrogen radicals at a position on each of A and X such that a single or double bond is formed that results in a 5- or 6-membered aliphatic or aromatic ring fused with the pyridine;

Y is —OCH₂—, —N(R′)CH₂—, —CH₂CH₂—, —CH₂—, —N(R′)—, or —O—, where R′ is hydrogen or C₁-C₃ alkyl; and

R is an aromatic ring, an aromatic ring comprising nitrogen, an aliphatic ring or a fused ring, optionally substituted with one, two or three of independently selected U4;

U1, U2, U3 and U4 are independently hydrogen, —CF₃, C₁₋₃ alkyl, C₁₋₃ haloalkyl, C₂₋₄ heteroalkyl, halogen, ═O, =NR′=N—OR′—NR′R″, —SR′, —CN, C₂₋₅ alkynyl, C₂₋₅ alkenyl, C₃₋₅ cycloalkyl, C₃₋₅ heterocycloalkyl and —NO₂, where R′, R″ independently refer to hydrogen, unsubstituted C₁₋₃ alkyl and C₂₋₃ heteroalkyl that can be connected by bonds to form rings, cycloalkyl or heterocycloalkyl;

or a pharmaceutically acceptable salt thereof, or a solvate thereof, or a solvate of the pharmaceutically acceptable salt thereof.

In a further preferred embodiment, the invention pertains to the compound of formula I according to any of the preceding embodiments, wherein Y is —OCH₂—.

In a further preferred embodiment, the invention pertains to the compound of formula I according to any of the preceding embodiments, wherein R is a bicyclic fused ring composed of a 1,4-dioxane and an aromatic ring.

In a further preferred embodiment, the invention pertains to the compound of formula I according to any of the preceding embodiments, wherein R is

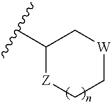

where the ring is optionally substituted with —CF₃, C₁₋₃ alkyl or C₂₋₄ heteroalkyl, and wherein
Z is —O—, —CH₂—, —NH— or N—(CH₂)₀₋₂—CH₃,
W is —O—, —NH—, —N(CH₂)₀₋₂CH₃ or —CH₂—, and
n is 0, 1 or 2.

In a further preferred embodiment, the invention pertains to the compound of formula I according to any of the preceding embodiments, wherein n=1.

In a further preferred embodiment, the invention pertains to the compound of formula I according to any of the preceding embodiments, wherein Z═O.

In a further preferred embodiment, the invention pertains to the compound of formula I according to any of the preceding embodiments, wherein W=—O—.

In a further preferred embodiment, the invention pertains to the compound of formula I according to any of the preceding embodiments, wherein W=—CH₂—.

In a further preferred embodiment, the invention pertains to the compound of formula I according to any of the preceding embodiments, wherein Y is —OCH₂—, and R is

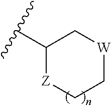

where n=1, Z═O and W=O.

In a further preferred embodiment, the invention pertains to the compound of formula I according to any of the preceding embodiments, wherein X is C₁₋₇ alkyl, C₂₋₇ heteroalkyl optionally substituted with one or two of independently selected U3, halogen, CN, or —CF₃.

In a further preferred embodiment, the invention pertains to the compound of formula I according to any of the preceding embodiments, wherein X is C₁₋₃ alkyl.

In a further preferred embodiment, the invention pertains to the compound of formula I according to any of the preceding embodiments, wherein X is methyl or ethyl.

In a further preferred embodiment, the invention pertains to the compound of formula I according to any of the preceding embodiments, wherein
j=1, k=1, l=1, and m=1, resulting in A being -A1-B1-A2-B2-H,
j=1, k=1, l=1, and m=0, resulting in A being -A1-B1-A2-H.

In a further preferred embodiment, the invention pertains to the compound of formula I according to any of the preceding embodiments, wherein A1 is C₁₋₇ alkylene or C₁₋₇ heteroalkylene, optionally substituted with one or two of independently selected U1.

In a further preferred embodiment, the invention pertains to the compound of formula I according to any of the preceding embodiments, wherein A1 is ethylene.

In a further preferred embodiment, the invention pertains to the compound of formula I according to any of the preceding embodiments, wherein A1 is —CD₂CD₂—.

In a further preferred embodiment, the invention pertains to the compound of formula I according to any of the preceding embodiments, wherein A1 is ethylene or —CD₂CD₂-substituted by one or two of independently selected U1, and B1 is benzene substituted by one, two, three or four of independently selected U2.

In a further preferred embodiment, the invention pertains to the compound of formula I according to any of the preceding embodiments, wherein A2 is C₁₋₁₄ alkylene or C₂₋₁₄ heteroalkylene, optionally substituted with one, two, three or four of independently selected U1.

In a further preferred embodiment, the invention pertains to the compound of formula I according to any of the preceding embodiments, wherein B1 and/or B2 is an aromatic ring, optionally substituted with one or two of independently selected U2.

In a further preferred embodiment, the invention pertains to the compound of formula I according to any of the preceding embodiments, wherein A is -A1-B1-A2-H, where A1 is C₁₋₅ alkylene or C₂₋₅ heteroalkylene, B1 is aryl, and A2 is is C₁₋₅ alkylene or C₂₋₁₄ heteroalkylene optionally substituted with one, two, three or four of independently selected U1.

In a further preferred embodiment, the invention pertains to the compound of formula I according to any of the preceding embodiments, wherein X is C₁₋₇ alkyl or halogen and A is A1-B1-A2-H, where A1 and A2 are C₁₋₅ alkylene, and B1 is —(C₆H₄)—.

In a further preferred embodiment, the invention pertains to the compound of formula I according to any of the preceding embodiments, wherein A is -A1-B1-A2-B2-H, where A1 and A2 are C₁₋₅ alkylene or C₂₋₅ heteroalkylene optionally substituted with one, two, three or four of independently selected U1, and B1 and B2 are aryl optionally substituted with one, two, three or four of independently selected U2.

In a further preferred embodiment, the invention pertains to the compound of formula I according to any of the preceding embodiments, wherein A is -A1-B1-H, where A1 is C₁₋₅ alkylene or C₂₋₅ heteroalkylene optionally substituted with one, two, three or four of independently selected U1, and B1 is aryl optionally substituted with one or two of independently selected U2.

In a further preferred embodiment, the invention pertains to the compound of formula I according to any of the preceding embodiments, wherein ------ is absent.

In a further preferred embodiment, the invention pertains to the compound of formula I according to any of the preceding embodiments, wherein ------ connects the second carbon of X and A1 to form a 5 or 6 membered carbocyclic or heterocyclic ring.

In a further preferred embodiment, the invention pertains to the compound of formula I according to any of the preceding embodiments, wherein ------ connects the X and A1 to form a quinoline.

In a further preferred embodiment, the invention pertains to the compound of formula I according to any of the preceding embodiments, wherein A is —(CH₂)₂—(C₆H₄)—(CH₂)₂—CH₃.

In a further preferred embodiment, the invention pertains to the compound of formula I according to any of the preceding embodiments, wherein A is —(CH₂)₂—(C₆H₄)—OCH₂CH₃.

In a further preferred embodiment, the invention pertains to the compound of formula I according to any of the preceding embodiments, wherein A is —(CH₂)₂—(C₆H₄)—O(CH₂)₂₋₄NHCO₂R′, wherein R′ is a branched or straight C₁₋₆ alkyl.

In a further preferred embodiment, the invention pertains to the compound of formula I according to any of the preceding embodiments, wherein A is —(CH₂)₂—(C₆H₄)—O(CH₂)₂₋₄NHCOR′, wherein R′ is a branched or straight C₁₋₆ alkyl.

In a further preferred embodiment, the invention pertains to the compound of formula I according to any of the preceding embodiments, wherein A is —(CH₂)₂—(C₆H₄)—O(CH₂)₂₋₄O(CH₂)₂NHCO₂R′, wherein R′ is a branched or straight C₁₋₄ alkyl.

In a further preferred embodiment, the invention pertains to the compound of formula I according to any of the preceding embodiments, wherein A is —(CH₂)₂—(C₆H₄)—O(CH₂)₂₋₄SO₂R′, wherein R′ is a branched or straight C₁₋₄ alkyl.

In a further preferred embodiment, the invention pertains to the compound of formula I according to any of the preceding embodiments, wherein X is methyl or ethyl, wherein ------ is absent, and wherein Y is —OCH₂—, and R is

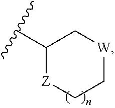

where n=1, Z═O and W=O.

In a further preferred embodiment, the invention pertains to the compound of formula I according to any of the preceding embodiments, wherein the compound is selected from the group consisting of:
3-methyl-2-(4-propylphenethyl)-6-((tetrahydro-2H-pyran-2-yl)methoxy)pyridin-4-ol,
3-ethyl-2-(4-propylphenethyl)-6-((tetrahydro-2H-pyran-2-yl)methoxy)pyridin-4-ol,
6-((1,4-dioxan-2-yl)methoxy)-3-methyl-2-(4-propylphenethyl)pyridin-4-ol,
(S)-6-((1,4-dioxan-2-yl)methoxy)-2-(4-ethoxyphenethyl)-3-methylpyridin-4-ol,
6-((1,4-dioxan-2-yl)methoxy)-3-ethyl-2-(4-propylphenethyl)pyridin-4-ol,
6-((1,4-dioxan-2-yl)methoxy)-2-(4-ethoxyphenethyl)-3-ethylpyridin-4-ol, tert-butyl (2-(4-(2-(6-((1,4-dioxan-2-yl)methoxy)-4-hydroxy-3-methylpyridin-2-yl)ethyl)phenoxy)ethyl)carbamate,
2-(4-(2-(6-((1,4-dioxan-2-yl)methoxy)-4-hydroxy-3-methylpyridin-2-yl)ethyl)phenoxy)ethan-1-aminium chloride,
N-(2-(4-(2-(6-((1,4-dioxan-2-yl)methoxy)-4-hydroxy-3-methylpyridin-2-yl)ethyl)phenoxy)ethyl)acetamide,
ethyl 2-(4-(2-(6-((1,4-dioxan-2-yl)methoxy)-4-hydroxy-3-methylpyridin-2-yl)ethyl) phenoxy)acetate,
tert-butyl (2-(4-(2-(6-((1,4-dioxan-2-yl)methoxy)-3-ethyl-4-hydroxypyridin-2-yl)ethyl) phenoxy)ethyl)carbamate,
tert-butyl (3-(4-(2-(6-((1,4-dioxan-2-yl)methoxy)-3-ethyl-4-hydroxypyridin-2-yl)ethyl) phenoxy)propyl)carbamate,
methyl 2-((4-(2-(6-((1,4-dioxan-2-yl)methoxy)-3-ethyl-4-hydroxypyridin-2-yl)ethyl)phenoxy)methyl)oxazole-4-carboxylate,
tert-butyl (2-(2-(4-(2-(6-((1,4-dioxan-2-yl)methoxy)-3-ethyl-4-hydroxypyridin-2-yl)ethyl) phenoxy)ethoxy)ethyl)carbamate,
6-((1,4-dioxan-2-yl)methoxy)-3-ethyl-2-(4-hydroxyphenethyl)pyridin-4-ol,
2-(2-(4-(2-(6-((1,4-dioxan-2-yl)methoxy)-3-ethyl-4-hydroxypyridin-2-yl)ethyl)phenoxy)ethyl)isoindoline-1,3-dione,
6-((1,4-dioxan-2-yl)methoxy)-2-(2-(6-ethoxypyridin-3-yl)ethyl)-3-ethylpyridin-4-ol,
6-((1,4-dioxan-2-yl)methoxy)-2-(2-(6-ethoxy-5-fluoropyridin-3-yl)ethyl)-3-ethylpyridin-4-ol,
6-((1,4-dioxan-2-yl)methoxy)-2-(4-(2-aminoethoxy)phenethyl)-3-ethylpyridin-4-ol hydrochloride,
6-((1,4-dioxan-2-yl)methoxy)-2-(4-(3-aminopropoxy)phenethyl)-3-ethylpyridin-4-ol hydrochloride,
6-((1,4-dioxan-2-yl)methoxy)-3-ethyl-2-(4-(3-(methylsulfonyl)propoxy)phenethyl)pyridin-4-ol,
6-((1,4-dioxan-2-yl)methoxy)-3-ethyl-2-(4-(2-phenoxyethoxy)phenethyl)pyridin-4-ol,
6-((1,4-dioxan-2-yl)methoxy)-3-ethyl-2-(4-((3-methyl-1,2,4-oxadiazol-5-yl)methoxy)phenethyl)pyridin-4-ol,
6-((1,4-dioxan-2-yl)methoxy)-2-(2-(4-(ethoxy-d5)phenyl)ethyl-1,1,2,2-d4)-3-ethylpyridin-4-ol,
(S)-6-((1,4-dioxan-2-yl)methoxy)-2-(4-butylphenyl)-3-ethylpyridin-4-ol,
(S)-6-((1,4-dioxan-2-yl)methoxy)-2-(3,4-dimethoxyphenethyl)-3-ethylpyridin-4-ol,
(S)-6-((1,4-dioxan-2-yl)methoxy)-2-(2,4-dimethoxyphenethyl)-3-ethylpyridin-4-ol,
(S)-5-(2-(6-((1,4-dioxan-2-yl)methoxy)-3-ethyl-4-hydroxypyridin-2-yl)ethyl)-2-methoxybenzonitrile,
(S)-2-(2-(6-((1,4-dioxan-2-yl)methoxy)-3-ethyl-4-hydroxypyridin-2-yl)ethyl)-5-methoxybenzonitrile,
ethyl 5-(4-(2-(6-((1,4-dioxan-2-yl)methoxy)-3-ethyl-4-hydroxypyridin-2-yl)ethyl)phenoxy)pentanoate,
6-((1,4-dioxan-2-yl)methoxy)-3-ethyl-2-(4-(2-hydroxyethoxy)phenethyl)pyridin-4-ol,
2-(4-(2-(6-((1,4-dioxan-2-yl)methoxy)-3-ethyl-4-hydroxypyridin-2-yl)ethyl)phenoxy)acetamide,
sodium 3-(4-hydroxy-2-(4-propylphenethyl)-6-((tetrahydro-2H-pyran-2-yl)methoxy)pyridin-3-yl)propanoate, and
ethyl 3-(4-hydroxy-2-(4-propylphenethyl)-6-((tetrahydro-2H-pyran-2-yl)methoxy)pyridin-3-yl)propanoate.

In a further preferred aspect, the invention concerns a pharmaceutical composition for use as a medicament, said pharmaceutical composition comprising a compound according to formula I and a pharmaceutically acceptable carrier, excipient or diluent.

In one preferred aspect, the pharmaceutical composition can be used in the treatment of inflammatory or diabetic disease.

### Definitions

The term “alkyl”, by itself or as part of another substituent, means, unless otherwise stated, a straight or branched chain, or cyclic hydrocarbon radical, or combination thereof, which is fully saturated, having the number of carbon atoms designated (e.g., C₁₋₇ means one to seven carbon atoms). Examples of alkyl groups include methyl, ethyl, n-propyl, isopropyl, n-butyl, t-butyl, isobutyl, sec-butyl, cyclohexyl, (cyclohexyl)methyl, cyclopropyl, cyclopropylmethyl, and homologs and isomers of, for example, n-pentyl, n-hexyl, n-heptyl, n-octyl, and the like.

The term “cycloalkyl” by itself or as part of another substituent, means, unless otherwise stated, a member of the subset of alkyl comprising cyclic hydrocarbon radicals.

The term “alkylene” by itself or as part of another substituent means a divalent radical derived from alkyl. The two valences may be on any carbon atom of the chain, including on the same carbon, resulting in an alkyl connected by a double bond. Typically, an alkyl (or alkylene) group will have from 1 to 24 carbon atoms, with those groups having 8 or fewer carbon atoms being preferred in the present invention. Typical examples of alkylene include —CH₂CH₂CH₂CH₂—, —CH(CH₃)— and =CHCH₂CH₃.

The term “alkenyl”, by itself or in combination with another, term means, unless otherwise stated, a monovalent unsaturated (olefinic) hydrocarbon chains having a specified number of carbon atoms (i.e. C₂₋₈ means two to eight carbons). Preferably, alkenyl has 2 to 7 carbon atoms, and more particularly, from 2 to 3 carbon atoms, which can be straight-chained or branched and having at least 1 and particularly from 1 to 2 sites of olefinic unsaturation. Preferred alkenyl groups include ethenyl (—CH═CH₂), n-propenyl (—CH₂CH═CH₂), isopropenyl (—C(CH₃)=CH₂) and the like.

The term “alkenylene”, by itself or as part of another substituent, means a straight or branched chain hydrocarbon radical, or combination thereof, which may be mono- or polyunsaturated, having the number of carbon atoms designated (i.e. C₂₋₈ means two to eight carbons) and one or more double bonds. Examples of alkenylene groups include ethenylene (—CH═CH—), —CH₂CH═CH—, —CH₂C(CH₃)=CH—, and higher homologs and isomers thereof.

The term “alkynyl”, by itself or as part of another substituent, means a straight or branched chain hydrocarbon radical, or combination thereof, which may be mono- or polyunsaturated, having the number of carbon atoms designated (i.e. C₂-C₈ means two to eight carbons) and one or more triple bonds. Examples of alkynyl groups include ethynyl, 1- and 2-propynyl, 3-butynyl, and higher homologs and isomers thereof.

The term “alkynylene” means a divalent alkyne radical groups having the number of carbon atoms and the number of triple bonds specified, in particular 2 to 7 carbon atoms and more particularly 2 to 3 carbon atoms which can be straight-chained or branched. This term is exemplified by groups such as —C≡C—, —CH₂—C≡C—, and —C(CH₃)H—C≡C—.

The term “heteroalkyl”, by itself or in combination with another term, means, unless otherwise stated, a stable straight or branched chain, or cyclic hydrocarbon radical, or combinations thereof, consisting of carbon atoms and from one to three heteroatoms selected from the group consisting of O, N, and S, and wherein the nitrogen and sulfur atoms may optionally be oxidized, and the nitrogen heteroatom may optionally be quaternized. The heteroatom(s) O, N, and S may be placed at any position of the heteroalkyl group. Examples include —CH₂CH₂OCH₃, —CH₂CH₂NHCH₃, —CH₂CH₂N(CH₃)CH₃, —CH₂SCH₂CH₃, —CH₂CH₂S(O)CH₃, —CH₂CH₂S(O)₂CH₃, —OCH₃ and —CH₂CH═N—OCH₃. Up to two heteroatoms may be consecutive, such as, for example, —CH₂NHS(O)₂CH₃. When a prefix such as C₂₋₇ is used to refer to a heteroalkyl group, the number of carbons (2 to 7, in this example) is meant to include the heteroatoms as well. For example, a C₂-heteroalkyl group is meant to include, for example, —CH₂OH (one carbon atom and one heteroatom replacing a carbon atom), —SCH₃ and —CH₂SH, and a C₃-heteroalkyl group is meant to include —N(CH₃)₂.

To further illustrate the definition of a heteroalkyl group, where the heteroatom is oxygen, a heteroalkyl group is an, oxyalkyl group. For instance, (C₂-C₈)oxyalkyl is meant to include, for example —CH₂O—CH₃ (a C₃-oxyalkyl group with two carbon atoms and one oxygen replacing a carbon atom), —CH₂CH₂CH₂CH₂OH, and the like.

The term “heteroalkylene” by itself or as part of another substituent means a divalent radical derived from heteroalkyl, as exemplified by —CH₂CH₂SCH₂CH₂— and —CH₂SCH₂CH₂NHCH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied. Heteroalkylene groups such as oxymethyl groups (—CH₂O—) may be substituted or unsubstituted. In some embodiments, heteroalkylene groups may be substituted with an alkyl group. For example, the carbon atom of an oxymethylene group may be substituted with a methyl group in a group of formula —CH(CH₃)O—. Still further, C1 heteroalkylene may be a divalent radical derived from a heteroatom, as exemplified by —O—, —N—, —S—.

The term “heteroalkenyl” by itself or as part of another term, means a straight or branched chain hydrocarbon radical, or combination thereof, which may be mono- or polyunsaturated, having the number of carbon atoms and from one to three heteroatoms selected from the group consisting of O, N, and S, and wherein the nitrogen and sulfur atoms may optionally be oxidized, and the nitrogen heteroatom may optionally be quaternized (i.e. C₂₋₈ means the sum of two to eight carbons and heteroatoms) and one or more double bonds. When a prefix such as C₂₋₇ is used to refer to a heteroalkyl group, the number of carbons (2 to 7, in this example) is meant to include the heteroatoms as well. Examples of heteroalkenylene groups include —CH═CHCH₂OCH₃, —OCH₂CH═CH₂, —CH═N—CH₃, —N═CHCH₂OCH₃ and —CH₂CH═C(CH₃)CH₂OCH₃.

The terms “cycloalkyl” and “heterocycloalkyl” by themselves or in combination with other terms, represent, unless otherwise stated, monocyclic versions of “alkyl” and “heteroalkyl” respectively. Thus, the terms “cycloalkyl” and “heterocycloalkyl” are meant to be included in the terms “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 1-pyrrolidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 4,5-dihydroisoxazol-3-yl, and the like. The term “heterocycloalkyl” includes fully saturated compounds such as piperidine and compounds with partial saturation that are not aromatic. Examples of such groups include, but are not limited to, an imidazoline, oxazoline, or isoxazoline.

The term “cycloalkylene” and “heterocycloalkylene,” by themselves or in combination with other terms, represent, unless otherwise stated, monocyclic versions of “alkylene” and “heteroalkylene,” respectively. Thus, the terms “cycloalkylene” and “heterocycloalkylene” are meant to be included in the terms “alkylene” and “heteroalkylene,” respectively. Additionally, for heterocycloalkylene, one or more heteroatoms can occupy positions at which the heterocycle is attached to the remainder of the molecule. Typically, a cycloalkylene or heterocycloalkylene will have from 3 to 9 atoms forming the ring, more typically, 3 to 7 atoms forming the ring, and even more typically, 5 or 6 atoms will form the cycloalkylene or heterocycloalkylene ring.

The term “aliphatic ring” by itself or as part of another substituent means a cycloalkyl, a heterocycloalkyl, a cycloalkylene or a heterocycloalkylene of any valency, but typically mono- or divalent. Examples of such groups include cyclopentyl, 1,4-dioxanyl, or piperidinyl.

The term “fused ring” means, unless otherwise stated, a cyclic aromatic or C₃₋₇ aliphatic ring which shares bonds with one or two other cyclic aromatic or C₃₋₇ aliphatic ring. The term “fused aryl” means, unless otherwise stated, a fused ring where at least one of the rings is an aryl. The term “fused heteroaryl” means, unless otherwise stated, a fused ring system where at least one of the rings is a heteroaryl. Examples of rings, fused aryl and fused heteroaryl groups include, 1-naphthyl, 1-tetrahydronaphthyl, 1-decahydronaphthyl, 2-naphthyl, dibenzofuryl, 5-benzothiazolyl, 2-benzoxazolyl, 5-benzoxazolyl, benzooxadiazolyl, purinyl, 2-benzimidazolyl, 5-indolyl, 1H-indazolyl, indanyl, carbazolyl, carbolinyl, 1-isoquinolyl, 5-isoquinolyl, 2-quinoxalinyl, 5-quinoxalinyl, 2-quinolyl, 3-quinolyl, 4-quinolyl, 5-quinolyl, 6-quinolyl, 7-quinolyl, and 8-quinolyl.

The term “aryl” refers to a monovalent aromatic hydrocarbon group derived by the removal of one hydrogen atom from a single carbon atom of a parent aromatic ring system. In particular aryl refers to an aromatic ring structure, monocyclic or fused polycyclic, with the number of ring atoms specified. Specifically, the term includes groups that include from 6 to 10 ring members. Particular aryl groups include phenyl, and naphthyl.

When the term “hetero” is used, it describes a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, and the like having from 1 to 4, and particularly from 1 to 3 heteroatoms, more typically 1 or 2 heteroatoms, for example a single heteroatom.

The term “heteroaryl” means an aromatic ring structure, monocyclic or fused polycyclic, that includes one or more heteroatoms independently selected from O, N and S and the number of ring atoms specified.

The term “arylalkyl” refers to aryl or heteroaryl attached via a straight or branched alkylene group. Examples of arylalkyl include benzyl, 2-phenylethyl, and (S)-3-(2-pyridyl)butyl.

The term “aromatic ring” by itself or as part of another substituent means aryl or heteroaryl of any valency, but typically mono- or divalent.

In particular, the aromatic ring structure may have from 5 to 11 ring members. Preferably, the heteroaryl group is a five membered or six membered monocyclic ring or a fused bicyclic structure formed from fused five and six membered rings or two fused six membered rings or, by way of a further example, two fused five membered rings. Each ring may contain up to four heteroatoms typically selected from nitrogen, sulphur and oxygen. Typically, the heteroaryl ring will contain up to 4 heteroatoms, more typically up to 3 heteroatoms, more usually up to 2, for example a single heteroatom.

In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group (Amino refers to —NH2) substituents of the ring, will be less than five.

Examples of five membered monocyclic heteroaryl groups include but are not limited to pyrrolyl, furanyl, thiophenyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered monocyclic heteroaryl groups include but are not limited to pyridinyl, pyrazinyl, pyridazinyl, pyrimidinyl and triazinyl.

Particular examples of bi cyclic heteroaryl groups containing a five membered ring fused to another five-membered ring include but are not limited to imidazothiazolyl and imidazoimidazolyl.

Particular examples of bicyclic heteroaryl groups containing a six membered ring fused to a five membered ring include but are not limited to benzofuranyl, benzothiophenyl, benzoimidazolyl, benzoxazolyl, isobenzoxazolyl, benzisoxazolyl, benzothiazolyl, benzoisothiazolyl, isobenzofuranyl, indolyl, isoindolyl, indolizinyl, purinyl (e.g. adenine, guanine), indazolyl, pyrazolopyrimidinyl, triazolopyrimidinyl, and pyrazolopyridinyl groups.

Particular examples ofbicyclic heteroaryl groups containing two fused six membered rings include but are not limited to quinolinyl, isoquinolinyl, pyridopyridinyl, quinoxalinyl, quinazolinyl, cinnolinyl, phthalazinyl, naphthyridinyl, and pteridinyl groups. Particular heteroaryl groups are those derived from thiophenyl, pyrrolyl, benzothiophenyl, benzofuranyl, indolyl, pyridinyl, quinolinyl, imidazolyl, oxazolyl and pyrazinyl.

‘Hydroxyl’ refers to the radical —OH.

‘Oxo’ refers to the radical ═O.

‘Substituted’ refers to a group in which one or more hydrogen atoms are each independently replaced with the same or different substituent(s). As used herein, term ‘substituted with one or more’ refers to one to four substituents. In one embodiment it refers to one to three substituents. In further embodiments it refers to one or two substituents. In a yet further embodiment it refers to one substituent.

The term ‘substituent’, which may be present on alkyl or heteroalkyi radicals, as well as those groups referred to as alkylene, alkenyl, heteroalkylene, heteroalkenyl, alkynyl, cycloalkyl, heterocycloalkyl, cycloalkenyl and heterocycloalkenyl, or on other groups indicated as “optionally substituted”, can be a variety of groups selected from: —C₁₋₅ alkyl, —OR′, ═O, =NR′═N—OR′—NR′R″, —SR′, halogen, —OC(O)R′—C(O)R′, —CO₂R′, —CONR′R″, —OC(O)NR′R″, —NR″C(O)R′, —NR′C(O)NR″R′″, —NR′SO₂NR″R′″, —NR″CO₂R′, —NR′C(NR″R′″)=NR^{IV}, —SiR′R″R′″, —S(O)R′, —SO₂R′, —SO₂NR′R″, —NR″SO₂R′, —CN, —(C₂-C₅)alkynyl, —(C₂-C₅)alkenyl, and —NO₂, in a number ranging from zero to three, with those groups having zero, one or two substituents being particularly preferred. Other suitable substituents include aryl and heteroaryl groups. R′, R″, R′″ and R^{IV} each independently refer to hydrogen, unsubstituted (C₁-C₃)alkyl and (C₂-C₃)heteroalkyl, unsubstituted aryl, aryl substituted with one to three halogens, unsubstituted (C₁-C₄)-alkyl, (C₁-C₄)-alkoxy or (C₁-C₄)-thioalkoxy groups, halo(C₁-C₄)alkyl, or aryl-(C₁-C₄)alkyl groups. When R′ and R″ are attached to the same nitrogen atom, they can be combined with the nitrogen atom to form a 5-, 6- or 7-membered ring. For example, —NR′R″ is meant to include 1-pyrrolidinyl and 4-morpholinyl.

‘Sulfo’ or ‘sulfonic acid’ refers to a radical such as —SO₃H.

‘Thiol’ refers to the group —SH.

‘Thioalkoxy’ refers to the group —S-alkyl where the alkyl group has the number of carbon atoms specified. In particular the term refers to the group —S—C₁₋₆ alkyl. Particular thioalkoxy groups are thiomethoxy, thioethoxy, n-thiopropoxy, isothiopropoxy, n-thiobutoxy, tert-thiobutoxy, secthiobutoxy, n-thiopentoxy, n-thiohexoxy, and 1,2-dimethylthiobutoxy. Particular thioalkoxy groups are lower thioalkoxy, i.e. with between 1 and 6 carbon atoms. Further particular alkoxy groups have between 1 and 4 carbon atoms.

One having ordinary skill in the art of organic synthesis will recognize that the maximum number of heteroatoms in a stable chemically feasible heterocyclic ring, whether it is aromatic or aliphatic, is determined by the size of the ring, the degree of unsaturation and the valence of the heteroatoms. In general, a heterocyclic ring may have one to four heteroatoms so long as the heteroaromatic ring is chemically feasible and stable.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in viva. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, EtOH, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans, where the terms ‘human’, ‘patient’ and ‘subject’ are used interchangeably herein.

‘Effective amount’ means the amount of a compound of the invention that, when administered to a subject for treating a disease, is sufficient to effect such treatment for the disease. The “effective amount” can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘fibrotic diseases’ refers to diseases characterized by excessive scarring due to excessive production, deposition, and contraction of extracellular matrix, and are that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to idiopathic pulmonary fibrosis (IPF); cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, Langerhans cell granulomatosis, lymphangioleiomyomatosis, inherited diseases (Hermansky-Pudlak Syndrome, tuberous sclerosis, neurofibromatosis, metabolic storage diseases, familial interstitial lung disease); radiation induced fibrosis; chronic obstructive pulmonary disease; scleroderma; bleomycin induced pulmonary fibrosis; chronic asthma; silicosis; asbestos induced pulmonary fibrosis; acute respiratory distress syndrome (ARDS); kidney fibrosis; tubulointerstitium fibrosis; glomerular nephritis; diabetic nephropathy, focal segmental glomerular sclerosis; IgA nephropathy; hypertension; Alport; gut fibrosis; liver fibrosis; cirrhosis; alcohol induced liver fibrosis; toxic/drug induced liver fibrosis; hemochromatosis; alcoholic steato hepatitis (ASH), nonalcoholic steatohepatitis (NASH), nonalcoholic fatty liver disease (NAFLD); cholestasis, biliary duct injury; primary sclerosing cholangitis (PSC), primary biliary cirrhosis (PBC); infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; Duchenne muscular dystrophy (DMD) associated musculoskeletal fibrosis, vascular restenosis; atherosclerosis; arteriosclerosis; W egener's granulomatosis; Peyronie's disease, or chronic lymphocytic. More particularly, the term “fibrotic diseases” refers to idiopathic pulmonary fibrosis (IPF), Dupuytren disease, nonalcoholic steatohepatitis (NASH), nonalcoholic fatty liver disease (NAFLD), Alcoholic steato hepatitis, (ASH), portal hypertension, systemic sclerosis, renal fibrosis, and cutaneous fibrosis. Most particularly, the term “fibrotic diseases” refers to nonalcoholic steatohepatitis (NASH), and/or nonalcoholic fatty liver disease (NAFLD). Alternatively, most particularly, the term “fibrotic diseases” refers to IPF.

‘Compound(s) of the invention’, and equivalent expressions, are meant to embrace compounds of the Formula(e) as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

When ranges are referred to herein, for example but without limitation, C₁₋₇ alkyl, the citation of a range should be considered a representation of each member of said range.

Other derivatives of the compounds of this invention have activity in both their acid and acid derivative forms, but the acid sensitive form often offers advantages of solubility, tissue compatibility, or delayed release in the mammalian organism (Bundgard, H, 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particularly useful prodrugs. In some cases it is desirable to prepare double ester type prodrugs such as (acyloxy)alkyl esters or ((alkoxycarbonyl)oxy)alkylesters. Particular such prodrugs are the C₁₋₇ alkyl, C₂₋₈ alkenyl, C₆₋₁₀ optionally substituted aryl, and (C₆₋₁₀ aryl)-(C₁₋₄ alkyl) esters of the compounds of the invention.

The present disclosure includes all isotopic forms of the compounds of the invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (2H or D), carbon-11 (1 1C), carbon-13 (13C), carbon-14 (14C), nitrogen-13 (13N), nitrogen-15 (15N), oxygen-15 (15O), oxygen-17 (17O), oxygen-18 (1 80), phosphorus-32 (32P), sulphur-35 (35S), chlorine-36 (36C), chlorine-37 (37C), fluorine-18 (1 8F) iodine-123 (1 23I), iodine-125 (1 25I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. 3H, and carbon-14, i.e. 14C, are particularly useful for this purpose in view of their ease of incorporation and ready means of detection. Unnatural variant isotopic forms which incorporate deuterium i.e 2H or D may afford certain therapeutic advantages resulting from greater metabolic stability, for example, increased in viva halflife or reduced dosage requirements, and hence may be preferred in some circumstances. Further, unnatural variant isotopic forms may be prepared which incorporate positron emitting isotopes, such as 11C, 18F, 150 and 13N, and would be useful in Positron Emission Topography (PET) studies for examining substrate receptor occupancy.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+)- or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

The compounds of the invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

It will be appreciated that compounds of the invention may be metabolized to yield biologically active metabolites.

### DETAILED DESCRIPTION

The inventors have found that the compounds of the present invention are useful for modulating the GPR84 receptor, a G-protein-coupled receptor which may be useful in the treatment of inflammatory or diabetic or metabolic disease. The compounds can be used in a pharmaceutical composition, which further comprises a pharmaceutically acceptable carrier, excipient, or diluent.

The pharmaceutical composition of the invention can be used in the treatment of inflammatory or diabetic disease such as for the treatment of fibrosis in the kidney, liver, lung, pancreas and skin.

The present invention pertains to a compound of the formula I:

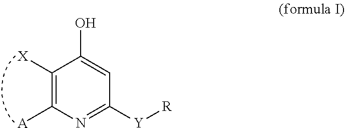

wherein
A is -(A1)ⱼ—(B1)ₖ-(A2)ₗ—(B2)ₘ—H, wherein
   A1 and A2 independently are C₁₋₇ alkylene, C₂₋₇ alkenylene, C₂₋₇ alkynylene, or C₁₋₇ heteroalkylene, optionally substituted with one or two of independently selected U1;
   B1 and B2 are independently an aliphatic ring, an aromatic ring or a fused ring, optionally substituted with one or two of independently selected U2; H is hydrogen;
   j, k, l, and m are independently 0 or 1;
X is C₁₋₇ alkyl, C₂₋₇ heteroalkyl, C₂₋₇ alkenyl, C₂₋₇ heteroalkenyl, C₂₋₇ alkynyl, halogen, —CN, —NO₂, CF₃, —OH or —NH₂, optionally substituted with one, two or three independently selected U3;
------ defines an optional bond between X and A which is formed by substitution of one or two hydrogen radicals at a position on each of A and X such that a single or double bond is formed that results in a 5- or 6-membered aliphatic or aromatic ring fused with the pyridine;
R is an aromatic ring, an aromatic ring comprising nitrogen, an aliphatic ring or a fused ring, optionally substituted with one, two or three of independently selected U4;
U1, U2, U3 and U4 are independently hydrogen, —CF₃, C₁₋₃ alkyl, C₁₋₃ haloalkyl, C₂₋₄ heteroalkyl, halogen, ═O, =NR′=N—OR′—NR′R″, —SR′, —CN, C₂₋₅ alkynyl, C₂₋₅ alkenyl, C₃₋₅ cycloalkyl, C₃₋₅ heterocycloalkyl and —NO₂, where R′, R″, R′″ and R″″ independently refer to hydrogen, unsubstituted C₁₋₃ alkyl and C₂₋₃ heteroalkyl that can be connected by bonds to form rings, cycloalkyl or heterocycloalkyl;

or a pharmaceutically acceptable salt thereof, or a solvate thereof, or a solvate of the pharmaceutically acceptable salt thereof.

In a second aspect, the compound of formula I pertains to:

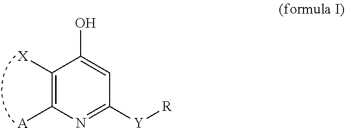

wherein

A is
-A1-B1-A2-B2-H;
-A1-B1-A2-H;
—B1-A2-H;
-A1-B2-H; or
-A1-A2-H;
where A1 and A2 are independently C₁₋₇ alkylene, C₁₋₇ alkenylene, C₁₋₇ alkynylene or C₁₋₇ heteroalkylene; B1 is an aliphatic ring, an aromatic ring or a fused ring; B2 is an aliphatic ring, an aromatic ring or a fused ring, and H is hydrogen;

X is C₁₋₇ alkyl, C₂₋₇ heteroalkyl, C₂₋₇ alkenyl, C₂₋₇ heteroalkenyl, C₂₋₇ alkynyl, halogen, —CN, —NO₂, CF₃, —OH or —NH₂, optionally substituted with one, two or three independently selected U3;

------ defines an optional bond between X and A which is formed by substitution of one or two hydrogen radicals at a position on each of A and X such that a single or double bond is formed that results in a 5- or 6-membered aliphatic or aromatic ring fused with the pyridine;

Y is —OCH₂—, —N(R′)CH₂—, —CH₂CH₂—, —CH₂—, —N(R′)—, or —O—, where R′ is hydrogen or C₁-C₃ alkyl; and

R is
an aromatic ring comprising nitrogen, an aliphatic ring or a fused ring, where the ring is optionally substituted with —CF₃, C₁₋₃ alkyl, C₂₋₄ heteroalkyl or

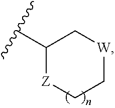

where the ring is optionally substituted with —CF₃, C₁₋₃ alkyl, C₂₋₄ heteroalkyl,
   wherein Z is —O—, —CH₂—, —NH— or N—(CH₂)₀₋₂—CH₃,
   W is —O— or —CH₂—; and
   n is 0, l or 2;

or a pharmaceutically acceptable salt thereof, or a solvate thereof, or a solvate of the pharmaceutically acceptable salt thereof.

The invention comprises compounds that can treat diseases by modulating GPR84-mediated signaling, in particular reducing GPR84-mediated signaling by acting as antagonists, partial agonists or inverse agonists at GPR84.

Several of the therapeutic applications depend on a certain plasma half-life of the compounds. Conjugation reactions of the aromatic hydroxy group, such as glucuronidations or sulfonations, may lead to a shorter half-life than desired. Introduction of the substituent X in formula I increases steric hindrance around the hydroxy group and may hinder interaction with metabolic enzymes, and thereby increase the plasma half-life of the compound. Surprisingly, it was also observer that X can significantly increase the potency of the compound at GPR84.

According to one embodiment, the compounds of the invention provide a new class of GPR84 antagonists that modulate the decanoic acid binding site; in contrast to the antagonist ligands previously reported by Mahmud et. al. (2017) that are believed to bind at a site that is distinct from the decanoic acid and DIM agonist binding sites.

The compounds of the invention have the advantage that they are small and ionizable which potentially results in a better uptake of the compounds and a better distribution of the compounds in the body.

X in formula I has a profound positive impact on the antagonistic activity of the compound.

In one embodiment of the invention, X is halogen or alkyl.

In a preferred embodiment of the invention, X is methyl or ethyl.

In one embodiment of the invention, A is -A1-B1-A2-B2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, B1 is an aliphatic ring, an aromatic ring or a fused ring, and B2 is an aliphatic ring, an aromatic ring or a fused ring, and H is hydrogen. Preferably, A is A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, B1 is an aliphatic ring, an aromatic ring or a fused ring, and B2 is an aliphatic ring, an aromatic ring or a fused ring. More preferably, A is -A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynyl or C₁₋₅ heteroalkylene, B1 is —(C₆H₄)— and B2 is —(C₆H₄)—.

In another embodiment of the invention, A is -A1-B1-A2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, A is -A1-B1-A2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, and B1 is an aliphatic ring, an aromatic ring or a fused ring. More preferably, A is -A1-B1-A2, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene and B1 is —(C₆H₄)—. In a most preferred embodiment, A is -A1-B1-A2-H is —(CH₂)₂—(C₆H₄)—(CH₂)₂—CH₃.

In another embodiment, A is —B1-A2-H, where A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, A is —B1-A2-H, where A2 is C₁₋₅ alkyl, C₁₋₅ alkenyl, C₁₋₅ alkynylene or C₁₋₅ heteroalkylene and B1 is an aliphatic ring, an aromatic ring or a fused ring. More preferably, A is —B1-A2-H, where A2 is C₁₋₅ alkyl, C₁₋₅ alkenyl, C₁₋₅ alkynylene or C₁₋₅ heteroalkylene and B1 is —(C₆H₄)—.

In another embodiment, A is -A1-B2-H, where A1 is C₁₋₇ alkylene, C₁₋₇ alkenylene, C₁₋₇ alkynylene or C₁₋₇ heteroalkylene and B2 is an aliphatic ring, an aromatic ring or a fused ring, and H is hydrogen. Preferably, A is -A1-B2-H, where A1 is C₁₋₅ alkyl, C₁₋₅ alkenyl, C₁₋₅ alkynylene or C₁₋₅ heteroalkylene and B2 is an aliphatic ring, an aromatic ring or a fused ring. More preferably, A is -A1-B2-H, where A1 is C₁₋₅ alkyl, C₁₋₅ alkenyl, C₁₋₅ alkynylene or C₁₋₅ heteroalkylene and B2 is —(C₆H₄)—.

In another embodiment, A is -A1-A2-H, where A1 is C₁₋₇ alkylene, C₁₋₇ alkenylene, C₁₋₇ alkynylene or C₁₋₇ heteroalkylene and A2 is C₁₋₇ alkylene, C₁₋₇ alkenylene, C₁₋₇ alkynylene or C₁₋₇ heteroalkylene, and H is hydrogen. In a preferred embodiment of the invention, A is -A1-A2-H, where A1 is C₁₋₅ alkylene or C₁₋₅ alkenylene and A2 is C₁₋₅ alkylene or C₁₋₅ alkenylene, or more preferred A1 is C₁₋₅ alkylene and A2 is C₁₋₅ alkylene. In a most preferred embodiment of the invention, -A is —(CH₂)₈—CH₃.

In one embodiment, X is Br and A is -A1-B1-A2-B2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, B1 is an aliphatic ring, an aromatic ring or a fused ring, and B2 is an aliphatic ring, an aromatic ring or a fused ring, and H is hydrogen. Preferably, X is —NH— and A is A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, B1 is an aliphatic ring, an aromatic ring or a fused ring, and B2 is an aliphatic ring, an aromatic ring or a fused ring. More preferably, X is —NH— and A is -A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynyl or C₁₋₅ heteroalkylene, B1 is —(C₆H₄)— and B2 is —(C₆H₄)—.

In one embodiment, X is Br and A is -A1-B1-A2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, X is —NH— and A is -A1-B1-A2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, and B1 is an aliphatic ring, an aromatic ring or a fused ring. More preferably, X is —NH— and A is -A1-B1-A2, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene and B1 is —(C₆H₄)—.

In another embodiment, X is Br and A is B1-A2, where A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, X is —NH— and A is —B1-A2-H, where A2 is C₁₋₅ alkyl, C₁₋₅ alkenyl, C₁₋₅ alkynylene or C₁₋₅ heteroalkylene and B1 is an aliphatic ring, an aromatic ring or a fused ring. More preferably, X is —NH— and A is —B1-A2-H, where A2 is C₁₋₅ alkyl, C₁₋₅ alkenyl, C₁₋₅ alkynylene or C₁₋₅ heteroalkylene and B1 is —(C₆H₄)—.

In another embodiment, X is Br and A is -A1-B2-H, where A1 is C₁₋₇ alkylene, C₁₋₇ alkenylene, C₁₋₇ alkynylene or C₁₋₇ heteroalkylene and B2 is an aliphatic ring, an aromatic ring or a fused ring, and H is hydrogen. Preferably, X is —NH— and A is -A1-B2-H, where A1 is C₁₋₅ alkyl, C₁₋₅ alkenyl, C₁₋₅ alkynylene or C₁₋₅ heteroalkylene and B2 is an aliphatic ring, an aromatic ring or a fused ring. More preferably, X is —NH— and A is -A1-B2-H, where A1 is C₁₋₅ alkyl, C₁₋₅ alkenyl, C₁₋₅ alkynylene or C₁₋₅ heteroalkylene and B2 is —(C₆H₄)—.

In another embodiment, X is Br and A is -A1-A2-H, where A1 is C₁₋₇ alkylene, C₁₋₇ alkenylene, C₁₋₇ alkynylene or C₁₋₇ heteroalkylene and A2 is C₁₋₇ alkylene, C₁₋₇ alkenylene, C₁₋₇ alkynylene or C₁₋₇ heteroalkylene, and H is hydrogen. In a preferred embodiment of the invention, X is —NH— and A is -A1-A2-H, where A1 is C₁₋₅ alkylene or C₁₋₅ alkenylene and A2 is C₁₋₅ alkylene or C₁₋₅ alkenylene, or more preferred X is —NH— and A1 is C₁₋₅ alkylene and A2 is C₁₋₅ alkylene.

In one embodiment, X is methyl and A is -A1-B1-A2-B2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, B1 is an aliphatic ring, an aromatic ring or a fused ring, and B2 is an aliphatic ring, an aromatic ring or a fused ring, and H is hydrogen. Preferably, X is methyl and A is A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, B1 is an aliphatic ring, an aromatic ring or a fused ring, and B2 is an aliphatic ring, an aromatic ring or a fused ring. More preferably, X is methyl and A is -A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynyl or C₁₋₅ heteroalkylene, B1 is —(C₆H₄)— and B2 is —(C₆H₄)—.

In one embodiment, X is methyl and A is -A1-B1-A2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, X is methyl and A is -A1-B1-A2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, and B1 is an aliphatic ring, an aromatic ring or a fused ring. More preferably, X is —O— and A is -A1-B1-A2, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene and B1 is —(C₆H₄)—.

In another embodiment, X is methyl and A is —B1-A2, where A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, X is methyl and A is —B1-A2-H, where A2 is C₁₋₅ alkyl, C₁₋₅ alkenyl, C₁₋₅ alkynylene or C₁₋₅ heteroalkylene and B1 is an aliphatic ring, an aromatic ring or a fused ring. More preferably, X is methyl and A is —B1-A2-H, where A2 is C₁₋₅ alkyl, C₁₋₅ alkenyl, C₁₋₅ alkynylene or C₁₋₅ heteroalkylene and B1 is —(C₆H₄)—.

In another embodiment, X is methyl and A is -A1-B2-H, where A1 is C₁₋₇ alkylene, C₁₋₇ alkenylene, C₁₋₇ alkynylene or C₁₋₇ heteroalkylene and B2 is an aliphatic ring, an aromatic ring or a fused ring, and H is hydrogen. Preferably, X is methyl and A is -A1-B2-H, where A1 is C₁₋₅ alkyl, C₁₋₅ alkenyl, C₁₋₅ alkynylene or C₁₋₅ heteroalkylene and B2 is an aliphatic ring, an aromatic ring or a fused ring. More preferably, X is methyl and A is -A1-B2-H, where A1 is C₁₋₅ alkyl, C₁₋₅ alkenyl, C₁₋₅ alkynylene or C₁₋₅ heteroalkylene and B2 is —(C₆H₄)—.

In another embodiment, X is methyl and A is -A1-A2-H, where A1 is C₁₋₇ alkylene, C₁₋₇ alkenylene, C₁₋₇ alkynylene or C₁₋₇ heteroalkylene and A2 is C₁₋₇ alkylene, C₁₋₇ alkenylene, C₁₋₇ alkynylene or C₁₋₇ heteroalkylene, and H is hydrogen. In a preferred embodiment of the invention, X is methyl and A is -A1-A2-H, where A1 is C₁₋₅ alkylene or C₁₋₅ alkenylene and A2 is C₁₋₅ alkylene or C₁₋₅ alkenylene, or more preferred X is methyl and A1 is C₁₋₅ alkylene and A2 is C₁₋₅ alkylene.

In one embodiment, X is ethyl and A is -A1-B1-A2-B2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, B1 is an aliphatic ring, an aromatic ring or a fused ring, and B2 is an aliphatic ring, an aromatic ring or a fused ring, and H is hydrogen. Preferably, X is ethyl and A is A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, B1 is an aliphatic ring, an aromatic ring or a fused ring, and B2 is an aliphatic ring, an aromatic ring or a fused ring. More preferably, X is ethyl and A is -A1-B1-A2-B2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynyl or C₁₋₅ heteroalkylene, B1 is —(C₆H₄)— and B2 is —(C₆H₄)—.

In one embodiment, X is ethyl and A is -A1-B1-A2-H, where A1 is C₁₋₇ alkylene, C₁₋₇ alkenylene, C₁₋₇ alkynylene or C₁₋₇ heteroalkylene, A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, X is ethyl and A is -A1-B1-A2-H, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene, and B1 is an aliphatic ring, an aromatic ring or a fused ring. More preferably, X is —O— and A is -A1-B1-A2, where A1 is C₁₋₅ alkylene, C₁₋₅ alkenylene, C₁₋₅ alkynylene or C₁₋₅ heteroalkylene, A2 is C₁₋₅ alkyl, C₁₋₅ alkenyl, C₁₋₅ alkynylene or C₁₋₅ heteroalkylene and B1 is —(C₆H₄)—.

In another embodiment, X is ethyl and A is —B1-A2, where A2 is C₁₋₇ alkylene, C₁₋₇ alkenylene, C₁₋₇ alkynylene or C₁₋₇ heteroalkylene, and B1 is an aliphatic ring, an aromatic ring or a fused ring, and H is hydrogen. Preferably, X is ethyl and A is —B1-A2-H, where A2 is C₁₋₅ alkyl, C₁₋₅ alkenyl, C₁₋₅ alkynylene or C₁₋₅ heteroalkylene and B1 is an aliphatic ring, an aromatic ring or a fused ring. More preferably, X is ethyl and A is —B1-A2-H, where A2 is C₁₋₅ alkyl, C₁₋₅ alkenyl, C₁₋₅ alkynylene or C₁₋₅ heteroalkylene and B1 is —(C₆H₄)—.

In another embodiment, X is ethyl and A is -A1-B2-H, where A1 is C₁₋₇ alkylene, C₁₋₇ alkenylene, C₁₋₇ alkynylene or C₁₋₇ heteroalkylene and B2 is an aliphatic ring, an aromatic ring or a fused ring, and H is hydrogen. Preferably, X is ethyl and A is -A1-B2-H, where A1 is C₁₋₅ alkyl, C₁₋₅ alkenyl, C₁₋₅ alkynylene or C₁₋₅ heteroalkylene and B2 is an aliphatic ring, an aromatic ring or a fused ring. More preferably, X is ethyl and A is -A1-B2-H, where A1 is C₁₋₅ alkyl, C₁₋₅ alkenyl, C₁₋₅ alkynylene or C₁₋₅ heteroalkylene and B2 is —(C₆H₄)—.

In another embodiment, X is ethyl and A is -A1-A2-H, where A1 is C₁₋₇ alkylene, C₁₋₇ alkenylene, C₁₋₇ alkynylene or C₁₋₇ heteroalkylene and A2 is C₁₋₇ alkylene, C₁₋₇ alkenylene, C₁₋₇ alkynylene or C₁₋₇ heteroalkylene, and H is hydrogen. In a preferred embodiment of the invention, X is ethyl and A is -A1-A2-H, where A1 is C₁₋₅ alkylene or C₁₋₅ alkenylene and A2 is C₁₋₅ alkylene or C₁₋₅ alkenylene, or more preferred X is ethyl and A1 is C₁₋₅ alkylene and A2 is C₁₋₅ alkylene.

In one embodiment, X is methyl or ethyl, and A is -A1-B1-A2-B2-H, where A1 is C₁₋₇ alkylene, C₁-7 alkenylene, C₁₋₇ alkynylene or C₁₋₇ heteroalkylene optionally substituted with one or two of independently selected U1, A2 is C₁₋₁₄ alkylene, C₂₋₁₄ alkenylene, C₂₋₁₄ alkynylene, or C₂₋₁₄ heteroalkylene optionally substituted with one, two, three or four of independently selected U1, B1 is an a C₃₋₇ aliphatic ring, a mono- or bicyclic aromatic ring or a fused ring optionally substituted with one, two, three or four of independently selected U2, B2 is an a C₃₋₇ aliphatic ring, a mono- or bicyclic aromatic ring or a fused ring optionally substituted with one, two, three or four of independently selected U2, and H is hydrogen.

In one embodiment, X is methyl or ethyl, and A is -A1-B1-A2-H, where A1 is C₁₋₇ alkylene, C₁₋₇ alkenylene, C₁₋₇ alkynylene or C₁₋₇ heteroalkylene optionally substituted with one or two of independently selected U1, A2 is C₁₋₁₄ alkylene, C₂₋₁₄ alkenylene, C₂₋₁₄ alkynylene, or C₂₋₁₄ heteroalkylene optionally substituted with one, two, three or four of independently selected U1, B1 is an a C₃₋₇ aliphatic ring, a mono- or bicyclic aromatic ring or a fused ring optionally substituted with one, two, three or four of independently selected U2, and H is hydrogen.

In another embodiment, X is methyl or ethyl, and A is —B1-A2-H, where A2 is C₁₋₁₄ alkylene, C₂₋₁₄ alkenylene, C₂₋₁₄ alkynylene, or C₂₋₁₄ heteroalkylene optionally substituted with one, two, three or four of independently selected U1, B1 is an a C₃₋₇ aliphatic ring, a mono- or bicyclic aromatic ring or a fused ring optionally substituted with one, two, three or four of independently selected U2, and H is hydrogen.

In another embodiment, X is methyl or ethyl, and A is -A1-B2-H, where A1 is C₁₋₇ alkylene, C₁₋₇ alkenylene, C₁₋₇ alkynylene or C₁₋₇ heteroalkylene optionally substituted with one or two of independently selected U1, B2 is an a C₃₋₇ aliphatic ring, a mono- or bicyclic aromatic ring or a fused ring optionally substituted with one, two, three or four of independently selected U2, and H is hydrogen.

In another embodiment, X is methyl or ethyl and A is -A1-A2-H, where A1 is C₁₋₇ alkylene, C₁₋₇ alkenylene, C₁₋₇ alkynylene or C₁₋₇ heteroalkylene optionally substituted with one or two of independently selected U1, A2 is C₁₋₁₄ alkylene, C₂₋₁₄ alkenylene, C₂₋₁₄ alkynylene, or C₂₋₁₄ heteroalkylene optionally substituted with one, two, three or four of independently selected U1, and H is hydrogen.

In one embodiment of the invention, Y is —OCH₂—, —N(R′)CH₂—, —CH₂CH₂—, —CH₂—, —N(R′)—, or —O—, where R′ is hydrogen or C₁-C₃ alkyl; preferably Y is —OCH₂— or —N(R′)CH₂— where R′ is hydrogen or C₁-C₃ alkyl; more preferably Y is —OCH₂—.

In one embodiment of the invention, R is
an aliphatic ring or a fused ring, where the ring is optionally substituted with —CF₃, C₁₋₃ alkyl or C₂₋₄ heteroalkyl or

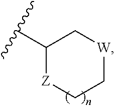

where the ring is optionally substituted with —CF₃, C₁₋₃ alkyl or C₂₋₄ heteroalkyl;
Wherein Z is —O—, —CH₂—, —NH— or N—(CH₂)₀₋₂—CH₃;
   W is —O—, or —CH₂—; and
   n is 0, 1 or 2.

In one embodiment of the invention, R is an aromatic ring comprising nitrogen, preferably the aromatic ring comprises 1, 2 or 3 nitrogen atoms.

In a preferred embodiment the aromatic ring is an aromatic heterocyclic ring having 3 to 5 carbon atoms and 1 to 3 nitrogen atoms, such as an aromatic ring having 3 carbon atoms and 3 nitrogen atoms, or an aromatic ring having 4 carbon atoms and 2 nitrogen atoms or an aromatic ring having 5 carbon atoms and 1 nitrogen atom. In a preferred embodiment the aromatic heterocyclic ring is pyridine, pyrimidine.

In one embodiment of the invention, the aromatic ring can be substituted with —CF₃, C₁₋₃ alkyl or C₂₋₄ heteroalkyl. In one embodiment, the aromatic ring is substituted with —(CH₂)₁—CH₃, —(CH₂)₂—CH₃ or —CH₃.

In one embodiment, the aromatic ring is an aromatic heterocyclic ring having 3 to 6 carbon atoms and 1 to 3 nitrogen atoms, where the aromatic ring is substituted with —CF₃. According to the invention, the aromatic ring substituted with —CF₃ can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with —CF₃.

In one embodiment, the aromatic ring is an aromatic heterocyclic ring having 3 to 6 carbon atoms and 1 to 3 nitrogen atoms, where the aromatic ring is substituted with C₁₋₃ alkyl, e.g. —(CH₂)₀₋₂—CH₃. The aromatic ring can be substituted with C₁₋₃ alkyl and can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with C₁₋₃ alkyl.

In one embodiment, the aromatic ring is an aromatic heterocyclic ring having 3 to 6 carbon atoms and 1 to 3 nitrogen atoms, where the aromatic ring is substituted with —O—CH₃. The aromatic ring can be substituted with C₂₋₄ heteroalkyl and can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with C₂₋₄ heteroalkyl.

In one embodiment of the invention, Y is —OCH₂— and R is an aromatic ring comprising nitrogen, preferably the aromatic ring comprises 1, 2 or 3 nitrogen atoms.

In a preferred embodiment, Y is —OCH₂— and the aromatic ring is an aromatic heterocyclic ring having 3 to 6 carbon atoms and 1 to 3 nitrogen atoms, such as an aromatic ring having 3 carbon atoms and 3 nitrogen atoms, or an aromatic ring having 4 carbon atoms and 2 nitrogen atoms or an aromatic ring having 5 carbon atoms and 1 nitrogen atom. In a preferred embodiment the aromatic heterocyclic ring is pyridine, pyrimidine.

In one embodiment of the invention, Y is —OCH₂—, and the aromatic ring can be substituted with —CF₃, C₁₋₃ alkyl or C₂₋₄ heteroalkyl. In one embodiment, the aromatic ring is substituted with —(CH₂)₁—CH₃, —(CH₂)₂—CH₃ or —CH₃.

In one embodiment, Y is —OCH₂— and the aromatic ring is an aromatic heterocyclic ring having 3 to 6 carbon atoms and 1 to 3 nitrogen atoms, where the aromatic ring is substituted with —CF₃. According to the invention, the aromatic ring substituted with —CF₃ can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with —CF₃.

In one embodiment, Y is —OCH₂— and the aromatic ring is an aromatic heterocyclic ring having 3 to 6 carbon atoms and 1 to 3 nitrogen atoms, where the aromatic ring is substituted with C₁₋₃ alkyl, e.g. —(CH₂)₀₋₂—CH₃. The aromatic ring can be substituted with —(CH₂)₀₋₂—CH₃ and can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with C₁₋₃ alkyl.

In one embodiment, Y is —OCH₂— and the aromatic ring is an aromatic heterocyclic ring having 3 to 6 carbon atoms and 1 to 3 nitrogen atoms, where the aromatic ring is substituted with C₂₋₄ heteroalkyl. The aromatic ring can be substituted with C₂₋₄ heteroalkyl and can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with C₂₋₄ heteroalkyl.

In one embodiment of the invention, Y is —NHCH₂— and R is an aromatic ring comprising nitrogen, preferably the aromatic ring comprises 1, 2 or 3 nitrogen atoms.

In a preferred embodiment, Y is —NHCH₂— and the aromatic ring is an aromatic heterocyclic ring having 3 to 6 carbon atoms and 1 to 3 nitrogen atoms, such as an aromatic ring having 3 carbon atoms and 3 nitrogen atoms, or an aromatic ring having 4 carbon atoms and 2 nitrogen atoms or an aromatic ring having 5 carbon atoms and 1 nitrogen atom. In a preferred embodiment the aromatic heterocyclic ring is pyridine, pyrimidine.

In one embodiment of the invention, Y is —NHCH₂— and the aromatic ring can be substituted with —CF₃, C₁₋₃ alkyl or C₂₋₄ heteroalkyl. In one embodiment, the aromatic ring is substituted with —(CH₂)₁—CH₃, —(CH₂)₂—CH₃ or —CH₃.

In one embodiment, Y is —NHCH₂— and the aromatic ring is an aromatic heterocyclic ring having 3 to 6 carbon atoms and 1 to 3 nitrogen atoms, where the aromatic ring is substituted with —CF₃. According to the invention, the aromatic ring substituted with —CF₃ can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with —CF₃.

In one embodiment, Y is —NHCH₂— and the aromatic ring is an aromatic heterocyclic ring having 3 to 6 carbon atoms and 1 to 3 nitrogen atoms, where the aromatic ring is substituted with —(CH₂)₀₋₂—CH₃. The aromatic ring can be substituted with —(CH₂)₀₋₂—CH₃ and can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with C₁₋₃ alkyl.

In one embodiment, Y is —NHCH₂— and the aromatic ring is an aromatic heterocyclic ring having 3 to 6 carbon atoms and 1 to 3 nitrogen atoms, where the aromatic ring is substituted with —O—CH₃. The aromatic ring can be substituted with —O—CH₃ and can have 3 carbon atoms and 3 nitrogen atoms, 4 carbon atoms and 2 nitrogen atoms or 5 carbon atoms and 1 nitrogen atom. Preferably, the aromatic ring has 5 carbon atoms and 1 nitrogen atom and is substituted with C₂₋₄ heteroalkyl.

In one embodiment of the invention, R is an aliphatic ring, which may be carbocyclic or heterocyclic.

The aliphatic ring can be a 5-7 membered ring, such as aliphatic rings having 5, 6 or 7 carbon atoms or a heterocyclic aliphatic ring having 4, 5 or 6 carbon and which may further comprise one or more oxygen atoms. In a preferred embodiment of the invention, the aliphatic ring has 4 or 5 carbon atoms and 1 or 2 oxygen, such as 4 carbon atoms and 2 oxygen atoms or 5 carbon atoms and 1 oxygen atom.

In one embodiment of the invention, the aliphatic ring can be substituted with —CF₃, C₁₋₃ alkyl or C₂₋₄ heteroalkyl. In one embodiment, the aliphatic ring is substituted with —(CH₂)₁—CH₃, —(CH₂)₂—CH₃ or —CH₃.

The aliphatic ring may be a 5-7 membered ring, which is substituted with —CF₃. In an embodiment, the aliphatic ring has 5 carbon, 6 carbon, or 7 carbon and is substituted with —CF₃.

The ring may be a heterocyclic aliphatic ring having 4, 5 or 6 carbon atoms, and may further comprise one or more oxygen atoms, which ring is substituted with —CF₃. In a preferred embodiment of the invention, the aliphatic ring is substituted with —CF₃ and the ring has 4 or 5 carbon atoms and 1 or 2 oxygen, such as 4 carbon atoms and 2 oxygen atoms or 5 carbon atoms and 1 oxygen atom.

The aliphatic ring may be a 5-7 membered ring, which is substituted with C₁₋₃ alkyl. In an embodiment, the aliphatic ring has 5 carbon, 6 carbon, or 7 carbon and is substituted with C₁₋₃ alkyl, e.g. —(CH₂)₀₋₂—CH₃. The ring may be a heterocyclic aliphatic ring having 4, 5 or 6 carbon atoms, and may further comprise one or more oxygen atoms, which ring is substituted with C₁₋₃ alkyl. In a preferred embodiment of the invention, the aliphatic ring is substituted with C₁₋₃ alkyl and the ring has 4 or 5 carbon atoms and 1 or 2 oxygen, such as 4 carbon atoms and 2 oxygen atoms or 5 carbon atoms and 1 oxygen atom.

The aliphatic ring may be a 5-7 membered ring, which is substituted with C₂₋₄ heteroalkyl. In an embodiment, the aliphatic ring has 5 carbon, 6 carbon, or 7 carbon and is substituted with C₂₋₄ heteroalkyl. The ring may be a heterocyclic aliphatic ring having 4, 5 or 6 carbon atoms, and may further comprise one or more oxygen atoms, which ring is substituted with C₂₋₄ heteroalkyl. In a preferred embodiment of the invention, the aliphatic ring is substituted with C₂₋₄ heteroalkyl and the ring has 4 or 5 carbon atoms and 1 or 2 oxygen, such as 4 carbon atoms and 2 oxygen atoms or 5 carbon atoms and 1 oxygen atom.

In one embodiment of the invention, Y is —OCH₂— and R is an aliphatic ring, which may be carbocyclic or heterocyclic.

In one embodiment Y is —OCH₂— and the aliphatic ring can be a 5-7 membered ring, such as aliphatic rings having 5, 6 or 7 carbon atoms or a heterocyclic aliphatic ring having 4, 5 or 6 carbon and which may further comprise one or more oxygen atoms. In a preferred embodiment of the invention, the aliphatic ring has 4 or 5 carbon atoms and 1 or 2 oxygen, such as 4 carbon atoms and 2 oxygen atoms or 5 carbon atoms and 1 oxygen atom.

In one embodiment of the invention, Y is —OCH₂— and the aliphatic ring can be substituted with —CF₃, C₁₋₃ alkyl or C₂₋₄ heteroalkyl. In one embodiment, the aliphatic ring is substituted with —(CH₂)₁—CH₃, —(CH₂)₂—CH₃ or —CH₃.

In one embodiment Y is —OCH₂— and the aliphatic ring may be a 5-7 membered ring, which is substituted with —CF₃. In an embodiment, the aliphatic ring has 5 carbon, 6 carbon, or 7 carbon and is substituted with —CF₃. The ring may be a heterocyclic aliphatic ring having 4, 5 or 6 carbon atoms, and may further comprise one or more oxygen atoms, which ring is substituted with —CF₃.

In a preferred embodiment of the invention, the aliphatic ring is substituted with —CF₃ and the ring has 4 or 5 carbon atoms and 1 or 2 oxygen, such as 4 carbon atoms and 2 oxygen atoms or 5 carbon atoms and 1 oxygen atom.

In one embodiment, Y is —OCH₂— and the aliphatic ring may be a 5-7 membered ring, which is substituted with C₁₋₃ alkyl, e.g. —(CH₂)₀₋₂—CH₃. In an embodiment, the aliphatic ring has 5 carbon, 6 carbon, or 7 carbon and is substituted with C₁₋₃ alkyl. The ring may be a heterocyclic aliphatic ring having 4, 5 or 6 carbon atoms, and may further comprise one or more oxygen atoms, which ring is substituted with C₁₋₃ alkyl. In a preferred embodiment of the invention, the aliphatic ring is substituted with C₁₋₃ alkyl and the ring has 4 or 5 carbon atoms and 1 or 2 oxygen, such as 4 carbon atoms and 2 oxygen atoms or 5 carbon atoms and 1 oxygen atom.

In one embodiment, Y is —OCH₂— and the aliphatic ring may be a 5-7 membered ring, which is substituted with C₂₋₄ heteroalkyl. In an embodiment, the aliphatic ring has 5 carbon, 6 carbon, or 7 carbon and is substituted with C₂₋₄ heteroalkyl. The ring may be a heterocyclic aliphatic ring having 4, 5 or 6 carbon atoms, and may further comprise one or more oxygen atoms, which ring is substituted with C₂₋₄ heteroalkyl. In a preferred embodiment of the invention, the aliphatic ring is substituted with C₂₋₄ heteroalkyl and the ring has 4 or 5 carbon atoms and 1 or 2 oxygen, such as 4 carbon atoms and 2 oxygen atoms or 5 carbon atoms and 1 oxygen atom.

In one embodiment of the invention, Y is —NHCH₂— and R is an aliphatic ring, which may be carbocyclic or heterocyclic.

In one embodiment Y is —NHCH₂— and the aliphatic ring can be a 5-7 membered ring, such as aliphatic rings having 5, 6 or 7 carbon atoms or a heterocyclic aliphatic ring having 4, 5 or 6 carbon and which may further comprise one or more oxygen atoms. In a preferred embodiment of the invention, the aliphatic ring has 4 or 5 carbon atoms and 1 or 2 oxygen, such as 4 carbon atoms and 2 oxygen atoms or 5 carbon atoms and 1 oxygen atom.

In one embodiment of the invention, Y is —NHCH₂— and the aliphatic ring can be substituted with —CF₃, C₁₋₃ alkyl or C₂₋₄ heteroalkyl. In one embodiment, the aliphatic ring is substituted with —(CH₂)₁—CH₃, —(CH₂)₂—CH₃ or —CH₃.

In one embodiment Y is —NHCH₂— and the aliphatic ring may be a 5-7 membered ring, which is substituted with —CF₃. In an embodiment, the aliphatic ring has 5 carbon, 6 carbon, or 7 carbon and is substituted with —CF₃. The ring may be a heterocyclic aliphatic ring having 4, 5 or 6 carbon atoms, and may further comprise one or more oxygen atoms, which ring is substituted with —CF₃.

In a preferred embodiment of the invention, the aliphatic ring is substituted with —CF₃ and the ring has 4 or 5 carbon atoms and 1 or 2 oxygen, such as 4 carbon atoms and 2 oxygen atoms or 5 carbon atoms and 1 oxygen atom.

In one embodiment, Y is —NHCH₂— and the aliphatic ring may be a 5-7 membered ring, which is substituted with —(CH₂)₀₋₂—CH₃. In an embodiment, the aliphatic ring has 5 carbon, 6 carbon, or 7 carbon and is substituted with C₁₋₃ alkyl, e.g. —(CH₂)₀₋₂—CH₃. The ring may be a heterocyclic aliphatic ring having 4, 5 or 6 carbon atoms, and may further comprise one or more oxygen atoms, which ring is substituted with C₁₋₃ alkyl. In a preferred embodiment of the invention, the aliphatic ring is substituted with C₁₋₃ alkyl and the ring has 4 or 5 carbon atoms and 1 or 2 oxygen, such as 4 carbon atoms and 2 oxygen atoms or 5 carbon atoms and 1 oxygen atom.

In one embodiment, Y is —NHCH₂— and the aliphatic ring may be a 5-7 membered ring, which is substituted with C₂₋₄ heteroalkyl. In an embodiment, the aliphatic ring has 5 carbon, 6 carbon, or 7 carbon and is substituted with C₂₋₄ heteroalkyl. The ring may be a heterocyclic aliphatic ring having 4, 5 or 6 carbon atoms, and may further comprise one or more oxygen atoms, which ring is substituted with C₂₋₄ heteroalkyl. In a preferred embodiment of the invention, the aliphatic ring is substituted with C₂₋₄ heteroalkyl and the ring has 4 or 5 carbon atoms and 1 or 2 oxygen, such as 4 carbon atoms and 2 oxygen atoms or 5 carbon atoms and 1 oxygen atom.

In one embodiment of the invention, R is a fused ring, where the ring is optionally substituted with —CF₃, C₁₋₃ alkyl or C₂₋₄ heteroalkyl.

In one embodiment of the invention, R is

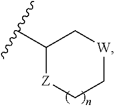

where the ring is optionally substituted with —CF₃, C₁₋₃ alkyl or C₂₋₄ heteroalkyl
wherein Z is —O—, —CH₂—, —NH— or N—(CH₂)₀₋₂—CH₃;
W is —O—, or —CH₂—; and
n is 0, 1 or 2.

In one embodiment of the invention, Y is —OCH₂— and R is

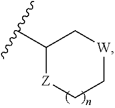

where the ring is optionally substituted with —CF₃, C₁₋₃ alkyl or C₂₋₄ heteroalkyl
wherein Z is —O—, —CH₂—, —NH— or N—(CH₂)₀₋₂—CH₃;
W is —O—, or —CH₂—; and
n is 0, 1 or 2.

In one embodiment of the invention, Y is —NHCH₂— and R is

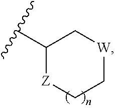

where the ring is optionally substituted with —CF₃, C₁₋₃ alkyl or C₂₋₄ heteroalkyl
wherein Z is —O—, —CH₂—, —NH— or N—(CH₂)₀₋₂—CH₃;
W is —O—, or —CH₂—; and
n is 0, 1 or 2.

In one embodiment R is

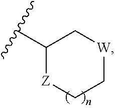

Z is —O—, W is —O—, or —CH₂—, and n is 0, 1 or 2, where the ring is optionally substituted with —CF₃, C₁₋₃ alkyl or C₂₋₄ heteroalkyl.

In one embodiment R is

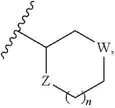

Z is —O—, W is —O—, or —CH₂—, and n is 1, where the ring is optionally substituted with —CF₃, C₁₋₃ alkyl or C₂₋₄ heteroalkyl.

In one embodiment R is

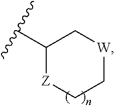

Z is —O—, W is —O—, or —CH₂—, and n is 2, where the ring is optionally substituted with —CF₃, C₁₋₃ alkyl or C₂₋₄ heteroalkyl.

In one embodiment R is

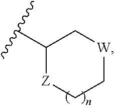

Z is —O—, W is —CH₂—, and n is 0, 1 or 2, where the ring is optionally substituted with —CF₃, C₁₋₃ alkyl or C₂₋₄ heteroalkyl.

In one embodiment R is

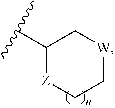

Z is —O—, W is —CH₂—, and n is 1, where the ring is optionally substituted with —CF₃, C₁₋₃ alkyl or C₂₋₄ heteroalkyl.

In one embodiment R is

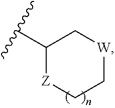

Z is —O—, W is —CH₂—, and n is 2, where the ring is optionally substituted with —CF₃, C₁₋₃ alkyl or C₂₋₄ heteroalkyl.

In one embodiment, Y is —OCH₂— and R is

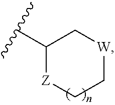

Z is —O—, W is —O—, or —CH₂—, and n is 0, 1 or 2, where the ring is optionally substituted with —CF₃, C₁₋₃ alkyl or C₂₋₄ heteroalkyl.

In one embodiment, Y is —OCH₂— and R is

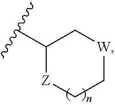

Z is —O—, W is —O—, or —CH₂—, and n is 1, where the ring is optionally substituted with —CF₃, C₁₋₃ alkyl or C₂₋₄ heteroalkyl.

In one embodiment, Y is —OCH₂— and R is

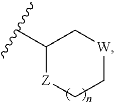

Z is —O—, W is —O—, or —CH₂—, and n is 2, where the ring is optionally substituted with —CF₃, C₁₋₃ alkyl or C₂₋₄ heteroalkyl.

In one embodiment, Y is —OCH₂— and R is

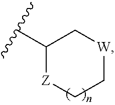

Z is —O—, W is —CH₂—, and n is 0, 1 or 2, where the ring is optionally substituted with —CF₃, C₁₋₃ alkyl or C₂₋₄ heteroalkyl.

In one embodiment, Y is —OCH₂— and R is

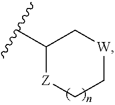

Z is —O—, W is —CH₂—, and n is 1, where the ring is optionally substituted with —CF₃, C₁₋₃ alkyl or C₂₋₄ heteroalkyl.

In one embodiment, Y is —OCH₂— and R is

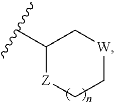

Z is —O—, W is —CH₂—, and n is 2, where the ring is optionally substituted with —CF₃, C₁₋₃ alkyl or C₂₋₄ heteroalkyl.

In one embodiment, Y is —NHCH₂— and R is

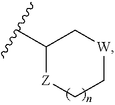

Z is —O—, W is —O—, or —CH₂—, and n is 0, 1 or 2, where the ring is optionally substituted with —CF₃, C₁₋₃ alkyl or C₂₋₄ heteroalkyl.

In one embodiment, Y is —NHCH₂— and R is

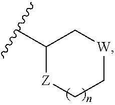

Z is —O—, W is —O—, or —CH₂—, and n is 1, where the ring is optionally substituted with —CF₃, C₁₋₃ alkyl or C₂₋₄ heteroalkyl.

In one embodiment, Y is —NHCH₂— and R is

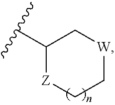

Z is —O—, W is —O—, or —CH₂—, and n is 2, where the ring is optionally substituted with —CF₃, C₁₋₃ alkyl or C₂₋₄ heteroalkyl.

In one embodiment, Y is —NHCH₂— and R is

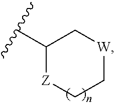

Z is —O—, W is —CH₂—, and n is 0, 1 or 2, where the ring is optionally substituted with —CF₃, C₁₋₃ alkyl or C₂₋₄ heteroalkyl.

In one embodiment, Y is —NHCH₂— and R is

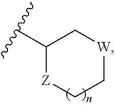

Z is —O—, W is —CH₂—, and n is 1, where the ring is optionally substituted with —CF₃, C₁₋₃ alkyl or C₂₋₄ heteroalkyl.

In one embodiment, Y is —NHCH₂— and R is

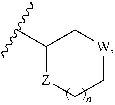

Z is —O—, W is —CH₂—, and n is 2, where the ring is optionally substituted with —CF₃, C₁₋₃ alkyl or C₂₋₄ heteroalkyl.

In one embodiment R is

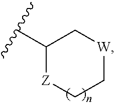

Z is —NH—, W is —O—, or —CH₂—, and n is 0, 1 or 2, where the ring is optionally substituted with —CF₃, C₁₋₃ alkyl or C₂₋₄ heteroalkyl.

In one embodiment R is

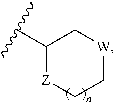

Z is —NH—, W is —O—, or —CH₂—, and n is 1, where the ring is optionally substituted with —CF₃, C₁₋₃ alkyl or C₂₋₄ heteroalkyl.

In one embodiment R is

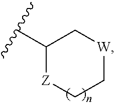

Z is —NH—, W is —O—, or —CH₂—, and n is 2, where the ring is optionally substituted with —CF₃, C₁₋₃ alkyl or C₂₋₄ heteroalkyl.

In one embodiment R is

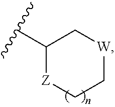

Z is —NH—, W is —CH₂—, and n is 0, 1 or 2, where the ring is optionally substituted with —CF₃, C₁₋₃ alkyl or C₂₋₄ heteroalkyl.

In one embodiment R is

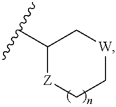

Z is —NH—, W is —CH₂—, and n is 1, where the ring is optionally substituted with —CF₃, C₁₋₃ alkyl or C₂₋₄ heteroalkyl.

In one embodiment R is

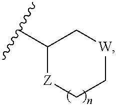

Z is —NH—, W is —CH₂—, and n is 2, where the ring is optionally substituted with —CF₃, C₁₋₃ alkyl or C₂₋₄ heteroalkyl.

In one embodiment, Y is —OCH₂— and R is

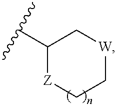

Z is —NH—, W is —O—, or —CH₂—, and n is 0, 1 or 2, where the ring is optionally substituted with —CF₃, C₁₋₃ alkyl or C₂₋₄ heteroalkyl.

In one embodiment, Y is —OCH₂— and R is

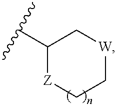

Z is —NH—, W is —O—, or —CH₂—, and n is 1, where the ring is optionally substituted with —CF₃, C₁₋₃ alkyl or C₂₋₄ heteroalkyl.

In one embodiment, Y is —OCH₂— and R is

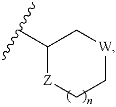

Z is —NH—, W is —O—, or —CH₂—, and n is 2, where the ring is optionally substituted with —CF₃, C₁₋₃ alkyl or C₂₋₄ heteroalkyl.

In one embodiment, Y is —OCH₂— and R is

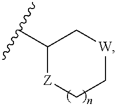

Z is —NH—, W is —CH₂—, and n is 0, 1 or 2, where the ring is optionally substituted with —CF₃, C₁₋₃ alkyl or C₂₋₄ heteroalkyl.

In one embodiment, Y is —OCH₂— and R is

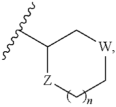

Z is —NH—, W is —CH₂—, and n is 1, where the ring is optionally substituted with —CF₃, C₁₋₃ alkyl or C₂₋₄ heteroalkyl.

In one embodiment, Y is —OCH₂— and R is

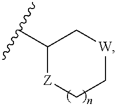

Z is —NH—, W is —CH₂—, and n is 2, where the ring is optionally substituted with —CF₃, C₁₋₃ alkyl or C₂₋₄ heteroalkyl.

In one embodiment, Y is —NHCH₂— and R is

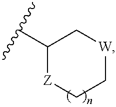

Z is —NH—, W is —O—, or —CH₂—, and n is 0, 1 or 2, where the ring is optionally substituted with —CF₃, C₁₋₃ alkyl or C₂₋₄ heteroalkyl.

In one embodiment, Y is —NHCH₂— and R is

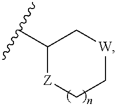

Z is —NH—, W is —O—, or —CH₂—, and n is 1, where the ring is optionally substituted with —CF₃, C₁₋₃ alkyl or C₂₋₄ heteroalkyl.

In one embodiment, Y is —NHCH₂— and R is

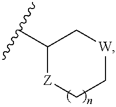

Z is —NH—, W is —O—, or —CH₂—, and n is 2, where the ring is optionally substituted with —CF₃, C₁₋₃ alkyl or C₂₋₄ heteroalkyl.

In one embodiment, Y is —NHCH₂— and R is

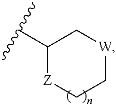

Z is —NH—, W is —CH₂—, and n is 0, 1 or 2, where the ring is optionally substituted with —CF₃, C₁₋₃ alkyl or C₂₋₄ heteroalkyl.

In one embodiment, Y is —NHCH₂— and R is

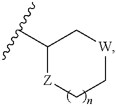

Z is —NH—, W is —CH₂—, and n is 1, where the ring is optionally substituted with —CF₃, C₁₋₃ alkyl or C₂₋₄ heteroalkyl.

In one embodiment, Y is —NHCH₂— and R is

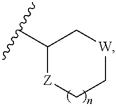

Z is —NH—, W is —CH₂—, and n is 2, where the ring is optionally substituted with —CF₃, C₁₋₃ alkyl or C₂₋₄ heteroalkyl.

In a preferred embodiment of the invention, the invention pertains to a compound of formula II:

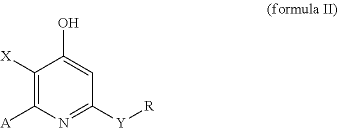

wherein

A is
-A1-B1-A2-H; or
-A1-A2-H;
where A1 and A2 are independently C₁₋₇ alkylene, C₁₋₇ alkenylene, C₁₋₇ alkynylene or C₁₋₇ heteroalkylene; B1 is an aromatic ring, and H is hydrogen;

X is halogen or alkyl;

Y is —OCH₂— or —NHCH₂—, and

R is

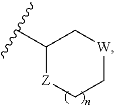

where the ring is optionally substituted with —CF₃, C₁₋₃ alkyl or C₂₋₄ heteroalkyl,
   wherein Z is —O— or —NH—,
   W is —O— or —CH₂—; and
   n is 1 or 2;

or a pharmaceutically acceptable salt thereof, or a solvate thereof, or a solvate of the pharmaceutically acceptable salt thereof.

In another preferred embodiment of the invention, the invention pertains to a compound of formula II:

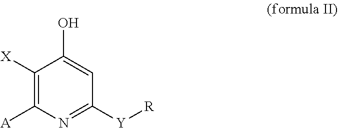

wherein

A is
-A1-A2-H;
where A1 is C₁₋₅ alkylene or C₁₋₅ alkenylene and A2 is C₁₋₅ alkylene or C₁₋₅ alkenylene and H is hydrogen, or more preferred A1 is C₁₋₅ alkylene and A2 is C₁₋₅ alkylene, such as -A1-A2-H is —(CH₂)₅—CH₃, —(CH₂)₆—CH₃, —(CH₂)₇—CH₃, —(CH₂)₈—CH₃, —(CH₂)₉—CH₃ or —(CH₂)₁₀—CH₃;

X is bromo or methyl;

Y is —OCH₂—, and

R is

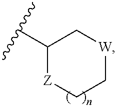

where the ring is optionally substituted with —CF₃, C₁₋₃ alkyl or C₂₋₄ heteroalkyl,
   Wherein Z is —O— or —NH—,
   W is —O— or —CH₂—; and
   n is 1 or 2;
   preferably Z is —O—, W is —CH₂—, and n is 1;

or a pharmaceutically acceptable salt thereof, or a solvate thereof, or a solvate of the pharmaceutically acceptable salt thereof.

In one preferred embodiment the invention provides is a compound of formula I, wherein
A1 is selected from ethylene, C₂₋₄ alkylene and C₂₋₄ heteroalkylene
A2 is heteroalkylene
B1 is selected from 1,4-benzene (optionally substituted), heterocycle, aromatic ring
B2 is not present (m=0) or is heterocycle,
X is selected from ethyl, methyl, substituted C₁₋₇ alkyl, C₂₋₇ heteroalkyl, halogen, nitrile, trifluromethyl
Y is —OCH₂—
R is selected from dioxane, tetrahydropyrane, heterocycloalkyl, fused ring

In one preferred embodiment the invention provides is a compound of formula I, wherein A is A1-B1-A2, wherein A1 is ethylene, B1 is 1,4-benzene, A2 is heteroalkyl, and wherein X is ethyl, Y is —OCH₂— and R is dioxane.

In one preferred embodiment the invention provides a compound of formula I, wherein A is A1-B1-A2, wherein A1 is ethylene, B1 is 1,4-benzene substituted with one U2, A2 is heteroalkyl, and wherein X is ethyl, Y is —OCH₂— and R is dioxane.

In one preferred embodiment the invention provides a compound of formula I, wherein A is A1-B1-A2, wherein A1 is ethylene, B1 is 1,4-benzene substituted with two U2, A2 is heteroalkyl, and wherein X is ethyl, Y is —OCH₂— and R is dioxane.

In one preferred embodiment the invention provides a compound of formula I, wherein A is A1-B1-A2, wherein A1 is ethylene, B1 is pyridine, A2 is heteroalkyl, and wherein X is ethyl, Y is —OCH₂— and R is dioxane.

In one preferred embodiment the invention provides a compound of formula I, wherein A is A1-B1-A2, wherein A1 is ethylene, B1 is pyridine substituted with one U2, A2 is heteroalkyl, and wherein X is ethyl, Y is —OCH₂— and R is dioxane.

In one preferred embodiment the invention provides a compound of formula I, wherein A is A1-B1-A2, wherein A1 is ethylene, B1 is pyridine substituted with two U2, A2 is heteroalkyl, and wherein X is ethyl, Y is —OCH₂— and R is dioxane.

In one preferred embodiment the invention provides a compound of formula I, wherein A is A1-B1-A2, wherein A1 is ethylene, B1 is pyridine substituted with —CN, A2 is heteroalkyl, and wherein X is ethyl, Y is —OCH₂— and R is dioxane. In one preferred embodiment the invention provides a compound of formula I, wherein A is A1-B1-A2-B2, wherein A1 is ethylene, B1 is benzene, A2 is heteroalkyl, B2 is heterocycle, and wherein X is ethyl, Y is —OCH₂— and R is dioxane.

In one preferred embodiment the invention provides is a compound of formula I, wherein A is A1-B1-A2, wherein A1 is -CD₂CD₂-, B1 is 1,4-benzene, A2 is heteroalkyl, and wherein X is ethyl, Y is —OCH₂— and R is dioxane.

In one preferred embodiment the invention provides a compound of formula I, wherein A is A1-B1-A2, wherein A1 is -CD₂CD₂-, B1 is 1,4-benzene substituted with one U2, A2 is heteroalkyl, and wherein X is ethyl, Y is —OCH₂— and R is dioxane.

In one preferred embodiment the invention provides a compound of formula I, wherein A is A1-B1-A2, wherein A1 is -CD₂CD₂-, B1 is 1,4-benzene substituted with two U2, A2 is heteroalkyl, and wherein X is ethyl, Y is —OCH₂— and R is dioxane.

In one preferred embodiment the invention provides a compound of formula I, wherein A is A1-B1-A2, wherein A1 is -CD₂CD₂-, B1 is 1,4-benzene substituted with —CN, A2 is heteroalkyl, and wherein X is ethyl, Y is —OCH₂— and R is dioxane.

In one preferred embodiment the invention provides a compound of formula I, wherein A is A1-B1-A2, wherein A1 is -CD₂CD₂-, B1 is pyridine, A2 is heteroalkyl, and wherein X is ethyl, Y is —OCH₂— and R is dioxane.

In one preferred embodiment the invention provides a compound of formula I, wherein A is A1-B1-A2, wherein A1 is -CD₂CD₂-, B1 is pyridine substituted with one U2, A2 is heteroalkyl, and wherein X is ethyl, Y is —OCH₂— and R is dioxane.

In one preferred embodiment the invention provides a compound of formula I, wherein A is A1-B1-A2, wherein A1 is CD₂CD₂-, B1 is pyridine substituted with two U2, A2 is heteroalkyl, and wherein X is ethyl, Y is —OCH₂— and R is dioxane.

In one preferred embodiment the invention provides a compound of formula I, wherein A is A1-B1-A2, wherein A1 is CD₂CD₂-, B1 is pyridine substituted with two —CN, A2 is heteroalkyl, and wherein X is ethyl, Y is —OCH₂— and R is dioxane.

In one preferred embodiment the invention provides a compound of formula I, wherein A is A1-B1-A2-B2, wherein A1 is -CD₂CD₂-, B1 is benzene, A2 is heteroalkyl, B2 is heterocycle, and wherein X is ethyl, Y is —OCH₂— and R is dioxane.

In one preferred embodiment the invention provides is a compound of formula I, wherein A is A1-B1-A2, wherein A1 is ethylene, B1 is 1,4-benzene, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is 1,4-benzene substituted with one U2, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is 1,4-benzene substituted with two U2, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is 1,4-benzene substituted with —CN, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is pyridine, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is pyridine substituted with one U2, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is pyridine substituted with two U2, A2 is heteroalkyl, and wherein X is methyl, Y is —OCH₂— and R is dioxane.

In one preferred embodiment the invention provides a compound of formula I, wherein A is A1-B1-A2, wherein A1 is ethylene, B1 is pyridine substituted with —CN, A2 is heteroalkyl, and wherein X is methyl, Y is —OCH₂— and R is dioxane.

In one preferred embodiment the invention provides a compound of formula I, wherein A is A1-B1-A2-B2, wherein A1 is ethylene, B1 is benzene, A2 is heteroalkyl, B2 is heterocycle, and wherein X is methyl, Y is —OCH₂— and R is dioxane.

In one preferred embodiment the invention provides is a compound of formula I, wherein A is A1-B1-A2, wherein A1 is -CD₂CD₂-, B1 is 1,4-benzene, A2 is heteroalkyl, and wherein X is methyl, Y is —OCH₂— and R is dioxane.

In one preferred embodiment the invention provides a compound of formula I, wherein A is A1-B1-A2, wherein A1 is -CD₂CD₂-, B1 is 1,4-benzene substituted with one U2, A2 is heteroalkyl, and wherein X is methyl, Y is —OCH₂— and R is dioxane.

In one preferred embodiment the invention provides a compound of formula I, wherein A is A1-B1-A2, wherein A1 is -CD₂CD₂-, B1 is 1,4-benzene substituted with two U2, A2 is heteroalkyl, and wherein X is methyl, Y is —OCH₂— and R is dioxane.

In one preferred embodiment the invention provides a compound of formula I, wherein A is A1-B1-A2, wherein A1 is -CD₂CD₂-, B1 is 1,4-benzene substituted —CN, A2 is heteroalkyl, and wherein X is methyl, Y is —OCH₂— and R is dioxane.

In one preferred embodiment the invention provides a compound of formula I, wherein A is A1-B1-A2, wherein A1 is -CD₂CD₂-, B1 is pyridine, A2 is heteroalkyl, and wherein X is methyl, Y is —OCH₂— and R is dioxane.

In one preferred embodiment the invention provides a compound of formula I, wherein A is A1-B1-A2, wherein A1 is -CD₂CD₂-, B1 is pyridine substituted with one U2, A2 is heteroalkyl, and wherein X is methyl, Y is —OCH₂— and R is dioxane.

In one preferred embodiment the invention provides a compound of formula I, wherein A is A1-B1-A2, wherein A1 is CD₂CD₂-, B1 is pyridine substituted with two U2, A2 is heteroalkyl, and wherein X is methyl, Y is —OCH₂— and R is dioxane.

In one preferred embodiment the invention provides a compound of formula I, wherein A is A1-B1-A2, wherein A1 is CD₂CD₂-, B1 is pyridine substituted with —CN, A2 is heteroalkyl, and wherein X is methyl, Y is —OCH₂— and R is dioxane.

In a preferred embodiment of the invention, the compound is 3-bromo-2-(4-propylphenethyl)-6-((tetrahydro-2H-pyran-2-yl)methoxy)pyridin-4-ol:

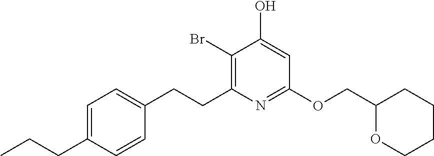 [SMILES: CCCc1ccc(CCc2nc(OCC3CCCCO3)cc(O)c2Br)cc1]

In another preferred embodiment of the invention, the compound is 3-methyl-2-(4-propylphenethyl)-6-((tetrahydro-2H-pyran-2-yl)methoxy)pyridin-4-ol:

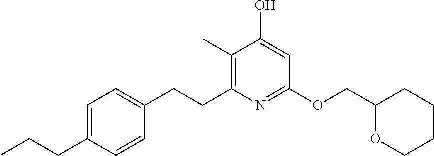 [SMILES: CCCc1ccc(CCc2nc(OCC3CCCCO3)cc(O)c2C)cc1]

In one preferred embodiment of the invention, the compound is 3-ethyl-2-(4-propylphenethyl)-6-((tetrahydro-2H-pyran-2-yl)methoxy)pyridin-4-ol:

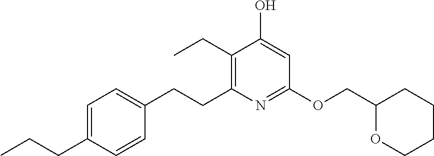 [SMILES: CCCc1ccc(CCc2nc(OCC3CCCCO3)cc(O)c2CC)cc1]

In another preferred embodiment of the invention, the compound is 6-((1,4-dioxan-2-yl)methoxy)-3-methyl-2-(4-propylphenethyl)pyridin-4-ol:

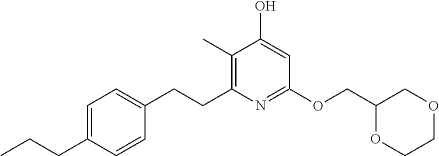 [SMILES: CCCc1ccc(CCc2nc(OCC3COCCO3)cc(O)c2C)cc1]

In another preferred embodiment of the invention, the compound is (S)-6-((1,4-dioxan-2-yl)methoxy)-2-(4-ethoxyphenethyl)-3-methylpyridin-4-ol:

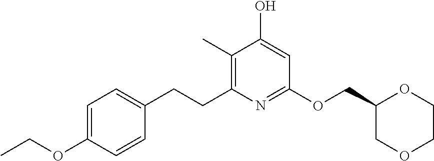 [SMILES: CCOc1ccc(CCc2nc(OC[C@@H]3COCCO3)cc(O)c2C)cc1]

In another preferred embodiment of the invention, the compound is 6-((1,4-dioxan-2-yl)methoxy)-2-(4-ethoxyphenethyl)-3-methylpyridin-4-ol:

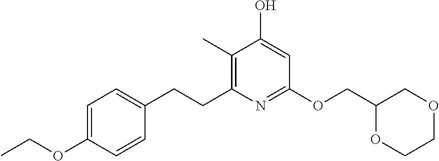 [SMILES: CCOc1ccc(CCc2nc(OCC3COCCO3)cc(O)c2C)cc1]

In another preferred embodiment of the invention, the compound is 6-((1,4-dioxan-2-yl)methoxy)-3-ethyl-2-(4-propylphenethyl)pyridin-4-ol:

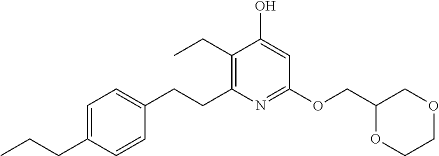 [SMILES: CCCc1ccc(CCc2nc(OCC3COCCO3)cc(O)c2CC)cc1]

In another preferred embodiment of the invention, the compound is 6-((1,4-dioxan-2-yl)methoxy)-2-(4-ethoxyphenethyl)-3-ethylpyridin-4-ol:

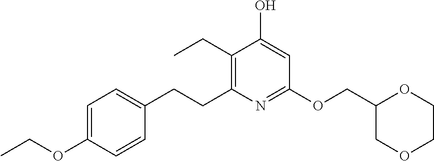 [SMILES: CCOc1ccc(CCc2nc(OCC3COCCO3)cc(O)c2CC)cc1]

In another preferred embodiment of the invention, the compound is tert-butyl (2-(4-(2-(6-((1,4-dioxan-2-yl)methoxy)-4-hydroxy-3-methylpyridin-2-yl)ethyl)phenoxy)ethyl)carbamate:

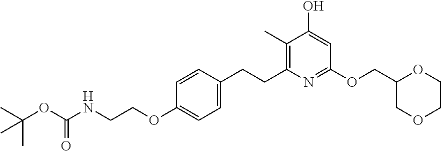 [SMILES: Cc1c(O)cc(OCC2COCCO2)nc1CCc1ccc(OCCNC(=O)OC(C)(C)C)cc1]

In another preferred embodiment of the invention, the compound is 2-(4-(2-(6-((1,4-dioxan-2-yl)methoxy)-4-hydroxy-3-methylpyridin-2-yl)ethyl)phenoxy)ethan-1-amine:

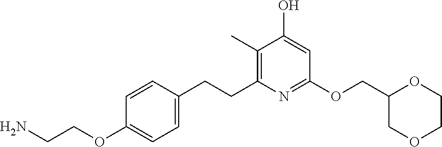 [SMILES: Cc1c(O)cc(OCC2COCCO2)nc1CCc1ccc(OCCN)cc1]

In another preferred embodiment of the invention, the compound is N-(2-(4-(2-(6-((1,4-dioxan-2-yl)methoxy)-4-hydroxy-3-methylpyridin-2-yl)ethyl)phenoxy)ethyl)acetamide:

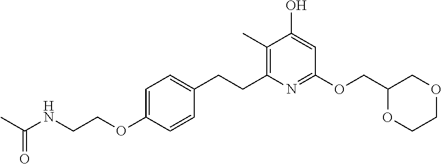 [SMILES: CC(=O)NCCOc1ccc(CCc2nc(OCC3COCCO3)cc(O)c2C)cc1]

In another preferred embodiment of the invention, the compound is ethyl 2-(4-(2-(6-((1,4-dioxan-2-yl)methoxy)-4-hydroxy-3-methylpyridin-2-yl)ethyl)phenoxy)acetate:

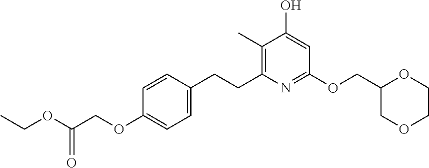 [SMILES: CCOC(=O)COc1ccc(CCc2nc(OCC3COCCO3)cc(O)c2C)cc1]

In another preferred embodiment of the invention, the compound is tert-butyl (2-(4-(2-(6-((1,4-dioxan-2-yl)methoxy)-3-ethyl-4-hydroxypyridin-2-yl)ethyl)phenoxy)ethyl)carbamate:

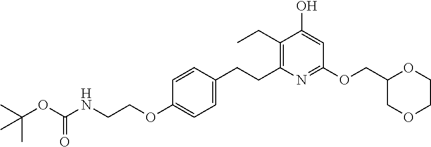 [SMILES: CCc1c(O)cc(OCC2COCCO2)nc1CCc1ccc(OCCNC(=O)OC(C)(C)C)cc1]

In another preferred embodiment of the invention, the compound is tert-butyl (3-(4-(2-(6-((1,4-dioxan-2-yl)methoxy)-3-ethyl-4-hydroxypyridin-2-yl)ethyl)phenoxy)propyl)carbamate:

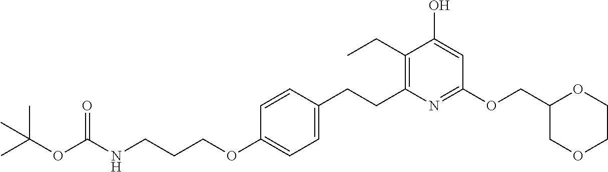 [SMILES: CCc1c(O)cc(OCC2COCCO2)nc1CCc1ccc(OCCCNC(=O)OC(C)(C)C)cc1]

In another preferred embodiment of the invention, the compound is methyl 2-((4-(2-(6-((1,4-dioxan-2-yl)methoxy)-3-ethyl-4-hydroxypyridin-2-yl)ethyl)phenoxy)methyl)oxazole-4-carboxylate:

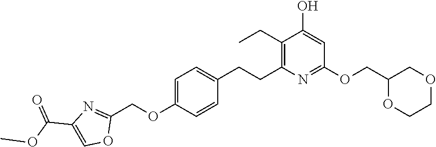 [SMILES: CCc1c(O)cc(OCC2COCCO2)nc1CCc1ccc(OCc2nc(C(=O)OC)co2)cc1]

In another preferred embodiment of the invention, the compound is tert-butyl (2-(2-(4-(2-(6-((1,4-dioxan-2-yl)methoxy)-3-ethyl-4-hydroxypyridin-2-yl)ethyl)phenoxy)ethoxy)-ethyl)carbamate:

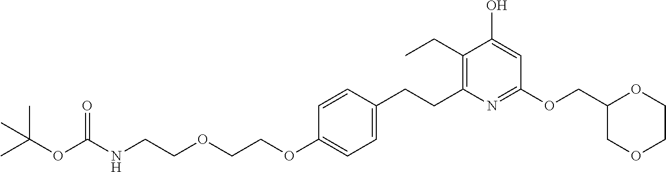 [SMILES: CCc1c(O)cc(OCC2COCCO2)nc1CCc1ccc(OCCOCCNC(=O)OC(C)(C)C)cc1]

In another preferred embodiment of the invention, the compound is 6-((1,4-dioxan-2-yl)methoxy)-3-ethyl-2-(4-hydroxyphenethyl)pyridin-4-ol:

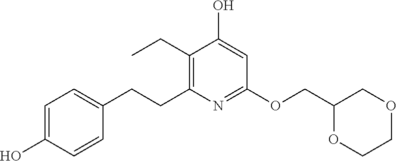 [SMILES: CCc1c(O)cc(OCC2COCCO2)nc1CCc1ccc(O)cc1]

In another preferred embodiment of the invention, the compound is 2-(2-(4-(2-(6-((1,4-dioxan-2-yl)methoxy)-3-ethyl-4-hydroxypyridin-2-yl)ethyl)phenoxy)ethyl)isoindoline-1,3-dione:

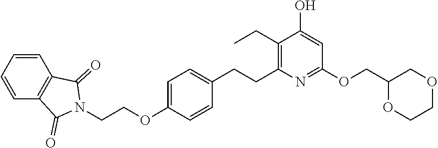 [SMILES: CCc1c(O)cc(OCC2COCCO2)nc1CCc1ccc(OCCN2C(=O)c3ccccc3C2=O)cc1]

In another preferred embodiment of the invention, the compound is 6-((1,4-dioxan-2-yl)methoxy)-2-(2-(6-ethoxypyridin-3-yl)ethyl)-3-ethylpyridin-4-ol:

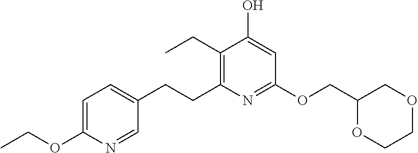 [SMILES: CCOc1ccc(CCc2nc(OCC3COCCO3)cc(O)c2CC)cn1]

In another preferred embodiment of the invention, the compound is 6-((1,4-dioxan-2-yl)methoxy)-2-(2-(6-ethoxy-5-fluoropyridin-3-yl)ethyl)-3-ethylpyridin-4-ol:

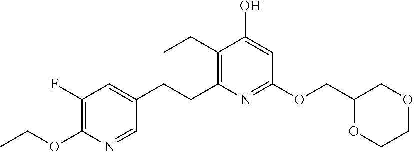 [SMILES: CCOc1ncc(CCc2nc(OCC3COCCO3)cc(O)c2CC)cc1F]

In another preferred embodiment of the invention, the compound is 6-((1,4-dioxan-2-yl)methoxy)-2-(4-(2-aminoethoxy)phenethyl)-3-ethylpyridin-4-ol:

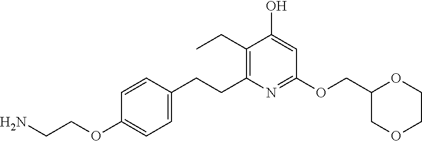 [SMILES: CCc1c(O)cc(OCC2COCCO2)nc1CCc1ccc(OCCN)cc1]

In another preferred embodiment of the invention, the compound is 6-((1,4-dioxan-2-yl)methoxy)-2-(4-(3-aminopropoxy)phenethyl)-3-ethylpyridin-4-ol:

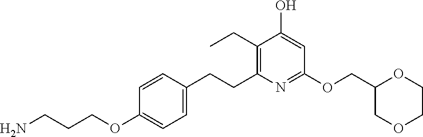 [SMILES: CCc1c(O)cc(OCC2COCCO2)nc1CCc1ccc(OCCCN)cc1]

In another preferred embodiment of the invention, the compound is 6-((1,4-dioxan-2-yl)methoxy)-3-ethyl-2-(4-(3-(methylsulfonyl)propoxy)phenethyl)pyridin-4-ol:

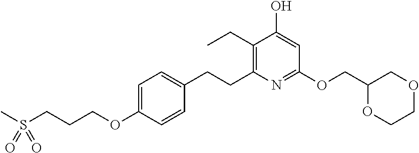 [SMILES: CCc1c(O)cc(OCC2COCCO2)nc1CCc1ccc(OCCCS(C)(=O)=O)cc1]

In another preferred embodiment of the invention, the compound is 6-((1,4-dioxan-2-yl)methoxy)-3-ethyl-2-(4-(2-phenoxyethoxy)phenethyl)pyridin-4-ol:

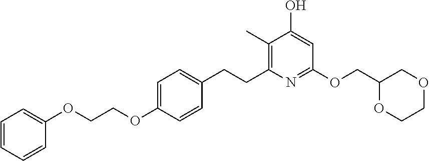 [SMILES: Cc1c(O)cc(OCC2COCCO2)nc1CCc1ccc(OCCOc2ccccc2)cc1]

In another preferred embodiment of the invention, the compound is 6-((1,4-dioxan-2-yl)methoxy)-3-ethyl-2-(4-((3-methyl-1,2,4-oxadiazol-5-yl)methoxy)phenethyl)pyridin-4-ol:

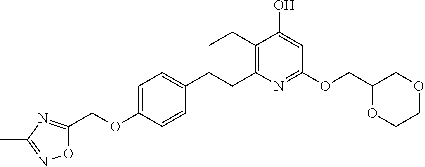 [SMILES: CCc1c(O)cc(OCC2COCCO2)nc1CCc1ccc(OCc2nc(C)no2)cc1]

In another preferred embodiment of the invention, the compound is 6-((1,4-dioxan-2-yl)methoxy)-2-(2-(4-(ethoxy-d5)phenyl)ethyl-1,1,2,2-d4)-3-ethylpyridin-4-ol:

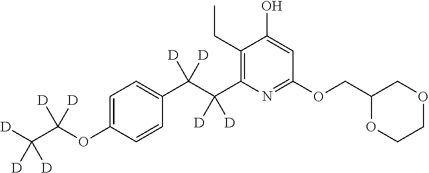 [SMILES: [2H]C([2H])([2H])C([2H])([2H])Oc1ccc(C([2H])([2H])C([2H])([2H])c2nc(OCC3COCCO3)cc(O)c2CC)cc1]

In another preferred embodiment of the invention, the compound is (S)-6-((1,4-dioxan-2-yl)methoxy)-2-(3,4-dimethoxyphenethyl)-3-ethylpyridin-4-ol:

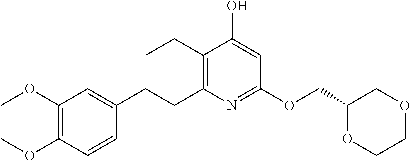 [SMILES: CCc1c(O)cc(OC[C@@H]2COCCO2)nc1CCc1ccc(OC)c(OC)c1]

In another preferred embodiment of the invention, the compound is (S)-6-((1,4-dioxan-2-yl)methoxy)-2-(2,4-dimethoxyphenethyl)-3-ethylpyridin-4-ol:

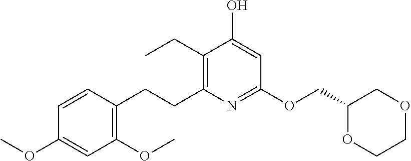 [SMILES: CCc1c(O)cc(OC[C@@H]2COCCO2)nc1CCc1ccc(OC)cc1OC]

In another preferred embodiment of the invention, the compound is (S)-5-(2-(6-((1,4-dioxan-2-yl)methoxy)-3-ethyl-4-hydroxypyridin-2-yl)ethyl)-2-methoxybenzonitrile:

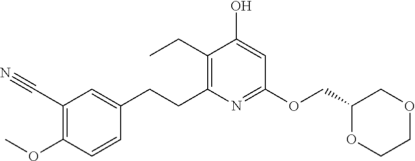 [SMILES: CCc1c(O)cc(OC[C@@H]2COCCO2)nc1CCc1ccc(OC)c(C#N)c1]

In another preferred embodiment of the invention, the compound is (S)-2-(2-(6-((1,4-dioxan-2-yl)methoxy)-3-ethyl-4-hydroxypyridin-2-yl)ethyl)-5-methoxybenzonitrile:

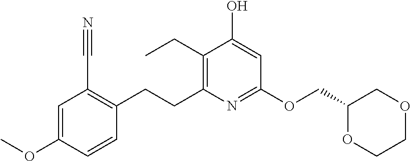 [SMILES: CCc1c(O)cc(OC[C@@H]2COCCO2)nc1CCc1ccc(OC)cc1C#N]

In another preferred embodiment of the invention, the compound is ethyl 5-(4-(2-(6-((1,4-dioxan-2-yl)methoxy)-3-ethyl-4-hydroxypyridin-2-yl)ethyl)phenoxy)pentanoate:

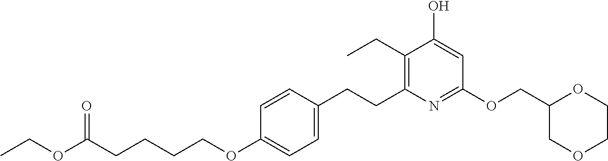 [SMILES: CCOC(=O)CCCCOc1ccc(CCc2nc(OCC3COCCO3)cc(O)c2CC)cc1]

In another preferred embodiment of the invention, the compound is 6-((1,4-dioxan-2-yl)methoxy)-3-ethyl-2-(4-(2-hydroxyethoxy)phenethyl)pyridin-4-ol:

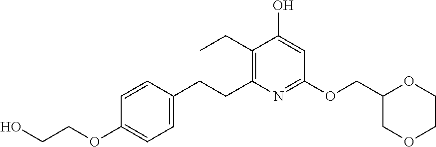 [SMILES: CCc1c(O)cc(OCC2COCCO2)nc1CCc1ccc(OCCO)cc1]

In another preferred embodiment of the invention, the compound is 2-(4-(2-(6-((1,4-dioxan-2-yl)methoxy)-3-ethyl-4-hydroxypyridin-2-yl)ethyl)phenoxy)acetamide:

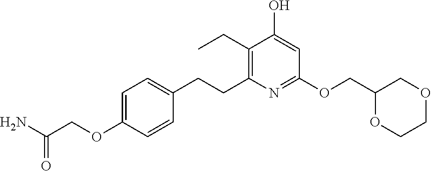 [SMILES: CCc1c(O)cc(OCC2COCCO2)nc1CCc1ccc(OCC(N)=O)cc1]

In another preferred embodiment of the invention, the compound is 3-(4-hydroxy-2-(4-propylphenethyl)-6-((tetrahydro-2H-pyran-2-yl)methoxy)pyridin-3-yl)propanoic acid:

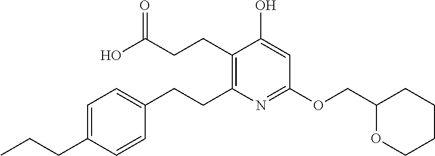 [SMILES: CCCc1ccc(CCc2nc(OCC3CCCCO3)cc(O)c2CCC(=O)O)cc1]

In another preferred embodiment of the invention, the compound is ethyl 3-(4-hydroxy-2-(4-propylphenethyl)-6-((tetrahydro-2H-pyran-2-yl)methoxy)pyridin-3-yl)propanoate:

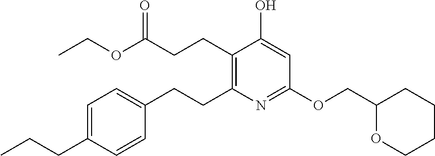 [SMILES: CCCc1ccc(CCc2nc(OCC3CCCCO3)cc(O)c2CCC(=O)OCC)cc1]

In yet another preferred embodiment of the invention, the compound is 2-((4-(2-(6-((1,4-dioxan-2-yl)methoxy)-3-ethyl-4-hydroxypyridin-2-yl)ethyl)phenoxy)methyl)oxazole-4-carboxylic acid:

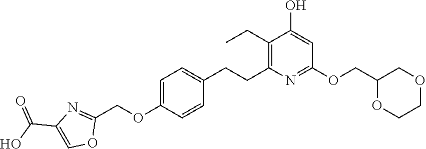 [SMILES: CCc1c(O)cc(OCC2COCCO2)nc1CCc1ccc(OCc2nc(C(=O)O)co2)cc1]

### Pharmaceutical Compositions

The term “composition” as used herein is intended to encompass a product comprising the specified ingredients (and in the specified amounts, if indicated), as well as any product which results, directly or indirectly, from combination of the specified ingredients in the specified amounts.

By “pharmaceutically acceptable” it is meant that the carrier, excipient, or diluent is compatible with the other ingredients of the formulation and is not deleterious to the recipient thereof.

Composition formulations may improve one or more pharmacokinetic properties (eg oral bioavailability, membrane permeability) of a compound of the invention (herein referred to as the active ingredient).

The pharmaceutical compositions for the administration of the compounds of this invention may conveniently be presented in unit dosage form and may be prepared by any of the methods well known in the art. All methods include the step of bringing the active ingredient into association with the carrier which constitutes one or more accessory ingredients.

In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Such compositions may contain one or more agents selected from sweetening agents, flavouring agents, colouring agents and preserving agents in order to provide pharmaceutically elegant and palatable preparations.

Tablets may contain the active ingredient in admixture with other non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,160,452, and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil, or coconut oil, or in a mineral oil such as liquid paraffin. The oily suspensions may contain a thickening agent, for example beeswax, hard paraffin, or cetyl alcohol.

Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or diglycerides. In addition, fatty acids such as oleic acid find use in the preparation of injectables. The pharmaceutical compositions may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

For topical use, creams, ointments, jellies, solutions, or suspensions etc containing the compounds of the invention are employed. As used herein, topical application is also meant to include the use of mouthwashes and gargles.

### PREFERRED NUMBERED EMBODIMENTS OF THE INVENTION

Preferred embodiment 1: A compound of the formula I:

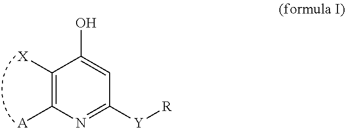

wherein
A is -(A1)ⱼ—(B1)ₖ-(A2)-(B2)ₘ—H, wherein
A1 and A2 independently are C₁₋₇ alkylene, C₂₋₇ alkenylene, C₂₋₇ alkynylene, or C₁₋₇ heteroalkylene, optionally substituted with one or two of independently selected U1;
B1 and B2 are independently an aliphatic ring, an aromatic ring or a fused ring, optionally substituted with one or two of independently selected U2; H is hydrogen;
j, k, l, and m are independently 0 or 1, wherein at least one of j, k, l and m is 1;
X is C₁₋₇ alkyl, C₂₋₇ heteroalkyl, C₂₋₇ alkenyl, C₂₋₇ heteroalkenyl, C₂₋₇ alkynyl, halogen, —CN, —NO₂, CF₃, —OH or —NH₂, optionally substituted with one, two or three independently selected U3;
------ defines an optional bond between X and A which is formed by substitution of one or two hydrogen radicals at a position on each of A and X such that a single or double bond is formed that results in a 5- or 6-membered aliphatic or aromatic ring fused with the pyridine;
Y is —OCH₂—, —N(R′)CH₂—, —CH₂—, or —O—, where R′ is hydrogen or C₁-C₃ alkyl; and
R is an aromatic ring, an aliphatic ring or a fused ring, optionally substituted with one, two or three of independently selected U4;
U1, U2, U3 and U4 are independently hydrogen, C₁₋₃ alkyl, C₁₋₃ haloalkyl, C₂₋₄ heteroalkyl, halogen, hydroxyl, ═O, =NR′=N—OR′—NR′R″, —SR′, —CN, C₂₋₅ alkynyl, C₂₋₅ alkenyl, C₃₋₅ cycloalkyl, C₃₋₅ heterocycloalkyl or —NO₂, where R′, R″, R′″ and R″″ independently refer to hydrogen, unsubstituted C₁₋₃ alkyl and C₂₋₃ heteroalkyl that can be connected by bonds to form rings, cycloalkyl or heterocycloalkyl;
or a pharmaceutically acceptable salt thereof, or a solvate thereof, or a solvate of the pharmaceutically acceptable salt thereof.

Preferred embodiment 2: The compound according to embodiment 1, which compound is of the formula I:

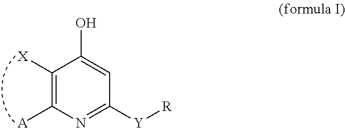

wherein
A is
   -A1-B1-A2-B2-H,
   -A1-B1-A2-H,
   —B1-A2-H,
   -A1-B2-H, or
   -A1-A2-H,
where A1 and A2 are independently C₁₋₇ alkylene, C₁₋₇ alkenylene, C₁₋₇ alkynylene or C₁₋₇ heteroalkylene; B1 and B2 are independently an aliphatic ring, an aromatic ring or a fused ring, and H is hydrogen;
X is C₁₋₇ alkyl, C₂₋₇ heteroalkyl, C₂₋₇ alkenyl, C₂₋₇ heteroalkenyl, C₂₋₇ alkynyl, halogen, —CN, —NO₂, CF₃, —OH or —NH₂, optionally substituted with one, two or three independently selected U3;
------ defines an optional bond between X and A which is formed by substitution of one or two hydrogen radicals at a position on each of A and X such that a single or double bond is formed that results in a 5- or 6-membered aliphatic or aromatic ring fused with the pyridine;
Y is —OCH₂—, —N(R′)CH₂—, —CH₂—, or —O—, where R′ is hydrogen or C₁-C₃ alkyl; and
R is
   an aromatic ring comprising nitrogen, an aliphatic ring or a fused ring, where the ring is optionally substituted with —CF₃, C₁₋₃ alkyl or C₂₋₄ heteroalkyl, or

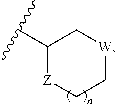

where the ring is optionally substituted with —CF₃, C₁₋₃ alkyl or C₂₋₄ heteroalkyl,
   wherein Z is —O—, —CH₂—, —NH— or N—(CH₂)₀₋₂—CH₃,
   W is —O— or —CH₂—, and
   n is 0, 1 or 2;
or a pharmaceutically acceptable salt thereof, or a solvate thereof, or a solvate of the pharmaceutically acceptable salt thereof.

Preferred embodiment 3: The compound according to any of the numbered embodiment, wherein X is halogen or C₁₋₇ alkyl or C₂₋₇ heteroalkyl.

Preferred embodiment 4: The compound according to any of the numbered embodiments, wherein X is C₁₋₃ alkyl or halogen.

Preferred embodiment 5: The compound according to any of the numbered embodiments, wherein A is -A1-B1-A2-H, where A1 and A2 are C₁₋₅ alkylene or C₁₋₅ heteroalkylene and B1 is aryl.

Preferred embodiment 6: The compound according to any one of the numbered embodiments, wherein X is C₁₋₇ alkyl or halogen and A is A1-B1-A2-H, where A1 and A2 are C₁₋₅ alkylene, and B1 is —(C₆H₄)—.

Preferred embodiment 7: The compound according to any on of the numbered embodiments, wherein A is —(CH₂)₂—(C₆H₄)—(CH₂)₂—CH₃.

Preferred embodiment 8: The compound according to any of the numbered embodiments, wherein Y is —OCH₂—.

Preferred embodiment 9: The compound according to any of the numbered embodiments, wherein R is

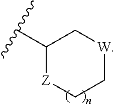

Preferred embodiment 10: The compound according to numbered embodiment 9, wherein n=1.

Preferred embodiment 11: The compound according to numbered embodiment 9 or 10, wherein Z═O.

Preferred embodiment 12: The compound according to any one of numbered embodiments 9-11, wherein W=CH₂ or O.

Preferred embodiment 13: The compound according to any of the numbered embodiments, wherein the compound is selected from the group consisting of:
3-iodo-2-(4-propylphenethyl)-6-((tetrahydro-2H-pyran-2-yl)methoxy)pyridin-4-ol,
3-chloro-2-(4-propylphenethyl)-6-((tetrahydro-2H-pyran-2-yl)methoxy)pyridin-4-ol,
3-bromo-2-(4-ethylphenethyl)-6-((tetrahydro-2H-pyran-2-yl)methoxy)pyridin-4-ol,
3-bromo-2-(4-butylphenethyl)-6-((tetrahydro-2H-pyran-2-yl)methoxy)pyridin-4-ol,
3-fluoro-2-(4-propylphenethyl)-6-((tetrahydro-2H-pyran-2-yl)methoxy)pyridin-4-ol,
4-hydroxy-2-(4-propylphenethyl)-6-((tetrahydro-2H-pyran-2-yl)methoxy)nicotinonitrile,
3-bromo-2-(4-ethoxyphenethyl)-6-((tetrahydro-2H-pyran-2-yl)methoxy)pyridin-4-ol,
3-bromo-2-nonyl-6-((tetrahydro-2H-pyran-2-yl)methoxy)pyridin-4-ol,
2-(4-butylphenethyl)-3-methyl-6-((tetrahydro-2H-pyran-2-yl)methoxy)pyridin-4-ol,
2-(4-ethoxyphenethyl)-3-methyl-6-((tetrahydro-2H-pyran-2-yl)methoxy)pyridin-4-ol,
(R)-2-(4-ethoxyphenethyl)-3-methyl-6-((tetrahydro-2H-pyran-2-yl)methoxy)pyridin-4-ol,
(S)-2-(4-ethoxyphenethyl)-3-methyl-6-((tetrahydro-2H-pyran-2-yl)methoxy)pyridin-4-ol,
2-(4-ethylphenethyl)-3-methyl-6-((tetrahydro-2H-pyran-2-yl)methoxy)pyridin-4-ol,
3-methyl-2-(4-methylphenethyl)-6-((tetrahydro-2H-pyran-2-yl)methoxy)pyridin-4-ol,
6-((1,4-dioxan-2-yl)methoxy)-2-(4-ethoxyphenethyl)-3-methylpyridin-4-ol,
2-(4-propylphenethyl)-6-((tetrahydro-2H-pyran-2-yl)methoxy)-3-(trifluoromethyl) pyridin-4-ol,
3-methyl-2-nonyl-6-((tetrahydro-2H-pyran-2-yl)methoxy)pyridin-4-ol,
3-methyl-2-(((4-propylbenzyl)oxy) methyl)-6-((tetrahydro-2H-pyran-2-yl)methoxy)pyridin-4-ol,
3-methyl-2-(((3-propylbenzyl)oxy) methyl)-6-((tetrahydro-2H-pyran-2-yl)methoxy)pyridin-4-ol,
2-(2-(6-ethoxypyridin-3-yl)ethyl)-3-methyl-6-((tetrahydro-2H-pyran-2-yl)methoxy)pyridin-4-ol,
3-ethyl-2-(4-propylphenethyl)-6-((tetrahydro-2H-pyran-2-yl)methoxy)pyridin-4-ol,
3-methoxy-2-(4-propylphenethyl)-6-((tetrahydro-2H-pyran-2-yl)methoxy)pyridin-4-ol,
3-propyl-2-(4-propylphenethyl)-6-((tetrahydro-2H-pyran-2-yl)methoxy)pyridin-4-ol,
3-isopropyl-2-(4-propylphenethyl)-6-((tetrahydro-2H-pyran-2-yl)methoxy)pyridin-4-ol,
3-butyl-2-(4-propylphenethyl)-6-((tetrahydro-2H-pyran-2-yl)methoxy)pyridin-4-ol,
3-isobutyl-2-(4-propylphenethyl)-6-((tetrahydro-2H-pyran-2-yl)methoxy)pyridin-4-ol,
3-pentyl-2-(4-propylphenethyl)-6-((tetrahydro-2H-pyran-2-yl)methoxy)pyridin-4-ol,
3-(methylamino)-2-(4-propylphenethyl)-6-((tetrahydro-2H-pyran-2-yl)methoxy)pyridin-4-ol,
3-(methoxymethyl)-2-(4-propylphenethyl)-6-((tetrahydro-2H-pyran-2-yl)methoxy)pyridin-4-ol,
3-((methylamino)methyl)-2-(4-propylphenethyl)-6-((tetrahydro-2H-pyran-2-yl)methoxy)pyridin-4-ol,
2-(4-propylphenethyl)-6-((tetrahydro-2H-pyran-2-yl)methoxy)-3-(o-tolyl)pyridin-4-ol,
2-(4-propylphenethyl)-6-((tetrahydro-2H-pyran-2-yl)methoxy)-3-(m-tolyl) pyridin-4-ol,
2-(4-propylphenethyl)-6-((tetrahydro-2H-pyran-2-yl)methoxy)-3-(p-tolyl)pyridin-4-ol,
3-mesityl-2-(4-propylphenethyl)-6-((tetrahydro-2H-pyran-2-yl)methoxy)pyridin-4-ol,
3-(2,6-dimethylphenyl)-2-(4-propylphenethyl)-6-((tetrahydro-2H-pyran-2-yl)methoxy)pyridin-4-ol,
3-(2,3-dimethylphenyl)-2-(4-propylphenethyl)-6-((tetrahydro-2H-pyran-2-yl)methoxy)pyridin-4-ol,
3-(3,5-dimethylphenyl)-2-(4-propylphenethyl)-6-((tetrahydro-2H-pyran-2-yl)methoxy)pyridin-4-ol,
3-(4-ethylphenyl)-2-(4-propylphenethyl)-6-((tetrahydro-2H-pyran-2-yl)methoxy)pyridin-4-ol,
3-(4-methoxyphenyl)-2-(4-propylphenethyl)-6-((tetrahydro-2H-pyran-2-yl)methoxy)pyridin-4-ol,
2-(4-propylphenethyl)-3-(4-propylphenyl)-6-((tetrahydro-2H-pyran-2-yl)methoxy)pyridin-4-ol,
3-(4-(methoxymethyl)phenyl)-2-(4-propylphenethyl)-6-((tetrahydro-2H-pyran-2-yl)methoxy)pyridin-4-ol,
3-(4-butylphenyl)-2-(4-propylphenethyl)-6-((tetrahydro-2H-pyran-2-yl)methoxy)pyridin-4-ol,
2-(4-propylphenethyl)-6-((tetrahydro-2H-pyran-2-yl)methoxy)-3-(4-(trifluoromethyl)phenyl)pyridin-4-ol,
2-(4-propylphenethyl)-6-((tetrahydro-2H-pyran-2-yl)methoxy)-3-(4-(trifluoromethoxy) phenyl) pyridin-4-ol,
2′-(4-propylphenethyl)-6′-((tetrahydro-2H-pyran-2-yl)methoxy)-[2,3′-bipyridin]-4′-ol,
2-(4-propylphenethyl)-6-((tetrahydro-2H-pyran-2-yl)methoxy)-[3,3′-bipyridin]-4-ol,
2-(4-propylphenethyl)-6-((tetrahydro-2H-pyran-2-yl)methoxy)-[3,4′-bipyridin]-4-ol,
6′-methoxy-2-(4-propylphenethyl)-6-((tetrahydro-2H-pyran-2-yl)methoxy)-[3,3′-bipyridin]-4-ol,
3-(4-chlorophenyl)-2-(4-propylphenethyl)-6-((tetrahydro-2H-pyran-2-yl)methoxy)pyridin-4-ol,
3-(4-fluorophenyl)-2-(4-propylphenethyl)-6-((tetrahydro-2H-pyran-2-yl)methoxy)pyridin-4-ol,
3-(3-fluorophenyl)-2-(4-propylphenethyl)-6-((tetrahydro-2H-pyran-2-yl)methoxy)pyridin-4-ol,
3-(2-fluorophenyl)-2-(4-propylphenethyl)-6-((tetrahydro-2H-pyran-2-yl)methoxy)pyridin-4-ol,
3-cyclopentyl-2-(4-propylphenethyl)-6-((tetrahydro-2H-pyran-2-yl)methoxy)pyridin-4-ol,
3-cyclohexyl-2-(4-propylphenethyl)-6-((tetrahydro-2H-pyran-2-yl)methoxy)pyridin-4-ol,
3-(furan-3-yl)-2-(4-propylphenethyl)-6-((tetrahydro-2H-pyran-2-yl)methoxy)pyridin-4-ol,
3-(1-methyl-1H-pyrrol-3-yl)-2-(4-propylphenethyl)-6-((tetrahydro-2H-pyran-2-yl)methoxy)pyridin-4-ol,
2-(4-propylphenethyl)-6-((tetrahydro-2H-pyran-2-yl)methoxy)-3-(thiophen-3-yl)pyridin-4-ol,
3-(4-methylthiophen-3-yl)-2-(4-propylphenethyl)-6-((tetrahydro-2H-pyran-2-yl)methoxy)pyridin-4-ol,
3-phenethyl-2-(4-propylphenethyl)-6-((tetrahydro-2H-pyran-2-yl)methoxy)pyridin-4-ol,
3-(4-methylphenethyl)-2-(4-propylphenethyl)-6-((tetrahydro-2H-pyran-2-yl)methoxy)pyridin-4-ol,
3-(benzyloxy)-2-(4-propylphenethyl)-6-((tetrahydro-2H-pyran-2-yl)methoxy)pyridin-4-ol,
3-(benzylamino)-2-(4-propylphenethyl)-6-((tetrahydro-2H-pyran-2-yl)methoxy)pyridin-4-ol,
3-(phenoxymethyl)-2-(4-propylphenethyl)-6-((tetrahydro-2H-pyran-2-yl)methoxy)pyridin-4-ol,
4-hydroxy-2-(4-propylphenethyl)-6-((tetrahydro-2H-pyran-2-yl)methoxy)nicotinamide,
4-hydroxy-N-methyl-2-(4-propylphenethyl)-6-((tetrahydro-2H-pyran-2-yl)methoxy)nicotinamide,
N-ethyl-4-hydroxy-2-(4-propylphenethyl)-6-((tetrahydro-2H-pyran-2-yl)methoxy)nicotinamide,
4-hydroxy-N-phenyl-2-(4-propylphenethyl)-6-((tetrahydro-2H-pyran-2-yl)methoxy)nicotinamide,
2-(4-ethoxyphenethyl)-4-hydroxy-6-((tetrahydro-2H-pyran-2-yl)methoxy)-N-(p-tolyl)nicotinamide,
2-(4-ethoxyphenethyl)-4-hydroxy-N-(6-methoxypyridin-3-yl)-6-((tetrahydro-2H-pyran-2-yl)methoxy)nicotinamide,
2-(4-ethoxyphenethyl)-4-hydroxy-N-(1H-pyrrol-2-yl)-6-((tetrahydro-2H-pyran-2-yl)methoxy)nicotinamide,
2-(4-ethoxyphenethyl)-4-hydroxy-N-(1-methyl-1H-pyrrol-2-yl)-6-((tetrahydro-2H-pyran-2-yl)methoxy)nicotinamide,
7-(4-methoxyphenyl)-2-((tetrahydro-2H-pyran-2-yl)methoxy)quinolin-4-ol,
7-(3-ethoxyphenyl)-2-((tetrahydro-2H-pyran-2-yl)methoxy)quinolin-4-ol,
7-(4-propylphenyl)-2-((tetrahydro-2H-pyran-2-yl)methoxy)quinolin-4-ol,
7-(6-ethoxypyridin-3-yl)-2-((tetrahydro-2H-pyran-2-yl)methoxy)quinolin-4-ol,
7-hexyl-2-((tetrahydro-2H-pyran-2-yl)methoxy)quinolin-4-ol},
7-(hexyloxy)-2-((tetrahydro-2H-pyran-2-yl)methoxy)quinolin-4-ol,
2-((1,4-dioxan-2-yl)methoxy)-7-(4-ethoxyphenyl)quinolin-4-ol,
2-((1,4-dioxan-2-yl)methoxy)-7-(6-ethoxypyridin-3-yl)quinolin-4-ol,
7-(hexylamino)-2-((tetrahydro-2H-pyran-2-yl)methoxy)quinolin-4-ol,
7-(furan-3-yl)-2-((tetrahydro-2H-pyran-2-yl)methoxy)quinolin-4-ol,
7-(1-methyl-1H-pyrrol-3-yl)-2-((tetrahydro-2H-pyran-2-yl)methoxy)quinolin-4-ol,
7-(4-methylthiophen-3-yl)-2-((tetrahydro-2H-pyran-2-yl)methoxy)quinolin-4-ol,
7-cyclohexyl-2-((tetrahydro-2H-pyran-2-yl)methoxy)quinolin-4-ol,
7-cyclopentyl-2-((tetrahydro-2H-pyran-2-yl)methoxy)quinolin-4-ol,
2-(4-ethoxyphenethyl)-3-ethyl-6-((tetrahydro-2H-pyran-2-yl)methoxy)pyridin-4-ol,
3-propyl-2-(4-propylphenethyl)-6-((tetrahydro-2H-pyran-2-yl)methoxy)pyridin-4-ol,
6-((1,4-dioxan-2-yl)methoxy)-3-bromo-2-(4-ethoxyphenethyl) pyridin-4-ol,
3-chloro-2-(4-ethoxyphenethyl)-6-((tetrahydro-2H-pyran-2-yl)methoxy)pyridin-4-ol
3-methyl-2-nonyl-6-((tetrahydro-2H-pyran-2-yl)methoxy)pyridin-4-ol,
2-(6-cyclohexylhexyl)-3-methyl-6-((tetrahydro-2H-pyran-2-yl)methoxy)pyridin-4-ol,
2-(2-(6-propylpyridin-3-yl)ethyl)-6-((tetrahydro-2H-pyran-2-yl)methoxy)-3-(trifluoromethyl) pyridin-4-ol,
2-(2-(6-propylpyridin-3-yl)ethyl)-6-((tetrahydro-2H-pyran-2-yl)methoxy)-3-(trifluoromethyl) pyridin-4-ol,
7-(4-propylphenyl)-2-((tetrahydro-2H-pyran-2-yl)methoxy)quinolin-4-ol,
7-heptyl-2-((tetrahydro-2H-pyran-2-yl)methoxy)quinolin-4-ol,
7-(hept-1-yn-1-yl)-2-((tetrahydro-2H-pyran-2-yl)methoxy)quinolin-4-ol,
7-propyl-2-((tetrahydro-2H-pyran-2-yl)methoxy)-1,6-naphthyridin-4-ol,
6-(2-(pyridin-2-yl)ethyl)-2-((tetrahydro-2H-pyran-2-yl)methoxy)quinolin-4-ol
7-phenethyl-2-((tetrahydro-2H-pyran-2-yl)methoxy)-1,5-naphthyridin-4-ol
1-methyl-2-(4-propylphenyl)-6-((tetrahydro-2H-pyran-2-yl)methoxy)-1H-pyrrolo[2,3-b]pyridin-4-ol
8-(4-methoxyphenethyl)-2-((tetrahydro-2H-pyran-2-yl)methoxy)quinolin-4-ol
6-ethyl-4-hydroxy-7-(4-propylphenyl)-2-((tetrahydro-2H-pyran-2-yl)methoxy)-7,8-dihydro-1,6-naphthyridin-5 (6H)-one
7-(4-propylphenyl)-2-((tetrahydro-2H-pyran-2-yl)methoxy)-5,6,7,8-tetrahydroquinolin-4-ol,
6-(4-propylphenyl)-2-((tetrahydro-2H-pyran-2-yl)methoxy)-6,7-dihydro-5H-cyclopenta[b]pyridin-4-ol,
2-((1,4-dioxan-2-yl)methoxy)-7-(4-ethoxyphenyl)-5,7-dihydrofuro[3,4-b]pyridin-4-ol, and
3-(6-(chroman-2-ylmethoxy)-4-hydroxy-2-(4-propylphenethyl)pyridin-3-yl)propanoic acid.

Preferred embodiment 14: A pharmaceutical composition for use as a medicament, said pharmaceutical composition comprising a compound according to any of the numbered embodiments and a pharmaceutically acceptable carrier, excipient or diluent.

Preferred embodiment 15: The pharmaceutical composition according to numbered embodiment 14, wherein the composition is for use in the treatment of fibrotic, inflammatory, diabetic or cognitive disease.

### EXAMPLES

Aq Aqueous
cAMP Cyclic adenosine monophosphate
DCM Dichloromethane
DEE Diethyl ether
DMF Dimethylformamide
DMSO Dimethylsulfoxide
Et Ethylt
FBS Fetal bovine serum
HEPES 4-(2-HydroxyEthyl)-1-Piperazine EthaneSulfonic acid
HPLC High Performance Liquid Chromatography
IBMX 3-Isobutyl-1-methylxanthine
Me Methyl
MS Mass Spectrometry
MW Microwave
ND Not determined
NMR Nuclear Magnetic Resonance
PE Petroleum ether
PIntB 2-(Di-tert-butylphosphino)-N-phenylindole
rt Room temperature
TFA Trifluoroacetic acid
THF Tetrahydrofuran
TMEDA Tetramethylethylenediamine
TMSA Ethynyltrimethylsilane

### Synthetic Procedures

Chemicals were used without further purification. Anhydrous reactions were carried out in oven or flame-dried glassware under nitrogen atmosphere. Dry chromatographic grade DCM, THF and DMF were obtained from a Waters SG solvent purification system. MeCN was dried over molecular sieves (3 Å). Thin layer chromatography (TLC) Silica gel 60 F254, Merck pre-coated plates were used, visualized under UV light (254 or 365 nm), developed in the system stated for each compound. Flash chromatography of compounds was performed using silica gel 60 (40-64 μm), where loading of the compounds was done after dry mixing with Celite. Nuclear Magnetic Resonance (NMR) spectra were recorded on 400 or 600 MHz Bruker instruments. The obtained FID files were processed with Mnova 14 software. Signals are reported in ppm (δ) using the solvent as reference. Multiplet patterns are designated the following abbreviations, or combinations of these: m—multiplet, s—singlet, d—doublet, t—triplet, q—quartet, p—pentet, h—sextet. Signal assignments were made from chemical shifts. Mass spectrometry (MS) was performed on an Aquity UPLC instrument connected to an Aquity TUV detector and an Aquity QDa detector. Gradient: 100% A to 100% B over 5 min. Mobile phase A: MeCN 5%, Formic acid 0.1% in water, Mobile Phase B: MeCN 99.9%, Formic acid 0.1%. Flow rate: 0.5 mL/min. Samples used in this system were dissolved in 1:1 MeCN:water or MeOH. Analytical High-Performance Liquid Chromatography (HPLC) was performed on a Dionex UltiMate HPLC system consisting of an LPG-3400A pump (1 mL/min), a WPS-3000L autosampler and a DAD-3000D diode array detector (210, 254, 290, 365 nm), using a Gemini-NX C18 column (4.6×250 mm, 3 μm, 110 Å); Mobile phase A: water:TFA, 100:0.1, v/v. Mobile phase B: MeCN:water:TFA, 90:10:0.1, v/v/v. Data were acquired and processed using the Chromeleon Software v. 6.80. Methods used for analysis: Method A: Gradient 0-15 min, 50-100% Mobile phase B. Method B: Gradient 0-15 min, 30-100% Mobile phase B.

Mass analysis by matrix-assisted laser desorption/ionization high-resolution mass spectrometry (MALDI-HRMS) was performed on a QExactive Orbitrap mass spectrometer (Thermo Scientific, Bremen, Germany) equipped with a SMALDI5 ion source (TransMIT GmbH, Giessen, Germany). The sample was analyzed in the positive ion mode using a peak from the DHB matrix for internal mass calibration whereby a mass accuracy of 2 ppm or better was achieved. The samples (1 mg) were mixed with a 20 mg/mL solution of 2,5-dihydroxybenzoic acid in MeOH as MALDI matrix, 2 μL of the mixture was deposited on a 30 well glass plate (Electron Microscopy Sciences, Hatfield, PA, USA) and analysis followed upon evaporation. Infrared (IR) spectra were recorded neat on a Perkin-Elmer Spectrum One fourier-transform spectrometer with a universal ATR accesory. The signals (λₘₐₓ) are reported in wavenumbers (cm⁻¹). Peak intensity is designated the following abbreviations or combination of these: broad (br), weak (w), medium (m) and strong (s). Samples were dissolved in DCM, added dropwise onto the analysis plate and the spectra were recorded upon evaporation of the solvent.

### Synthetic Routes

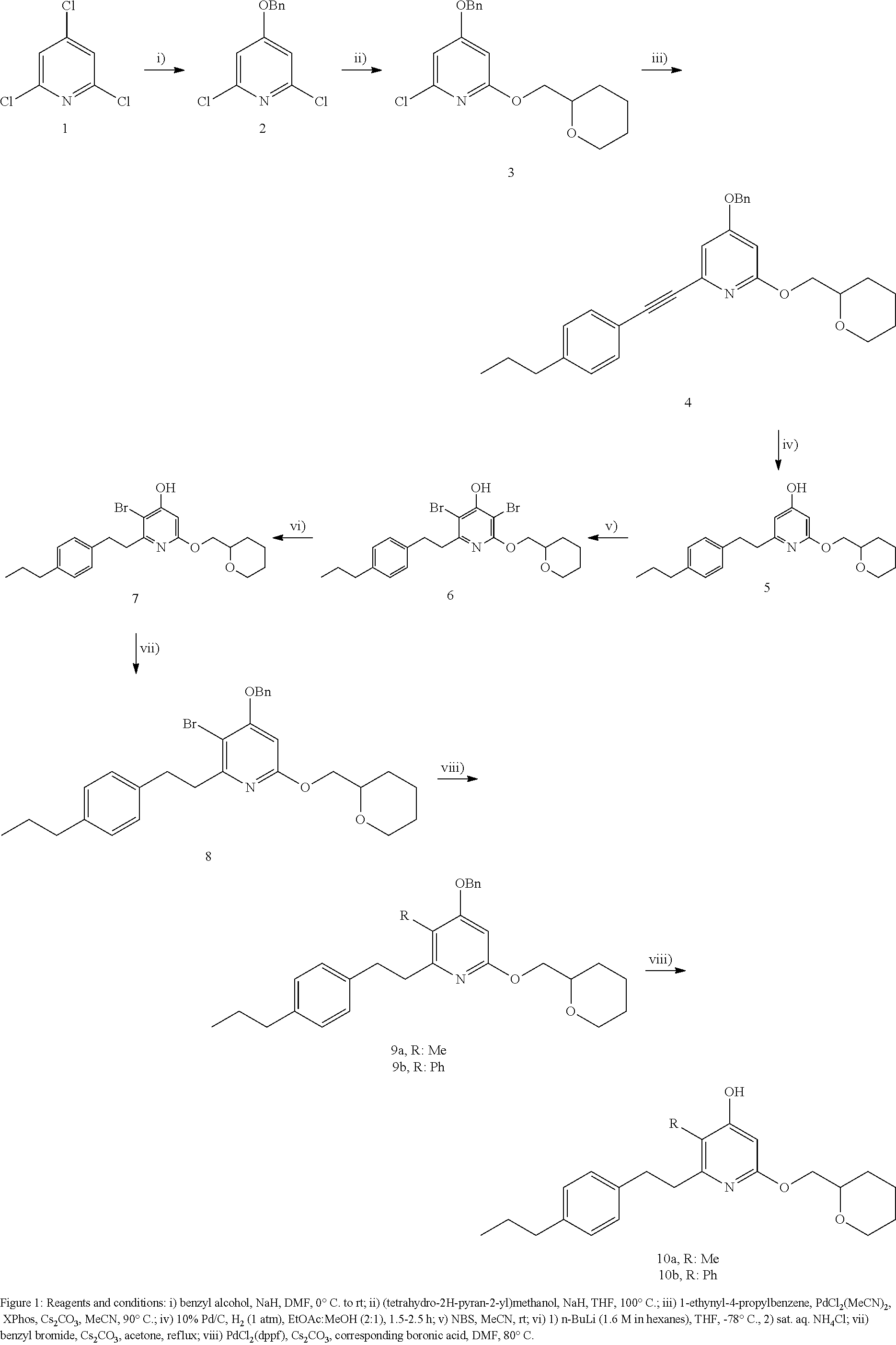

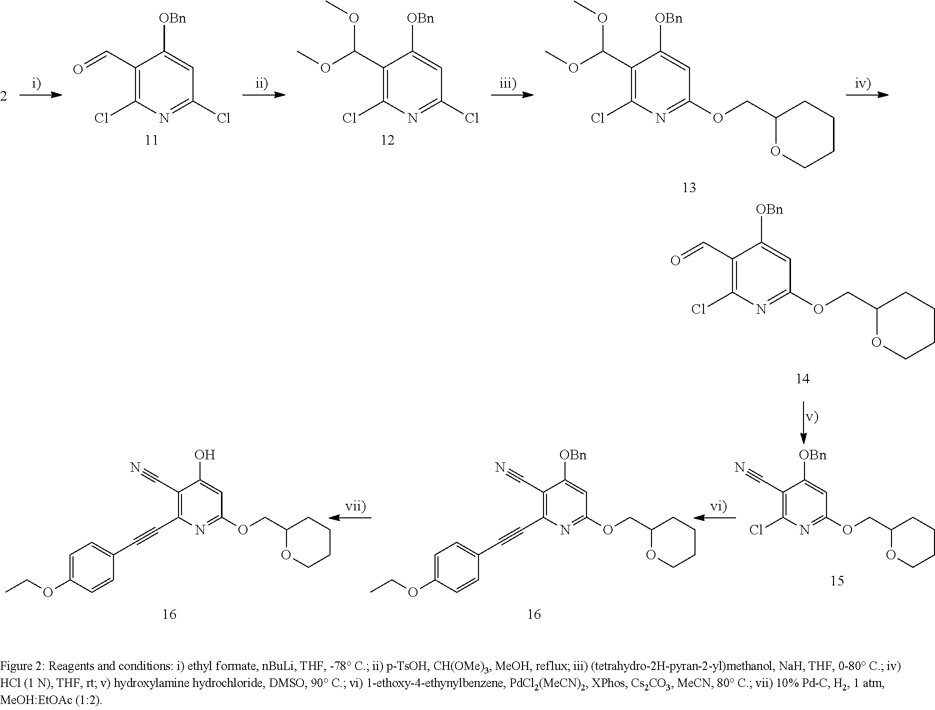

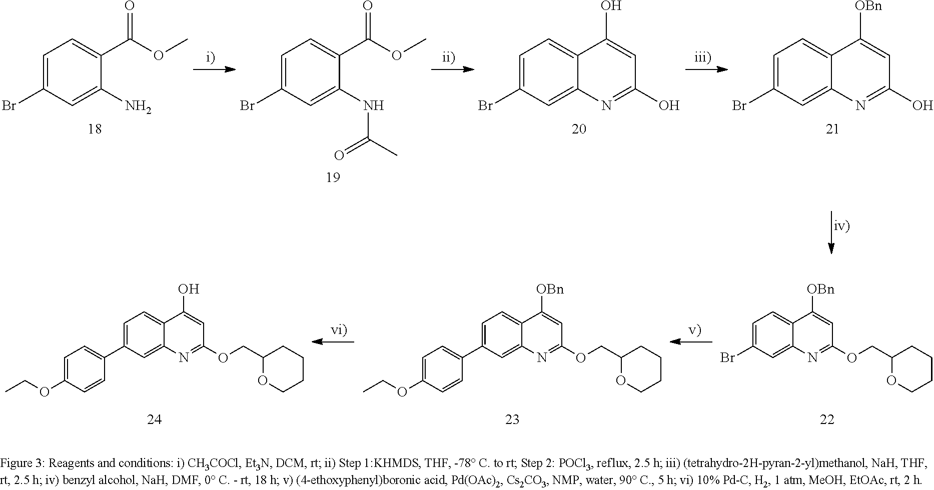

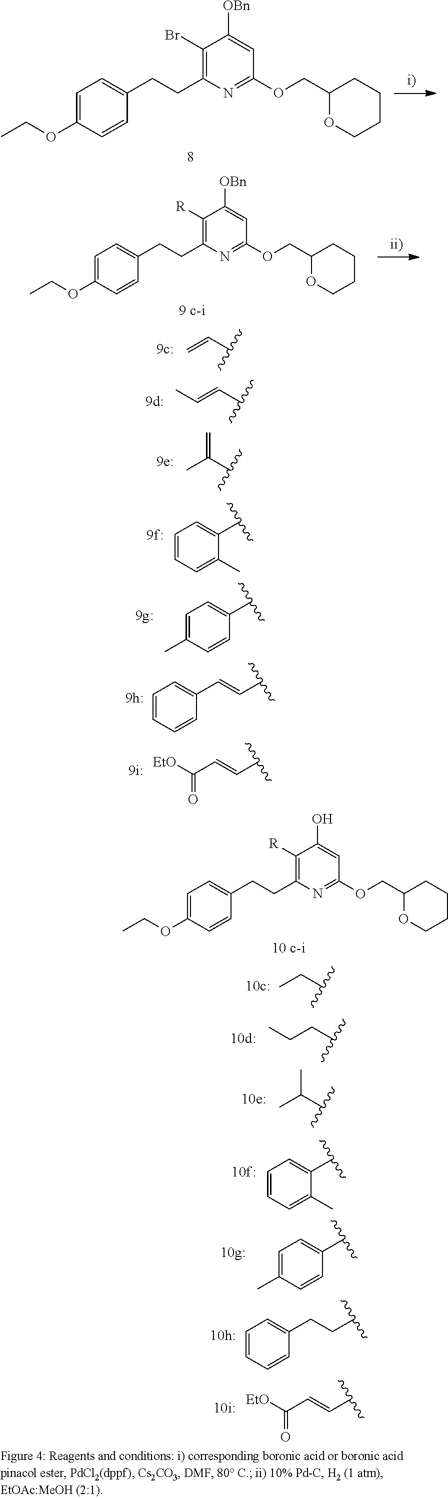

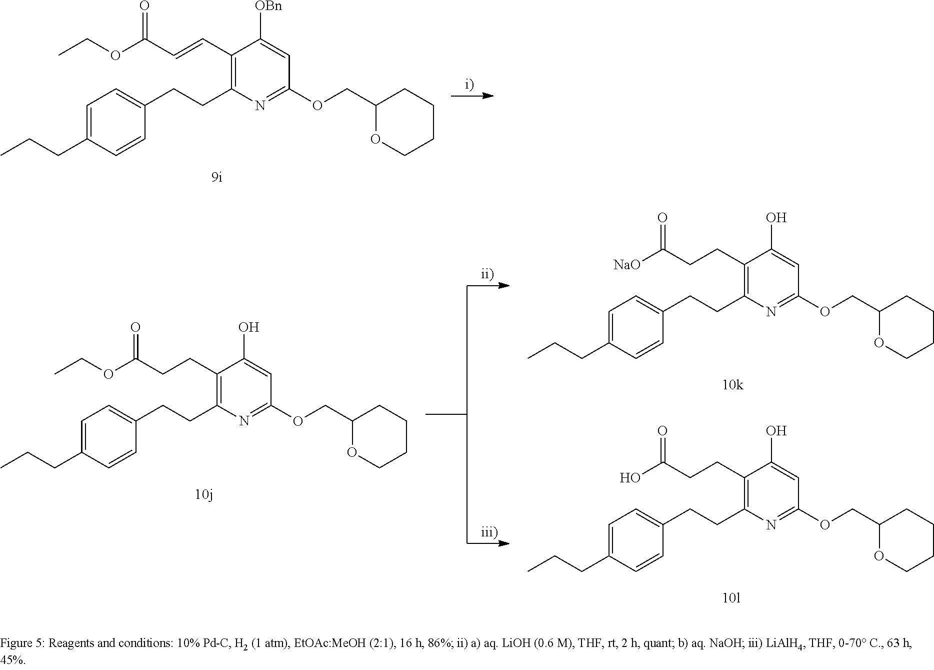

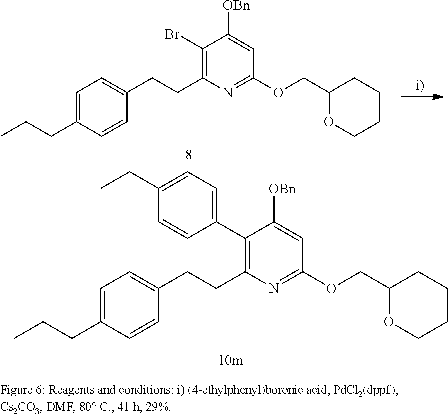

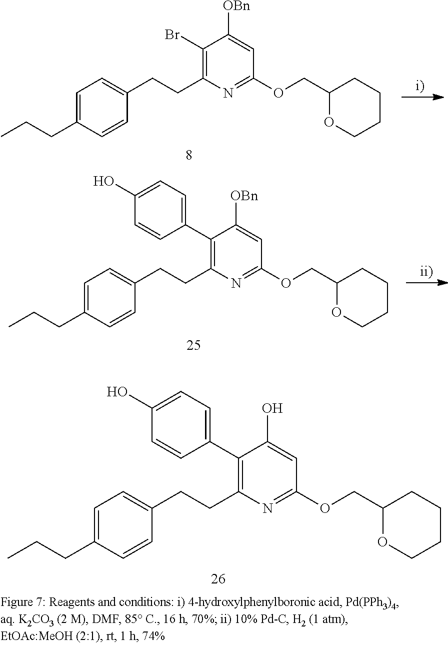 [SMILES: CCCc1ccc(CCc2nc(OCC3CCCCO3)cc(OCc3ccccc3)c2Br)cc1 ; CCCc1ccc(CCc2nc(OCC3CCCCO3)cc(OCc3ccccc3)c2-c2ccc(O)cc2)cc1 ; CCCc1ccc(CCc2nc(OCC3CCCCO3)cc(O)c2-c2ccc(O)cc2)cc1]

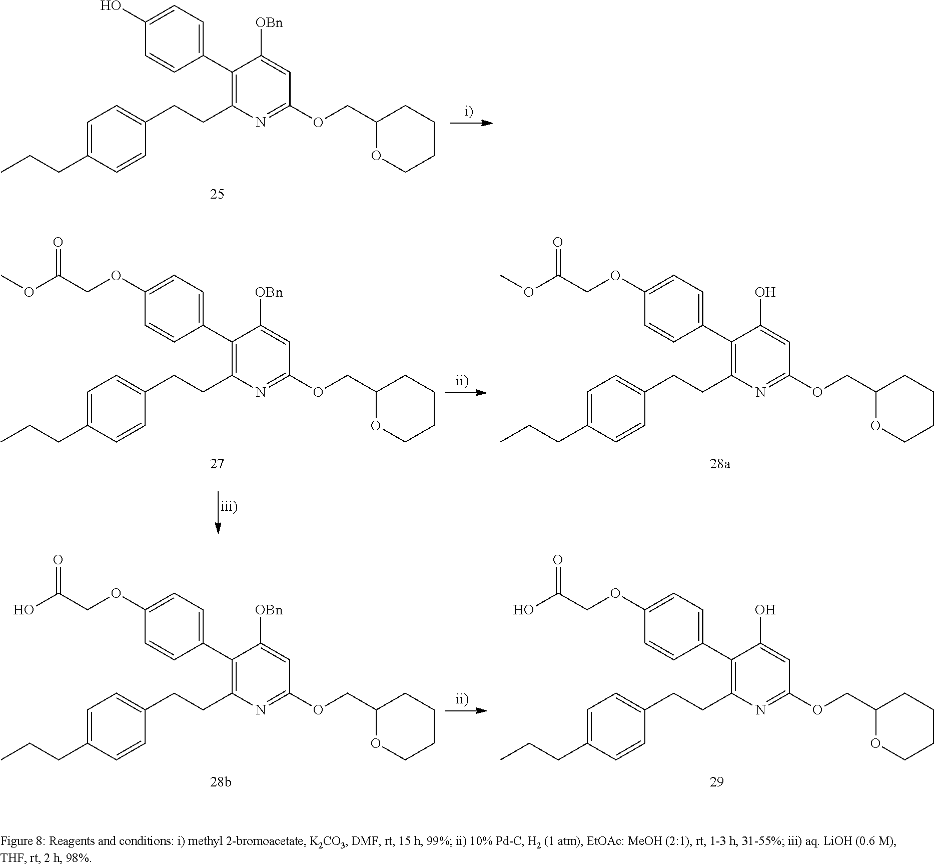 [SMILES: CCCc1ccc(CCc2nc(OCC3CCCCO3)cc(OCc3ccccc3)c2-c2ccc(O)cc2)cc1 ; CCCc1ccc(CCc2nc(OCC3CCCCO3)cc(OCc3ccccc3)c2-c2ccc(OCC(=O)OC)cc2)cc1 ; CCCc1ccc(CCc2nc(OCC3CCCCO3)cc(O)c2-c2ccc(OCC(=O)OC)cc2)cc1 ; CCCc1ccc(CCc2nc(OCC3CCCCO3)cc(OCc3ccccc3)c2-c2ccc(OCC(=O)O)cc2)cc1 ; CCCc1ccc(CCc2nc(OCC3CCCCO3)cc(O)c2-c2ccc(OCC(=O)O)cc2)cc1]

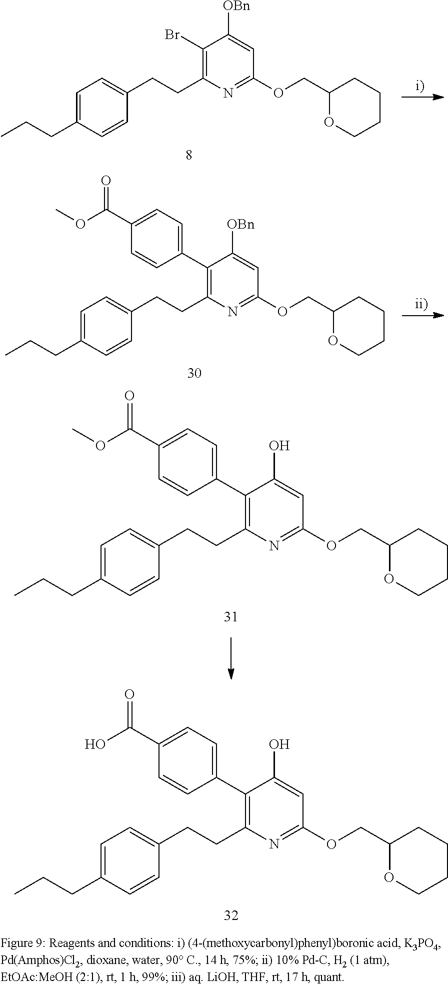

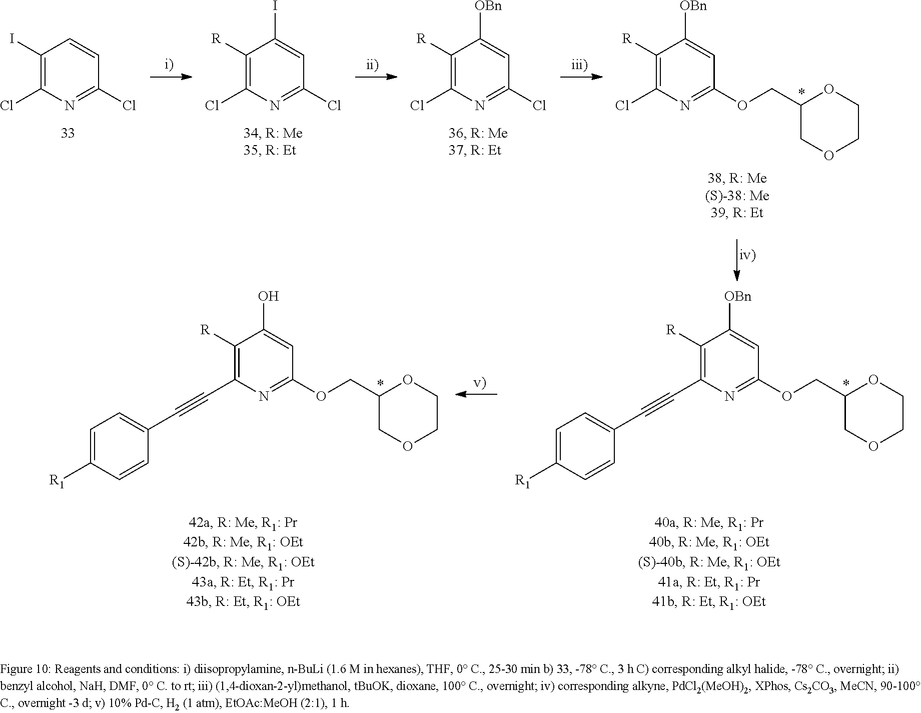

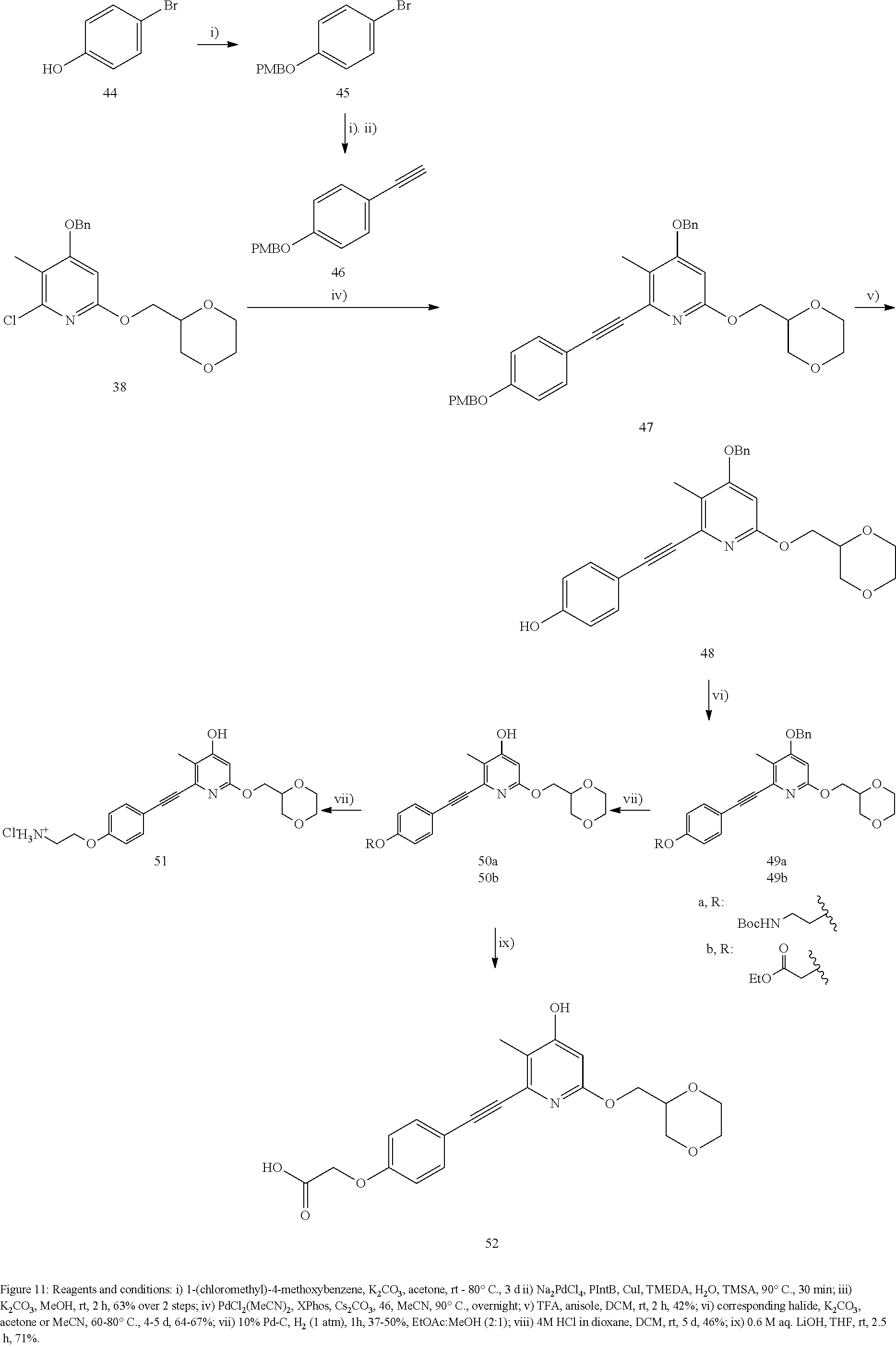

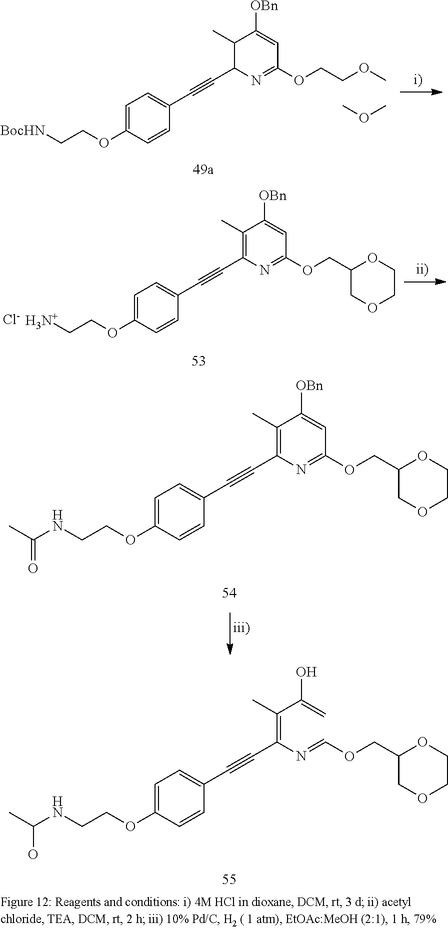

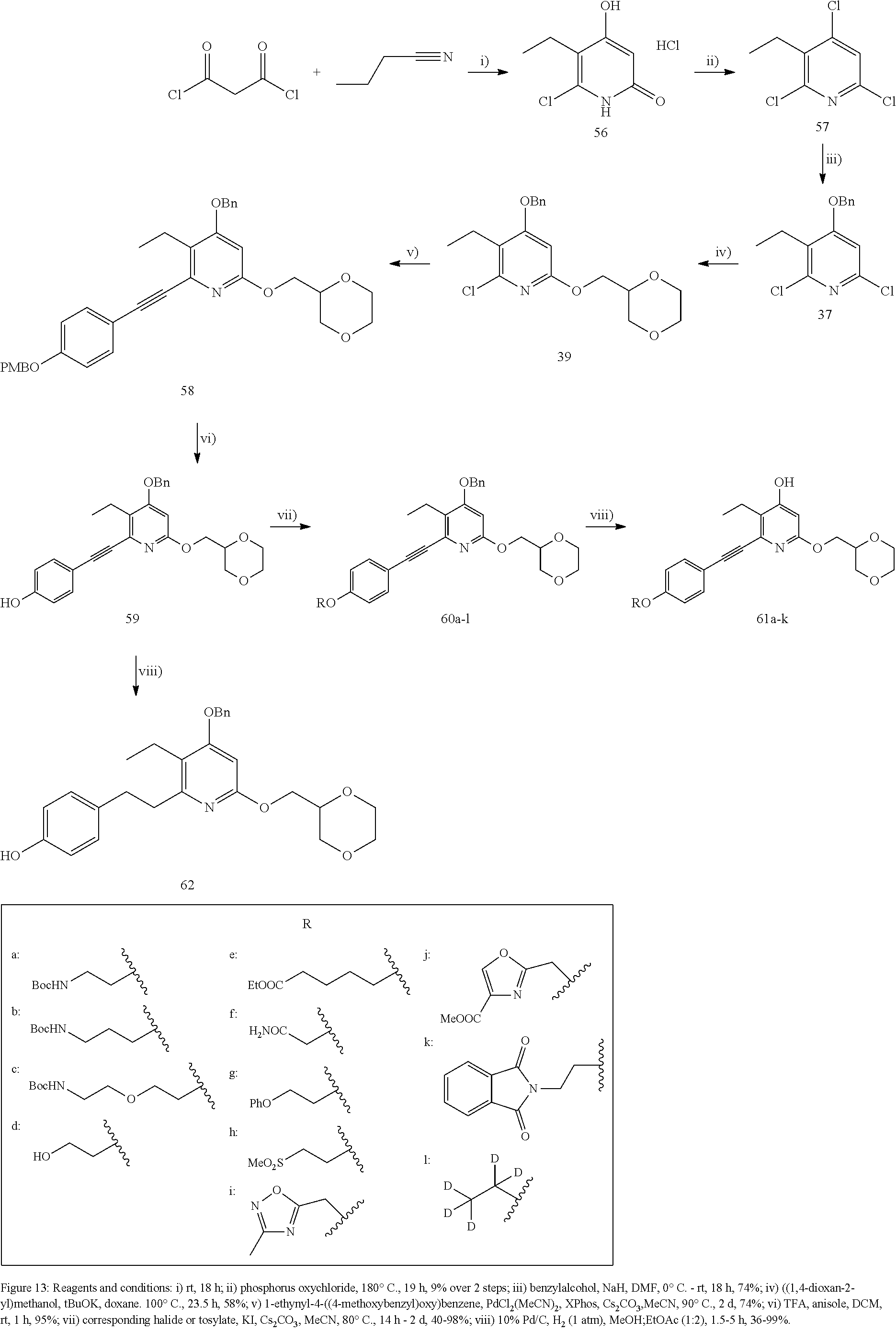

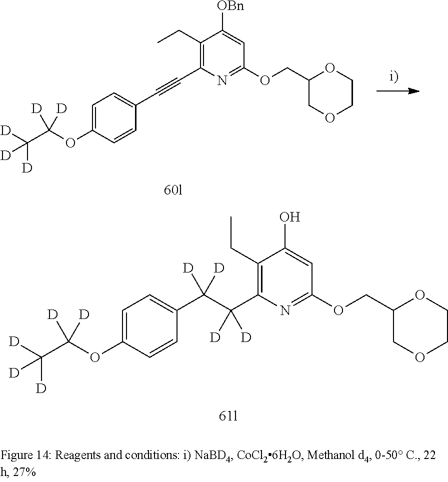 [SMILES: [2H]C([2H])([2H])C([2H])([2H])Oc1ccc(C#Cc2nc(OCC3COCCO3)cc(OCc3ccccc3)c2CC)cc1 ; [2H]C([2H])([2H])C([2H])([2H])Oc1ccc(C([2H])([2H])C([2H])([2H])c2nc(OCC3COCCO3)cc(O)c2CC)cc1]

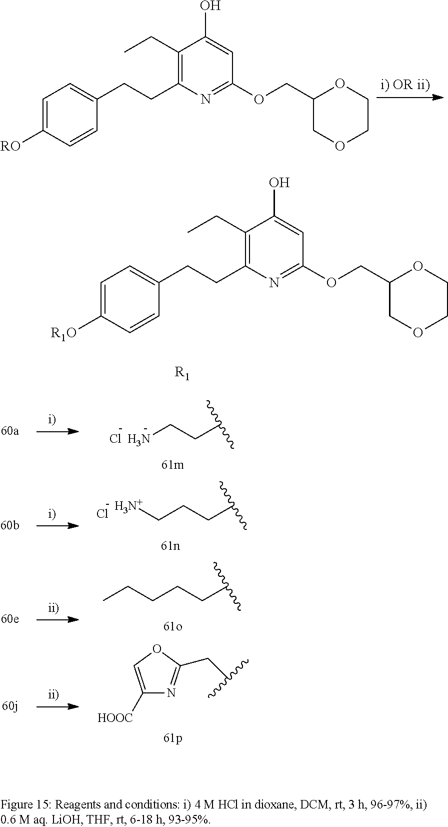

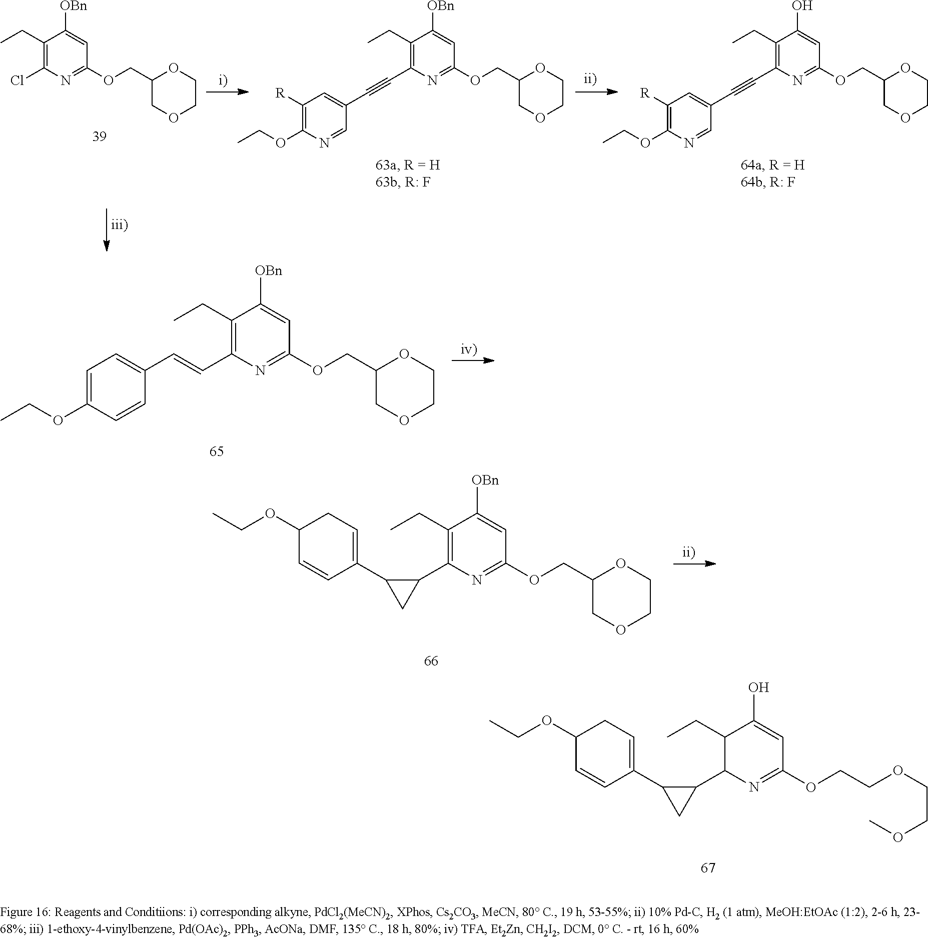

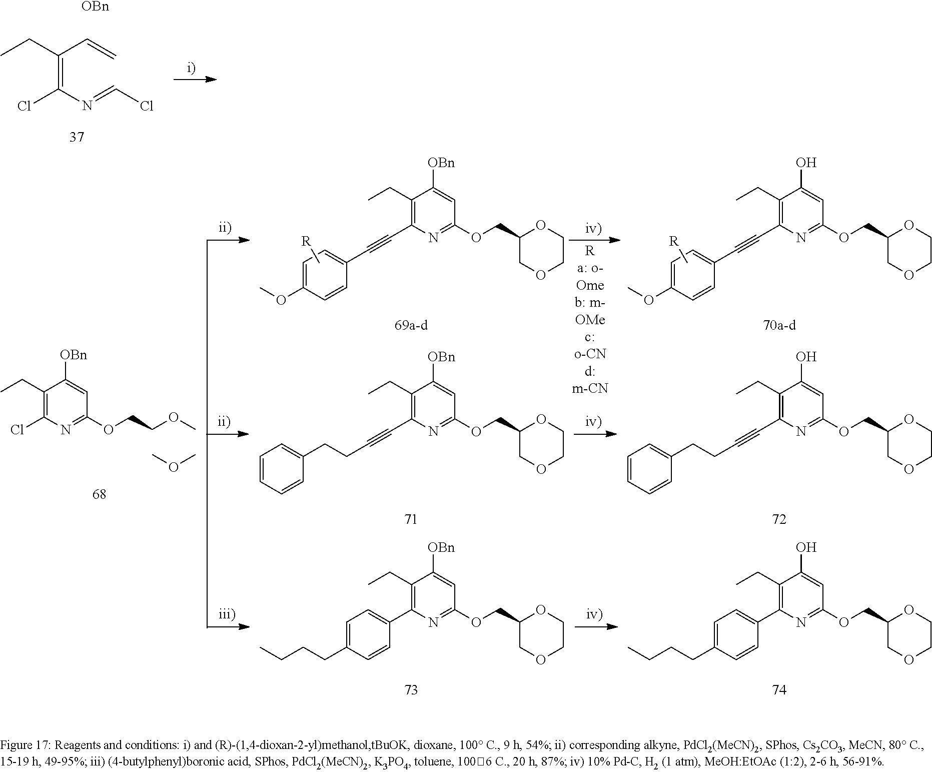

### EXPERIMENTAL PROCEDURES

4-(Benzyloxy)-2,6-dichloropyridine (2). NaH (60% dispersion in mineral oil, 535.8 mg, 13.4 mmol) was added to a solution of 2,4,6-trichloropyridine (1) (1.88 g, 10.3 mmol) in anhydrous DMF (11.5 mL) at 0° C. Benzyl alcohol (1.07 mL, 10.3 mmol) was added dropwise in 5 min (effervescence was observed). The reaction mixture was stirred at 0° C. for 3 h and then at rt for 18.5 h. The reaction was quenched by addition of sat. aq. NH₄Cl (5.0 mL). Then, 50 mL CaCl₂ solution (3.0 M) and 5 mL water was added to the reaction mixture and extracted with EtOAc (3×60 mL). The combined organic phase was dried over anhydrous Na₂SO₄, filtered, evaporated onto Celite, and purified by flash chromatography (3% EtOAc in heptane) to afford 2 as a white solid (1.77 g, 68%). R_{f}=0.40 (EtOAc:heptane; 1:9); ¹H NMR (600 MHz, CDCl₃) δ 7.44-7.40 (m, 2H), 7.40-7.37 (m, 3H), 6.86 (s, 2H), 5.11 (s, 2H); ¹³C NMR (151 MHz, CDCl₃) δ 167.7, 151.5, 134.6, 129.1, 129.0, 127.7, 110.0, 71.1.

4-(Benzyloxy)-2-chloro-6-((tetrahydro-2H-pyran-2-yl)methoxy)pyridine (3). NaH (60% dispersion in mineral oil, 208.5 mg, 5.21 mmol) was added to a solution of (tetrahydro-2H-pyran-2-yl)methanol (490 μL, 4.34 mmol) in anhydrous THF (2.5 mL) at 0° C. and stirred for 15 min. Then, a solution of 2 (1.1 g, 4.34 mmol) in anhydrous THF (7.5 mL) was added. The vial was sealed and stirred at 100° C. for 4 h. The cooled reaction mixture was poured onto water (50 mL) and extracted with DCM (3×50 mL). The combined organic phase was washed with brine and dried over Na₂SO₄, filtered, and purified by flash chromatography (5-8% EtOAc in heptane) to afford 3 as a colorless oil (1.022 g, 70%). R_{f}=0.5 (EtOAc:heptane; 1:4); ¹H NMR (600 MHz, CDCl₃) δ 7.43-7.30 (m, 5H), 6.57 (d, J=1.9 Hz, 1H), 6.28 (d, J=1.9 Hz, 1H), 5.04 (s, 2H), 4.31 (dd, J=11.4, 3.0 Hz, 1H), 4.21 (dd, J=11.3, 7.1 Hz, 1H), 4.08-4.02 (m, 1H), 3.71-3.63 (m, 1H), 3.49 (td, J=11.7, 2.2 Hz, 1H), 1.93-1.86 (m, 1H), 1.67-1.49 (m, 4H), 1.42 (qd, J=12.5, 3.9 Hz, 1H); ¹³C NMR (151 MHz, CDCl₃) δ 168.3, 164.9, 149.0, 135.5, 128.9, 128.6, 127.6, 106.1, 94.3, 76.0, 70.5, 70.0, 68.6, 28.0, 26.0, 23.2.

4-(Benzyloxy)-2-((4-propylphenyl)ethynyl)-6-((tetrahydro-2H-pyran-2-yl)methoxy)pyridine (4). A flame-dried microwave vial was charged with PdCl₂(MeCN)₂ (2.9 mg, 11.3 μmol), XPhos (16.1 mg, 33.8 μmol), and Cs₂CO₃ (477.1 mg, 1.46 mmol). The vial was evacuated and backfilled with argon three times. Then, a solution of 1-ethynyl-4-propylbenzene (99.5 mg, 0.69 mmol) in anhydrous MeCN (0.7 mL) was added and stirred for 15 min. After that, a solution of 3 (188 mg, 0.56 mmol) in anhydrous MeCN (7.5 mL) was added to the reaction mixture. The vial was capped and stirred at 90° C. for 16 h. The cooled reaction mixture was filtered, evaporated onto celite, and purified by flash chromatography (60% DCM in heptane) to afford 4 as a yellow oil (195 mg, 78%). R_{f}=0.2 (DCM:heptane; 7:3); ¹H NMR (400 MHz, CDCl₃) δ 7.49 (d, J=8.2 Hz, 2H), 7.43-7.31 (m, 5H), 7.16 (d, J=8.1 Hz, 2H), 6.82 (d, J=2.1 Hz, 1H), 6.35 (d, J=2.1 Hz, 1H), 5.07 (s, 2H), 4.41 (dd, J=11.5, 3.1 Hz, 1H), 4.28 (dd, J=11.5, 7.1 Hz, 1H), 4.14-4.00 (m, 1H), 3.78-3.64 (m, 1H), 3.50 (td, J=11.5, 2.3 Hz, 1H), 2.60 (t, J=7.6 Hz, 2H), 1.98-1.82 (m, 1H), 1.71-1.38 (m, 7H), 0.94 (t, J=7.4 Hz, 3H); ¹³C NMR (101 MHz, CDCl₃) δ 166.9, 165.4, 143.9, 141.0, 135.8, 132.1, 128.8, 128.6, 128.5, 127.6, 119.7, 110.9, 95.6, 88.7, 88.4, 76.2, 70.1, 69.6, 68.6, 38.1, 28.0, 26.0, 24.4, 23.3, 13.9.

### General Procedure for Hydrogenation/Debenzylation for Compound 5 and 10a,b as Exemplified by Compound 5

2-(4-Propylphenethyl)-6-((tetrahydro-2H-pyran-2-yl)methoxy)pyridin-4-ol (5). 10% Pd—C (23.5 mg, 22.1 μmol) was added to a solution of 4 (195 mg, 0.44 mmol) in MeOH:EtOAc (1:2; 4.5 mL). The suspension was evacuated and backfilled with argon three times and then with hydrogen three times, and left stirring at RT under an atmosphere of hydrogen (1.0 bar). After 2.25 h, the reaction mixture was evaporated onto celite and purified by flash chromatography (6% MeOH in DCM) to afford 5 as a slightly yellow oil (154.5 mg, 98%). R_{f}=0.49 (MeOH:DCM; 5:95); ¹H NMR (600 MHz, DMSO) δ 10.32 (br s, 1H), 7.09 (d, J=8.1 Hz, 2H), 7.06 (d, J=8.1 Hz, 2H), 6.24 (d, J=1.9 Hz, 1H), 5.91 (d, J=1.9 Hz, 1H), 4.12 (d, J=5.2 Hz, 2H), 3.90-3.84 (m, 1H), 3.57 (m, 1H), 3.38-3.34 (m, 1H), 2.92-2.86 (m, 2H), 2.81-2.75 (m, 2H), 2.49-2.47 (m, 2H), 1.83-1.77 (m, 1H), 1.63-1.58 (m, 1H), 1.55 (h, J=7.5 Hz, 2H), 1.51-1.41 (m, 3H), 1.31-1.22 (m, 1H), 0.87 (t, J=7.3 Hz, 3H); ¹³C NMR (151 MHz, DMSO-d₆) δ 166.3, 164.3, 159.3, 139.4, 138.7, 128.1, 128.1, 105.2, 93.1, 75.4, 68.2, 67.2, 38.7, 36.9, 34.0, 27.8, 25.5, 24.1, 22.6, 13.6.

3,5-Dibromo-2-(4-propylphenethyl)-6-((tetrahydro-2H-pyran-2-yl)methoxy)pyridin-4-ol (6). N-bromosuccinimide (447.2 mg, 2.51 mmol) was added to a solution of 5 (406 mg, 1.14 mmol) in anhydrous MeCN (4.3 mL) and stirred under an atmosphere of argon at rt for 18 h. 25 mL water was added to the reaction mixture and extracted with DCM (3×25 mL). The combined organic phase was dried over anhydrous MgSO₄, filtered, evaporated onto celite, and purified by flash chromatography (15% EtOAc in heptane) to afford 6 as a yellow oil (484 mg, 83%). R_{f}=0.37 (EtOAc:heptane; 1:4); ¹H NMR (600 MHz, CDCl₃) δ 7.14 (d, J=8.1 Hz, 2H), 7.10 (d, J=8.0 Hz, 2H), 6.35 (s, 1H), 4.34 (dd, J=11.2, 6.0 Hz, 1H), 4.26 (dd, J=11.2, 4.5 Hz, 1H), 4.07-4.01 (m, 1H), 3.74-3.68 (m, 1H), 3.49 (td, J=11.7, 2.2 Hz, 1H), 3.13-3.05 (m, 2H), 3.00-2.95 (m, 2H), 2.56 (t, J=7.7 Hz, 2H), 1.93-1.87 (m, 1H), 1.74-1.68 (m, 1H), 1.67-1.50 (m, 5H), 1.49-1.41 (m, 1H), 0.94 (t, J=7.3 Hz, 3H); ¹³C NMR (151 MHz, CDCl₃) δ 158.7, 157.4, 157.3, 155.7, 140.5, 138.6, 128.6, 128.4, 101.6, 91.2, 75.9, 70.4, 68.7, 38.6, 37.8, 33.5, 28.4, 26.1, 24.8, 23.2, 14.0.

Example 1, 3-bromo-2-(4-propylphenethyl)-6-((tetrahydro-2H-pyran-2-yl)methoxy)pyridin-4-ol (7). A flame-dried flask under an argon atmosphere was charged with a solution of 6 (220.4 mg, 0.43 mmol) in anhydrous THF (2.6 mL) and cooled down to −78° C. nBuLi (1.6 M in hexanes, 800 μL, 1.28 mmol) was added dropwise and stirred at the same temperature. After 3 h, the reaction was quenched by adding sat. aq. NH₄Cl (15 mL) and extracted with DCM (3×15 mL). The combined organic phase was dried over anhydrous MgSO₄, filtered, concentrated under reduced pressure, and purified by preparative HPLC (50-80% solvent B in 10 min) to afford 7 as a slightly yellow oil that solidifies upon standing (80.8 mg, 43%). R_{f}=0.43 (MeOH:DCM; 5:95); ¹H NMR (600 MHz, CDCl₃) δ 10.05 (br s, 1H), 7.11 (d, J=8.1 Hz, 2H), 7.08 (d, J=7.9 Hz, 2H), 6.53 (s, 1H), 4.05 (d, J=4.8 Hz, 2H), 3.99-3.92 (m, 1H), 3.66-3.59 (m, 1H), 3.42 (td, J=11.5, 2.7 Hz, 1H), 3.20-3.09 (m, 2H), 2.92 (t, J=8.2 Hz, 2H), 2.54 (t, J=7.4 Hz, 2H), 1.91-1.84 (m, 1H), 1.65-1.46 (m, 6H), 1.36 (qd, J=12.2, 3.9 Hz, 1H), 0.93 (t, J=7.3 Hz, 3H); ¹³C NMR (151 MHz, CDCl₃) δ 170.4, 160.7, 152.5, 141.1, 137.1, 128.8, 128.4, 105.5, 94.1, 75.5, 73.1, 68.6, 37.8, 37.0, 33.7, 27.5, 25.7, 24.7, 22.9, 14.0; HPLC: t_{R}=7.99 min, Method A, Purity: 99.84%.

4-(Benzyloxy)-3-bromo-2-(4-propylphenethyl)-6-((tetrahydro-2H-pyran-2-yl)methoxy)pyridine (8). A mixture of 7 (70 mg, 0.16 mmol) and Cs₂CO₃ (78.8 mg, 0.24 mmol) in acetone (650 μL) was stirred at rt for 15 min and then benzyl bromide (19.2 μL, 0.16 mmol) was added. The vial was capped and heated under reflux. After 17 h, the reaction mixture was cooled down and the solvent was evaporated in vacuo. The residue was partitioned between water (10 mL), brine (10 mL) and EtOAc (20 mL) and the phases were separated. The aqueous phase was further extracted with EtOAc (2×20 mL), and the combined organic phase was dried over MgSO₄, filtered, and concentrated under reduced pressure to afford 8 as a slightly yellow oil (67.4 mg, 80%). R_{f}=0.48 (EtOAc:heptane; 1:4); ¹H NMR (400 MHz, CDCl₃) δ 7.48-7.32 (m, 5H), 7.18 (d, J=8.0 Hz, 2H), 7.11 (d, J=8.0 Hz, 2H), 6.23 (s, 1H), 5.13 (s, 2H), 4.31 (dd, J=11.5, 3.3 Hz, 1H), 4.21 (dd, J=11.5, 6.9 Hz, 1H), 4.10-4.03 (m, 1H), 3.72-3.63 (m, 1H), 3.49 (td, J=11.6, 2.4 Hz, 1H), 3.19-3.11 (m, 2H), 3.04-2.95 (m, 2H), 2.57 (t, J=8.2, 7.5 Hz, 2H), 1.95-1.86 (m, 1H), 1.70-1.48 (m, 6H), 1.47-1.36 (m, 1H), 0.95 (t, J=7.3 Hz, 3H); ¹³C NMR (101 MHz, CDCl₃) δ 163.3, 162.8, 157.5, 140.3, 139.2, 135.7, 128.8, 128.5, 128.4, 128.3, 127.1, 103.4, 93.0, 76.2, 70.6, 69.4, 68.7, 39.1, 37.8, 33.7, 28.2, 26.0, 24.8, 22.8, 14.0.

General Procedure for the Suzuki Coupling for the synthesis of compounds 9a, b as exemplified by compound 9a.

4-(Benzyloxy)-3-methyl-2-(4-propylphenethyl)-6-((tetrahydro-2H-pyran-2-yl)methoxy)pyridine (9a). A vial, charged with 8 (25 mg, 47.7 μmol), methylboronic acid (4.3 mg, 71.8 μmol), Cs₂CO₃ (62.1 mg, 0.19 mmol), and PdCl₂(dppf) (10.5 mg, 14.4 μmol), was evacuated and backfilled with argon three times. Then, anhydrous DMF (450 μL) was added and the vial was capped and stirred at 80° C. for 18 h. The cooled reaction mixture was filtered, evaporated onto celite, and purified by flash chromatography (10% EtOAc in heptane) to afford 9a as a colorless oil (19.1 mg, impure).

4-(Benzyloxy)-3-phenyl-2-(4-propylphenethyl)-6-((tetrahydro-2H-pyran-2-yl)methoxy)pyridine (9b). The target compound was synthesized as described for compound 9a by the Suzuki coupling of 8 (25 mg, 47.6 μmol) with phenylboronic acid (8.7 mg, 71.4 μmol) in the presence of Cs₂CO₃ (62.1 mg, 0.19 mmol) and PdCl₂(dppf) (10.5 mg, 14.4 μmol) in anhydrous DMF (450 μL) for 16 h. The crude was purified by flash chromatography (8% EtOAc in heptane) to afford 9b as a colorless oil (19.8 mg 80%). R_{f}=0.49 (EtOAc:heptane; 1:4); ¹H NMR (400 MHz, CDCl₃) δ 7.40-7.19 (m, 6H), 7.17-7.12 (m, 2H), 7.11-7.05 (m, 2H), 7.01 (d, J=8.1 Hz, 2H), 6.90 (d, J=8.1 Hz, 2H), 6.26 (s, 1H), 5.02 (s, 2H), 4.41 (dd, J=11.4, 3.4 Hz, 1H), 4.31 (dd, J=11.4, 6.8 Hz, 1H), 4.14-4.04 (m, 1H), 3.79-3.70 (m, 1H), 3.52 (td, J=11.6, 2.4 Hz, 1H), 2.95-2.87 (m, 2H), 2.79-2.69 (m, 2H), 2.53 (t, J=7.5 Hz, 2H), 1.97-1.86 (m, 1H), 1.74-1.40 (m, 7H), 0.93 (t, J=7.3 Hz, 3H); ¹³C NMR (101 MHz, CDCl₃) δ 164.5, 163.7, 156.8, 140.1, 139.5, 136.5, 135.8, 130.7, 128.5, 128.4, 128.4, 128.1, 127.7, 127.0, 126.4, 121.0, 91.6, 76.4, 69.5, 69.1, 68.7, 37.8, 37.1, 35.0, 28.3, 26.1, 24.8, 23.4, 14.0.

Example 2, 3-Methyl-2-(4-propylphenethyl)-6-((tetrahydro-2H-pyran-2-yl)methoxy)pyridin-4-ol (10a). The target compound was synthesized as described for compound 5 by debenzylation of 9a (19.1 mg, impure) in MeOH:EtOAc (1:2; 1.5 mL), catalyzed by 10% Pd—C (2.2 mg, 2.08 μmol) under hydrogen atmosphere for 1.5 h. The crude was purified by preparative HPLC (35-55% solvent B in 12.5 min) and further purified by flash chromatography (6% MeOH in DCM) to afford 10a as a colorless oil (9.5 mg, 54% over 2 steps). R_{f}=0.21 (MeOH:DCM; 5:95); ¹H NMR (600 MHz, DMSO) δ 10.28 (br s, 1H), 7.09 (d, J=8.1 Hz, 2H), 7.06 (d, J=8.1 Hz, 2H), 5.99 (s, 1H), 4.11-4.05 (m, 2H), 3.89-3.84 (m, 1H), 3.58-3.52 (m, 1H), 3.36-3.34 (m, 1H), 2.89-2.79 (m, 4H), 2.49-2.48 (m, 2H), 1.90 (s, 3H), 1.83-1.76 (m, 1H), 1.61-1.51 (m, 3H), 1.50-1.41 (m, 3H), 1.29-1.22 (m, 1H), 0.87 (t, J=7.3 Hz, 3H); ¹³C NMR (151 MHz, DMSO) δ 164.2, 161.4, 156.2, 139.3, 139.1, 128.1, 111.8, 92.8, 75.5, 67.9, 67.2, 36.9, 36.1, 33.5, 27.8, 25.6, 24.1, 22.6, 13.6, 9.8; HPLC: t_{R}=4.37 min, Method A, Purity: 98.95%.

Example 3, 3-Phenyl-2-(4-propylphenethyl)-6-((tetrahydro-2H-pyran-2-yl)methoxy)pyridin-4-ol (10b). The target compound was synthesized as described for compound 5 by debenzylation of 9b (19.8 mg, 37.9 μmol) in MeOH:EtOAc (1:2; 1.5 mL), catalyzed by 10% Pd—C (2.0 mg, 1.88 μmol) under hydrogen atmosphere for 2.5 h. The crude was purified by flash chromatography (5-6% MeOH in DCM) to afford 10b as a colorless oil (14.9 mg, 91%). R_{f}=0.34 (MeOH:DCM; 5:95); ¹H NMR (400 MHz, DMSO) δ 10.29 (s, 1H), 7.41-7.22 (m, 3H), 7.07-6.93 (m, 4H), 6.83 (d, J=8.0 Hz, 2H), 6.11 (s, 1H), 4.25-4.11 (m, 2H), 3.93-3.86 (m, 1H), 3.66-3.58 (m, 1H), 3.41-3.35 (m, 1H), 2.85-2.74 (m, 2H), 2.62-2.54 (m, 2H), 2.46 (t, J=7.5 Hz, 2H), 1.87-1.76 (m, 1H), 1.67-1.60 (m, 1H), 1.58-1.42 (m, 5H), 1.37-1.25 (m, 1H), 0.85 (t, J=7.3 Hz, 3H); ¹³C NMR (101 MHz, DMSO) δ 163.8, 162.6, 156.1, 139.3, 138.8, 135.7, 130.3, 128.1, 127.9, 127.9, 126.7, 119.5, 93.0, 75.4, 68.1, 67.3, 36.8, 36.5, 34.0, 27.8, 25.6, 24.1, 22.6, 13.6; HPLC: t_{R}=5.38 min, Method A, Purity: 99.06%.

4-(Benzyloxy)-2,6-dichloronicotinaldehyde (11). To a solution of 2 (250 mg, 0.98 mmol) in anhydrous THF (3 mL), nBuLi (1.6 M in hexanes, 0.64 mL, 1.02 mmol) was added dropwise at −78° C. After 30 min ethyl formate (0.1 mL, 1.24 mmol) was added dropwise and the reaction mixture was stirred at −78° C. for 1 h. The solution was quenched with sat. aq. NH₄Cl (10 mL) and extracted with EtOAc (3×10 mL). The combined organic phase was dried over Na₂SO₄, filtered and the solvents were evaporated under reduced pressure. The residue was purified by silica gel column chromatography (6-10% EtOAc in heptane) to afford 11 (161 mg, 58%) as white solid. R_{f}=0.36 (PE:EtOAc; 9:1). ¹H NMR (400 MHz, CDCl₃) δ 10.43 (s, 1H), 7.46-7.36 (m, 5H), 6.98 (s, 1H), 5.25 (s, 2H). ¹³C NMR (101 MHz, CDCl₃) δ 186.8, 168.0, 154.9, 153.0, 133.9, 129.2, 129.1, 127.3, 118.0, 108.5, 72.0.

4-(Benzyloxy)-2,6-dichloro-3-(dimethoxymethyl)pyridine (12). Trimethyl orthoformate (0.22 mL, 2.01 mmol), p-toluene sulphonic acid monohydrate (4.7 mg, 0.025 mmol) and 11 (140.0 mg, 0.496 mmol) were dissolved in dry MeOH (5 mL) and the mixture was refluxed. After 12 h, EtOAc (10 mL) was added and the mixture was washed with sat. NaHCO₃ (2×10 mL) and water (1×10 mL). The combined aq. phase was extracted with EtOAc (2×10 mL). The combined organic extract was dried over Na₂SO₄ and the solvents were evaporated under reduced pressure to afford 12 (163 mg, quant.) as colorless oil. R_{f}=0.49 (PE:EtOAc; 8:2). ¹H NMR (400 MHz, CDCl₃) δ 7.43-7.33 (m, 5H), 6.83 (s, 1H), 5.71 (s, 1H), 5.22 (s, 2H), 3.47 (s, 6H). ¹³C NMR (101 MHz, CDCl₃) δ 166.1, 151.1, 150.3, 134.9, 129.0, 128.7, 127.0, 120.1, 108.1, 102.4, 71.3, 56.2.

4-(Benzyloxy)-2-chloro-3-(dimethoxymethyl)-6-((tetrahydro-2H-pyran-2-yl)methoxy)pyridine (13). NaH (60% in mineral oil, 39.7 mg, 0.993 mmol) was added to a solution of 12 (163 mg, 0.497 mmol) and (tetrahydro-2H-pyran-2-yl)methanol (0.06 mL, 0.531 mmol) in THF (1.5 mL) at 0° C. and the reaction was stirred at the same temperature for 30 min before moving the vial to a heating block at 80° C. After 5 h the mixture was allowed to cool to rt, water (5 mL) and brine (5 mL) were added, and the mixture was extracted with EtOAc (3×10 mL). The combined organic phase was dried over Na₂SO₄, filtered and the solvents were evaporated under reduced pressure. The residue was purified by silica gel column chromatography (9-12% EtOAc in heptane) to afford 13 (76 mg, 38%) as colorless oil. R_{f}=0.38 (PE:EtOAc; 8:2). ¹H NMR (600 MHz, CDCl₃) δ 7.43-7.35 (m, 4H), 7.32 (tt, J=7.2, 1.5 Hz, 1H), 6.28 (s, 1H), 5.69 (s, 1H), 5.14 (s, 2H), 4.31 (dd, J=11.4, 2.9 Hz, 1H), 4.19 (dd, J=11.4, 7.2 Hz, 1H), 4.04 (ddt, J=11.4, 3.8, 1.6 Hz, 1H), 3.65 (ddt, J=12.0, 7.1, 2.5 Hz, 1H), 3.50-3.40 (m, 7H), 1.91-1.85 (m, 1H), 1.62-1.48 (m, 4H), 1.43-1.36 (m, 1H). ¹³C NMR (151 MHz, CDCl₃) δ 166.7, 163.8, 148.3, 135.7, 128.8, 128.2, 126.9, 114.6, 102.7, 93.8, 75.9, 70.6, 69.9, 68.6, 55.9, 27.9, 25.9, 23.2.

4-(Benzyloxy)-2-chloro-6-((tetrahydro-2H-pyran-2-yl)methoxy)nicotinaldehyde (14). A solution of 13 (76 mg, 0.186 mmol) in THF (0.3 mL) and HCl (1 N, 0.3 mL), was stirred for 2 h at rt. Then it quenched with sat. NaHCO₃ (1 mL), water (4 mL) was added and the mixture was extracted with EtOAc (3×5 mL). The combined organic layers were dried over Na₂SO₄ and the solvents were evaporated under reduced pressure to afford 14 as a white solid (64 mg, 95%). R_{f}=0.38 (PE:EtOAc; 8:2). ¹H NMR (400 MHz, CDCl₃) δ 10.40 (s, 1H), 7.45-7.32 (m, 5H), 6.36 (s, 1H), 5.17 (s, 2H), 4.40 (dd, J=11.5, 2.9 Hz, 1H), 4.29 (dd, J=11.5, 7.1 Hz, 1H), 4.07-4.02 (m, 1H), 3.68 (ddt, J=11.8, 7.1, 2.6 Hz, 1H), 3.48 (td, J=11.5, 2.4 Hz, 1H), 1.94-1.86 (m, 1H), 1.63-1.53 (m, 4H), 1.47-1.37 (m, 1H). ¹³C NMR (101 MHz, CDCl₃) δ 187.2, 168.9, 166.2, 153.1, 134.8, 129.0, 128.6, 127.1, 114.3, 93.7, 75.7, 71.2, 70.6, 68.6, 27.8, 25.9, 23.2.

4-(Benzyloxy)-2-chloro-6-((tetrahydro-2H-pyran-2-yl)methoxy)nicotinonitrile (15). A solution of 14 (64.0 mg, 0.177 mmol) and hydroxylamine hydrochloride (13.5 mg, 0.194 mmol) in DMSO (1 mL) was stirred at 90° C. for 24 h. Then water (15 mL) was added and the mixture was extracted with DEE (3×10 mL). The combined organic phase was dried over Na₂SO₄ and the solvent was evaporated under reduced pressure. The residue was purified by silica gel column chromatography (12-15% EtOAc in heptane) to afford 15 (36 mg, 57%) as white solid. R_{f}=0.35 (PE:EtOAc; 8:2). ¹H NMR (400 MHz, CDCl₃) δ 7.44-7.34 (m, 5H), 6.31 (s, 1H), 5.18 (s, 2H), 4.36 (dd, J=11.5, 2.9 Hz, 1H), 4.26 (dd, J=11.5, 7.1 Hz, 1H), 4.07-4.01 (m, 1H), 3.69-3.62 (m, 1H), 3.47 (td, J=11.5, 2.6 Hz, 1H), 1.93-1.87 (m, 1H), 1.65-1.52 (m, 4H), 1.45-1.36 (m, 1H). ¹³C NMR (101 MHz, CDCl₃) δ 168.8, 166.2, 153.1, 134.2, 129.1, 128.9, 127.2, 113.1, 95.0, 93.2, 75.6, 71.6, 70.9, 68.6, 27.8, 25.8, 23.1.

4-(Benzyloxy)-2-((4-ethoxyphenyl)ethynyl)-6-((tetrahydro-2H-pyran-2-yl)methoxy)nicotinonitrile (16). A vial was charged with PdCl₂(MeCN)₂ (1.0 mg, 3.9 μmol), XPhos (5.7 mg, 12.0 μmol) and Cs₂CO₃ (85.0 mg, 0.261 mmol). The vial was capped, evacuated and backfilled with Ar (×3) and the contents were suspended in anhydrous and degassed MeCN (0.2 mL). 1-Ethoxy-4-ethynylbenzene (13.8 mg, 0.094 mmol) was added and the reaction was allowed to stir for 15 min after which a solution of 15 (36 mg, 0.100 mmol) in anhydrous and degassed MeCN (0.4 mL) was added. The vial was heated to 80° C. for 17 h. The reaction mixture was filtered through a celite pad, washed with DEE (15 mL), the solvents were evaporated under reduced pressure and the residue was purified by silica gel column chromatography (13-17% EtOAc in heptane) to afford 16 (9 mg, 19%) as a yellow oil. R_{f}=0.25 (EtOAc:PE; 2:8). ¹H NMR (400 MHz, CDCl₃) δ 7.66-7.56 (m, 2H), 7.45-7.33 (m, 5H), 6.91-6.86 (m, 2H), 6.32 (s, 1H), 5.19 (s, 2H), 4.43 (dd, J=11.5, 2.9 Hz, 1H), 4.30 (dd, J=11.5, 7.2 Hz, 1H), 4.15-3.94 (m, 3H), 3.72-3.64 (m, 1H), 3.49 (td, J=11.6, 2.4 Hz, 1H), 1.94-1.86 (m, 1H), 1.66-1.51 (m, 6H), 1.46-1.39 (m, 4H). ¹³C NMR (101 MHz, CDCl₃) δ 167.3, 166.9, 160.5, 146.4, 134.6, 134.4, 129.0, 128.7, 127.2, 114.8, 114.6, 113.2, 98.1, 96.3, 93.8, 85.4, 75.8, 71.1, 70.4, 68.6, 63.8, 27.9, 25.9, 23.2, 14.8.

Example 4, 2-(4-Ethoxyphenethyl)-4-hydroxy-6-((tetrahydro-2H-pyran-2-yl)methoxy)nicotinonitrile (17). 10% Pd—C (1.0 mg, 5%) was added in a solution of 16 (9.0 mg, 0.019 mmol) in MeOH:EtOAc (1:2, 0.9 mL). The flask was evacuated under vacuum and backfilled with Ar three times, then a balloon with H₂ was attached. The flask was evacuated under vacuum and backfilled with H₂ three times and the mixture was stirred at rt for 2 h. After removal of the H₂, the reaction mixture was evaporated on celite and chromatographed (6% MeOH in DCM) to afford 2-(4-ethoxyphenethyl)-4-hydroxy-6-((tetrahydro-2H-pyran-2-yl)methoxy)nicotinonitrile (5 mg, 68%) as slightly yellow oil. R_{f}=0.55 (DCM:MeOH; 9:1). 1H NMR (600 MHz, CDCl₃) δ 7.10 (d, J=8.4 Hz, 2H), 6.80 (d, J=8.4 Hz, 2H), 6.42 (s, 1H), 4.35-4.27 (m, 1H), 4.23-4.12 (m, 1H), 4.07-3.97 (m, 3H), 3.83-3.73 (m, 1H), 3.55-3.48 (m, 1H), 3.17 (t, J=7.1 Hz, 2H), 3.01 (t, J=7.7 Hz, 2H), 1.96-1.88 (m, 1H), 1.66-1.37 (m, 8H). ¹³C NMR (151 MHz, CDCl₃) δ 169.9, 164.5, 163.7, 157.7, 132.2, 129.6, 114.7, 114.6, 94.5, 94.1, 76.4, 70.8, 68.6, 63.6, 37.7, 34.0, 27.6, 25.8, 22.9, 15.0.

Methyl 2-acetamido-4-bromobenzoate (19). A flame dried round bottom flask was sequentially charged with methyl 2-amino-4-bromobenzoate (18) (500 mg, 2.17 mmol, 1 eq), DCM (10 mL), and triethylamine (0.34 mL, 2.44 mmol, 1.1 eq). Then acetyl chloride (0.23 mL, 3.23 mmol, 1.5 eq) was added at rt. After 17 h, DCM was added (10 mL) and the reaction mixture was washed with sat. NaHCO₃ (2×20 mL), HCl (1 M, 2×20 mL) and brine (20 mL). The organic phase was dried over Na₂SO₄, filtered and the solvent was evaporated under reduced pressure to afford methyl 2-acetamido-4-bromobenzoate (570 mg, 96%) as white solid. R_{f}=0.42 (EtOAc:PE; 2:8). ¹H NMR (400 MHz, CDCl₃) δ 11.06 (s, 1H), 8.96 (d, J=2.0 Hz, 1H), 7.85 (d, J=8.6 Hz, 1H), 7.20 (dd, J=8.6, 2.0 Hz, 1H), 3.92 (s, 3H), 2.23 (s, 3H). ¹³C NMR (101 MHz, CDCl₃) δ 169.2, 168.4, 142.5, 132.0, 129.8, 125.8, 123.3, 113.5, 52.6, 25.6.

7-Bromo-2,4-dichloroquinoline (20). Step I: To a solution of 19 (570 mg, 2.09 mmol) in anhydrous THF (5.6 mL), KHMDS (1 M in THF, 6.28 mL, 6.28 mmol) was added dropwise at −78° C. After 1 h the cooling bath was removed and the mixture was let to warm up to rt. After 18 h, water (15 mL) was added and the mixture was washed with EtOAc (2×5 mL). The aq. phase was acidified with sat. aq. KHSO₄ and the precipitate was filtered and washed with EtOAc (10 mL) and water (10 mL). The solid was dried under reduced pressure to afford 20 (400 mg, 80%) as white solid. ¹H NMR (400 MHz, DMSO) δ 11.51 (s, 1H), 11.25 (s, 1H), 7.69 (d, J=8.5 Hz, 1H), 7.44 (d, J=1.9 Hz, 1H), 7.29 (dd, J=8.6, 1.9 Hz, 1H), 5.74 (s, 1H). ¹³C NMR (101 MHz, DMSO) δ 163.3, 162.0, 140.2, 124.7, 123.9, 123.9, 117.2, 114.2, 98.6.

Step II: POCl₃ (2.5 mL) was added to a flask containing 7-bromoquinoline-2,4-diol (130 mg, 0.542 mmol) and the mixture was set under reflux conditions. After 2.5 h, the mixture was cooled down, quenched with ice and the mixture was extracted with DCM (3×10 mL). The combined organic phase was washed with NaOH (1 M, 2×10 mL), dried over Na₂SO₄ and evaporated under reduced pressure to give 20 (148 mg, 99%) as white solid. ¹H NMR (400 MHz, CDCl₃) δ 8.21 (d, J=1.9 Hz, 1H), 8.05 (d, J=8.9 Hz, 1H), 7.73 (dd, J=8.9, 1.9 Hz, 1H), 7.51 (s, 1H). ¹³C NMR (101 MHz, CDCl₃) δ 151.2, 148.7, 144.6, 131.6, 131.5, 126.3, 125.7, 124.2, 122.5.

7-Bromo-4-chloro-2-((tetrahydro-2H-pyran-2-yl)methoxy)quinoline (21). To a solution of (tetrahydro-2H-pyran-2-yl)methanol (0.035 mL, 0.309 mmol) in THF (0.2 mL), NaH (60% in mineral oil, 22.8 mg, 0.570 mmol) was added at 0° C., the ice-water bath was removed and the mixture was stirred for 20 min. Then a solution of 20 (78.1 mg, 0.282 mmol) in THF (0.4 mL) was added and the mixture was stirred at rt for 2.5 h. Then sat. NH₄Cl (5 mL) and water (5 mL) were added, the phases were separated and the aq. phase was further extracted with EtOAc (2×10 mL). The combined org. phase was dried over Na₂SO₄, filtered and the solvents were evaporated under reduced pressure. The residue was purified by silica gel column chromatography (6-10% EtOAc in heptane) to afford 21 (67 mg, 67%) as white solid. Rf=0.62 (EtOAc:PE, 0.5:9.5). ¹H NMR (600 MHz, CDCl₃) δ 8.02 (d, J=2.0 Hz, 1H), 7.94 (d, J=8.8 Hz, 1H), 7.53 (dd, J=8.8, 1.9 Hz, 1H), 7.10 (s, 1H), 4.48 (dd, J=11.5, 3.2 Hz, 1H), 4.39 (dd, J=11.5, 7.0 Hz, 1H), 4.09-4.04 (m, 1H), 3.78-3.72 (m, 1H), 3.51 (td, J=11.7, 2.2 Hz, 1H), 1.95-1.89 (m, 1H), 1.71-1.52 (m, 5H), 1.51-1.44 (m, 1H). ¹³C NMR (151 MHz, CDCl₃) δ 162.3, 147.7, 143.8, 130.1, 128.3, 125.6, 124.9, 122.4, 113.7, 75.9, 69.8, 68.7, 28.1, 26.0, 23.3.

4-(Benzyloxy)-7-bromo-2-((tetrahydro-2H-pyran-2-yl)methoxy)quinoline (22). To a solution of benzyl alcohol (0.02 mL, 0.193 mmol) in DMF (0.2 mL), NaH (60% in mineral oil, 11.3 mg, 0.283 mmol) was added at 0° C., the ice-water bath was removed and the mixture was stirred for 20 min. Then a solution of 21 (67.0 mg, 0.188 mmol) in DMF (0.5 mL) was added and the mixture was stirred at rt for 18 h. Then sat. NH₄Cl (5 mL), water (5 mL) and EtOAc (10 mL) were added, the phases were separated and the aq. phase was further extracted with EtOAc (2×10 mL). The combined org. phase was dried over Na₂SO₄, filtered and the solvents were evaporated under reduced pressure. The residue was purified by silica gel column chromatography (6-8% EtOAc in heptane) to afford 22 (50 mg, 62%) as colorless oil. Rf=0.53 (EtOAc:PE, 1:9). ¹H NMR (600 MHz, CDCl₃) δ 8.05 (s, 1H), 7.95 (d, J=8.7 Hz, 1H), 7.56-7.31 (m, 6H), 6.42 (s, 1H), 5.21 (s, 2H), 4.55 (dd, J=11.5, 3.0 Hz, 1H), 4.40 (dd, J=11.5, 7.1 Hz, 1H), 4.09-4.05 (m, 1H), 3.80-3.72 (m, 1H), 3.51 (td, J=11.6, 2.1 Hz, 1H), 1.94-1.89 (m, 1H), 1.71-1.45 (m, 5H). ¹³C NMR (151 MHz, CDCl₃) δ 164.1, 163.3, 147.4, 135.5, 129.1, 128.9, 128.7, 127.7, 127.0, 124.6, 123.6, 118.1, 92.6, 76.1, 70.7, 70.0, 68.7, 28.0, 26.0, 23.3.

4-(Benzyloxy)-7-(4-ethoxyphenyl)-2-((tetrahydro-2H-pyran-2-yl)methoxy)quinoline (23). Degassed NMP (0.86 mL) and water (0.14 mL) were added to a vial containing 22 (36.0 mg, 0.084 mmol), (4-ethoxyphenyl)boronic acid (22.3 mg, 0.134 mmol), palladium acetate (1.0 mg, 4.5 μmol) and cesium carbonate (109.5 mg, 0.110 mmol), the vial was sealed and transferred to a heating block at 90° C. After 5 h, water (10 mL) was added and the mixture was extracted with DEE (3×10 mL). The combined organic phase was dried over MgSO₄, filtered and the solvents were evaporated under reduced pressure at rt. The residue was purified by silica gel column chromatography (8-9% EtOAc in heptane) to afford 23 (28 mg, 71%) as colorless oil. R_{f}=0.55 (PE:EtOAc, 8:2). ¹H NMR (600 MHz, CDCl₃) δ 8.14 (d, J=8.5 Hz, 1H), 7.96 (s, 1H), 7.73-7.62 (m, 2H), 7.59-7.55 (m, 1H), 7.50 (d, J=7.4 Hz, 2H), 7.44 (t, J=7.8 Hz, 2H), 7.39 (tt, J=7.4, 2.1 Hz, 1H), 7.06-6.92 (m, 2H), 6.40 (s, 1H), 5.24 (s, 2H), 4.64-4.52 (m, 1H), 4.49-4.35 (m, 1H), 4.16-4.02 (m, 3H), 3.83-3.72 (m, 1H), 3.52 (td, J=11.6, 2.1 Hz, 1H), 1.96-1.88 (m, 1H), 1.74-1.49 (m, 5H), 1.45 (t, J=7.0 Hz, 3H). ¹³C NMR (151 MHz, CDCl₃) δ 163.8, 163.2, 159.0, 147.3, 142.7, 135.9, 133.0, 128.9, 128.6, 128.5, 127.6, 124.0, 122.7, 122.6, 118.0, 115.0, 92.0, 76.2, 70.5, 69.4, 68.7, 63.7, 28.1, 26.0, 23.3, 15.0.

Example 5, 7-(4-Ethoxyphenyl)-2-((tetrahydro-2H-pyran-2-yl)methoxy)quinolin-4-ol (24). 10% Pd—C (3.2 mg, 0.003 mmol) was added in a solution of 4-(benzyloxy)-7-(4-ethoxyphenyl)-2-((tetrahydro-2H-pyran-2-yl)methoxy)quinoline (28.0 mg, 0.060 mmol) in MeOH:EtOAc (2:1, 1.2 mL). The flask was evacuated under vacuum and backfilled with Ar three times, then a balloon with H₂ was attached. The flask was evacuated under vacuum and backfilled with H₂ three times and the mixture was stirred at rt for 2 h. After removal of the H₂, the reaction mixture was evaporated on Celite and chromatographed (5-6% MeOH in DCM) to afford 24 (10.3 mg, 46%) as white solid. R_{f}=0.48 (DCM:MeOH, 9:1). ¹H NMR (600 MHz, MeOD) δ 8.16 (d, J=8.5 Hz, 1H), 7.70-7.60 (m, 3H), 7.59 (dd, J=8.5, 1.7 Hz, 1H), 7.07-7.01 (m, 2H), 5.93 (s, 1H), 4.24-4.17 (m, 2H), 4.10 (q, J=7.0 Hz, 2H), 4.02-3.99 (m, 1H), 3.80-3.76 (m, 1H), 3.52 (td, J=11.2, 3.0 Hz, 1H), 1.96-1.92 (m, 1H), 1.74-1.70 (m, 1H), 1.65-1.48 (m, 4H), 1.42 (t, J=7.0 Hz, 3H). ¹³C NMR (151 MHz, MeOD) δ 165.2, 163.3, 160.9, 146.2, 141.2, 133.1, 129.4, 126.3, 123.3, 121.9, 116.1, 116.1, 90.7, 76.9, 73.2, 69.4, 64.6, 28.6, 26.9, 24.0, 15.1. HPLC: t_{R}=8.04 min, Method C, Purity: 99.99%.

4-(Benzyloxy)-2-(4-propylphenethyl)-6-((tetrahydro-2H-pyran-2-yl)methoxy)-3-vinylpyridine (9c). The target compound was synthesized as described for compound 9a by the Suzuki coupling of 8 (19.4 mg, 37.0 μmol) with 4,4,5,5-tetramethyl-2-vinyl-1,3,2-dioxaborolane (18.8 μL, 0.11 mmol) in the presence of Cs₂CO₃ (48.2 mg, 0.15 mmol), and PdCl₂(dppf) (8.1 mg, 11.1 μmol) in anhydrous DMF (350 μL) for 17 h. The crude was purified by flash chromatography (12% EtOAc in heptane) to afford 9b as a colorless oil (12.2 mg, 70%). R_{f}=0.20 (EtOAc:heptane; 1:9); ¹H NMR (600 MHz, CDCl₃) δ 7.41-7.36 (m, 4H), 7.35-7.30 (m, 1H), 7.14 (d, J=8.0 Hz, 2H), 7.09 (d, J=8.0 Hz, 2H), 6.67 (dd, J=17.8, 11.7 Hz, 1H), 6.24 (s, 1H), 5.59 (dd, J=17.8, 2.1 Hz, 1H), 5.40 (dd, J=11.7, 2.1 Hz, 1H), 5.07 (s, 2H), 4.36 (dd, J=11.4, 3.4 Hz, 1H), 4.25 (dd, J=11.4, 6.9 Hz, 1H), 4.10-4.03 (m, 1H), 3.73-3.66 (m, 1H), 3.49 (td, J=11.8, 2.2 Hz, 1H), 3.09-3.03 (m, 2H), 3.00-2.96 (m, 2H), 2.56 (t, J=7.5, 6.9 Hz, 2H), 1.93-1.87 (m, 1H), 1.69-1.56 (m, 4H), 1.55-1.49 (m, 2H), 1.48-1.38 (m, 1H), 0.94 (t, J=7.3 Hz, 3H); ¹³C NMR (151 MHz, CDCl₃) δ 165.2, 163.0, 157.1, 140.2, 139.6, 136.3, 129.5, 128.8, 128.5, 128.4, 128.2, 127.4, 119.2, 116.2, 91.6, 76.4, 70.1, 69.0, 68.7, 37.8, 37.2, 34.7, 28.3, 26.1, 24.8, 23.3, 14.0.

(E)-4-(benzyloxy)-3-(prop-1-en-1-yl)-2-(4-propylphenethyl)-6-((tetrahydro-2H-pyran-2-yl)methoxy)pyridine (9d). The target compound was synthesized as described for compound 9a by the Suzuki coupling of 8 (15.4 mg, 29.4 μmol) with (E)-prop-1-en-1-ylboronic acid (3.8 mg, 44.2 μmol) in the presence of Cs₂CO₃ (38.3 mg, 0.12 mmol), and PdCl₂(dppf) (6.5 mg, 8.88 mol) in anhydrous DMF (280 μL) for 34 h. The crude was purified by flash chromatography (10% EtOAc in heptane) to afford 9d as a as a slightly yellow oil (10.3 mg). R_{f}=0.46 (EtOAc:heptane; 1:4)

4-(Benzyloxy)-3-(prop-1-en-2-yl)-2-(4-propylphenethyl)-6-((tetrahydro-2H-pyran-2-yl)methoxy)pyridine (9e). The target compound was synthesized as described for compound 9a by the Suzuki coupling of 8 (30.0 mg, 57.2 μmol) with 4,4,5,5-tetramethyl-2-(prop-1-en-2-yl)-1,3,2-dioxaborolane (30 μL, 0.16 mmol) in the presence of Cs₂CO₃ (74.5 mg, 0.23 mmol), and PdCl₂(dppf) (12.6 mg, 17.2 μmol) in anhydrous DMF (450 μL) for 15 h. The crude was purified by flash chromatography (6% EtOAc in heptane) to afford 9e as a colorless oil (11.0 mg, 39%). ¹H NMR (600 MHz, CDCl₃) δ 7.40-7.28 (m, 5H), 7.12-7.04 (m, 4H), 6.21 (s, 1H), 5.23 (s, 1H), 5.06 (s, 2H), 4.73 (s, 1H), 4.38 (dd, J=11.4, 3.5 Hz, 1H), 4.27 (dd, J=11.4, 6.8 Hz, 1H), 4.09-4.03 (m, 1H), 3.75-3.68 (m, 1H), 3.53-3.46 (m, 1H), 3.02-2.92 (m, 4H), 2.57-2.52 (m, 2H), 1.94-1.87 (m, 4H), 1.72-1.38 (m, 7H), 0.93 (t, J=7.3 Hz, 3H).

(4-(Benzyloxy)-2-(4-propylphenethyl)-6-((tetrahydro-2H-pyran-2-yl)methoxy)-3-(o-tolyl)pyridine (9f). The target compound was synthesized as described for compound 9a by the Suzuki coupling of 8 (14.2 mg, 27.1 μmol) with o-tolylboronic acid (11.0 mg, 80.9 μmol) in the presence of Cs₂CO₃ (35.3 mg, 0.11 mmol), and PdCl₂(dppf) (5.9 mg, 8.06 μmol) in anhydrous DMF (260 μL) for 21.5 h. The crude was purified by flash chromatography (8% EtOAc in heptane) to afford 9f as a as a slightly yellow oil (12.6 mg). R_{f}=0.58 (EtOAc:heptane; 1:4)

(4-(Benzyloxy)-2-(4-propylphenethyl)-6-((tetrahydro-2H-pyran-2-yl)methoxy)-3-(p-tolyl)pyridine (9g). The target compound was synthesized as described for compound 9a by the Suzuki coupling of 8 (15.8 mg, 30.1 μmol) with p-tolylboronic acid (12.3 mg, 90.5 μmol) in the presence of Cs₂CO₃ (39.3 mg, 0.12 mmol), and PdCl₂(dppf) (6.6 mg, 9.02 μmol) in anhydrous DMF (280 μL) for 21.5 h. The crude was purified by flash chromatography (8% EtOAc in heptane) to afford 9g as a as a slightly yellow oil (12.8 mg, 79%). R_{f}=0.56 (EtOAc:heptane; 1:4); ¹H NMR (400 MHz, CDCl₃) δ 7.34-7.26 (m, 2H), 7.26-7.20 (m, 1H), 7.19-7.13 (m, 4H), 7.01 (d, J=8.0 Hz, 2H), 6.98 (d, J=7.9 Hz, 2H), 6.92 (d, J=7.9 Hz, 2H), 6.23 (s, 1H), 5.02 (s, 2H), 4.40 (dd, J=11.5, 3.4 Hz, 1H), 4.29 (dd, J=11.4, 6.8 Hz, 1H), 4.11-4.04 (m, 1H), 3.78-3.69 (m, 1H), 3.51 (td, J=11.7, 2.3 Hz, 1H), 2.96-2.86 (m, 2H), 2.78-2.70 (m, 2H), 2.57-2.49 (m, 2H), 2.39 (s, 3H), 1.95-1.87 (m, 1H), 1.72-1.57 (m, 5H), 1.55-1.39 (m, 2H), 0.93 (t, J=7.3 Hz, 3H); ¹³C NMR (101 MHz, CDCl₃) δ 164.6, 163.5, 156.9, 140.1, 139.6, 136.6, 136.5, 132.7, 130.5, 128.9, 128.6, 128.5, 128.3, 127.7, 126.5, 120.9, 91.6, 76.4, 69.5, 69.1, 68.7, 37.8, 37.0, 34.9, 28.3, 26.1, 24.8, 23.3, 21.4, 14.0.

(E)-4-(benzyloxy)-2-(4-propylphenethyl)-3-styryl-6-((tetrahydro-2H-pyran-2-yl)methoxy)pyridine (9h). The target compound was synthesized as described for compound 9a by the Suzuki coupling of 8 (14.3 mg, 27.2 μmol) with (E)-styrylboronic acid (6.0 mg, 40.6 mol) in the presence of (35.5 mg, 0.11 mmol) and PdCl₂(dppf) (6.0 mg, 8.20 μmol) in anhydrous DMF (260 μL) for 34 h. The crude was purified by flash chromatography (10% EtOAc in heptane) to afford 9h as a as a slightly yellow oil (8.4 mg). R_{f}=0.42 (EtOAc:heptane; 1:4)

Ethyl (E)-3-(4-(benzyloxy)-2-(4-propylphenethyl)-6-((tetrahydro-2H-pyran-2-yl)methoxy)pyridin-3-yl)acrylate (9i). The target compound was synthesized as described for compound 9a by the Suzuki coupling of 8 (151.1 mg, 0.29 μmol) with ethyl (E)-3-(4,4,5,5-tetramethyl-1,3,2-dioxaborolan-2-yl)acrylate (195.4 mg, 0.86 mmol) in the presence of Cs₂CO₃ (374.5 mg, 1.15 mmol), and PdCl₂(dppf) (63.2 mg, 86.4 μmol) in anhydrous DMF (2.5 mL) for 15 h. The crude was purified by preparative HPLC 65% solvent B) to afford 9i as a colorless oil (116.2 mg, 74%). R_{f}=0.54 (EtOAc:heptane; 1:2); ¹H NMR (600 MHz, CDCl₃) δ 7.72 (d, J=16.1 Hz, 1H), 7.45-7.33 (m, 5H), 7.12 (d, J=7.8 Hz, 2H), 7.08 (d, J=7.8 Hz, 2H), 6.56 (d, J=16.1 Hz, 1H), 6.36 (s, 1H), 5.20 (s, 2H), 4.35-4.27 (m, 1H), 4.27-4.20 (m, 3H), 4.05-3.99 (m, 1H), 3.70 (t, J=8.8 Hz, 1H), 3.51-3.44 (m, 1H), 3.20-3.14 (m, 2H), 3.00-2.95 (m, 2H), 2.57-2.52 (m, 2H), 1.94-1.87 (m, 1H), 1.68-1.49 (m, 6H), 1.46-1.37 (m, 1H), 1.32 (t, J=7.1 Hz, 3H), 0.93 (t, J=7.3 Hz, 3H).

Example 6, 3-Ethyl-2-(4-propylphenethyl)-6-((tetrahydro-2H-pyran-2-yl)methoxy)pyridin-4-ol (10c). The target compound was synthesized as described for compound 5 by debenzylation and hydrogenation of 9c (12.2 mg, 25.9 μmol) in MeOH:EtOAc (1:2; 1.5 mL), catalyzed by 10% Pd—C (1.4 mg, 1.32 μmol) under hydrogen atmosphere for 4.5 h. The crude was purified by flash chromatography (6% MeOH in DCM) to afford 10c as a slightly yellow oil (8.7 mg, 88%). R_{f}=0.23 (MeOH:DCM; 5:95); ¹H NMR (600 MHz, DMSO) δ 10.24 (s, 1H), 7.09 (d, J=8.2 Hz, 2H), 7.06 (d, J=8.1 Hz, 2H), 5.99 (s, 1H), 4.13-4.06 (m, 2H), 3.89-3.85 (m, 1H), 3.60-3.54 (m, 1H), 3.37-3.32 (m, 1H), 2.92-2.87 (m, 2H), 2.84-2.80 (m, 2H), 2.49-2.47 (m, 2H), 2.41 (q, J=7.4 Hz, 2H), 1.83-1.77 (m, 1H), 1.62-1.52 (m, 3H), 1.49-1.42 (m, 3H), 1.30-1.25 (m, 1H), 0.92 (t, J=7.4 Hz, 3H), 0.87 (t, J=7.3 Hz, 3H); ¹³C NMR (151 MHz, DMSO) δ 164.6, 161.9, 156.1, 139.8, 139.6, 128.6, 128.6, 118.7, 93.5, 75.9, 68.4, 67.7, 37.3, 35.9, 34.6, 28.3, 26.1, 24.6, 23.1, 18.1, 14.6, 14.1; HPLC: t_{R}=5.06 min, Method A, Purity: 95.80%.

Example 7, 3-Propyl-2-(4-propylphenethyl)-6-((tetrahydro-2H-pyran-2-yl)methoxy)pyridin-4-ol (10d). The target compound was synthesized as described for compound 5 by debenzylation and hydrogenation of 9d (10.3 mg, 21.2 μmol) in MeOH:EtOAc (1:2; 1.5 mL), catalyzed by 10% Pd—C (1.4 mg, 1.32 μmol) under hydrogen atmosphere for 4.0 h. The crude was filtered through Celite, the filtrate was concentrated and purified by preparative HPLC (50-75% solvent B in 8.8 min) to afford 10d as a slightly yellow oil (6.2 mg, 53% over 2 steps). R_{f}=0.47 (MeOH:DCM; 1:9); ¹H NMR (600 MHz, DMSO) δ 7.13-7.06 (m, 4H), 6.30 (br s, 1H), 4.23-4.11 (m, 2H), 3.91-3.86 (m, 1H), 3.67-3.63 (m, 1H), 3.39-3.36 (m, 1H), 2.94-2.83 (m, 4H), 2.52-2.51 (m, 2H), 2.41-2.35 (m, 2H), 1.85-1.80 (m, 1H), 1.64-1.59 (m, 1H), 1.59-1.52 (m, 2H), 1.52-1.44 (m, 3H), 1.39-1.31 (m, 3H), 0.87 (t, J=7.3 Hz, 6H); ¹³C NMR (151 MHz, DMSO) δ 160.4, 139.8, 138.0, 128.3, 128.2, 118.3, 92.7, 74.9, 71.3, 67.2, 36.8, 34.4, 33.8, 27.2, 26.0, 25.4, 24.1, 22.5, 22.2, 14.0, 13.6; HPLC: t_{R}=5.51 min, Method A, Purity: 98.57%.

Example 8, 3-Isopropyl-2-(4-propylphenethyl)-6-((tetrahydro-2H-pyran-2-yl)methoxy)pyridin-4-ol (10e). The target compound was synthesized as described for compound 5 by debenzylation and hydrogenation of 9e (12.0 mg, 24.7 μmol) in MeOH:EtOAc (1:2; 0.45 mL), catalyzed by 10% Pd—C (1.3 mg, 1.2 μmol) under hydrogen atmosphere for 5 h. The crude was filtered through Celite, the filtrate was concentrated and purified by preparative HPLC to afford 10e as a colorless oil (4.9 mg, 50%). R_{f}=0.40 (EtOAc:heptane; 2:1); ¹H NMR (400 MHz, MeOD) δ 7.09-7.04 (m, 4H), 5.84 (s, 1H), 4.10-3.93 (m, 3H), 3.74-3.64 (m, 1H), 3.54-3.43 (m, 1H), 3.11-3.01 (m, 1H), 2.93-2.80 (m, 4H), 2.58-2.49 (m, 2H), 1.95-1.86 (m, 1H), 1.71-1.52 (m, 6H), 1.50-1.36 (m, 1H), 1.23 (d, J=7.0 Hz, 6H), 0.92 (t, J=7.4 Hz, 3H); HPLC: t_{R}=12.39 min, Method C, Purity: 95.52%.

Example 9, 2-(4-Propylphenethyl)-6-((tetrahydro-2H-pyran-2-yl)methoxy)-3-(o-tolyl)pyridin-4-ol (10f). The target compound was synthesized as described for compound 5 by debenzylation of 9f (12.6 mg, 23.5 μmol) in MeOH:EtOAc (1:2; 1.5 mL), catalyzed by 10% Pd—C (1.3 mg, 1.22 μmol) under hydrogen atmosphere for 4.0 h. The crude was filtered through Celite, the filtrate was concentrated and purified by preparative HPLC (50-75% solvent B in 8.8 min) to afford 10d as a slightly yellow oil (5.7 mg, 47% over 2 steps). R_{f}=0.48 (MeOH:DCM; 1:9); ¹H NMR (600 MHz, DMSO) δ 7.26 (d, J=4.3 Hz, 2H), 7.20-7.15 (m, 1H), 6.99 (d, J=7.9 Hz, 2H), 6.88 (d, J=7.5 Hz, 1H), 6.79 (d, J=7.9 Hz, 2H), 6.30 (br s, 1H), 4.28-4.17 (m, 2H), 3.93-3.89 (m, 1H), 3.69-3.67 (m, 1H), 3.41-3.38 (m, 1H), 2.79-2.67 (m, 2H), 2.49-2.41 (m, 4H), 1.92 (s, 3H), 1.87-1.81 (m, 1H), 1.68-1.62 (m, 1H), 1.57-1.45 (m, 5H), 1.38-1.31 (m, 1H), 0.85 (t, J=7.3 Hz, 3H); ¹³C NMR (151 MHz, DMSO) δ 162.0, 139.6, 138.2, 136.9, 130.3, 129.6, 128.2, 127.9, 127.7, 125.6, 119.1, 92.9, 75.1, 67.3, 36.8, 35.3, 33.7, 27.5, 25.5, 24.1, 22.6, 19.2, 13.6; HPLC: t_{R}=5.89 min, Method A, Purity: 99.97%.

Example 10, 2-(4-Propylphenethyl)-6-((tetrahydro-2H-pyran-2-yl)methoxy)-3-(p-tolyl)pyridin-4-ol (10g). The target compound was synthesized as described for compound 5 by debenzylation of 9g (12.7 mg, 23.7 μmol) in MeOH:EtOAc (1:2; 1.5 mL), catalyzed by 10% Pd—C (1.3 mg, 1.22 μmol) under hydrogen atmosphere for 4.0 h. The crude was filtered through Celite, the filtrate was concentrated and purified by preparative HPLC (50-75% solvent B in 8.8 min) to afford 10g as a colorless oil (6.6 mg, 49% over 2 steps). R_{f}=0.48 (MeOH:DCM; 1:9); ¹H NMR (600 MHz, DMSO) δ 7.17 (d, J=7.8 Hz, 2H), 7.01 (d, J=7.9 Hz, 2H), 6.93 (d, J=7.6 Hz, 2H), 6.84 (d, J=8.0 Hz, 2H), 6.29 (br s, 1H), 4.27-4.16 (m, 2H), 3.93-3.87 (m, 1H), 3.69-3.63 (m, 1H), 3.69-3.62 (m, 1H), 2.80-2.74 (m, 2H), 2.66-2.61 (m, 2H), 2.48-2.45 (m, 2H), 2.33 (s, 3H), 1.86-1.80 (m, 1H), 1.67-1.61 (m, 1H), 1.58-1.43 (m, 5H), 1.37-1.29 (m, 1H), 0.85 (t, J=7.3 Hz, 3H); ¹³C NMR (151 MHz, DMSO) δ 161.6, 139.7, 138.2, 136.4, 130.1, 128.7, 128.2, 128.0, 120.0, 92.9, 75.2, 67.3, 36.9, 35.0, 34.1, 27.5, 25.5, 24.1, 22.6, 20.8, 13.6; HPLC: t_{R}=6.02 min, Method A, Purity: 99.99%.

Example 11, 3-Phenethyl-2-(4-propylphenethyl)-6-((tetrahydro-2H-pyran-2-yl)methoxy)pyridin-4-ol (10h). The target compound was synthesized as described for compound 5 by debenzylation and hydrogenation of 9h (8.4 mg, 15.3 μmol) in MeOH:EtOAc (1:2; 1.5 mL), catalyzed by 10% Pd—C (1.6 mg, 1.50 μmol) under hydrogen atmosphere for 4.0 h. The crude was filtered through Celite, the filtrate was concentrated and purified by preparative HPLC (50-75% solvent B in 8.8 min) to afford 10h as a colorless oil (3.6 mg, 29% over 2 steps). R_{f}=0.47 (MeOH:DCM; 1:9); ¹H NMR (600 MHz, DMSO) δ 7.28-7.23 (m, 2H), 7.19-7.14 (m, 1H), 7.14-7.10 (m, 2H), 7.06 (d, J=8.0 Hz, 2H), 7.04 (d, J=7.9 Hz, 2H), 6.23 (s, 1H), 4.18 (dd, J=10.9, 3.4 Hz, 1H), 4.13 (dd, J=10.8, 6.7 Hz, 1H), 3.91-3.86 (m, 1H), 3.66-3.60 (m, 1H), 3.38-3.34 (m, 1H), 2.79-2.71 (m, 4H), 2.67-2.62 (m, 2H), 2.60-2.55 (m, 2H), 2.48-2.46 (m, 2H), 1.85-1.76 (m, 1H), 1.63-1.57 (m, 1H), 1.56-1.42 (m, 5H), 1.35-1.26 (m, 1H), 0.83 (t, J=7.3 Hz, 3H); ¹³C NMR (151 MHz, DMSO) δ 160.9, 141.6, 139.9, 138.2, 128.4, 128.4, 128.4, 128.3, 126.1, 117.3, 92.9, 75.2, 67.4, 36.9, 34.8, 34.2, 27.5, 26.8, 25.5, 24.2, 22.6, 13.7; HPLC: t_{R}=6.27 min, Method C, Purity: 98.19%.

Example 12, Ethyl (E)-3-(4-hydroxy-2-(4-propylphenethyl)-6-((tetrahydro-2H-pyran-2-yl)methoxy)pyridin-3-yl)acrylate (10i). The target compound was synthesized as described for compound 5 by debenzylation of 9i (26.9 mg, 49.5 μmol) in MeOH:EtOAc (1:2; 0.66 mL), catalyzed by 10% Pd—C (2.6 mg, 2.5 μmol) under hydrogen atmosphere for 2 h. The crude was purified by flash chromatography (15-25% EtOAc in heptane) to afford 10i as a colorless oil (14.2 mg, 63%). R_{f}=0.32 (EtOAc:heptane; 1:2); ¹H NMR (600 MHz, MeOD) δ 7.64 (d, J=15.8 Hz, 1H), 7.15-6.81 (m, 5H), 5.94 (s, 1H), 4.22 (q, J=7.1 Hz, 2H), 4.18-4.04 (m, 2H), 4.03-3.93 (m, 1H), 3.73-3.65 (m, 1H), 3.49 (td, J=11.2, 3.1 Hz, 1H), 3.07-2.99 (m, 2H), 2.93-2.87 (m, 2H), 2.53 (t, J=7.6 Hz, 2H), 1.94-1.86 (m, 1H), 1.69-1.64 (m, 1H), 1.64-1.52 (m, 5H), 1.48-1.39 (m, 1H), 1.32 (t, J=7.1 Hz, 3H), 0.91 (t, J=7.3 Hz, 3H); HPLC: t_{R}=12.58 min, Method C, Purity: >99.99%.

Example 13, Ethyl 3-(4-hydroxy-2-(4-propylphenethyl)-6-((tetrahydro-2H-pyran-2-yl)methoxy)pyridin-3-yl)propanoate (10j). The target compound was synthesized as described for compound 5 by debenzylation of 9i (70.8 mg, 130 μmol) in MeOH:EtOAc (1:2; 2.0 mL), catalyzed by 10% Pd—C (6.9 mg, 6.5 μmol) under hydrogen atmosphere for 16 h. The crude was filtered through a Celite pad, and the filtrate was concentrated to dryness to afford 10j as a slightly yellow oil (51.2 mg, 86%). R_{f}=0.56 (EtOAc:heptane; 4:1); ¹H NMR (600 MHz, MeOD) δ 7.08-7.02 (m, 4H), 5.97 (s, 1H), 4.14-4.04 (m, 4H), 4.01-3.96 (m, 1H), 3.76-3.69 (m, 1H), 3.49 (td, J=11.2, 3.3 Hz, 1H), 2.96-2.86 (m, 4H), 2.64 (t, J=7.8 Hz, 2H), 2.53 (t, J=7.7 Hz, 2H), 2.29-2.24 (m, 2H), 1.94-1.87 (m, 1H), 1.70-1.65 (m, 1H), 1.65-1.53 (m, 5H), 1.50-1.39 (m, 1H), 1.21 (t, J=7.1 Hz, 3H), 0.92 (t, J=7.3 Hz, 3H); HPLC: t_{R}=11.89 min, Method C, Purity: 99.41%.

Example 14, Sodium 3-(4-hydroxy-2-(4-propylphenethyl)-6-((tetrahydro-2H-pyran-2-yl)methoxy)pyridin-3-yl)propanoate (10k). To a solution of 10j (29.5 mg, 63.7 μmol) in THF (0.5 mL) was added aq. LiOH (0.6 M, 320 μL, 0.19 mmol) and stirred at rt for 2 h. The mixture was diluted with water (25 mL), acidified with aq. 1M HCl and extracted with EtOAc (3×25 mL). The combined organic phase was poured onto an aqueous solution of NaOH (6.37 mM, 10 mL) and the mixture was freeze-dried to afford 10k as a yellow solid (28.6 mg, quant.). R_{f}=0.45 (MeOH:DCM+1% acetic acid; 1:9); ¹H NMR (400 MHz, MeOD) δ 7.11 (d, J=8.1 Hz, 2H), 7.05 (d, J=8.1 Hz, 2H), 5.92 (s, 1H), 4.12-3.94 (m, 3H), 3.74-3.65 (m, 1H), 3.52-3.44 (m, 1H), 2.96-2.84 (m, 4H), 2.74 (t, J=7.4 Hz, 2H), 2.58-2.49 (m, 2H), 2.35 (t, J=7.4 Hz, 2H), 1.96-1.82 (m, 1H), 1.72-1.51 (m, 6H), 1.48-1.36 (m, 1H), 0.92 (t, J=7.3 Hz, 3H); HPLC: t_{R}=10.93 min, Method C, Purity: 94.14%.

Example 15, 3-(3-Hydroxypropyl)-2-(4-propylphenethyl)-6-((tetrahydro-2H-pyran-2-yl)methoxy)pyridin-4-ol (10l). A vial, charged with 10j (14.8 mg, 32.5 μmol) and THF (1.0 mL) was cooled down to 0° C. and LiAlH₄ (12.3 mg, 325 μmol) was added. The vial was capped and then evacuated and backfilled with argon three times. After 10 min, the mixture was heated to 70° C. and left stirring at the same temperature for 63 h. The mixture was quenched with water (1 mL), with aqueous 1M NaOH (1 mL), diluted with water (24 mL) and extracted with EtOAc (3×25 mL). The combined organic phase was evaporated onto Celite and purified with flash chromatography (0-6% MeOH in DCM) to afford 101 as a colorless oil (6.1 mg, 45%). R_{f}=0.28 (MeOH:DCM; 5:95); ¹H NMR (600 MHz, MeOD) δ 7.12-7.03 (m, 4H), 5.85 (s, 1H), 4.12-3.95 (m, 3H), 3.75-3.68 (m, 1H), 3.54-3.41 (m, 3H), 2.95-2.77 (m, 4H), 2.54 (t, J=7.4 Hz, 2H), 2.50-2.41 (m, 2H), 1.96-1.86 (m, 1H), 1.71-1.66 (m, 1H), 1.64-1.53 (m, 7H), 1.51-1.41 (m, 1H), 0.92 (t, J=7.4 Hz, 3H); HPLC: t_{R}=11.05 min, Method C, Purity: >99.99%.

Example 16, 3-(4-Ethylphenyl)-2-(4-propylphenethyl)-6-((tetrahydro-2H-pyran-2-yl)methoxy)pyridin-4-ol (10m). The target compound was synthesized as described for compound 9a by the Suzuki coupling of 8 (30.8 mg, 58.7 μmol) with (4-ethylphenyl)boronic acid (26.5 mg, 176 μmol) in the presence of Cs₂CO₃ (76.5 mg, 0.24 mmol), and PdCl₂(dppf) (12.9 mg, 17.6 μmol) in anhydrous DMF (450 μL) for 41 h. The crude was purified by flash chromatography (30% EtOAc in heptane) to afford 10m as a colorless oil (9.4 mg, 29%). R_{f}=0.72 (EtOAc:heptane; 2:1); ¹H NMR (400 MHz, CDCl₃) δ 7.25 (d, J=7.9 Hz, 2H), 7.04-6.94 (m, 4H), 6.88 (d, J=7.8 Hz, 2H), 6.26 (s, 1H), 4.38-4.24 (m, 2H), 4.12-4.03 (m, 1H), 3.80-3.70 (m, 1H), 3.57-3.46 (m, 1H), 2.93-2.84 (m, 2H), 2.74-2.64 (m, 3H), 2.56-2.48 (m, 2H), 1.95-1.87 (m, 1H), 1.75-1.39 (m, 7H), 1.28 (t, J=7.6 Hz, 3H), 0.93 (t, J=7.3 Hz, 3H).

4-(4-(Benzyloxy)-2-(4-propylphenethyl)-6-((tetrahydro-2H-pyran-2-yl)methoxy)pyridin-3-yl)phenol (25). A vial, charged with 8 (59.0 mg, 113 μmol), 4-hydroxylphenylboronic acid (23.2 mg, 169 μmol) and Pd(PPh₃)₄ (6.5 mg, 5.6 μmol), was evacuated and backfilled with argon (3×). Then, anhydrous DMF (1.0 mL) was added to the vial followed by degassed aq. K₂CO₃ (2 M, 0.3 mL). The vial was capped and stirred at 85° C. for 16 h. The cooled reaction mixture was filtered, evaporated onto Celite, and purified by flash chromatography (6-9% EtOAc in heptane) to afford 25 as a slightly red oil (42.2 mg, 70%). R_{f}=0.29 (EtOAc:heptane; 1:1); ¹H NMR (400 MHz, CDCl₃) δ 7.49-7.37 (m, 4H), 7.39-7.31 (m, 1H), 7.19 (d, J=8.0 Hz, 2H), 7.12 (d, J=8.1 Hz, 2H), 6.24 (s, 1H), 5.14 (s, 2H), 4.32 (dd, J=6.8, 3.6 Hz, 1H), 4.22 (dd, J=11.5, 6.8 Hz, 1H), 4.14-4.03 (m, 1H), 3.77-3.64 (m, 1H), 3.56-3.44 (m, 1H), 3.21-3.12 (m, 2H), 3.05-2.95 (m, 2H), 2.62-2.52 (m, 2H), 1.97-1.86 (m, 1H), 1.70-1.36 (m, 7H), 0.96 (t, J=7.3 Hz, 3H).

Example 17, 3-(4-Hydroxyphenyl)-2-(4-propylphenethyl)-6-((tetrahydro-2H-pyran-2-yl)methoxy)pyridin-4-ol (26). The target compound was synthesized as described for compound 5 by debenzylation of 25 (11.6 mg, 21.6 μmol) in MeOH:EtOAc (1:2; 0.75 mL), catalyzed by 10% Pd—C (1.2 mg, 1.10 μmol) under hydrogen atmosphere for 1 h. The crude was filtered through a silica pad and the filtrate was concentrated to dryness to afford 26 as a white solid (7.1 mg 74%); R_{f}=0.08 (EtOAc:heptane; 3:2); ¹H NMR (600 MHz, CDCl₃) δ 7.00 (d, 2H), 6.90-6.81 (m, 6H), 6.15 (s, 1H), 4.24-4.19 (m, 2H), 4.09-4.03 (m, 1H), 3.77-3.70 (m, 1H), 3.51 (td, J=11.7, 2.2 Hz, 1H), 2.86-2.80 (m, 2H), 2.69-2.64 (m, 2H), 2.54-2.48 (m, 2H), 1.94-1.87 (m, 1H), 1.70-1.38 (m, 7H), 0.91 (t, J=7.4 Hz, 3H); HPLC: t_{R}=9.21 min, Method D, Purity: 99.34%.

Methyl 2-(4-(4-(benzyloxy)-2-(4-propylphenethyl)-6-((tetrahydro-2H-pyran-2-yl)methoxy)pyridin-3-yl)phenoxy)acetate (27). To a solution of 25 (42.4 mg, 78.9 μmol) in anhydrous DMF (0.5 mL) was added K₂CO₃ (13.1 mg, 94.6 μmol) and stirred at rt for 10 min. Then, methyl 2-bromoacetate (10 μL, 110 μmol) was added and the reaction mixture was continued stirring at rt. After 15 h, aq. CaCl₂ solution (3.0 M, 20 mL) and 5 mL water was added to the reaction mixture and extracted with EtOAc (3×25 mL). The combined organic phase was dried over anhydrous Na₂SO₄ and filtered. The filtrated was concentrated to dryness to afford 27 as a slightly yellow oil (47.4 mg, 99%). R_{f}=0.29 (EtOAc:heptane; 1:1); ¹H NMR (400 MHz, CDCl₃) δ 7.33-7.21 (m, 4H), 7.18-7.11 (m, 2H), 6.99 (dd, J=13.3, 8.3 Hz, 3H), 6.94-6.85 (m, 4H), 6.24 (s, 1H), 5.00 (s, 2H), 4.66 (s, 2H), 4.40 (dd, J=11.4, 3.4 Hz, 1H), 4.30 (dd, J=11.4, 6.8 Hz, 1H), 4.12-4.02 (m, 1H), 3.82 (s, 3H), 3.79-3.69 (m, 1H), 3.57-3.46 (m, 1H), 2.95-2.86 (m, 2H), 2.78-2.69 (m, 2H), 2.56-2.48 (m, 2H), 1.95-1.86 (m, 1H), 1.73-1.25 (m, 7H), 0.93 (t, J=7.3 Hz, 3H).

Example 18, Methyl 2-(4-(4-hydroxy-2-(4-propylphenethyl)-6-((tetrahydro-2H-pyran-2-yl)methoxy)pyridin-3-yl)phenoxy)acetate (28a). The target compound was synthesized as described for compound 5 by debenzylation of 27 (23.5 mg, 38.5 μmol) in MeOH:EtOAc (1:2; 0.45 mL), catalyzed by 10% Pd—C (2.1 mg, 1.9 μmol) under hydrogen atmosphere for 3 h. The crude was filtered through a silica pad and the filtrate was purified by preparative HPLC (20-60% solvent B in 15 min) to afford 28a as a colorless oil (11.0 mg, 55%). R_{f}=0.19 (EtOAc:heptane; 1:1); ¹H NMR (600 MHz, CDCl₃) δ 6.99 (d, J=7.9 Hz, 2H), 6.87 (dd, J=9.1, 5.8 Hz, 4H), 6.80 (d, J=7.9 Hz, 2H), 6.76 (s, 1H), 4.65 (s, 2H), 3.99 (d, J=4.7 Hz, 2H), 3.97-3.91 (m, 1H), 3.83 (s, 3H), 3.63-3.56 (m, 1H), 3.44-3.37 (m, 1H), 2.82-2.70 (m, 4H), 2.53-2.47 (m, 2H), 2.03 (dd, J=10.0, 3.0 Hz, 1H), 1.89-1.82 (m, 1H), 1.62-1.43 (m, 7H), 0.91 (t, J=7.3 Hz, 3H); HPLC: t_{R}=12.25 min, Method C, Purity: 99.14%.

2-(4-(4-(Benzyloxy)-2-(4-propylphenethyl)-6-((tetrahydro-2H-pyran-2-yl)methoxy)pyridin-3-yl)phenoxy)acetic acid (28b). To a solution of 27 (23.5 mg, 38.5) in THF (0.5 mL) was added aq. LiOH (0.6 M, 190 μL, 115 μmol) and stirred at rt for 2 h. The mixture was diluted with water (25 mL), acidified with aqueous 1M HCl and extracted with EtOAc (3×25 mL). The combined organic phase was washed with brine, dried over anhydrous Na₂SO₄, filtered, and concentrated in vacuo to afford 28b as a colorless oil (22.6 mg, 98%). R_{f}=0.57 (EtOAc:heptane; 2:1+acetic acid); ¹H NMR (400 MHz, CDCl₃) δ 7.32-7.17 (m, 4H), 7.18-7.09 (m, 2H), 7.04-6.97 (m, 2H), 6.98-6.87 (m, 4H), 6.85-6.77 (m, 2H), 6.23 (s, 1H), 5.05 (s, 1H), 4.99 (s, 2H), 4.40 (dd, J=11.5, 3.4 Hz, 1H), 4.30 (dd, J=11.4, 6.8 Hz, 1H), 4.13-4.03 (m, 1H), 3.80-3.69 (m, 1H), 3.57-3.46 (m, 1H), 2.92-2.85 (m, 2H), 2.74 (dd, J=9.4, 6.2 Hz, 2H), 2.52 (dd, J=8.6, 6.7 Hz, 2H), 1.96-1.86 (m, 1H), 1.75-1.25 (m, 7H), 0.92 (t, J=7.4 Hz, 3H).

Example 19, 2-(4-(4-Hydroxy-2-(4-propylphenethyl)-6-((tetrahydro-2H-pyran-2-yl)methoxy)pyridin-3-yl)phenoxy)acetic acid (29). The target compound was synthesized as described for compound 5 by debenzylation and hydrogenation of 28b (23.5 mg, 38.8 μmol) in MeOH:EtOAc (1:2; 0.45 mL), catalyzed by 10% Pd—C (2.1 mg, 1.9 μmol) under hydrogen atmosphere for 1 h. The crude was filtered through a silica pad and the filtrate was purified by preparative HPLC (20-60% Solvent B) to afford 29 as a colorless oil (6.1 mg, 31%). R_{f}=0.74 (Methanol+acetic acid); ¹H NMR (600 MHz, CDCl₃) δ 6.98 (d, J=8.1 Hz, 2H), 6.93 (d, J=8.4 Hz, 2H), 6.88 (d, J=8.1 Hz, 2H), 6.80 (d, J=8.0 Hz, 2H), 5.99 (s, 1H), 4.52 (s, 2H), 4.19-4.09 (m, 2H), 4.03-3.97 (m, 1H), 3.78-3.71 (m, 1H), 3.55-3.48 (m, 1H), 2.78-2.71 (m, 2H), 2.67-2.59 (m, 2H), 2.54-2.48 (m, 2H), 1.95-1.90 (m, 1H), 1.76-1.66 (m, 1H), 1.65-1.54 (m, 5H), 1.51-1.44 (m, 1H), 0.93-0.89 (m, 3H); ¹³C NMR (151 MHz, CDCl₃) δ 162.8, 159.2, 141.5, 139.5, 132.8, 129.5, 129.4, 128.7, 115.5, 94.2, 77.3, 71.8, 69.4, 38.7, 36.2, 35.9, 28.9, 27.0, 25.9, 24.1, 14.1; HPLC: t_{R}=11.55 min, Method C, Purity: >99.99%.

Methyl 4-(4-(benzyloxy)-2-(4-propylphenethyl)-6-((tetrahydro-2H-pyran-2-yl)methoxy)pyridin-3-yl)benzoate (30). A vial, charged with 8 (42.1 mg, 80.3 μmol), (4-(methoxycarbonyl)phenyl)boronic acid (17.3 mg, 96.3 μmol), K₃PO₄ (42.6 mg, 201 μmol), and Pd(Amphos)Cl₂ (5.7 mg, 8.0 μmol), was evacuated and backfilled with argon three times. Then dioxane (1.0 mL) and water (0.1 mL) were added. The vial was capped and stirred at 90° C. for 14 h. The cooled reaction mixture was filtered, evaporated onto Celite, and purified by flash chromatography (10-13% EtOAc in heptane) and further purified by preparative HPLC (55-75% solvent B) to afford 30 as a colorless oil (35.1 mg, 75%). R_{f}=0.78 (EtOAc:heptane; 1:1); 1H NMR (600 MHz, CDCl₃) δ 8.06 (d, J=8.7 Hz, 2H), 7.36-7.28 (m, 3H), 7.16-7.10 (m, 2H), 7.06 (d, J=8.0 Hz, 2H), 6.99 (d, J=8.7 Hz, 2H), 6.79 (d, J=8.7 Hz, 2H), 6.56 (s, 1H), 5.14 (s, 2H), 4.33-4.24 (m, 2H), 4.01-3.95 (m, 4H), 3.80-3.73 (m, 1H), 3.53-3.45 (m, 1H), 2.88-2.85 (m, 4H), 2.54-2.49 (m, 2H), 1.96-1.90 (m, 1H), 1.74-1.39 (m, 7H), 0.92 (t, J=7.3 Hz, 3H).

Example 20, Methyl 4-(4-hydroxy-2-(4-propylphenethyl)-6-((tetrahydro-2H-pyran-2-yl)methoxy)pyridin-3-yl)benzoate (31). The target compound was synthesized as described for compound 5 by debenzylation of 30 (35.1 mg, 60.5 μmol) in MeOH:EtOAc (1:2; 0.45 mL), catalyzed by 10% Pd—C (3.2 mg, 3.0 μmol) under hydrogen atmosphere for 1 h. The crude was filtered through a silica pad and the filtrate was concentrated to dryness to afford 31 as a colorless oil (29.4 mg, 99%). R_{f}=0.56 (EtOAc:heptane; 2:1); ¹H NMR (600 MHz, MeOD) δ 7.96 (d, J=8.4 Hz, 2H), 7.02 (d, J=8.2 Hz, 2H), 6.98 (d, J=7.9 Hz, 2H), 6.77 (d, J=7.9 Hz, 2H), 6.04 (s, 1H), 4.20 (dd, J=10.8, 3.5 Hz, 1H), 4.15 (dd, J=10.8, 6.5 Hz, 1H), 4.04-3.98 (m, 1H), 3.92 (s, 3H), 3.79-3.73 (m, 1H), 3.52 (td, J=11.2, 2.8 Hz, 1H), 2.78 (t, J=7.8, 7.3 Hz, 2H), 2.64 (t, J=7.8 Hz, 2H), 2.52 (t, J=7.4 Hz, 2H), 1.96-1.89 (m, 1H), 1.74-1.68 (m, 1H), 1.67-1.54 (m, 5H), 1.52-1.43 (m, 1H), 0.92 (t, J=7.3 Hz, 3H); ¹³C NMR (151 MHz, MeOD) δ 173.3, 168.5, 163.2, 153.1, 141.8, 141.6, 139.2, 132.1, 130.2, 130.0, 129.5, 129.4, 123.1, 94.3, 77.2, 71.9, 69.4, 52.6, 38.7, 36.1, 35.8, 28.9, 27.0, 25.9, 24.1, 14.1; HPLC: t_{R}=12.35 min, Method C, Purity >99.99%.

Example 21, 4-(4-Hydroxy-2-(4-propylphenethyl)-6-((tetrahydro-2H-pyran-2-yl)methoxy)pyridin-3-yl)benzoic acid (32). To a solution of 31 (13.0 mg, 26.6 μmol) in THF (0.5 mL) was added aq. LiOH (0.6 M, 130 μL, 79.0 μmol) and stirred at rt for 17 h. The solution was diluted with water (25 mL) acidified with aq. HCl (1.0 M) and extracted with EtOAc (3×25 mL). The combined organic phase was washed with brine, dried over anhydrous Na₂SO₄, filtered, and concentrated in vacuo to afford 32 as a colorless oil (12.6 mg, quant.). R_{f}=0.26 (EtOAc:heptane; 2:1+acetic acid); ¹H NMR (400 MHz, MeOD) δ 7.97 (d, J=7.2 Hz, 2H), 7.05-6.96 (m, 4H), 6.78 (d, J=6.9 Hz, 2H), 6.03 (s, 1H), 4.25-4.11 (m, 2H), 4.06-3.97 (m, 1H), 3.82-3.72 (m, 1H), 3.58-3.47 (m, 1H), 2.84-2.76 (m, 2H), 2.69-2.61 (m, 2H), 2.57-2.49 (m, 2H), 1.97-1.87 (m, 1H), 1.75-1.44 (m, 7H), 0.93 (t, J=7.4 Hz, 3H); ¹³C NMR (151 MHz, MeOD) δ 169.9, 163.1, 153.3, 141.7, 141.6, 139.3, 132.0, 130.7, 130.5, 129.5, 129.4, 123.2, 94.3, 77.3, 71.8, 69.4, 38.7, 36.1, 35.8, 28.9, 27.0, 25.9, 24.1, 14.1; HPLC: t_{R}=12.34 min, Method C, Purity: >99.99%.

2,6-Dichloro-4-iodo-3-methylpyridine (34) To a flame dried vial under an argon atmosphere was added anhydrous THF (6 mL) and the vial was cooled down to 0° C. Diisopropylamine (0.26 mL, 1.83 mmol) was added, followed by dropwise addition of a solution of n-BuLi (1.42 M in hexanes, 1.3 mL, 1.83 mmol). After 25 min, the reaction mixture was cooled to −78° C. and a solution of 2,6-dichloro-3-iodopyridine (33) (500 mg, 1.83 mmol) in anhydrous THF (3 mL) was added dropwise. The reaction was stirred at the same temperature for 3 h, then methyl iodide (0.13 mL, 2.0 mmol) was added dropwise. The reaction mixture was left stirring at −78° C. overnight. A mixture of water:THF (1:1, 1 mL) was added to the reaction mixture, which was then allowed to warm to 0° C. Then water (2 mL) was added, and the reaction mixture was allowed to warm up to rt. Additional water (8 mL) was added to the reaction and the phases were separated. The aqueous phase was further extracted with DCM (3×10 mL). The combined organic phase was dried over anhydrous MgSO₄, filtered, and concentrated under reduced pressure. The crude was purified by flash chromatography (1-10% EtOAc in heptane) to afford 34 as a white solid (372 mg). The product contained ˜15% of 2,6-dichloro-4-iodopyridine as an inseparable impurity. ¹H NMR (400 MHz, CDCl₃) δ 7.74 (s, 1H), 2.57 (s, 3H).

2,6-Dichloro-3-ethyl-4-iodopyridine (35) To a flame dried vial under an argon atmosphere was added anhydrous THF (30 mL) and the vial was cooled to 0° C. Diisopropylamine (1.0 mL, 7.30 mmol) was added to the vial, followed by dropwise addition of a solution of n-BuLi (1.47 M in hexanes, 5.0 mL, 7.30 mmol). After 30 min, the reaction mixture was cooled to −78° C. and a solution of 33 (2.0 g, 7.30 mmol) in anhydrous THF (6 mL) was added dropwise. The reaction was stirred at the same temperature for 3 h, then ethyl iodide (0.65 mL, 8.03 mmol) was added dropwise. The reaction mixture was left stirring at −78° C. overnight. Water (˜5 mL) was added to the reaction mixture, which was then allowed to slowly come to rt. Additional water (30 mL) was added and the phases were separated. The aqueous phase was further extracted with DCM (2×30 mL). The combined organic was dried over anhydrous MgSO₄, filtered, and concentrated under reduced pressure. The crude was purified by flash chromatography (1-20% EtOAc in heptane) to afford 35 as a white solid (1.14 g). The product contained 33% of 2,6-dichloro-4-iodopyridine as an inseparable impurity. ¹H NMR (400 MHz, CDCl₃) δ 7.66 (s, 1H), 2.97 (q, J=7.5 Hz, 2H), 1.17 (t, J=7.5 Hz, 3H).

4-(Benzyloxy)-2,6-dichloro-3-methylpyridine (36). To a flame dried vial under an argon atmosphere was added NaH (60%, 21 mg, 0.52 mmol). The vial was evacuated and backfilled with argon three times. Then anhydrous DMF (0.35 mL) was added, and the vial was cooled down to 0° C. Benzyl alcohol (36 μL, 0.35 mmol) was added and stirred for 10 min, after which 34 (100 mg, 0.35 mmol) was added. The vial was evacuated and backfilled with argon three times and left stirring at 0° C. slowly warming up to rt overnight. The reaction was quenched by adding sat. aq. NH₄Cl (1 mL), followed by water (1 mL). The crude was partitioned between aq. CaCl₂ (3 M, 8 mL) and DCM (10 mL). The phases were separated, and the aqueous phase was further extracted with DCM (2×10 mL). The combined organic phase was dried over anhydrous MgSO₄, filtered, evaporated onto Celite, and purified by flash chromatography (1-5% EtOAc in heptane) to afford 36 as a white solid (59.5 mg). The product contained ˜10% of 4-(benzyloxy)-2,6-dichloropyridine as an inseparable impurity. ¹H NMR (400 MHz, CDCl₃) δ 7.49-7.32 (m, 5H), 6.81 (s, 1H), 5.12 (s, 2H), 2.26 (s, 3H).

4-(Benzyloxy)-2,6-dichloro-3-ethylpyridine (37). To a flame dried vial under an argon atmosphere was added NaH (60%, 150 mg, 3.73 mmol). The vial was evacuated and backfilled with argon three times. Then anhydrous DMF (2.5 mL) was added, and the vial was cooled down to 0° C. Benzyl alcohol (0.26 mL, 2.48 mmol) was added and stirred for 10 min, after which 35 (100 mg, 0.35 mmol) was added. The vial was evacuated and backfilled with argon three times and left stirring at 0° C. slowly warming up to rt overnight. The reaction was quenched by adding sat. aq. NH₄Cl (2.0 mL), followed by water (2.0 mL). The crude was partitioned between aq. CaCl₂ (3 M, 16 mL) and DCM (20 mL). The phases were separated, and the aqueous phase was further extracted with DCM (2×20 mL). The combined organic phase was dried over anhydrous MgSO₄, filtered, evaporated onto Celite, and purified by flash chromatography (5-10% EtOAc in heptane) to afford 37 as a white solid (316 mg). The product contained ˜33% of 4-(benzyloxy)-2,6-dichloropyridine as an inseparable impurity. ¹H NMR (400 MHz, CDCl3) δ 7.43-7.34 (m, 5H), 6.81 (s, 1H), 2.77 (q, J=7.5 Hz, 2H), 1.14 (t, J=7.5 Hz, 3H).

6-((1,4-Dioxan-2-yl)methoxy)-4-(benzyloxy)-2-chloro-3-methylpyridine (38). To a flame dried vial was added tBuOK (42 mg, 0.37 mmol) and the vial was evacuated and backfilled with argon three times. A solution of 36 (100 mg, 0.37 mmol) and (1,4-dioxan-2-yl)methanol (44 mg, 0.37 mmol) in anhydrous dioxane (0.75 mL) was added. The vial was capped and stirred at 100° C. overnight. The cooled reaction mixture was partitioned between water (20 mL) and DCM (20 mL). The phases were separated, and the aqueous phase was further extracted with DCM (2×20 mL). The combined organic phase was dried over anhydrous MgSO₄, filtered, evaporated onto Celite, and purified by flash chromatography (5-10% EtOAc in heptane) to afford 38 as a white solid (52 mg). The product contained ˜10% of 4-(benzyloxy)-2,6-dichloropyridine as an inseparable impurity. ¹H NMR (600 MHz, CDCl3) δ 7.43-7.32 (m, 5H), 6.27 (s, 1H), 5.06 (s, 2H), 4.33-4.23 (m, 2H), 3.99-3.91 (m, 1H), 3.88-3.70 (m, 4H), 3.69-3.62 (m, 1H), 3.53-3.46 (m, 1H), 2.20 (s, 3H). ¹³C NMR (151 MHz, CDCl3) δ 166.3, 161.8, 148.0, 135.7, 128.9, 128.5, 127.3, 114.9, 92.7, 73.9, 70.6, 68.3, 66.9, 66.6, 65.9, 12.1

(S)-6-((1,4-dioxan-2-yl)methoxy)-4-(benzyloxy)-2-chloro-3-methylpyridine ((S)-38). To a flame dried vial was added tBuOK (210 mg, 1.86 mmol) and the vial was evacuated with argon three times. A solution of 36 (500 mg, 1.86 mmol) and (R)-(1,4-dioxan-2-yl)methanol (210 mg, 1.77 mmol) in anhydrous dioxane (4.0 mL) was added. The vial was sealed and stirred at 100° C. overnight. The cooled reaction mixture was partitioned between water (10 mL), brine (10 mL) and DCM (20 mL). The phases were separated, and the aqueous phase was further extracted with DCM (2×20 mL). The combined organic phase was dried over anhydrous MgSO₄, filtered, evaporated onto Celite, and purified by flash chromatography (5-15% EtOAc in heptane) to afford (S)-38 as a white solid (255 mg). The product contained ˜15% of (S)-6-((1,4-dioxan-2-yl)methoxy)-4-(benzyloxy)-2-chloro-3-methylpyridine as an inseparable impurity. ¹H NMR (400 MHz, CDCl3) δ 7.47-7.33 (m, 5H), 6.27 (s, 1H), 5.06 (s, 2H), 4.37-4.22 (m, 2H), 3.99-3.91 (m, 1H), 3.89-3.61 (m, 5H), 3.56-3.45 (m, 1H), 2.20 (s, 3H).

6-((1,4-Dioxan-2-yl)methoxy)-4-(benzyloxy)-2-chloro-3-ethylpyridine (39). To a flame dried vial was added tBuOK (110 mg, 0.97 mmol) and the vial was evacuated with argon three times. A solution of 37 (275 mg, 0.97 mmol) and (1,4-dioxan-2-yl)methanol (115 mg, 0.97 mmol) in anhydrous dioxane (2 mL) was added. The vial was sealed and stirred at 100° C. for 5 h. The cooled reaction mixture was partitioned between water (10 mL), brine (10 mL) and DCM (20 mL). The phases were separated, and the aqueous phase was further extracted with DCM (2×20 mL). The combined organic phase was dried over anhydrous MgSO₄, filtered, evaporated onto Celite, and purified by flash chromatography (7-10% EtOAc in heptane) to afford 39 as a white solid (210 mg). The product contained ˜40% of 6-((1,4-dioxan-2-yl)methoxy)-4-(benzyloxy)-2-chloro-3-ethylpyridine as an inseparable impurity. ¹H NMR (400 MHz, CDCl3) δ 7.45-7.31 (m, 5H), 6.26 (s, 1H), 5.07 (s, 2H), 4.32-4.25 (m, 2H), 4.01-3.90 (m, 1H), 3.90-3.59 (m, 5H), 3.54-3.44 (m, 1H), 2.72 (q, J=7.4 Hz, 2H), 1.12 (t, J=7.5 Hz, 3H).

General Procedure for Sonogashira Coupling for the synthesis of compounds 40a,b, (S)-40b, 41a,b, 47, 58, 63a,b, 69a-d as exemplified by compound 40a.

6-((1,4-Dioxan-2-yl)methoxy)-4-(benzyloxy)-3-methyl-2-((4-propylphenyl)ethynyl)pyridine (40a). A flame-dried vial was charged with PdCl₂(MeCN)₂ (1.8 mg, 6.9 μmol), XPhos (9.8 mg, 20.6 μmol), and Cs₂CO₃ (290 mg, 0.89 mmol). The vial was evacuated and backfilled with argon three times. Then, a solution of 38 (120 mg, 0.34 mmol) and 1-ethynyl-4-propylbenzene (70 μL, 0.45 mmol) in anhydrous MeCN (1.1 mL) was added. The vial was sealed and heated at 100° C. for 3 d. The cooled reaction mixture was filtered through a Celite pad, and the pad was repeatedly washed with DCM. The filtrate was evaporated onto Celite and purified by flash chromatography (10-15% EtOAc in heptane) to afford 40a as a yellow oil (146 mg). The product contained ˜10% of 2-((1,4-dioxan-2-yl)methoxy)-4-(benzyloxy)-6-((4-propylphenyl)ethynyl)pyridine as an inseparable impurity. ¹H NMR (400 MHz, CDCl3) δ 7.56-7.45 (m, 2H), 7.45-7.31 (m, 5H), 7.21-7.13 (m, 2H), 6.33 (s, 1H), 5.08 (s, 2H), 4.44-4.30 (m, 2H), 4.03-3.93 (m, 1H), 3.91-3.59 (m, 6H), 3.57-3.47 (m, 1H), 2.60 (t, J=7.4 Hz, 2H), 2.35 (s, 3H), 1.72-1.58 (m, 2H), 0.94 (t, J=7.4 Hz, 3H).

6-((1,4-Dioxan-2-yl)methoxy)-4-(benzyloxy)-2-((4-ethoxyphenyl)ethynyl)-3-methylpyridine (40b). The target compound was synthesized as described for compound 40a by Sonogashira Coupling of 38 (25 mg, 71.5 μmol) with 1-ethoxy-4-ethynylbenzene (14 mg, 92.9 μmol) in the presence of PdCl₂(MeCN)₂ (1.9 mg, 7.2 μmol), XPhos (10 mg, 21.4 μmol), and Cs₂CO₃ (60 mg, 0.19 mmol) in anhydrous MeCN (0.5 mL) overnight at 90° C. The crude was purified by flash chromatography (10-20% EtOAc in heptane) to afford 40b as a yellow oil (25 mg). The product contained ˜40% of an inseparable impurity. ¹H NMR (400 MHz, CDCl3) δ 7.52 (d, J=8.7 Hz, 2H), 7.44-7.30 (m, 5H), 6.87 (d, J=8.8 Hz, 2H), 6.31 (s, 1H), 5.07 (s, 2H), 4.42-4.26 (m, 2H), 4.06 (q, J=7.0 Hz, 2H), 4.02-3.93 (m, 1H), 3.91-3.59 (m, 4H), 3.59-3.44 (m, 2H), 2.34 (s, 3H), 1.43 (t, J=7.0 Hz, 3H).

(S)-6-((1,4-dioxan-2-yl)methoxy)-4-(benzyloxy)-2-((4-ethoxyphenyl)ethynyl)-3-methylpyridine ((S)-40b). The target compound was synthesized as described for compound 40a by Sonogashira Coupling of (S)-38 (330 mg, 0.94 mmol) with 1-ethoxy-4-ethynylbenzene (180 mg, 1.23 mmol) in the presence of PdCl₂(MeCN)₂ (4.9 mg, 18.9 μmol), XPhos (27 mg, 56.6 μmol), and Cs₂CO₃ (800 mg, 2.45 mmol) in anhydrous MeCN (3.0 mL) at 100° C. for 3 d. The crude was purified by flash chromatography (10-20% EtOAc in heptane) to afford (S)-40b as a yellow oil (370 mg). The product contained ˜15% of (S)-2-((1,4-dioxan-2-yl)methoxy)-4-(benzyloxy)-6-((4-ethoxyphenyl)ethynyl)pyridine as an inseparable impurity. ¹H NMR (400 MHz, CDCl3) δ 7.57-7.47 (m, 2H), 7.45-7.30 (m, 5H), 6.93-6.79 (m, 2H), 6.31 (s, 1H), 5.08 (s, 2H), 4.43-4.29 (m, 2H), 4.06 (q, J=7.0 Hz, 2H), 4.02-3.94 (m, 1H), 3.92-3.59 (m, 5H), 3.57-3.47 (m, 1H), 2.34 (s, 3H), 1.43 (t, J=7.0 Hz, 3H).

6-((1,4-Dioxan-2-yl)methoxy)-4-(benzyloxy)-3-ethyl-2-((4-propylphenyl)ethynyl)pyridine (41a). The target compound was synthesized as described for compound 40a by Sonogashira Coupling of 39 (103 mg, 0.28 mmol) with 1-ethynyl-4-propylbenzene (60 μL, 0.37 mmol) in the presence of PdCl₂(MeCN)₂ (3.7 mg, 14.2 μmol), XPhos (20 mg, 42.5 μmol), and Cs₂CO₃ (240 mg, 0.74 mmol) in anhydrous MeCN (1.0 mL) at 100° C. overnight. The crude was purified by flash chromatography (10-15% EtOAc in heptane) to afford 41a as a yellow oil (100 mg). The product contained ˜33% of 2-((1,4-dioxan-2-yl)methoxy)-4-(benzyloxy)-6-((4-propylphenyl)ethynyl)pyridine as an inseparable impurity. ¹H NMR (400 MHz, CDCl3) δ 7.53-7.47 (m, 2H), 7.38-7.25 (m, 5H), 7.14-7.03 (m, 2H), 6.31 (s, 1H), 5.05 (s, 2H), 4.46-4.23 (m, 2H), 4.01-3.87 (m, 1H), 3.85-3.55 (m, 5H), 3.51-3.41 (m, 1H), 2.80 (q, J=7.4 Hz, 2H), 2.58-2.49 (m, 2H), 1.67-1.51 (m, 2H), 1.13 (t, J=7.4 Hz, 3H), 0.93-0.85 (m, 3H)

6-((1,4-Dioxan-2-yl)methoxy)-4-(benzyloxy)-2-((4-ethoxyphenyl)ethynyl)-3-ethylpyridine (41b). The target compound was synthesized as described for compound 40a by Sonogashira Coupling of 39 (107 mg, 0.29 mmol) with 1-ethoxy-4-ethynylbenzene (56 mg, 0.38 mmol) in the presence of PdCl₂(MeCN)₂ (3.8 mg, 14.7 μmol), XPhos (21 mg, 44.1 μmol), and Cs₂CO₃ (250 mg, 0.76 mmol) in anhydrous MeCN (1.0 mL) at 100° C. overnight. The crude was purified by flash chromatography (10-20% EtOAc in heptane) to afford 41b as a yellow oil (110 mg). The product contained ˜33% of 2-((1,4-dioxan-2-yl)methoxy)-4-(benzyloxy)-6-((4-ethoxyphenyl)ethynyl)pyridine as an inseparable impurity. ¹H NMR (400 MHz, CDCl3) δ 7.58-7.46 (m, 2H), 7.46-7.29 (m, 5H), 6.91-6.83 (m, 2H), 6.33 (s, 1H), 5.10 (s, 2H), 4.47-4.31 (m, 2H), 4.11-3.57 (m, 8H), 3.57-3.47 (m, 1H), 2.86 (q, J=7.4 Hz, 2H), 1.47-1.40 (m, 3H), 1.20 (t, J=7.4 Hz, 3H).

Example 22, 6-((1,4-Dioxan-2-yl)methoxy)-3-methyl-2-(4-propylphenethyl)pyridin-4-ol (42a). The target compound was synthesized as described for compound 5 by debenzylation and hydrogenation of 40a (120 mg, 0.26 mmol) in MeOH:EtOAc (1:2; 6.0 mL), catalyzed by 10% Pd—C (14 mg, 13.1 μmol) under hydrogen atmosphere for 1 h. The crude was filtered through a celite pad, and the filtrate was purified by preparative HPLC (30-50% solvent B in 15 min) to afford 42a as a white solid (66 mg, 60%). ¹H NMR (600 MHz, DMSO) δ 7.13-7.07 (m, 4H), 6.37 (s, 1H), 4.26-4.18 (m, 2H), 3.92-3.85 (m, 1H), 3.83-3.74 (m, 2H), 3.70-3.58 (m, 2H), 3.53-3.46 (m, 1H), 3.44-3.37 (m, 1H), 2.93 (dd, J=9.5, 6.3 Hz, 2H), 2.83 (dd, J=9.5, 6.3 Hz, 2H), 2.54-2.51 (m, 2H), 1.92 (s, 3H), 1.60-1.51 (m, 2H), 0.87 (t, J=7.3 Hz, 3H); ¹³C NMR (151 MHz, DMSO) δ 159.8, 158.3, 158.1, 139.8, 137.8, 128.3, 128.2, 114.2, 92.4, 72.9, 67.0, 65.8, 65.7, 36.8, 33.6, 33.6, 24.1, 13.6, 9.5; HPLC: t_{R}=11.18 min, Method C, Purity: 99.09%.

Example 23, 6-((1,4-Dioxan-2-yl)methoxy)-2-(4-ethoxyphenethyl)-3-methylpyridin-4-ol (42b). The target compound was synthesized as described for compound 5 by debenzylation and hydrogenation of 40b (12 mg, 26.1 μmol) in MeOH:EtOAc (1:2; 0.6 mL), catalyzed by 10% Pd—C (4.0 mg, 3.90 μmol) under hydrogen atmosphere for 1 h. The crude was filtered through a celite pad, and the filtrate was purified by preparative HPLC (25-45% solvent B in 15 min) to afford 42b as a white solid (2.4 mg, 25%). ¹H NMR (600 MHz, DMSO) δ 7.12-7.07 (m, 2H), 6.84-6.79 (m, 2H), 6.25 (s, 1H), 4.22-4.19 (m, 2H), 3.97 (q, J=7.0 Hz, 2H), 3.88-3.84 (m, 1H), 3.82-3.74 (m, 2H), 3.69-3.33 (m, 4H), 2.98-2.85 (m, 2H), 2.83-2.78 (m, 2H), 1.92 (s, 3H), 1.30 (t, J=6.9 Hz, 3H); 13C NMR (151 MHz, DMSO) δ 159.9, 158.2, 158.0, 156.9, 132.3, 129.3, 114.2, 92.4, 72.9, 67.0, 65.8, 65.7, 62.9, 33.1, 14.7, 9.6; HPLC: t_{R}=10.02 min, Method C, Purity: 99.25%

Example 24, (S)-6-((1,4-dioxan-2-yl)methoxy)-2-(4-ethoxyphenethyl)-3-methylpyridin-4-ol ((S)-42b). The target compound was synthesized as described for compound 5 by debenzylation and hydrogenation of (S)-40b (345 mg, 0.74 mmol) in MeOH:EtOAc (1:2; 18 mL), catalyzed by 10% Pd—C (40 mg, 37.5 μmol) under hydrogen atmosphere for 1 h. The crude was filtered through a celite pad, and the filtrate was purified by preparative HPLC (20-45% solvent B in 20 min) to afford (S)-42b as a white solid (150 mg, 54%). ¹H NMR (600 MHz, DMSO) δ 7.12-7.07 (m, 2H), 6.85-6.79 (m, 2H), 6.34 (s, 1H), 4.25-4.18 (m, 2H), 3.97 (q, J=6.9 Hz, 2H), 3.92-3.84 (m, 1H), 3.82-3.74 (m, 2H), 3.70-3.58 (m, 2H), 3.53-3.46 (m, 1H), 3.43-3.37 (m, 1H), 2.91 (dd, J=9.4, 6.3 Hz, 2H), 2.80 (dd, J=9.4, 6.3 Hz, 2H), 1.93 (s, 3H), 1.30 (t, J=7.0 Hz, 3H); ¹³C NMR (151 MHz, DMSO) δ 159.9, 158.2, 158.0, 156.9, 132.3, 129.3, 114.2, 92.4, 72.9, 67.0, 65.8, 65.7, 62.9, 33.1, 14.7, 9.6; HPLC: t_{R}=10.06 min, Method C, Purity: 99.32%.

Example 25, 6-((1,4-Dioxan-2-yl)methoxy)-3-ethyl-2-(4-propylphenethyl)pyridin-4-ol (43a). The target compound was synthesized as described for compound 5 by debenzylation and hydrogenation of 41a (100 mg, 0.21 mmol) in MeOH:EtOAc (1:2; 6 mL), catalyzed by 10% Pd—C (11 mg, 10.6 μmol) under hydrogen atmosphere for 1 h. The crude was filtered through a celite pad, and the filtrate was purified by preparative HPLC (35-55% solvent B in 15 min) to afford 43a as a white solid (42.4 mg, 52%. ¹H NMR (600 MHz, DMSO) δ 7.14-7.07 (m, 4H), 6.36 (s, 1H), 4.26-4.19 (m, 2H), 3.93-3.86 (m, 1H), 3.83-3.75 (m, 2H), 3.70-3.59 (m, 2H), 3.54-3.46 (m, 1H), 3.44-3.37 (m, 1H), 2.95-2.89 (m, 2H), 2.89-2.83 (m, 2H), 2.54-2.51 (m, 2H), 2.44 (q, J=7.4 Hz, 2H), 1.60-1.51 (m, 2H), 0.96 (t, J=7.4 Hz, 3H), 0.87 (t, J=7.3 Hz, 3H); ¹³C NMR (151 MHz, DMSO) δ 160.0, 158.3, 158.0, 139.9, 137.8, 128.3, 128.2, 119.0, 92.7, 72.9, 67.0, 65.8, 65.7, 36.8, 34.5, 24.1, 17.4, 13.7, 13.6; HPLC: t_{R}=11.69 min, Method C, Purity: 99.28%.

Example 26, 6-((1,4-Dioxan-2-yl)methoxy)-2-(4-ethoxyphenethyl)-3-ethylpyridin-4-ol (43b). The target compound was synthesized as described for compound 5 by debenzylation and hydrogenation of 41b (110 mg, 0.23 mmol) in MeOH:EtOAc (1:2; 6 mL), catalyzed by 10% Pd—C (12 mg, 11.6 μmol) under hydrogen atmosphere for 1 h. The crude was filtered through a celite pad, and the filtrate was purified by preparative HPLC (25-45% solvent B in 15 min) to afford 43b as a white solid (42.4 mg, 47%). ¹H NMR (600 MHz, DMSO) δ 7.13-7.08 (m, 2H), 6.85-6.80 (m, 2H), 6.32 (s, 1H), 4.24-4.20 (m, 2H), 3.97 (q, J=7.0 Hz, 2H), 3.92-3.85 (m, 1H), 3.83-3.74 (m, 2H), 3.70-3.58 (m, 2H), 3.53-3.46 (m, 1H), 3.43-3.36 (m, 1H), 2.92-2.86 (m, 2H), 2.86-2.80 (m, 2H), 2.44 (q, J=7.4 Hz, 2H), 1.30 (t, J=7.0 Hz, 3H), 0.97 (t, J=7.4 Hz, 3H); ¹³C NMR (151 MHz, DMSO) δ 160.2, 158.2, 158.0, 156.9, 132.7, 129.3, 120.0, 114.3, 92.8, 72.9, 67.0, 65.8, 65.7, 62.9, 34.0, 17.5, 14.7, 13.7; HPLC: t_{R}=10.63 min, Method C, Purity: 99.45%.

1-bromo-4-((4-methoxybenzyl)oxy)benzene (45). To a vial containing 4-bromophenol (44) (500 mg, 2.89 mmol) and K₂CO₃ (800 mg, 5.78 mmol) was added 1-(chloromethyl)-4-methoxybenzene (0.43 mL, 3.18 mmol) and acetone (11 mL). The reaction mixture was allowed to stir at rt for 15 min, after which the vial was sealed and stirred at 80° C. for 3 d. The reaction was allowed to come to rt, after which the solvent was removed under reduced pressure. The resulting residue was redissolved in water (10 mL), brine (10 mL), and DCM (20 mL). The phases were separated, and the aqueous phase was further extracted with DCM (2×20 mL). The combined organic phase was dried over anhydrous MgSO₄, filtered, and concentrated to dryness to afford 45 as a pale reddish solid (770 mg, 91%). The product was used for the next step without further purification. ¹H NMR (400 MHz, CDCl3) δ 7.41-7.28 (m, 4H), 6.96-6.87 (m, 2H), 6.87-6.82 (m, 2H), 4.96 (s, 2H), 3.82 (s, 3H).

1-Ethynyl-4-((4-methoxybenzyl)oxy)benzene (46). A vial was charged with 45 (2.5 g, 8.53 mmol), Na₂PdCl₄ (25.1 mg, 85.3 mmol), 2-(di-tert-butylphosphino)-N-phenylindole (PIntB, 57.6 mg, 0.17 mmol), CuI (32.5 mg, 0.17 mmol), TMEDA (17.3 mL, 115.4 mmol) and H₂O (1.9 mL, 1.9 mmol). The vial was evacuated and backfilled with argon three times, and TMSA (2.36 mL, 17.1 mmol) was added. The vial was capped and stirred at 90° C. for 30 min. Water (50 mL) was added to the cooled reaction mixture and extracted with EtOAc (3×50 mL). The combined organic phase was dried over anhydrous Na₂SO₄, filtered, and concentrated to give brown oil. The crude was suspended in MeOH (60 mL) and K₂CO₃ (2.36 g, 17.1 mmol) was added and stirred at rt. After 2 h, the solvent was evaporated, and the crude was suspended in EtOAc (50 mL). Water (50 mL) was added, and the organic phase was separated. The aqueous phase was further extracted with EtOAc (2×50 mL). The combined organic phase was dried over anhydrous Na₂SO₄, filtered, concentrated, and purified by flash chromatography (5% EtOAc in heptane) to afford 46 as a light yellow solid (1.28 g, 63% over 2 steps).R_{f}=0.35 (EtOAc:heptane; 1:9); ¹H NMR (400 MHz, CDCl₃) δ 7.42 (d, J=8.8 Hz, 2H), 7.34 (d, J=8.6 Hz, 2H), 6.95-6.88 (m, 4H), 4.99 (s, 2H), 3.82 (s, 3H), 2.99 (s, 1H); ¹³C NMR (151 MHz, CDCl₃) δ 159.7, 159.3, 133.7, 129.4, 128.7, 115.0, 114.5, 114.2, 83.8, 76.0, 70.0, 55.5.

6-((1,4-Dioxan-2-yl)methoxy)-4-(benzyloxy)-2-((4-((4-methoxybenzyl)oxy)phenyl)ethynyl)-3-methylpyridine (47). The target compound was synthesized as described for compound 40a by Sonogashira Coupling of 38 (240 mg, 0.69 mmol) with 1-ethynyl-4-((4-methoxybenzyl)oxy)benzene (46) (212 mg, 0.89 mmol) in the presence of PdCl₂(MeCN)₂ (5.3 mg, 20.6 μmol), XPhos (30 mg, 61.8 μmol), and Cs₂CO₃ (580 mg, 1.78 mmol) in anhydrous MeCN (3.3 mL) at 90° C. overnight. The crude was purified by flash chromatography (10-15% EtOAc in heptane) to afford 47 as a yellow solid (240 mg). The product contained ˜15% of 2-((1,4-dioxan-2-yl)methoxy)-4-(benzyloxy)-6-((4-((4-methoxybenzyl)oxy)phenyl)ethynyl)pyridine as an inseparable impurity. ¹H NMR (400 MHz, CDCl3) δ 7.58-7.48 (m, 2H), 7.47-7.33 (m, 7H), 7.00-6.88 (m, 4H), 6.32 (s, 1H), 5.08 (s, 2H), 5.01 (s, 2H), 4.43-4.30 (m, 2H), 4.03-3.93 (m, 1H), 3.89-3.62 (m, 8H), 3.57-3.47 (m, 1H), 2.34 (s, 3H).

4-((6-((1,4-Dioxan-2-yl)methoxy)-4-(benzyloxy)-3-methylpyridin-2-yl)ethynyl)phenol (48) To a solution of 47 (228 mg, 0.41 mmol) in DCM (4.5 mL) was added TFA (0.55 mL, 12% final solution) and anisole (0.22 mL, 2.07 mmol) and stirred at rt. After 2 h saturated aq. NaHCO₃ (20 mL) was slowly added to the reaction mixture followed by DCM (15 mL). The phases were separated and the aqueous was further extracted with DCM (2×20 mL). The combined organic fractions were dried over anhydrous MgSO₄, filtered, and concentrated under reduced pressure. The crude was subsequently redissolved in 10% aq. NaOH (10 mL) and DCM (10 mL) resulting in an emulsion to which a few drops of 1 M HCl(aq) was added to facilitate phase separation.

Following phase separation, the aqueous phase was acidified with aq. HCl (1.0 M) and extracted with DCM (3×20 mL). The combined organic phase was dried over anhydrous MgSO₄, filtered, and concentrated under reduced pressure to afford 48 as a white solid (75 mg, 42%). The product contained ˜15% of 4-((6-((1,4-dioxan-2-yl)methoxy)-4-(benzyloxy)pyridin-2-yl)ethynyl)phenol as an inseparable impurity. ¹H NMR (400 MHz, DMSO) δ 9.99 (s, 1H), 7.49-7.32 (m, 7H), 6.84-6.79 (m, 2H), 6.51 (s, 1H), 5.21 (s, 2H), 4.26-4.12 (m, 2H), 3.87-3.56 (m, 5H), 3.56-3.43 (m, 1H), 3.43-3.34 (m, 1H), 2.25 (s, 3H).

tert-Butyl (2-(4-((6-((1,4-dioxan-2-yl)methoxy)-4-(benzyloxy)-3-methylpyridin-2-yl)ethynyl)phenoxy)ethyl)carbamate (49a). To a vial containing 48 (33 mg, 76.5 μmol), tert-butyl (2-bromoethyl)carbamate (26 mg, 114.7 μmol) and K₂CO₃ (21 mg, 153.0 μmol) under an argon atmosphere was added acetone (0.4 mL). The vial was stirred at rt for 15 min, after which the vial was sealed and stirred at 60° C. for 4 d. The reaction was allowed to come to rt, after which the solvent was removed under reduced pressure. The resulting residue was redissolved in water (20 mL) and DCM (20 mL). The phases were separated, and the aqueous phase was further extracted with DCM (2×20 mL). The combined organic phase was dried over anhydrous MgSO₄, filtered, and concentrated under reduced pressure to afford 49a as a white solid (28 mg, 64%). ¹H NMR (600 MHz, CDCl3) δ 7.48-7.40 (m, 2H), 7.36-7.30 (m, 4H), 7.30-7.24 (m, 1H), 6.82-6.77 (m, 2H), 6.23 (s, 1H), 4.99 (s, 2H), 4.32-4.21 (m, 2H), 3.99-3.94 (m, 2H), 3.93-3.87 (m, 1H), 3.82-3.75 (m, 2H), 3.75-3.68 (m, 1H), 3.68-3.62 (m, 1H), 3.62-3.52 (m, 1H), 3.50-3.41 (m, 3H), 2.26 (s, 3H), 1.38 (s, 9H).

Ethyl 2-(4-((6-((1,4-dioxan-2-yl)methoxy)-4-(benzyloxy)-3-methylpyridin-2-yl)ethynyl)phenoxy)acetate (49b). To a vial containing 48 (32.5 mg, 75.3 μmol), ethyl 2-bromoacetate (17 μL, 150.6 μmol) and K₂CO₃ (21 mg, 150.6 μmol) under an argon atmosphere was added MeCN (0.4 mL). The vial was stirred at rt for 15 minutes, after which the vial was sealed and stirred at 80° C. for 5 d. The reaction was allowed to come to rt, after which the reaction mixture was partitioned between water (20 mL) and DCM (20 mL). The phases were separated, and the aqueous phase was further extracted with DCM (2×20 mL). The combined organic phase was dried over anhydrous MgSO₄, filtered, and concentrated under reduced pressure to afford 49b as an off-white solid (26 mg, 67%). The product was used in the next step without further purification.

Example 27, Tert-butyl (2-(4-(2-(6-((1,4-dioxan-2-yl)methoxy)-4-hydroxy-3-methylpyridin-2-yl)ethyl)phenoxy)ethyl)carbamate (50a). The target compound was synthesized as described for compound 5 by debenzylation and hydrogenation of 49a (14.2 mg, 24.7 μmol) in MeOH:EtOAc (1:2; 0.6 mL), catalyzed by 10% Pd—C (4.0 mg, 3.7 μmol) under hydrogen atmosphere for 1 h. The crude was filtered through a celite pad; the filtrate was concentrated and purified by preparative HPLC (20-45% solvent B in 20 min) to afford 50a as a white solid (6 mg, 50%). ¹H NMR (600 MHz, MeOD) δ 7.07-7.02 (m, 2H), 6.87-6.81 (m, 2H), 6.36 (s, 1H), 4.24 (d, J=4.7 Hz, 2H), 4.00-3.94 (m, 3H), 3.88-3.79 (m, 2H), 3.79-3.69 (m, 2H), 3.66-3.57 (m, 1H), 3.52 (dd, J=11.5, 10.0 Hz, 1H), 3.40 (t, J=5.7 Hz, 2H), 3.00 (dd, J=8.4, 6.7 Hz, 2H), 2.88 (dd, J=8.4, 6.7 Hz, 2H), 1.95 (s, 3H), 1.44 (s, 9H); ¹³C NMR (151 MHz, MeOD) δ 174.5, 161.1, 159.1, 158.6, 152.0, 133.2, 130.6, 117.7, 115.7, 93.3, 80.2, 74.5, 70.6, 68.6, 68.0, 67.7, 67.5, 41.0, 34.9, 34.4, 28.7, 9.9; HPLC: t_{R}=10.77 min, Method C, Purity: 94.10%.

Example 28, Ethyl 2-(4-(2-(6-((1,4-dioxan-2-yl)methoxy)-4-hydroxy-3-methylpyridin-2-yl)ethyl)phenoxy)acetate (50b). The target compound was synthesized as described for compound 5 by debenzylation and hydrogenation of 49b (26 mg, 50.2 μmol) in MeOH:EtOAc (1:2; 1.2 mL), catalyzed by 10% Pd—C (8.0 mg, 7.5 μmol) under hydrogen atmosphere for 1 h. The crude was filtered through a celite pad; the filtrate was concentrated and purified by preparative HPLC (20-45% solvent B in 20 min) to afford 50b as a white solid (8.1 mg, 37%). ¹H NMR (600 MHz, DMSO) δ 7.14-7.09 (m, 2H), 6.85-6.80 (m, 2H), 6.21 (s, 1H), 4.72 (s, 2H), 4.21-4.12 (m, 4H), 3.94-3.31 (m, 7H), 2.91-2.85 (m, 2H), 2.85-2.79 (m, 2H), 1.92 (s, 3H), 1.21 (t, J=7.1 Hz, 3H); ¹³C NMR (151 MHz, DMSO) δ 171.2, 168.8, 158.0, 157.8, 156.0, 129.3, 114.3, 92.3, 73.1, 67.2, 65.8, 65.7, 64.7, 60.6, 33.1, 14.0, 9.6; HPLC: t_{R}=10.14 min, Method C, Purity: 99.07%.

Example 29, 2-(4-(2-(6-((1,4-Dioxan-2-yl)methoxy)-4-hydroxy-3-methylpyridin-2-yl)ethyl)phenoxy)ethan-1-aminium chloride (51). To a solution of 50a (4.0 mg, 8.2 mmol) in DCM (0.2 mL) was added 4 M HCl in dioxane (20 mL, 68.8 μmol). The reaction mixture was stirred at rt for 5 d, during which additional DCM and 4 M HCl were added. The solvent was decanted off and the precipitate was washed several times with DCM and dried under reduced pressure to afford 51 as a white solid (1.6 mg, 46%). ¹H NMR (600 MHz, DMSO) δ 8.22 (s, 3H), 7.19-7.14 (m, 2H), 6.93-6.88 (m, 2H), 6.58 (br s, 1H), 4.24-4.20 (m, 2H), 4.17-4.12 (m, 2H), 3.92-3.87 (m, 1H), 3.84-3.75 (m, 2H), 3.70-3.59 (m, 2H), 3.54-3.46 (m, 1H), 3.45-3.37 (m, 1H), 3.20-3.14 (m, 2H), 2.96-2.93 (m, 2H), 2.85-2.80 (m, 2H), 1.91 (s, 3H); ¹³C NMR (151 MHz, DMSO) δ 158.2, 157.9, 156.3, 133.1, 129.5, 114.6, 92.6, 72.8, 69.8, 66.9, 65.8, 65.7, 64.3, 48.6, 38.3, 33.2, 9.5; HPLC: t_{R}=7.35 min, Method C, Purity: 97.71%.

Example 30, 2-(4-(2-(6-((1,4-Dioxan-2-yl)methoxy)-4-hydroxy-3-methylpyridin-2-yl)ethyl)phenoxy)acetic acid (52). To a solution of 50b (6 mg, 13.9 μmol) in THF (140 μL) was added aq. LiOH (0.6 M. 70 μL, 41.7 μmol). The reaction mixture was stirred at rt for 2.5 h, after which it was diluted with aq. HCl (1.0M) and extracted with EtOAc. The combined organic phase was dried over anhydrous MgSO₄, filtered and concentrated under reduced pressure to afford 52 as a colourless oil (4 mg, 71%). ¹H NMR (600 MHz, Acetone) δ 7.49 (s, 1H), 7.22-7.17 (m, 2H), 6.89-6.84 (m, 2H), 4.70 (s, 2H), 4.35-4.29 (m, 2H), 4.00-3.94 (m, 1H), 3.90-3.87 (m, 1H), 3.82-3.78 (m, 1H), 3.68-3.65 (m, 2H), 3.59-3.47 (m, 2H), 3.26-3.20 (m, 2H), 3.01-2.96 (m, 2H), 2.03 (s, 3H). 2-(4-((6-((1,4-dioxan-2-yl)methoxy)-4-(benzyloxy)-3-methylpyridin-2-yl)ethynyl)phenoxy)ethan-1-amine hydrochloride (53). To a solution of 49a (11 mg, 19.1 mol) in DCM (0.4 mL) was added 4 M HCl in dioxane (80 μL, 0.32 mmol). The mixture was stirred at rt for 3 d, during which additional DCM and 4 M HCl were added. The solvent was decanted off and the precipitate was washed several times with DCM and dried under reduced pressure to afford 53 as a white solid (5.5 mg). The product contained ˜15% of 2-(4-((6-((1,4-dioxan-2-yl)methoxy)-4-(benzyloxy)pyridin-2-yl)ethynyl)phenoxy)ethan-1-amine hydrochloride as an inseparable impurity. ¹H NMR (400 MHz, MeOD) δ 7.70-7.61 (m, 2H), 7.56-7.37 (m, 5H), 7.18-7.07 (m, 2H), 7.03 (s, 1H), 5.44 (s, 2H), 4.51-4.45 (m, 2H), 4.36-4.28 (m, 2H), 4.06-3.98 (m, 1H), 3.93-3.86 (m, 1H), 3.84-3.70 (m, 3H), 3.67-3.52 (m, 2H), 3.43-3.39 (m, 2H), 2.38 (s, 3H).

N-(2-(4-((6-((1,4-dioxan-2-yl)methoxy)-4-(benzyloxy)-3-methylpyridin-2-yl)ethynyl)phenoxy)ethyl)acetamide (54). To a solution of 53 (5.5 mg, 10.8 μmol) in DCM (0.1 mL) was added TEA (10 μL, 71.6 μmol) and acetyl chloride (5 μL, 70.0 μmol) and stirred at rt for 2 h. The reaction mixture was diluted with water (2 mL) and DCM (2 mL). The phases were separated, and the organic phase was filtered over anhydrous MgSO₄ and concentrated under reduced pressure to afford 54 as a colorless oil (5.3 mg). The product contained ˜15% of N-(2-(4-((6-((1,4-dioxan-2-yl)methoxy)-4-(benzyloxy)pyridin-2-yl)ethynyl)phenoxy)ethyl)acetamide as an inseparable impurity. ¹H NMR (400 MHz, CDCl3) δ 7.58-7.48 (m, 2H), 7.45-7.30 (m, 5H), 6.92-6.83 (m, 2H), 6.31 (s, 1H), 5.93 (s, 1H), 5.08 (s, 2H), 4.42-4.29 (m, 2H), 4.11-4.03 (m, 2H), 4.03-3.93 (m, 1H), 3.90-3.60 (m, 7H), 3.57-3.47 (m, 1H), 2.34 (s, 3H), 2.02 (s, 3H).

Example 31, N-(2-(4-(2-(6-((1,4-dioxan-2-yl)methoxy)-4-hydroxy-3-methylpyridin-2-yl)ethyl)phenoxy)ethyl)acetamide (55). The target compound was synthesized as described for compound 5 by debenzylation and hydrogenation of 54 (5.3 mg, 10.3 μmol) in MeOH:EtOAc (1:2; 0.3 mL), catalyzed by 10% Pd—C (2.0 mg, 2.1 μmol) under hydrogen atmosphere for 1 h. The crude was filtered through a celite pad; the filtrate was concentrated and purified by preparative HPLC (20-45% solvent B in 20 min) to afford 55 as a colorless oil (3.5 mg, 79%). 1H NMR (600 MHz, DMSO) δ 8.07 (t, J=5.6 Hz, 1H), 7.14-7.08 (m, 2H), 6.87-6.81 (m, 2H), 6.24 (s, 1H), 4.21-4.17 (m, 2H), 3.95-3.89 (m, 2H), 3.88-3.83 (m, 1H), 3.81-3.73 (m, 2H), 3.71-3.58 (m, 2H), 3.53-3.46 (m, 1H), 3.41-3.35 (m, 3H), 2.91-2.84 (m, 2H), 2.84-2.79 (m, 2H), 1.92 (s, 3H), 1.82 (s, 3H); ¹³C NMR (151 MHz, DMSO) δ 174.5, 171.2, 169.4, 158.3, 158.0, 157.8, 157.6, 156.7, 129.3, 117.6, 115.7, 114.3, 92.5, 73.0, 72.4, 67.2, 66.3, 65.8, 65.7, 42.6, 40.1, 38.2, 33.1, 22.5, 9.6; HPLC: t_{R}=8.99 min, Method C, Purity: 97.08%.

6-Chloro-5-ethyl-4-hydroxypyridin-2 (1H)-one hydrochloride (56). A flame-dried flask under argon atmosphere was charged with malonyl chloride (5.0 mL, 51.4 mmol) and butyronitrile (8.95 mL, 90.9 mmol), and stirred at rt for 18 h. The precipitate was filtrated and washed repeatedly with DEE and dried to afford 56 with an unknown impurity as an off-white solid (1.60 g). The compound was used in the next step without further purification. 2,4,6-Trichloro-3-ethylpyridine (57). A pressure flask was charged with 56 (1.58 g) and phosphorus oxychloride (9.5 mL, 101.6 mmol). The flask was capped and stirred at 180° C. for 19 h. The cooled reaction mixture was concentrated, evaporated onto Celite, and purified by flash chromatography (5-8% EtOAc in heptane) to afford 57 as a colorless oil (1.02 g, 9% over 2 steps). R_{f}=0.64 (EtOAc:heptane; 1:4); ¹H NMR (400 MHz, CDCl₃) δ 7.30 (s, 1H), 2.91 (q, J=7.5 Hz, 2H), 1.19 (t, J=7.5 Hz, 3H); ¹³C NMR (101 MHz, CDCl₃) δ 151.0, 147.6, 146.9, 135.4, 124.0, 24.0, 12.1.

4-(Benzyloxy)-2,6-dichloro-3-ethylpyridine (37). A flame-dried vial under argon atmosphere was charged with 57 (978 mg, 4.65 mmol), NaH (60% wt/wt dispersion in oil, 223 mg, 5.58 mmol), anhydrous DMF (5 mL), and cooled down to 0° C. Benzylalcohol (480 μL, 4.64 mmol) was added dropwise (effervescence was observed). The suspension was stirred at 0° C. for 2 h and at rt for 16 h. The reaction was quenched by addition of sat. NH₄Cl (1.0 mL) and water (50 mL) and extracted with EtOAc (3×50 mL). The combined organic phase was washed with aqueous CaCl₂ (3.0 M, 150 mL), dried over anhydrous MgSO₄, filtered, evaporated onto Celite, and purified by flash chromatography (0-7% EtOAc in heptane) to afford 37 as a white solid (974 mg, 74%). R_{f}=0.40 (EtOAc:heptane; 1:9); ¹H NMR (600 MHz, CDCl₃) δ 7.49-7.36 (m, 5H), 6.81 (s, 1H), 5.13 (s, 2H), 2.77 (q, J=7.5 Hz, 2H), 1.14 (t, J=7.5 Hz, 3H); ¹³C NMR (151 MHz, CDCl₃) δ 165.3, 150.3, 148.4, 135.1, 129.0, 128.8, 127.4, 126.2, 107.0, 71.1, 20.2, 12.6.

6-((1,4-Dioxan-2-yl)methoxy)-4-(benzyloxy)-2-chloro-3-ethylpyridine (39). A flame-dried vial was charged with 37 (944 mg, 3.35 mmol) and tBuOK (450.5 mg, 4.01 mmol). The flask was evacuated and backfilled with argon three times. Then, ((1,4-dioxan-2-yl)methanol (370 μL, 3.35 mmol), and anhydrous dioxane (7.5 mL) were added. The vial was capped at stirred at 100° C. for 23.5 h. The reaction mixture was cooled down to rt and water (50 mL) was added and extracted with DCM (3×50 mL). The combined organic phase was dried over anhydrous MgSO₄, filtered, evaporated onto Celite, and purified by flash chromatography (5-8% EtOAc in heptane) to afford the 39 as a white solid (700 mg, 58%). R_{f}=0.08 (EtOAc:heptane; 1:9); ¹H NMR (400 MHz, CDCl₃) δ 7.50-7.30 (m, 5H), 6.26 (s, 1H), 5.06 (s, 2H), 4.27 (d, J=5.0 Hz, 2H), 4.02-3.92 (m, 1H), 3.90-3.61 (m, 5H), 3.57-3.44 (m, 1H), 2.72 (q, J=7.5 Hz, 2H), 1.12 (t, J=7.4 Hz, 3H); ¹³C NMR (101 MHz, CDCl₃) δ 166.1, 161.8, 147.6, 135.8, 128.9, 128.4, 127.2, 120.7, 92.9, 73.8, 70.5, 68.3, 66.9, 66.6, 65.8, 20.0, 13.1.

6-((1,4-Dioxan-2-yl)methoxy)-4-(benzyloxy)-3-ethyl-2-((4-((4-methoxybenzyl)oxy)phenyl)ethynyl)pyridine (58). The target compound was synthesized as described for compound 40a by Sonogashira Coupling of 39 (191 mg, 0.52 mmol) with 1-ethynyl-4-((4-methoxybenzyl)oxy)benzene (46) (150.1 mg, 0.63 mmol) in the presence of PdCl₂(MeCN)₂ (2.7 mg, 10.4 μmol), XPhos (15 mg, 31.5 μmol), and Cs₂CO₃ (444.7 mg, 1.36 mmol) in anhydrous MeCN (2.0 mL) at 90° C. for 2 d. The crude was purified by flash chromatography (10-25% EtOAc in heptane) to afford 58 as a yellow oil (219 mg, 74%). R_{f}=0.10 (EtOAc:heptane; 3:2); ¹H NMR (600 MHz, CDCl₃) δ 7.54-7.49 (m, 2H), 7.44-7.38 (m, 4H), 7.38-7.33 (m, 3H), 6.98-6.90 (m, 4H), 6.31 (s, 1H), 5.08 (s, 2H), 5.01 (s, 2H), 4.37 (dd, J=11.6, 4.0 Hz, 1H), 4.33 (dd, J=11.6, 6.0 Hz, 1H), 4.00-3.95 (m, 1H), 3.89-3.77 (m, 6H), 3.75-3.71 (m, 1H), 3.69-3.63 (m, 1H), 3.52 (dd, J=11.5, 10.1 Hz, 1H), 2.86 (q, J=7.4 Hz, 2H), 1.20 (t, J=7.5 Hz, 3H); ¹³C NMR (151 MHz, CDCl₃) δ 164.9, 162.7, 159.7, 159.4, 139.0, 136.1, 133.6, 129.4, 128.8, 128.7, 128.3, 127.3, 126.5, 115.2, 115.1, 114.2, 94.0, 91.8, 86.6, 74.1, 70.1, 70.0, 68.4, 66.9, 66.6, 65.5, 55.5, 20.5, 13.9.

4-((6-((1,4-Dioxan-2-yl)methoxy)-4-(benzyloxy)-3-ethylpyridin-2-yl)ethynyl)phenol (59). To a solution of 58 (213 mg, 0.38 mmol) and anisole (200 μL, 1.84 mmol) in DCM (4.0 mL) was added TFA (500 μL, 6.53 mmol). The reaction mixture was stirred at rt for 1 h. Water (2.0 mL) was added, and the mixture was neutralized by portion wise addition of Na₂CO₃. Additional water (13 mL) was added and extracted with DCM (3×15 mL). The combined organic phase was dried over anhydrous MgSO₄, filtered, evaporated onto Celite, and purified by flash chromatography (30-35% EtOAc in heptane) to afford 59 as an off-white solid (159 mg, 95%). R_{f}=0.2 (EtOAc:heptane; 4:1); ¹H NMR (600 MHz, CDCl₃) δ 7.44-7.38 (m, 6H), 7.38-7.32 (m, 1H), 6.81 (d, J=8.6 Hz, 2H), 6.30 (s, 1H), 5.91 (s, 1H), 5.07 (s, 2H), 4.37 (dd, J=11.6, 4.0 Hz, 1H), 4.33 (dd, J=11.6, 6.0 Hz, 1H), 4.02-3.95 (m, 1H), 3.86 (dd, J=11.6, 2.7 Hz, 2H), 3.80 (td, J=11.4, 2.8 Hz, 1H), 3.76-3.71 (m, 1H), 3.67 (td, J=11.3, 2.9 Hz, 1H), 3.55-3.49 (m, 1H), 2.85 (q, J=7.4 Hz, 2H), 1.19 (t, J=7.4 Hz, 3H); ¹³C NMR (151 MHz, CDCl₃) δ 165.0, 162.8, 156.6, 139.0, 136.1, 133.7, 128.8, 128.3, 127.3, 126.5, 115.8, 114.9, 94.0, 92.1, 86.2, 74.1, 70.1, 68.4, 66.9, 66.6, 65.6, 20.5, 13.9.

General Procedure for alkylation for the synthesis of compounds 60a-I as exemplified by compound 60a.

tert-Butyl (2-(4-((6-((1,4-dioxan-2-yl)methoxy)-4-(benzyloxy)-3-ethylpyridin-2-yl)ethynyl)phenoxy)ethyl)carbamate (60a). A flame-dried vial was charged with 59 (30 mg, 67.3 μmol), tert-butyl (2-bromoethyl)carbamate (30.2 mg, 0.13 mmol), Cs₂CO₃ (43.9 mg, 0.13 mmol) and KI (1.1 mg, 6.63 μmol). The vial was evacuated and backfilled with argon (3 x). Then, anhydrous MeCN (400 μL) was added. The vial was capped and stirred at 80° C. for 17.5 h. The cooled reaction mixture was filtered, and the filtrate was evaporated onto Celite, and purified by flash chromatography (15-35% EtOAc in heptane) to afford 60a as a colorless oil (35 mg, 88%). R_{f}=0.2 (EtOAc:heptane; 4:1); ¹H NMR (600 MHz, CDCl₃) δ 7.54-7.49 (m, 2H), 7.43-7.38 (m, 4H), 7.39-7.32 (m, 1H), 6.91-6.85 (m, 2H), 6.31 (s, 1H), 5.08 (s, 2H), 4.97 (s, 1H), 4.37 (dd, J=11.6, 4.1 Hz, 1H), 4.33 (dd, J=11.6, 6.0 Hz, 1H), 4.04 (t, J=5.1 Hz, 2H), 4.01-3.95 (m, 1H), 3.86 (dt, J=11.5, 3.1 Hz, 2H), 3.80 (td, J=11.3, 2.8 Hz, 1H), 3.73 (dd, J=11.7, 2.7 Hz, 1H), 3.66 (td, J=11.3, 2.9 Hz, 1H), 3.58-3.49 (m, 3H), 2.86 (q, J=7.4 Hz, 2H), 1.46 (s, 9H), 1.20 (t, J=7.4 Hz, 3H); ¹³C NMR (151 MHz, CDCl₃) δ 164.8, 162.6, 159.0, 155.9, 138.8, 136.0, 133.5, 128.7, 128.2, 127.1, 126.4, 115.3, 114.5, 93.9, 91.5, 86.6, 79.6, 73.9, 69.9, 68.3, 67.3, 66.8, 66.5, 65.4, 40.1, 28.4, 20.4, 13.8.

tert-Butyl (3-(4-((6-((1,4-dioxan-2-yl)methoxy)-4-(benzyloxy)-3-ethylpyridin-2-yl)ethynyl)phenoxy)propyl)carbamate (60b). The target compound was synthesized as described for compound 60a by alkylation of 59 (31 mg, 69.6 μmol) with tert-butyl (3-chloropropyl)carbamate (32.1 mg, 0.17 mmol) in the presence of Cs₂CO₃ (43.9 mg, 0.13 mmol) and KI (1.1 mg, 6.63 μmol) in anhydrous MeCN (400 μL) for 17.5 h. The crude was purified by flash chromatography (15-35% EtOAc in heptane) to afford 60b as a colorless oil (41 mg, 98%).

R_{f}=0.2 (EtOAc:heptane; 4:1); ¹H NMR (600 MHz, CDCl₃) δ 7.53-7.48 (m, 2H), 7.43-7.38 (m, 4H), 7.37-7.32 (m, 1H), 6.90-6.84 (m, 2H), 6.30 (s, 1H), 5.08 (s, 2H), 4.73 (br s, 1H), 4.37 (dd, J=11.6, 4.1 Hz, 1H), 4.33 (dd, J=11.6, 5.9 Hz, 1H), 4.04 (t, J=6.0 Hz, 2H), 4.00-3.95 (m, 1H), 3.86 (dt, J=11.5, 2.9 Hz, 2H), 3.80 (td, J=11.3, 2.8 Hz, 1H), 3.73 (dd, J=11.6, 2.7 Hz, 1H), 3.66 (td, J=11.3, 2.9 Hz, 1H), 3.52 (dd, J=11.5, 10.1 Hz, 1H), 3.33 (q, J=6.5 Hz, 2H), 2.86 (q, J=7.4 Hz, 2H), 1.99 (p, J=6.4 Hz, 2H), 1.44 (s, 9H), 1.20 (t, J=7.4 Hz, 3H); ¹³C NMR (151 MHz, CDCl₃) δ 164.9, 162.7, 159.3, 156.1, 139.0, 136.1, 133.6, 128.8, 128.3, 127.3, 126.5, 115.2, 114.6, 94.0, 91.7, 86.6, 79.4, 74.1, 70.1, 68.4, 66.9, 66.6, 66.0, 65.5, 38.1, 29.7, 28.6, 20.5, 13.9.

tert-Butyl (2-(2-(4-((6-((1,4-dioxan-2-yl)methoxy)-4-(benzyloxy)-3-ethylpyridin-2-yl)ethynyl)phenoxy)ethoxy)ethyl)carbamate (60c). The target compound was synthesized as described for compound 60a by alkylation of 59 (25 mg, 56.1 μmol) with 2-(2-((tert-butoxycarbonyl)amino)ethoxy)ethyl 4-methylbenzenesulfonate (40.3 mg, 0.11 mmol) in the presence of Cs₂CO₃ (29.3 mg, 89.9 μmol) and KI (1.0 mg, 6.02 μmol) in anhydrous MeCN (270 μL) for 16 h. The crude was purified by flash chromatography (35-40% EtOAc in heptane) to afford 60c as a colorless oil (34 mg, 96%). R_{f}=0.38 (EtOAc:heptane; 3:2); ¹H NMR (600 MHz, CDCl₃) δ 7.53-7.49 (m, 2H), 7.42-7.39 (m, 4H), 7.37-7.33 (m, 1H), 6.93-6.88 (m, 2H), 6.31 (s, 1H), 5.08 (s, 2H), 4.95 (br s, 1H), 4.37 (dd, J=11.6, 4.1 Hz, 1H), 4.33 (dd, J=11.6, 5.9 Hz, 1H), 4.15-4.13 (m, 2H), 4.00-3.95 (m, 1H), 3.88-3.77 (m, 5H), 3.73 (dd, J=11.7, 2.7 Hz, 1H), 3.67 (dd, J=11.2, 2.9 Hz, 1H), 3.63-3.59 (m, 2H), 3.52 (dd, J=11.5, 10.1 Hz, 1H), 3.35 (q, J=5.4 Hz, 2H), 2.86 (q, J=7.4 Hz, 2H), 1.45 (s, 9H), 1.20 (t, J=7.4 Hz, 3H).

2-(4-((6-((1,4-Dioxan-2-yl)methoxy)-4-(benzyloxy)-3-ethylpyridin-2-yl)ethynyl)phenoxy)ethan-1-ol (60d). The target compound was synthesized as described for compound 60a by alkylation of 59 (30 mg, 67.3 μmol) with 2-bromoethan-1-ol (29.6 μL, 0.41 mmol) in the presence of Cs₂CO₃ (65.8 mg, 0.20 mmol) and KI (1.1 mg, 6.63 μmol) in anhydrous MeCN (400 μL) for 32 h. The crude was purified by flash chromatography (35-55% EtOAc in heptane) to afford 60d as a colorless oil (13.1 mg, 40%). R_{f}=0.06 (EtOAc:heptane; 3:2); 1H NMR (600 MHz, CDCl₃) δ 7.51 (d, J=8.8 Hz, 2H), 7.43-7.32 (m, 5H), 6.90 (d, J=8.8 Hz, 2H), 6.31 (s, 1H), 5.08 (s, 2H), 4.40-4.29 (m, 2H), 4.11 (t, J=4.8, 4.3 Hz, 2H), 4.02-3.94 (m, 3H), 3.86 (dt, J=11.5, 2.6 Hz, 2H), 3.80 (td, J=11.3, 2.7 Hz, 1H), 3.73 (dd, J=11.6, 2.7 Hz, 1H), 3.66 (td, J=11.3, 2.9 Hz, 1H), 3.52 (dd, J=11.5, 10.1 Hz, 1H), 2.86 (q, J=7.4 Hz, 2H), 2.04 (br s, 1H), 1.20 (t, J=7.4 Hz, 3H); ¹³C NMR (151 MHz, CDCl₃) δ 164.9, 162.7, 159.1, 138.9, 136.1, 133.6, 128.8, 128.3, 127.3, 126.5, 115.5, 114.7, 94.0, 91.6, 86.7, 74.1, 70.1, 69.4, 68.4, 66.9, 66.6, 65.5, 61.5, 20.5, 13.9.

Ethyl 5-(4-((6-((1,4-dioxan-2-yl)methoxy)-4-(benzyloxy)-3-ethylpyridin-2-yl)ethynyl)phenoxy)pentanoate (60e). The target compound was synthesized as described for compound 60a by alkylation of 59 (30 mg, 67.3 μmol) with ethyl 5-bromopentanoate (32 μL, 0.20 mmol) in the presence of Cs₂CO₃ (65.8 mg, 0.20 mmol) and KI (1.1 mg, 6.63 μmol) in anhydrous MeCN (400 μL) for 14 h. The crude was purified by flash chromatography (20-25% EtOAc in heptane) to afford 60e as a colorless oil (27.5 mg, 71%). R_{f}=0.31 (EtOAc:heptane; 3:2); ¹H NMR (600 MHz, CDCl₃) δ 7.50 (d, J=8.8 Hz, 2H), 7.43-7.38 (m, 4H), 7.35 (ddd, J=8.7, 5.1, 3.8 Hz, 1H), 6.86 (d, J=8.8 Hz, 2H), 6.30 (s, 1H), 5.08 (s, 2H), 4.37 (dd, J=11.6, 4.0 Hz, 1H), 4.33 (dd, J=11.6, 6.0 Hz, 1H), 4.14 (q, J=7.1 Hz, 2H), 4.02-3.95 (m, 3H), 3.86 (dt, J=11.5, 2.6 Hz, 2H), 3.80 (td, J=11.2, 2.8 Hz, 1H), 3.75-3.70 (m, 1H), 3.66 (td, J=11.3, 2.9 Hz, 1H), 3.52 (dd, J=11.5, 10.1 Hz, 1H), 2.86 (q, J=7.4 Hz, 2H), 2.39 (t, J=7.0 Hz, 2H), 1.87-1.79 (m, 4H), 1.26 (t, J=7.2 Hz, 3H), 1.20 (t, J=7.4 Hz, 3H); ¹³C NMR (151 MHz, CDCl₃) δ 173.5, 164.9, 162.7, 159.5, 139.0, 136.1, 133.6, 128.8, 128.3, 127.3, 126.4, 114.9, 114.6, 93.9, 91.9, 86.5, 74.1, 70.1, 68.4, 67.6, 66.9, 66.6, 65.5, 60.5, 34.1, 28.7, 21.8, 20.5, 14.4, 13.9.

2-(4-((6-((1,4-Dioxan-2-yl)methoxy)-4-(benzyloxy)-3-ethylpyridin-2-yl)ethynyl)phenoxy)acetamide (60f). The target compound was synthesized as described for compound 60a by alkylation of 59 (30 mg, 67.3 μmol) with 2-chloroacetamide (25.2 mg, 0.27 mmol) in the presence of Cs₂CO₃ (87.8 mg, 0.27 mmol) and KI (1.1 mg, 6.63 μmol) in anhydrous MeCN (400 μL) for 14 h. The crude was purified by flash chromatography (70-80% EtOAc in heptane) to afford 60f as a white solid (22.5 mg, 80%). R_{f}=0.37 (EtOAc); ¹H NMR (600 MHz, CDCl₃) δ 7.55 (d, J=8.8 Hz, 2H), 7.44-7.30 (m, 5H), 6.91 (d, J=8.7 Hz, 2H), 6.51 (s, 1H), 6.32 (br s, 1H), 5.78 (br s, 1H), 5.08 (s, 2H), 4.52 (s, 2H), 4.40-4.30 (m, 2H), 4.01-3.92 (m, 1H), 3.86 (dt, J=11.5, 2.9 Hz, 2H), 3.80 (td, J=11.3, 2.7 Hz, 1H), 3.73 (dd, J=11.8, 2.7 Hz, 1H), 3.66 (td, J=11.3, 2.9 Hz, 1H), 3.52 (dd, J=11.5, 10.1 Hz, 1H), 2.85 (q, J=7.4 Hz, 2H), 1.20 (t, J=7.4 Hz, 3H); ¹³C NMR (151 MHz, CDCl₃) δ 170.4, 164.9, 162.8, 157.5, 138.7, 136.0, 133.8, 128.8, 128.3, 127.3, 126.6, 116.8, 114.8, 94.1, 91.0, 87.2, 74.0, 70.1, 68.4, 67.3, 66.9, 66.6, 65.5, 20.5, 13.9.

6-((1,4-Dioxan-2-yl)methoxy)-4-(benzyloxy)-3-ethyl-2-((4-(2-phenoxyethoxy)phenyl)ethynyl)pyridine (60g). The target compound was synthesized as described for compound 60a by alkylation of 59 (30 mg, 67.3 μmol) with (2-bromoethoxy)benzene (27.1 mg, 0.13 mmol) in the presence of Cs₂CO₃ (43.9 mg, 0.13 mmol) and KI (1.1 mg, 6.63 μmol) in anhydrous MeCN (400 μL) for 15 h. The crude was purified by flash chromatography (15-20% EtOAc in heptane) to afford 60g as a white solid (34 mg, 89%). R_{f}=0.37 (EtOAc:heptane; 3:2); ¹H NMR (400 MHz, CDCl₃) δ 7.56-7.51 (m, 2H), 7.45-7.27 (m, 7H), 7.01-6.91 (m, 5H), 6.31 (s, 1H), 5.08 (s, 2H), 4.40-4.31 (m, 6H), 4.01-3.95 (m, 1H), 3.89-3.84 (m, 2H), 3.80 (td, J=11.6, 11.1, 2.8 Hz, 1H), 3.76-3.71 (m, 1H), 3.70-3.62 (m, 1H), 3.52 (dd, J=11.5, 10.1 Hz, 1H), 2.87 (q, J=7.4 Hz, 2H), 1.21 (t, J=7.4 Hz, 3H); ¹³C NMR (101 MHz, CDCl₃) δ 164.9, 162.7, 159.2, 158.7, 139.0, 136.1, 133.6, 129.7, 128.8, 128.3, 127.2, 126.5, 121.3, 115.5, 114.9, 114.8, 94.0, 91.7, 86.7, 74.1, 70.1, 68.4, 66.9, 66.8, 66.6, 66.5, 65.5, 20.5, 13.9.

6-((1,4-Dioxan-2-yl)methoxy)-4-(benzyloxy)-3-ethyl-2-((4-(3-(methylsulfonyl)propoxy)phenyl)ethynyl)pyridine (60h). The target compound was synthesized as described for compound 60a by alkylation of 59 (30 mg, 67.3 μmol) with 3-(methylsulfonyl)propyl 4-methylbenzenesulfonate (39.4 mg, 0.13 mmol) in the presence of Cs₂CO₃ (43.9 mg, 0.13 mmol) and KI (1.1 mg, 6.63 μmol) in anhydrous MeCN (400 μL) for 15 h. The crude was purified by flash chromatography (15-60% EtOAc in heptane) to afford 60h as a colorless oil (29 mg, 76%). R_{f}=0.64 (EtOAc); ¹H NMR (400 MHz, CDCl₃) δ 7.51 (d, J=8.8 Hz, 2H), 7.45-7.31 (m, 5H), 6.87 (d, J=8.8 Hz, 2H), 6.31 (s, 1H), 5.07 (s, 2H), 4.39-4.29 (m, 2H), 4.14 (t, J=5.8 Hz, 2H), 4.02-3.92 (m, 1H), 3.89-3.83 (m, 2H), 3.79 (td, J=11.6, 11.1, 2.7 Hz, 1H), 3.75-3.70 (m, 1H), 3.69-3.60 (m, 1H), 3.51 (dd, J=11.5, 10.1 Hz, 1H), 3.30-3.21 (m, 2H), 2.96 (s, 3H), 2.85 (q, J=7.4 Hz, 2H), 2.42-2.30 (m, 2H), 1.20 (t, J=7.4 Hz, 3H); ¹³C NMR (101 MHz, CDCl₃) δ 164.9, 162.7, 158.8, 138.9, 136.1, 133.7, 128.8, 128.3, 127.2, 126.5, 115.7, 114.6, 94.0, 91.4, 86.8, 74.0, 70.1, 68.4, 66.9, 66.6, 65.7, 65.5, 51.8, 41.1, 22.6, 20.5, 13.9.

5-((4-((6-((1,4-Dioxan-2-yl)methoxy)-4-(benzyloxy)-3-ethylpyridin-2-yl)ethynyl)phenoxy)methyl)-3-methyl-1,2,4-oxadiazole (60i). The target compound was synthesized as described for compound 60a by alkylation of 59 (30 mg, 67.3 μmol) with 5-(chloromethyl)-3-methyl-1,2,4-oxadiazole (17.9 mg, 0.14 mmol) in the presence of Cs₂CO₃ (43.9 mg, 0.13 mmol) and KI (1.1 mg, 6.63 μmol) in anhydrous MeCN (400 μL) for 16 h. The crude was purified by flash chromatography (33-35% EtOAc in heptane) to afford 60i as a colorless oil (31.5 mg, 85%). R_{f}=0.18 (EtOAc:heptane; 3:2); ¹H NMR (600 MHz, CDCl₃) δ 7.54 (d, J=8.8 Hz, 2H), 7.43-7.38 (m, 4H), 7.37-7.33 (m, 1H), 6.98 (d, J=8.8 Hz, 2H), 6.31 (s, 1H), 5.28 (s, 2H), 5.08 (s, 2H), 4.38-4.29 (m, 2H), 4.00-3.95 (m, 1H), 3.88-3.83 (m, 2H), 3.83-3.76 (m, 1H), 3.73 (dd, J=11.7, 2.5 Hz, 1H), 3.66 (td, J=11.3, 2.9 Hz, 1H), 3.52 (dd, J=11.4, 10.0 Hz, 1H), 2.85 (q, J=7.4 Hz, 2H), 2.44 (s, 3H), 1.19 (t, J=7.4 Hz, 3H); ¹³C NMR (151 MHz, CDCl₃) δ 174.3, 167.7, 164.9, 162.8, 157.8, 138.7, 136.1, 133.7, 128.8, 128.3, 127.3, 126.6, 116.9, 115.0, 94.1, 91.1, 87.1, 74.1, 70.1, 68.4, 66.9, 66.6, 65.5, 61.1, 20.5, 13.9, 11.7.

Methyl 2-((4-((6-((1,4-dioxan-2-yl)methoxy)-4-(benzyloxy)-3-ethylpyridin-2-yl)ethynyl)phenoxy)methyl)oxazole-4-carboxylate (60j). The target compound was synthesized as described for compound 60a by alkylation of 59 (20 mg, 44.9 μmol) with methyl 2-(chloromethyl)oxazole-4-carboxylate (19.7 mg, 0.11 mmol) in the presence of Cs₂CO₃ (29.3 mg, 89.9 μmol) and KI (1.0 mg, 6.02 μmol) in anhydrous MeCN (270 μL) for 16 h. The crude was purified by flash chromatography (35-45% EtOAc in heptane) to afford 60j as a colorless oil (23 mg, 88%). R_{f}=0.29 (EtOAc:heptane; 3:2); ¹H NMR (600 MHz, CDCl₃) δ 8.27 (s, 1H), 7.55-7.49 (m, 2H), 7.42-7.38 (m, 4H), 7.37-7.33 (m, 1H), 7.00-6.97 (m, 2H), 6.31 (s, 1H), 5.21 (s, 2H), 5.08 (s, 2H), 4.36 (dd, J=11.6, 4.0 Hz, 1H), 4.33 (dd, J=11.6, 5.9 Hz, 1H), 4.00-3.95 (m, 1H), 3.93 (s, 3H), 3.86 (dt, J=11.4, 2.7 Hz, 2H), 3.79 (td, J=11.2, 2.8 Hz, 1H), 3.73 (dd, J=11.6, 2.6 Hz, 1H), 3.66 (td, J=11.4, 2.9 Hz, 1H), 3.51 (dd, J=11.5, 10.1 Hz, 1H), 2.85 (q, J=7.4 Hz, 2H), 1.19 (t, J=7.5 Hz, 3H); ¹³C NMR (151 MHz, CDCl₃) δ 165.0, 162.7, 161.4, 160.2, 158.1, 145.1, 138.7, 136.0, 133.8, 133.7, 128.8, 128.3, 127.3, 126.6, 116.6, 115.0, 94.1, 91.3, 87.0, 74.1, 70.1, 68.4, 66.9, 66.6, 65.6, 62.1, 52.5, 29.8, 20.5, 13.9.

2-(2-(4-((6-((1,4-Dioxan-2-yl)methoxy)-4-(benzyloxy)-3-ethylpyridin-2-yl)ethynyl)phenoxy)ethyl)isoindoline-1,3-dione (60k). The target compound was synthesized as described for compound 60a by alkylation of 59 (20 mg, 44.9 μmol) with 2-(2-bromoethyl)isoindoline-1,3-dione (57 mg, 0.22 mmol) in the presence of Cs₂CO₃ (58.6 mg, 0.18 mmol) and KI (2.0 mg, 12.0 μmol) in anhydrous MeCN (540 μL) for 2.5 d. The crude was purified by flash chromatography (15-35% EtOAc in heptane) to afford 60k as a colorless oil (20 mg, 72%). R_{f}=0.47 (EtOAc:heptane; 3:2); ¹H NMR (600 MHz, CDCl₃) δ 7.87 (dd, J=5.5, 3.1 Hz, 2H), 7.73 (dd, J=5.5, 3.0 Hz, 2H), 7.50-7.44 (m, 2H), 7.43-7.38 (m, 4H), 7.38-7.31 (m, 1H), 6.88-6.81 (m, 2H), 6.30 (s, 1H), 5.07 (s, 2H), 4.35 (dd, J=11.6, 4.1 Hz, 1H), 4.32 (dd, J=11.6, 5.9 Hz, 1H), 4.25 (t, J=5.8 Hz, 2H), 4.12 (t, J=5.8 Hz, 2H), 3.99-3.94 (m, 1H), 3.88-3.83 (m, 2H), 3.79 (td, J=11.3, 2.8 Hz, 1H), 3.72 (dd, J=11.7, 2.6 Hz, 1H), 3.66 (td, J=11.3, 2.9 Hz, 1H), 3.51 (dd, J=11.5, 10.1 Hz, 1H), 2.84 (q, J=7.4 Hz, 2H), 1.18 (t, J=7.4 Hz, 3H); ¹³C NMR (151 MHz, CDCl₃) δ 168.3, 164.9, 162.7, 158.8, 138.9, 136.1, 134.3, 134.2, 133.5, 132.2, 128.8, 128.3, 127.2, 126.5, 123.5, 115.5, 114.8, 94.0, 91.6, 86.7, 74.1, 70.0, 68.4, 66.9, 66.6, 65.5, 64.9, 37.4, 20.5, 13.9.

6-((1,4-Dioxan-2-yl)methoxy)-4-(benzyloxy)-2-((4-(ethoxy-ds)phenyl)ethynyl)-3-ethylpyridine (601). The target compound was synthesized as described for compound 60a by alkylation of 59 (60 mg, 0.13 mmol) with 1-bromoethane-1,1,2,2,2-d₅ (40.2 μL, 0.54 mmol) in the presence of Cs₂CO₃ (175.6 mg, 54 mmol) and KI (2.2 mg, 13.3 μmol) in anhydrous MeCN (800 μL) for 26 h. The crude was purified by flash chromatography (20% EtOAc in heptane) to afford 601 as a white solid (55 mg 85%). R_{f}=0.57 (EtOAc:heptane; 3:2); ¹H NMR (600 MHz, CDCl₃) δ 7.53-7.47 (m, 2H), 7.44-7.37 (m, 4H), 7.37-7.31 (m, 1H), 6.89-6.84 (m, 2H), 6.30 (s, 1H), 5.08 (s, 2H), 4.37 (dd, J=11.6, 4.0 Hz, 1H), 4.33 (dd, J=11.6, 6.0 Hz, 1H), 4.01-3.94 (m, 1H), 3.89-3.83 (m, 2H), 3.80 (td, J=11.3, 2.8 Hz, 1H), 3.73 (dd, J=11.4, 2.6 Hz, 1H), 3.66 (td, J=11.3, 2.9 Hz, 1H), 3.52 (dd, J=11.5, 10.1 Hz, 1H), 2.86 (q, J=7.5 Hz, 2H), 1.20 (t, J=7.4 Hz, 3H); ¹³C NMR (151 MHz, CDCl₃) δ 164.9, 162.7, 159.5, 139.1, 136.1, 133.6, 128.8, 128.3, 127.3, 126.4, 114.8, 114.6, 93.9, 91.9, 86.5, 74.1, 70.1, 68.4, 66.9, 66.6, 65.5, 20.5, 13.9.

Example 32, tert-Butyl (2-(4-(2-(6-((1,4-dioxan-2-yl)methoxy)-3-ethyl-4-hydroxypyridin-2-yl)ethyl)phenoxy)ethyl)carbamate (61a). The target compound was synthesized as described for compound 5 by debenzylation and hydrogenation of 60a (31 mg, 52.7 μmol) in MeOH:EtOAc (1:2; 3.0 mL), catalyzed by 10% Pd—C (2.8 mg, 2.63 μmol) under hydrogen atmosphere for 5 h. The crude was purified by flash chromatography (5-6% MeOH in DCM) to afford 61a as a colorless oil (26 mg, 98%). R_{f}=0.48 (MeOH:DCM; 1:9); ¹H NMR (600 MHz, DMSO) δ 10.30 (br s, 1H), 7.09 (d, J=8.7 Hz, 2H), 6.96 (t, J=5.7 Hz, 1H), 6.81 (d, J=8.7 Hz, 2H), 6.01 (s, 1H), 4.16 (dd, J=11.4, 5.8 Hz, 1H), 4.13 (dd, J=11.4, 4.4 Hz, 1H), 3.90 (t, J=5.9 Hz, 2H), 3.84-3.79 (m, 1H), 3.79-3.72 (m, 2H), 3.68-3.63 (m, 1H), 3.59 (td, J=11.2, 2.6 Hz, 1H), 3.48 (td, J=11.2, 2.7 Hz, 1H), 3.35 (dd, J=11.4, 10.0 Hz, 1H), 3.26 (q, J=5.9 Hz, 2H), 2.90-2.84 (m, 2H), 2.84-2.78 (m, 2H), 2.42 (q, J=7.4 Hz, 2H), 1.38 (s, 9H), 0.94 (t, J=7.4 Hz, 3H); ¹³C NMR (151 MHz, DMSO) δ 164.2, 161.0, 156.6, 155.6, 133.8, 129.2, 129.2, 118.6, 114.3, 93.0, 77.7, 73.3, 67.6, 66.3, 65.8, 65.8, 64.4, 40.1, 35.5, 33.7, 28.2, 17.6, 14.1; HPLC: t_{R}=11.12 min, Method C, Purity: 99.32%.

Example 33, tert-Butyl (3-(4-(2-(6-((1,4-dioxan-2-yl)methoxy)-3-ethyl-4-hydroxypyridin-2-yl)ethyl)phenoxy)propyl)carbamate(61b). The target compound was synthesized as described for compound 5 by debenzylation and hydrogenation of 60b (41 mg, 68.0 μmol) in MeOH:EtOAc (1:2; 3.0 mL), catalyzed by 10% Pd—C (3.6 mg, 3.38 μmol) under hydrogen atmosphere for 5 h. The crude was purified by flash chromatography (5-6% MeOH in DCM) to afford 61b as a colorless oil (34 mg, 97%). R_{f}=0.48 (MeOH:DCM; 1:9); ¹H NMR (600 MHz, DMSO) δ 10.29 (br s, 1H), 7.09 (d, J=8.5 Hz, 2H), 6.86 (t, J=5.7 Hz, 1H), 6.80 (d, J=8.6 Hz, 2H), 6.00 (s, 1H), 4.16 (dd, J=11.4, 5.8 Hz, 1H), 4.13 (dd, J=11.4, 4.4 Hz, 1H), 3.91 (t, J=6.3 Hz, 2H), 3.82 (s, 1H), 3.79-3.72 (m, 2H), 3.65 (dd, J=11.3, 2.6 Hz, 1H), 3.59 (td, J=11.3, 2.6 Hz, 1H), 3.48 (td, J=11.2, 2.7 Hz, 1H), 3.37-3.33 (m, 1H), 3.06 (q, J=6.6 Hz, 2H), 2.89-2.84 (m, 2H), 2.84-2.79 (m, 2H), 2.42 (q, J=7.4 Hz, 2H), 1.80 (p, J=6.6 Hz, 2H), 1.37 (s, 9H), 0.94 (t, J=7.4 Hz, 3H); ¹³C NMR (151 MHz, DMSO) δ 164.2, 161.1, 156.7, 155.6, 133.7, 129.2, 118.6, 114.2, 93.0, 77.5, 73.3, 67.6, 65.8, 65.8, 65.2, 64.3, 37.0, 35.6, 33.7, 29.2, 28.2, 17.6, 14.1; HPLC: t_{R}=11.29 min, Method C, Purity: 96.57%.

Example 34, tert-Butyl (2-(2-(4-(2-(6-((1,4-dioxan-2-yl)methoxy)-3-ethyl-4-hydroxypyridin-2-yl)ethyl)phenoxy)ethoxy)ethyl)carbamate (61c). The target compound was synthesized as described for compound 5 by debenzylation and hydrogenation of 60c (34 mg, 53.7 μmol) in MeOH:EtOAc (1:2; 3.0 mL), catalyzed by 10% Pd—C (2.9 mg, 2.73 μmol) under hydrogen atmosphere for 2.5 h. The crude was purified by flash chromatography (6% MeOH in DCM and the obtained product was further purified by preparative HPLC (40% solvent B) to afford 61c as a colorless oil (25.8 mg, 88%). R_{f}=0.41 (MeOH:DCM; 1:9); ¹H NMR (600 MHz, DMSO) δ 7.12 (d, J=8.5 Hz, 2H), 6.85 (d, J=8.6 Hz, 2H), 6.77 (t, J=5.8 Hz, 1H), 6.37 (br s, 1H), 4.23 (d, J=5.1 Hz, 2H), 4.03 (t, J=4.5 Hz, 2H), 3.92-3.86 (m, 1H), 3.82-3.75 (m, 2H), 3.71-3.59 (m, 4H), 3.50 (td, J=11.2, 2.7 Hz, 1H), 3.45-3.36 (m, 3H), 3.09 (q, J=6.0 Hz, 2H), 2.93-2.87 (m, 2H), 2.87-2.81 (m, 2H), 2.44 (q, J=7.3 Hz, 2H), 1.37 (s, 9H), 0.98 (t, J=7.4 Hz, 3H); ¹³C NMR (151 MHz, DMSO) δ 160.0, 156.9, 155.6, 133.9-131.5 (m), 129.3, 120.1, 114.3, 92.7, 77.6, 72.9, 69.3, 68.6, 67.0, 65.8, 65.7, 40.1, 34.0, 28.2, 17.5, 13.7; HPLC: t_{R}=10.90 min, Method C, Purity: 96.97%.

Example 35, 6-((1,4-Dioxan-2-yl)methoxy)-3-ethyl-2-(4-(2-hydroxyethoxy)phenethyl)pyridin-4-ol (61d). The target compound was synthesized as described for compound 5 by debenzylation and hydrogenation of 60d (12.8 mg, 26.2 μmol) in MeOH:EtOAc (1:2; 3.0 mL), catalyzed by 10% Pd—C (1.4 mg, 1.32 μmol) under hydrogen atmosphere for 1.5 h. The crude was purified by flash chromatography (6% MeOH in DCM) to afford 61d as a colorless oil (7.3 mg, 69%). R_{f}=0.15 (MeOH:DCM; 5:95); ¹H NMR (600 MHz, MeOD) δ 7.07 (d, J=8.6 Hz, 2H), 6.85 (d, J=8.6 Hz, 2H), 5.89 (br s, 1H), 4.15-4.07 (m, 2H), 4.03-3.98 (m, 2H), 3.96-3.89 (m, 1H), 3.88-3.83 (m, 3H), 3.83-3.79 (m, 1H), 3.77-3.69 (m, 2H), 3.65-3.58 (m, 1H), 3.50 (dd, J=11.5, 10.0 Hz, 1H), 2.90-2.81 (m, 4H), 2.43 (q, J=7.4 Hz, 2H), 0.99 (t, J=7.5 Hz, 3H); ¹³C NMR (151 MHz, MeOD) δ 161.3, 158.9, 134.6, 130.4, 122.8, 115.6, 93.9, 74.9, 70.6, 69.2, 68.2, 67.7, 67.5, 61.8, 35.8, 19.0, 14.4; HPLC: t_{R}=9.02 min, Method C, Purity: 99.29%.

Example 36, Ethyl 5-(4-(2-(6-((1,4-dioxan-2-yl)methoxy)-3-ethyl-4-hydroxypyridin-2-yl)ethyl)phenoxy)pentanoate (61e). The target compound was synthesized as described for compound 5 by debenzylation and hydrogenation of 60e (27.1 mg, 47.2 μmol) in MeOH:EtOAc (1:2; 3.0 mL), catalyzed by 10% Pd—C (2.5 mg, 2.35 μmol) under hydrogen atmosphere for 2 h. The crude was purified by flash chromatography (6% MeOH in DCM) to afford 61e as a colorless oil (23 mg, quant.). R_{f}=0.21 (MeOH:DCM; 5:95); ¹H NMR (600 MHz, MeOD) δ 7.06 (d, J=8.5 Hz, 2H), 6.80 (d, J=8.7 Hz, 2H), 5.89 (br s, 1H), 4.15-4.06 (m, 4H), 3.96-3.88 (m, 3H), 3.85 (dd, J=11.5, 2.7 Hz, 1H), 3.82-3.79 (m, 1H), 3.77-3.68 (m, 2H), 3.61 (td, J=11.5, 2.9 Hz, 1H), 3.49 (dd, J=11.5, 10.0 Hz, 1H), 2.89-2.79 (m, 4H), 2.43 (q, J=7.4 Hz, 2H), 2.40-2.35 (m, 2H), 1.82-1.73 (m, 4H), 1.24 (t, J=7.1 Hz, 3H), 1.00 (t, J=7.4 Hz, 3H); ¹³C NMR (151 MHz, MeOD) δ 175.3, 161.6, 158.9, 134.4, 130.4, 123.8, 115.5, 93.9, 74.9, 69.2, 68.5, 68.1, 67.7, 67.5, 61.4, 35.8, 34.8, 29.8, 22.8, 19.0, 14.5, 14.4.

Example 37, 2-(4-(2-(6-((1,4-Dioxan-2-yl)methoxy)-3-ethyl-4-hydroxypyridin-2-yl)ethyl)phenoxy)acetamide (61f). The target compound was synthesized as described for compound 5 by debenzylation and hydrogenation of 60f (22 mg, 43.8 μmol) in MeOH:EtOAc (1:2; 3.0 mL), catalyzed by 10% Pd—C (2.3 mg, 2.16 μmol) under hydrogen atmosphere for 1.5 h. The crude was purified by flash chromatography (5-6% MeOH in DCM) to afford 61f as a colorless oil (6.5 mg, 36%). R_{f}=0.17 (MeOH:DCM; 5:95); ¹H NMR (600 MHz, MeOD) δ 7.11 (d, J=8.7 Hz, 2H), 6.89 (d, J=8.7 Hz, 2H), 5.89 (br s, 1H), 4.45 (s, 2H), 4.16-4.06 (m, 2H), 3.95-3.88 (m, 1H), 3.86-3.78 (m, 2H), 3.76-3.68 (m, 2H), 3.64-3.58 (m, 1H), 3.49 (dd, J=11.5, 10.0 Hz, 1H), 2.92-2.82 (m, 4H), 2.43 (q, J=7.5 Hz, 2H), 1.00 (t, J=7.4 Hz, 3H); ¹³C NMR (151 MHz, MeOD) δ 174.2, 157.6, 135.5, 130.6, 115.8, 94.0, 74.9, 69.2, 68.1, 67.7, 67.5, 35.7, 19.0, 14.4; HPLC: t_{R}=8.81 min, Method C, Purity: 99.29%.

Example 38, 6-((1,4-Dioxan-2-yl)methoxy)-3-ethyl-2-(4-(2-phenoxyethoxy)phenethyl)pyridin-4-ol (61g). The target compound was synthesized as described for compound 5 by debenzylation and hydrogenation of 60g (35 mg, 61.9 μmol) in MeOH:EtOAc (1:2; 3.0 mL), catalyzed by 10% Pd—C (3.3 mg, 3.10 μmol) under hydrogen atmosphere for 3 h. The crude was purified by flash chromatography (6% MeOH in DCM) to afford 61g as a colorless oil (28.3 mg, 95%). R_{f}=0.29 (MeOH:DCM; 5:95); ¹H NMR (600 MHz, DMSO) δ 10.29 (s, 1H), 7.33-7.27 (m, 2H), 7.11 (d, J=8.6 Hz, 2H), 6.99-6.92 (m, 3H), 6.87 (d, J=8.5 Hz, 2H), 6.00 (s, 1H), 4.30-4.24 (m, 4H), 4.19-4.09 (m, 2H), 3.84-3.79 (m, 1H), 3.76 (s, 2H), 3.65 (dd, J=11.2, 2.6 Hz, 1H), 3.59 (td, J=11.3, 2.6 Hz, 1H), 3.48 (td, J=11.2, 2.7 Hz, 1H), 3.35 (dd, J=11.4, 10.0 Hz, 1H), 2.91-2.85 (m, 2H), 2.85-2.79 (m, 2H), 2.42 (q, J=7.4 Hz, 2H), 0.94 (t, J=7.4 Hz, 3H); ¹³C NMR (151 MHz, DMSO) δ 164.2, 161.1, 158.3, 156.5, 155.6, 134.1, 129.5, 129.3, 120.7, 118.5, 114.5, 114.3, 93.0, 73.4, 67.7, 66.3, 66.2, 65.8, 65.8, 64.3, 35.5, 33.7, 17.6, 14.1; HPLC: t_{R}=11.60 min, Method C, Purity: 99.52%.

Example 39, 6-((1,4-Dioxan-2-yl)methoxy)-3-ethyl-2-(4-(3-(methylsulfonyl)propoxy)phenethyl)pyridin-4-ol (61h). The target compound was synthesized as described for compound 5 by debenzylation and hydrogenation of 60h (25 mg, 44.2 μmol) in MeOH:EtOAc (1:2; 3.0 mL), catalyzed by 10% Pd—C (2.4 mg, 2.26 μmol) under hydrogen atmosphere for 3 h. The crude was purified by flash chromatography (6% MeOH in DCM) to afford 61h as a colorless oil (21 mg, 99%). R_{f}=0.22 (MeOH:DCM; 5:95); ¹H NMR (600 MHz, DMSO) δ 10.29 (s, 1H), 7.10 (d, J=8.7 Hz, 2H), 6.83 (d, J=8.6 Hz, 2H), 6.00 (s, 1H), 4.19-4.09 (m, 2H), 4.03 (t, J=6.2 Hz, 2H), 3.85-3.79 (m, 1H), 3.79-3.72 (m, 2H), 3.68-3.63 (m, 1H), 3.59 (td, J=11.3, 2.6 Hz, 1H), 3.48 (td, J=11.2, 2.7 Hz, 1H), 3.35 (dd, J=11.4, 9.9 Hz, 1H), 3.28-3.22 (m, 2H), 3.01 (s, 3H), 2.90-2.84 (m, 2H), 2.84-2.79 (m, 2H), 2.42 (q, J=7.4 Hz, 2H), 2.15-2.08 (m, 2H), 0.94 (t, J=7.4 Hz, 3H); ¹³C NMR (151 MHz, DMSO) δ 164.2, 161.1, 156.4, 155.6, 134.1, 129.2, 118.5, 114.3, 93.0, 73.4, 67.7, 65.8, 65.8, 65.5, 64.3, 50.6, 40.2, 35.5, 33.7, 22.0, 17.6, 14.1; HPLC: t_{R}=9.72 min, Method C, Purity: 99.03%.

Example 40, 6-((1,4-Dioxan-2-yl)methoxy)-3-ethyl-2-(4-((3-methyl-1,2,4-oxadiazol-5-yl)methoxy)phenethyl)pyridin-4-ol (61i). The target compound was synthesized as described for compound 5 by debenzylation and hydrogenation of 60i (31.1 mg, 57.4 μmol) in MeOH:EtOAc (1:2; 3.0 mL), catalyzed by 10% Pd—C (3.1 mg, 2.91 μmol) under hydrogen atmosphere for 3.5 h. The crude was purified by flash chromatography (6% MeOH in DCM) to afford 61i as a colorless oil (20.6 mg, 79%). ¹H NMR (600 MHz, MeOD) δ 7.08 (d, J=8.6 Hz, 2H), 6.82 (d, J=8.6 Hz, 2H), 5.89 (s, 1H), 4.66 (s, 2H), 4.15-4.06 (m, 2H), 3.95-3.89 (m, 1H), 3.85 (dd, J=11.5, 2.7 Hz, 1H), 3.83-3.79 (m, 1H), 3.78-3.69 (m, 5H), 3.65-3.57 (m, 1H), 3.50 (dd, J=11.5, 10.0 Hz, 1H), 2.91-2.80 (m, 4H), 2.43 (q, J=7.4 Hz, 2H), 1.00 (t, J=7.5 Hz, 3H); ¹³C NMR (151 MHz, MeOD) δ 171.5, 157.9, 135.5, 130.5, 115.6, 93.9, 74.9, 69.2, 67.7, 67.5, 66.2, 52.5, 35.8, 19.0, 14.4; HPLC: t_{R}=9.84 min, Method C, Purity: 95.16%.

Example 41, Methyl 2-((4-(2-(6-((1,4-dioxan-2-yl)methoxy)-3-ethyl-4-hydroxypyridin-2-yl)ethyl)phenoxy)methyl)oxazole-4-carboxylate (61j). The target compound was synthesized as described for compound 5 by debenzylation and hydrogenation of 60j (23 mg, 39.3 μmol) in MeOH:EtOAc (1:2; 3.0 mL), catalyzed by 10% Pd—C (2.1 mg, 1.97 μmol) under hydrogen atmosphere for 2.5 h. The crude was purified by flash chromatography (5-6% MeOH in DCM) to afford 61j as a colorless oil (16.3 mg, 83%). R_{f}=0.41 (MeOH:DCM; 1:9); ¹H NMR (600 MHz, DMSO) δ 10.29 (br s, 1H), 8.90 (s, 1H), 7.13 (d, J=8.7 Hz, 2H), 6.93 (d, J=8.8 Hz, 2H), 6.00 (s, 1H), 5.24 (s, 2H), 4.16 (dd, J=11.4, 5.8 Hz, 1H), 4.12 (dd, J=11.5, 4.5 Hz, 1H), 3.83-3.78 (m, 4H), 3.78-3.72 (m, 2H), 3.65 (dd, J=11.4, 2.5 Hz, 1H), 3.59 (td, J=11.3, 2.6 Hz, 1H), 3.48 (td, J=11.2, 2.7 Hz, 1H), 3.37-3.32 (m, 1H), 2.91-2.85 (m, 2H), 2.84-2.78 (m, 2H), 2.41 (q, J=7.5 Hz, 2H), 0.92 (t, J=7.4 Hz, 3H); ¹³C NMR (151 MHz, DMSO) δ 164.2, 161.1, 160.9, 160.4, 155.6, 155.5, 146.3, 135.0, 132.4, 129.3, 118.6, 114.6, 93.0, 73.3, 67.6, 65.8, 65.7, 64.3, 61.5, 51.9, 35.4, 33.6, 17.6, 14.1; HPLC: t_{R}=10.27 min, Method C, Purity: 99.50%.

Example 42, 2-(2-(4-(2-(6-((1,4-Dioxan-2-yl)methoxy)-3-ethyl-4-hydroxypyridin-2-yl)ethyl)phenoxy)ethyl)isoindoline-1,3-dione (61k). The target compound was synthesized as described for compound 5 by debenzylation and hydrogenation of 60k (20 mg, 32.3 μmol) in MeOH:EtOAc (1:2; 3.0 mL), catalyzed by 10% Pd—C (1.7 mg, 1.60 μmol) under hydrogen atmosphere for 3 h. The crude was purified by flash chromatography (6% MeOH in DCM) and the obtained product was further purified by preparative HPLC (35% solvent B) to afford 61k as a white solid (12.4 mg, 72%). R_{f}=0.24 (MeOH:DCM; 5:95); ¹H NMR (600 MHz, DMSO) δ 7.92-7.87 (m, 2H), 7.87-7.83 (m, 2H), 7.09 (d, J=8.5 Hz, 2H), 6.80 (d, J=8.5 Hz, 2H), 6.28 (br s, 1H), 4.23-4.19 (m, 2H), 4.17 (t, J=5.9 Hz, 2H), 3.94 (t, J=5.8 Hz, 2H), 3.90-3.83 (m, 1H), 3.81-3.73 (m, 2H), 3.66 (dd, J=11.4, 2.6 Hz, 1H), 3.61 (td, J=11.3, 2.7 Hz, 1H), 3.49 (td, J=11.2, 2.7 Hz, 1H), 3.39 (t, J=10.7 Hz, 1H), 2.91-2.76 (m, 4H), 2.43 (q, J=7.4 Hz, 2H), 0.96 (t, J=7.4 Hz, 3H); ¹³C NMR (151 MHz, DMSO) δ 167.7, 160.2, 156.4, 134.5, 133.1, 131.5, 129.3, 123.1, 119.9, 114.4, 92.8, 72.9, 67.0, 65.7, 65.7, 64.5, 37.0, 33.9, 17.5, 13.8; HPLC: t_{R}=10.96 min, Method C, Purity: 99.40%.

Example 43, 6-((1,4-dioxan-2-yl)methoxy)-3-ethyl-2-(4-hydroxyphenethyl)pyridin-4-ol (62). The target compound was synthesized as described for compound 5 by debenzylation and hydrogenation of 59 (17 mg, 38.2 μmol) in MeOH:EtOAc (1:2; 3.0 mL), catalyzed by 10% Pd—C (1.8 mg, 1.69 μmol) under hydrogen atmosphere for 3 h. The crude was purified by flash chromatography (6-8% MeOH in DCM) and the obtained product was further purified by preparative HPLC (30% solvent B) to afford 62 as a colorless oil (13 mg, 95%). ¹H NMR (600 MHz, DMSO) δ 9.18 (br s, 2H), 7.00 (d, J=8.4 Hz, 2H), 6.67 (d, J=8.4 Hz, 2H), 6.34 (br s, 1H), 4.23 (d, J=5.0 Hz, 2H), 3.92-3.85 (m, 1H), 3.83-3.75 (m, 2H), 3.68 (dd, J=11.8, 2.5 Hz, 1H), 3.62 (td, J=11.3, 2.7 Hz, 1H), 3.50 (td, J=11.2, 2.7 Hz, 1H), 3.41 (dd, J=11.4, 10.0 Hz, 1H), 2.92-2.84 (m, 2H), 2.81-2.75 (m, 2H), 2.44 (q, J=7.4 Hz, 2H), 0.97 (t, J=7.4 Hz, 3H); ¹³C NMR (151 MHz, DMSO) δ 160.0, 155.6, 130.7, 129.2, 120.0, 115.1, 92.7, 72.9, 67.0, 65.8, 65.7, 40.1, 34.1, 17.5, 13.7; HPLC: t_{R}=9.04 min, Method C, Purity: 99.76%.

Example 44, 6-((1,4-Dioxan-2-yl)methoxy)-2-(2-(4-(ethoxy-d₅)phenyl)ethyl-1,1,2,2-d₄)-3-ethylpyridin-4-ol (611). A flame-dried vial under argon atmosphere was charged with 601 (31 mg, 64.8 μmol). The vial was evacuated and backfilled with argon three times. Methanol d₄ (1.0 mL) was added and the solution was cooled down to 0° C. Then, CoCl₂·6H₂O (1.5 mg, 6.30 μmol) was added. To the resulting light pink solution was added NaBD₄ (5.40 mg, 0.13 mmol), which resulted in effervescence and change of colour to black. The ice bath was removed, the vial was capped and stirred at rt with a magnetic stir bar that contained traces of Pd/C. After 3 h, additional CoCl₂·6H₂O (3.0 mg, 12.6 μmol) and NaBD₄ (10.8 mg, 0.26 mmol) were added, and the reaction mixture was stirred at 50° C. for 19 h. Water (10 mL) was added to the cooled reaction mixture and extracted with EtOAc (3×10 mL). The combined organic phase was dried over anhydrous MgSO₄, filtered, evaporated onto Celite, and purified by flash chromatography (30-80% EtOAc in heptane) to afford 611 as a white solid (6.9 mg, 27%). ¹H NMR (600 MHz, MeOD) δ 7.05 (d, J=8.7 Hz, 2H), 6.79 (d, J=8.7 Hz, 2H), 5.88 (br s, 1H), 4.16-4.05 (m, 2H), 3.95-3.90 (m, 1H), 3.85 (dd, J=11.5, 2.7 Hz, 1H), 3.83-3.79 (m, 1H), 3.76-3.69 (m, 2H), 3.65-3.58 (m, 1H), 3.50 (dd, J=11.5, 10.0 Hz, 1H), 2.42 (q, J=7.4 Hz, 2H), 0.99 (t, J=7.5 Hz, 3H); ¹³C NMR (151 MHz, MeOD) δ 162.2, 158.9, 134.2, 130.4, 123.2, 115.5, 93.9, 74.9, 69.2, 68.2, 67.7, 67.5, 63.6 (p, J=22.2, 20.2 Hz), 35.0, 19.0, 14.3, 14.3-13.8 (in); HPLC: t_{R}=10.45 min, Method C, Purity: 98.66%.

Example 45, 6-((1,4-Dioxan-2-yl)methoxy)-2-(4-(2-aminoethoxy)phenethyl)-3-ethylpyridin-4-ol hydrochloride (61m). To a solution of 60a (7.9 mg, 15.7 μmol) in DCM (0.2 mL) was added 4 M HCl in dioxane (45.2 μL, 0.18 mmol). The mixture was briefly vortexed and stirred at rt. After 3 h, the reaction mixture was concentrated to dryness. The crude was washed with MeCN and dried to afford 61m as a white solid (6.6 mg, 96%). ¹H NMR (600 MHz, MeOD) δ 7.14 (d, J=8.4 Hz, 2H), 6.95 (d, J=8.7 Hz, 2H), 6.55 (s, 1H), 4.36-4.30 (m, 2H), 4.21 (t, J=5.0 Hz, 2H), 4.04-3.98 (m, 1H), 3.88 (dd, J=11.5, 2.7 Hz, 1H), 3.85-3.81 (m, 1H), 3.78-3.71 (m, 2H), 3.62 (td, J=11.5, 2.9 Hz, 1H), 3.55 (dd, J=11.5, 10.0 Hz, 1H), 3.36 (t, J=5.0 Hz, 2H), 3.08-3.02 (m, 2H), 2.96-2.90 (m, 2H), 2.50 (q, J=7.5 Hz, 2H), 1.06 (t, J=7.5 Hz, 3H); ¹³C NMR (151 MHz, MeOD) δ 173.7, 161.3, 158.4, 151.9, 133.9, 130.8, 123.4, 115.9, 93.6, 74.5, 71.4, 68.4, 67.8, 67.5, 65.3, 40.4, 35.7, 33.6, 18.8, 13.6; HPLC: t_{R}=7.66 min, Method C, Purity: 97.35%.

Example 46, 6-((1,4-Dioxan-2-yl)methoxy)-2-(4-(3-aminopropoxy)phenethyl)-3-ethylpyridin-4-ol hydrochloride (61n). To a solution of 60b (13.1 mg, 25.4 μmol) in DCM (0.2 mL) was added 4 M HCl in dioxane (72.9 μL, 0.29 mmol). The mixture was briefly vortexed and stirred at rt. After 3 h, the reaction mixture was concentrated to dryness. The crude was washed with MeCN and dried to afford 61n as a white solid (11.2 mg, 97%). ¹H NMR (600 MHz, MeOD) δ 7.11 (d, J=8.7 Hz, 2H), 6.89 (d, J=8.7 Hz, 2H), 6.52 (s, 1H), 4.36-4.28 (m, 2H), 4.09 (t, J=5.8 Hz, 2H), 4.03-3.97 (m, 1H), 3.88 (dd, J=11.5, 2.7 Hz, 1H), 3.85-3.80 (m, 1H), 3.79-3.71 (m, 2H), 3.62 (td, J=11.5, 2.9 Hz, 1H), 3.55 (dd, J=11.5, 10.1 Hz, 1H), 3.15 (t, J=7.3 Hz, 2H), 3.07-3.00 (m, 2H), 2.95-2.89 (m, 2H), 2.51 (q, J=7.5 Hz, 2H), 2.14 (p, J=7.4, 6.6, 6.0 Hz, 2H), 1.07 (t, J=7.5 Hz, 3H); ¹³C NMR (151 MHz, MeOD) δ 173.7, 161.2, 158.9, 152.0, 133.3, 130.6, 123.4, 115.8, 93.6, 74.5, 71.2, 68.4, 67.7, 67.5, 66.3, 38.7, 35.7, 33.7, 28.4, 18.8, 13.6; HPLC: t_{R}=7.92 min, Method C, Purity: 99.28%.

Example 47, 5-(4-(2-(6-((1,4-Dioxan-2-yl)methoxy)-3-ethyl-4-hydroxypyridin-2-yl)ethyl)phenoxy)pentanoic acid (610). To a solution of 60e (13.1 mg, 26.9 μmol) in THF (1.0 mL) was added aq. LiOH (0.6 M, 450 μL, 0.27 mmol) and stirred at RT. After 18 h, the reaction mixture was acidified by addition of aq. HCl (1.0 M, 450 μL). Then, water (1.5 mL) was added and extracted with EtOAc (3×2 mL). The combined organic phase was dried over anhydrous Na₂SO₄, filtered, and concentrated to afford the title compound as a colorless oil (11.5 mg, 93%). ¹H NMR (600 MHz, Acetone) δ 9.30 (br s, 1H), 7.13 (d, J=8.4 Hz, 2H), 6.83 (d, J=8.8 Hz, 2H), 6.07 (s, 1H), 4.27 (dd, J=11.4, 5.6 Hz, 1H), 4.20 (dd, J=11.4, 5.1 Hz, 1H), 3.98 (t, J=6.0 Hz, 2H), 3.90-3.80 (m, 2H), 3.76 (dd, J=11.4, 2.8 Hz, 1H), 3.70-3.62 (m, 2H), 3.53 (td, J=11.8, 2.8 Hz, 1H), 3.39 (dd, J=11.4, 9.9 Hz, 1H), 3.07-2.89 (m, 4H), 2.55 (q, J=7.4 Hz, 2H), 2.38 (t, J=7.1 Hz, 2H), 1.86-1.70 (m, 4H), 1.02 (t, J=7.4 Hz, 3H); HPLC: t_{R}=10.02 min, Method C, Purity: 99.59%.

Example 48, 2-((4-(2-(6-((1,4-Dioxan-2-yl)methoxy)-3-ethyl-4-hydroxypyridin-2-yl)ethyl)phenoxy)methyl)oxazole-4-carboxylic acid (61p). To a solution of 60j (8.7 mg, 17.5 μmol) in THF (400 μL) was added aq. LiOH (0.6 M, 300 μL, 0.18 mmol) and stirred at RT. After 6 h, the reaction mixture was acidified by addition of aq. HCl (1.0 M, 300 μL). Then, water (1.5 mL) was added and extracted with EtOAc (3×2 mL). The combined organic phase was dried over anhydrous Na₂SO₄, filtered, and concentrated to afford 61p as a white solid (8.1 mg, 95%). ¹H NMR (600 MHz, DMSO) δ 13.14 (br s, 1H), 10.08 (br s, 1H), 8.78 (s, 1H), 7.16 (d, J=8.6 Hz, 2H), 6.95 (d, J=8.6 Hz, 2H), 6.19 (br s, 1H), 5.23 (s, 2H), 4.22-4.15 (m, 2H), 3.88-3.81 (m, 1H), 3.81-3.72 (m, 3H), 3.68-3.64 (m, 1H), 3.63-3.58 (m, 1H), 3.49 (td, J=11.2, 2.8 Hz, 1H), 2.91-2.80 (m, 4H), 2.43 (q, J=7.4 Hz, 2H), 0.95 (t, J=7.5 Hz, 3H); HPLC: t_{R}=9.46 min, Method C, Purity: 99.35%.

6-((1,4-Dioxan-2-yl)methoxy)-4-(benzyloxy)-2-((6-ethoxypyridin-3-yl)ethynyl)-3-ethylpyridine (63a). The target compound was synthesized as described for compound 40a by Sonogashira Coupling of 39 (57.0 mg, 0.157 mmol) with 2-ethoxy-5-ethynylpyridine (34.0 mg, 0.231 mmol) in the presence of PdCl₂(MeCN)₂ (1.6 mg, 6.2 μmol), XPhos (9.0 mg, 18.9 μmol), and Cs₂CO₃ (127.6 mg, 0.392 mmol) in anhydrous MeCN (0.4 mL) at 80° C. for 19 h. The crude was purified by flash chromatography (12-15% EtOAc in heptane) to afford 63a as a yellow oil (41 mg, 55%). R_{f}=0.57 (EtOAc:PE; 4:6); ¹H NMR (400 MHz, CDCl₃) δ 8.38 (d, J=1.9 Hz, 1H), 7.73 (dd, J=8.6, 2.3 Hz, 1H), 7.40 (d, J=4.0 Hz, 4H), 7.38-7.33 (m, 1H), 6.72 (dd, J=8.6, 0.8 Hz, 1H), 6.33 (s, 1H), 5.08 (s, 2H), 4.45-4.32 (m, 4H), 4.01-3.94 (m, 1H), 3.89-3.83 (m, 2H), 3.83-3.76 (m, 1H), 3.75-3.70 (m, 1H), 3.69-3.62 (m, 1H), 3.52 (dd, J=11.5, 10.1 Hz, 1H), 2.85 (q, J=7.4 Hz, 2H), 1.40 (t, J=7.1 Hz, 3H), 1.19 (t, J=7.5 Hz, 3H); ¹³C NMR (101 MHz, CDCl₃) δ 165.0, 163.5, 162.8, 150.7, 141.5, 138.5, 136.0, 128.8, 128.4, 127.2, 126.7, 112.5, 111.0, 94.3, 88.9, 88.7, 74.0, 70.1, 68.4, 66.9, 66.6, 65.6, 62.3, 20.5, 14.7, 13.9.

6-((1,4-Dioxan-2-yl)methoxy)-4-(benzyloxy)-2-((6-ethoxy-5-fluoropyridin-3-yl)ethynyl)-3-ethylpyridine (63b). The target compound was synthesized as described for compound 40a by Sonogashira Coupling of 39 (53.3 mg, 0.146 mmol) with 2-ethoxy-5-ethynyl-3-fluoropyridine (38.0 mg, 0.230 mmol) in the presence of PdCl₂(MeCN)₂ (1.5 mg, 8.4 μmol), XPhos (8.4 mg, 17.6 μmol), and Cs₂CO₃ (119.3 mg, 0.366 mmol) in anhydrous MeCN (0.4 mL) at 80° C. for 19 h. The crude was purified by flash chromatography (11-14% EtOAc in heptane) to afford 63b as a yellow oil (38 mg, 53%). R_{f}=0.59 (EtOAc:PE, 4:6). ¹H NMR (400 MHz, CDCl₃) δ 8.14 (d, J=1.9 Hz, 1H), 7.48 (dd, J=10.5, 1.9 Hz, 1H), 7.42-7.33 (m, 5H), 6.33 (s, 1H), 5.08 (s, 2H), 4.49 (q, J=7.1 Hz, 2H), 4.38-4.29 (m, 2H), 4.01-3.95 (m, 1H), 3.89-3.83 (m, 2H), 3.83-3.76 (m, 1H), 3.75-3.71 (m, 1H), 3.69-3.62 (m, 1H), 3.52 (dd, J=11.5, 10.1 Hz, 1H), 2.84 (q, J=7.4 Hz, 2H), 1.45 (t, J=7.1 Hz, 3H), 1.19 (t, J=7.4 Hz, 3H). ¹³C NMR (101 MHz, CDCl₃) δ 164.9, 162.8, 153.3 (d, J=11.2 Hz), 146.7 (d, J=260.1 Hz), 145.1 (d, J=5.8 Hz), 138.1, 135.9, 128.9, 128.4, 127.3, 127.0, 125.5 (d, J=16.7 Hz), 113.0 (d, J=3.1 Hz), 94.5, 89.3, 87.2, 74.0, 70.2, 68.4, 66.9, 66.6, 65.6, 63.0, 20.5, 14.6, 13.9.

Example 49, 6-((1,4-Dioxan-2-yl)methoxy)-2-(2-(6-ethoxypyridin-3-yl)ethyl)-3-ethylpyridin-4-ol (64a). The target compound was synthesized as described for compound 5 by debenzylation and hydrogenation of 63a (41.0 mg, 0.086 mmol) in MeOH:EtOAc (1:2, 1.5 mL), catalyzed by 10% Pd—C (4.6 mg, 0.04 mmol) under hydrogen atmosphere for 4 h. The crude was purified by flash chromatography (5-7% MeOH in DCM) and the obtained product was further purified by preparative HPLC (20-45% solvent B in 9 min) to afford 64a as a colorless oil (18.9 mg, 56%). R_{f}=0.13 (EtOAc:PE; 4:6); ¹H NMR (600 MHz, CDCl₃) δ 7.90 (d, J=2.4 Hz, 1H), 7.45 (dd, J=8.5, 2.3 Hz, 1H), 6.64 (d, J=8.5 Hz, 1H), 6.24 (s, 1H), 4.28 (q, J=7.1 Hz, 2H), 4.13 (qd, J=11.1, 5.1 Hz, 2H), 3.94-3.89 (m, 1H), 3.85-3.75 (m, 2H), 3.75-3.67 (m, 2H), 3.58 (td, J=11.2, 2.9 Hz, 1H), 3.50-3.43 (m, 1H), 2.98-2.86 (m, 4H), 2.48 (q, J=7.4 Hz, 2H), 1.36 (t, J=7.1 Hz, 3H), 1.03 (t, J=7.5 Hz, 3H); ¹³C NMR (151 MHz, CDCl₃) δ 170.6, 162.8, 160.4, 151.6, 146.1, 139.4, 129.0, 121.5, 110.8, 93.6, 73.5, 68.2, 67.2, 66.8, 66.4, 61.9, 34.1, 31.8, 18.3, 14.8, 14.0. HPLC: t_{R}=3.69 min, Method B, Purity: 96.53%.

Example 50, 6-((1,4-Dioxan-2-yl)methoxy)-2-(2-(6-ethoxy-5-fluoropyridin-3-yl)ethyl)-3-ethylpyridin-4-ol (64b). The target compound was synthesized as described for compound 5 by debenzylation and hydrogenation of 63b (38 mg, 0.077 mmol) in MeOH:EtOAc (1:2, 1.5 mL), catalyzed by 10% Pd—C (4.1 mg, 0.004 mmol) under hydrogen atmosphere for 6 h. The crude was purified by flash chromatography (5-7% MeOH in DCM) to afford 64b as a colorless oil (21.4 mg, 68%). R_{f}=0.19 (EtOAc:PE; 4:6); ¹H NMR (600 MHz, DMSO) δ 10.40 (s, 1H), 7.74 (d, J=1.9 Hz, 1H), 7.54 (dd, J=11.6, 2.0 Hz, 1H), 6.07 (s, 1H), 4.34 (q, J=7.0 Hz, 2H), 4.13 (qd, J=11.3, 5.1 Hz, 2H), 3.83-3.79 (m, 1H), 3.75 (td, J=11.3, 2.7 Hz, 2H), 3.67-3.63 (m, 1H), 3.59 (td, J=11.3, 2.6 Hz, 1H), 3.48 (td, J=11.2, 2.7 Hz, 1H), 3.36-3.32 (m, 1H), 2.96-2.84 (m, 4H), 2.42 (q, J=7.4 Hz, 2H), 1.32 (t, J=7.0 Hz, 3H), 0.93 (t, J=7.4 Hz, 3H); ¹³C NMR (151 MHz, DMSO) δ 164.7, 160.9, 150.5 (d, J=11.2 Hz), 154.5, 146.3 (d, J=256.0 Hz), 140.5 (d, J=5.6 Hz), 131.1, 123.9 (d, J=14.8 Hz), 119.1, 93.0, 73.2, 67.5, 65.8, 65.7, 64.7, 61.6, 34.0, 30.0, 17.5, 14.4, 14.0; HPLC: t_{R}=5.71 min, Method B, Purity: 98.23%.

(E)-6-((1,4-dioxan-2-yl)methoxy)-4-(benzyloxy)-2-(4-ethoxystyryl)-3-ethylpyridine (65). A microwave vial, charged with 39 (62.7 mg, 0.172 mmol), sodium acetate (28.3 mg, 0.272 mmol), PPh₃ (18.1 mg, 0.069 mmol), and Pd(OAc)₂ (1.9 mg, 0.008 mmol), was capped, and evacuated and backfilled with argon three times. Then, degassed 1-ethoxy-4-vinylbenzene (90% w/w, 0.06 mL, 0.361 mmol) and anhydrous, degassed DMF (0.2 mL) were added, and the vial was stirred at 135° C. After 17 h, water (10 mL) was added to the cooled reaction mixture and extracted with DCM (3×10 mL). The combined organic phase was dried over anhydrous Na₂SO₄, filtered, evaporated onto Celite and purified by flash chromatography (8-20% EtOAc in heptane) to afford 65 as a white solid (75 mg, 92%). R_{f}=0.52 (EtOAc:PE; 3:7); ¹H NMR (600 MHz, CDCl₃) δ 7.71 (d, J=15.4 Hz, 1H), 7.54-7.49 (m, 2H), 7.43-7.39 (m, 4H), 7.37-7.33 (m, 1H), 7.19 (d, J=15.4 Hz, 1H), 6.93-6.88 (m, 2H), 6.22 (s, 1H), 5.08 (s, 2H), 4.46-4.39 (m, 2H), 4.09-4.02 (m, 3H), 3.93 (dd, J=11.5, 2.7 Hz, 1H), 3.90-3.86 (m, 1H), 3.85-3.80 (m, 1H), 3.76-3.73 (m, 1H), 3.71-3.66 (m, 1H), 3.56 (dd, J=11.5, 10.1 Hz, 1H), 2.79 (q, J=7.5 Hz, 2H), 1.43 (t, J=7.0 Hz, 3H), 1.17 (t, J=7.5 Hz, 3H); ¹³C NMR (151 MHz, CDCl₃) δ 165.5, 161.9, 159.2, 149.8, 136.4, 133.2, 130.1, 128.8, 128.6, 128.2, 127.2, 121.7, 120.6, 114.8, 92.2, 74.1, 70.0, 68.8, 67.0, 66.6, 65.1, 63.6, 17.9, 15.0, 14.9.

6-((1,4-Dioxan-2-yl)methoxy)-4-(benzyloxy)-2-(2-(4-ethoxyphenyl)cyclopropyl)-3-ethylpyridine (66). A solution of TFA (0.07 mL, 0.914 mmol) in anhydrous DCM (0.25 mL) was added at 5° C. very slowly to a solution of diethyl zinc (1 M in hexanes, 0.93 mL, 0.93 mmol) in anhydrous DCM (0.50 mL) and the mixture was stirred at 5° C. for 30 min. A solution of diiodomethane (0.08 mL, 0.993 mmol) in anhydrous DCM (0.25 mL) was added to the previous mixture at 5° C. The resulting mixture was stirred for 40 min at 5° C. Then, a solution of 65 (110.9 mg, 0.233 mmol) in anhydrous DCM (0.25 mL) was added to the mixture at 5° C. The resulting mixture was stirred for 15 h at rt, and then it was quenched with sat. NH₄Cl (10 mL). The aqueous layer was extracted with DCM (3×10 mL). The combined organic phase was washed with sat. NaHCO₃ (2×10 mL) and brine (10 mL). The solvent was evaporated under reduced pressure and the residue was purified by flash chromatography (9-12% EtOAc in heptane) to afford different diastereomers of 66 as a colorless oil (68 mg, 60%). R_{f}=0.61 (EtOAc:PE; 3:7).

Example 51, 6-((1,4-Dioxan-2-yl)methoxy)-2-(2-(4-ethoxyphenyl)cyclopropyl)-3-ethylpyridin-4-ol (67). The target compound was synthesized as described for compound 5 by debenzylation of 66 (68.0 mg, 0.139 mmol) in MeOH:EtOAc (1:2, 1.5 mL), catalyzed by 10% Pd—C (7.4 mg, 0.007 mmol) under hydrogen atmosphere for 2 h. The reaction mixture was filtered through a Celite pad and purified by preparative HPLC (35-40% solvent B in 9 min) to afford 67 as a colorless oil (13 mg, 23%). R_{f}=0.16 (EtOAc:PE; 3:7); ¹H NMR (600 MHz, CDCl₃) δ 6.93-6.84 (m, 3H), 6.77-6.68 (m, 2H), 4.02-3.98 (m, 1H), 3.97-3.90 (m, 3H), 3.87-3.77 (m, 2H), 3.77-3.68 (m, 3H), 3.62-3.55 (m, 1H), 3.43-3.33 (m, 1H), 2.76-2.70 (m, 1H), 2.59 (q, J=7.5 Hz, 2H), 2.51-2.45 (m, 1H), 1.76-1.62 (m, 2H), 1.37 (t, J=7.0 Hz, 3H), 1.03 (t, J=7.4 Hz, 3H); ¹³C NMR (151 MHz, CDCl₃) δ 173.4, 158.4 (d, J=2.4 Hz), 158.3, 147.1 (d, J=9.5 Hz), 129.0 (d, J=7.4 Hz), 126.8 (d, J=3.0 Hz), 125.2 (d, J=8.8 Hz), 114.9 (d, J=3.3 Hz), 94.3 (d, J=17.0 Hz), 72.9 (d, J=8.1 Hz), 70.5 (d, J=7.7 Hz), 67.2 (d, J=2.7 Hz), 66.8 (d, J=2.9 Hz), 66.2 (d, J=6.2 Hz), 63.6, 24.6 (d, J=6.4 Hz), 20.3 (d, J=10.3 Hz), 18.4 (d, J=2.5 Hz), 14.9, 12.6 (d, J=2.6 Hz), 11.8 (d, J=17.9 Hz); HPLC: t_{R}=5.93 min, Method B, Purity: 95.03%.

(S)-6-((1,4-dioxan-2-yl)methoxy)-4-(benzyloxy)-2-chloro-3-ethylpyridine (68). A flame-dried vial was charged with 37 (1.05 g, 3.72 mmol), tBuOK (501.1 mg, 4.47 mmol), and (R)-(1,4-dioxan-2-yl)methanol (440.4 mg, 3.73 mmol). The flask was evacuated and backfilled with argon three times. Then, anhydrous dioxane (8 mL) was added. The vial was capped at stirred at 100° C. for 9 h. The reaction mixture was cooled down to rt and water (50 mL) was added and extracted with EtOAc (3×50 mL). The combined organic phase was dried over anhydrous MgSO₄, filtered, evaporated onto Celite, and purified by flash chromatography (5-12% EtOAc in heptane) to afford 68 as a white solid (1.86 g, 54%). R_{f}=0.12 (EtOAc:heptane; 1:9); ¹H NMR (400 MHz, CDCl₃) δ 7.48-7.30 (m, 5H), 6.26 (s, 1H), 5.07 (s, 2H), 4.27 (d, J=4.9 Hz, 2H), 3.99-3.92 (m, 1H), 3.88-3.61 (m, 5H), 3.49 (dd, J=11.5, 10.1 Hz, 1H), 2.72 (q, J=7.4 Hz, 2H), 1.12 (t, J=7.4 Hz, 3H); ¹³C NMR (151 MHz, CDCl₃) δ 166.1, 161.8, 147.6, 135.8, 128.9, 128.4, 127.2, 120.7, 92.9, 73.9, 70.5, 68.3, 66.9, 66.6, 65.8, 20.0, 13.1.

(S)-6-((1,4-dioxan-2-yl)methoxy)-4-(benzyloxy)-2-((3,4-dimethoxyphenyl)ethynyl)-3-ethylpyridine (69a). The target compound was synthesized as described for compound 40a by Sonogashira Coupling of 68 (49.4 mg, 0.136 mmol) with 4-ethynyl-1,2-dimethoxybenzene (44.0 mg, 0.271 mmol) in the presence of PdCl₂(MeCN)₂ (1.4 mg, 5.4 μmol), XPhos (7.8 mg, 16.4 μmol), and Cs₂CO₃ (110.6 mg, 0.339 mmol) in anhydrous MeCN (0.4 mL) at 80° C. for 15 h. The crude was purified by flash chromatography (15-35% EtOAc in heptane) to afford 69a as a white solid (50 mg, 75%). R_{f}=0.18 (EtOAc:PE; 3:7); ¹H NMR (600 MHz, CDCl₃) δ 7.42-7.38 (m, 4H), 7.38-7.33 (m, 1H), 7.19 (dd, J=8.3, 1.9 Hz, 1H), 7.10 (d, J=1.9 Hz, 1H), 6.84 (d, J=8.3 Hz, 1H), 6.32 (s, 1H), 5.08 (s, 2H), 4.40-4.33 (m, 2H), 4.01-3.95 (m, 1H), 3.91 (s, 6H), 3.88-3.84 (m, 2H), 3.79 (td, J=11.1, 2.9 Hz, 1H), 3.73 (dd, J=11.8, 2.3 Hz, 1H), 3.66 (td, J=11.3, 2.9 Hz, 1H), 3.52 (dd, J=11.5, 10.1 Hz, 1H), 2.86 (q, J=7.4 Hz, 2H), 1.21 (t, J=7.4 Hz, 3H); ¹³C NMR (151 MHz, CDCl₃) δ 165.0, 162.7, 150.1, 148.8, 138.7, 136.0, 128.8, 128.4, 127.3, 126.5, 125.6, 115.0, 114.7, 111.1, 94.0, 92.2, 86.2, 74.0, 70.1, 68.4, 66.9, 66.6, 65.7, 56.1, 56.1, 20.5, 13.9.

(S)-6-((1,4-dioxan-2-yl)methoxy)-4-(benzyloxy)-2-((2,4-dimethoxyphenyl)ethynyl)-3-ethylpyridine (69b). The target compound was synthesized as described for compound 40a by Sonogashira Coupling of 68 (51.1 mg, 0.140 mmol) with 1-ethynyl-2,4-dimethoxybenzene (45.6 mg, 0.281 mmol) in the presence of PdCl₂(MeCN)₂ (1.5 mg, 5.8 μmol), XPhos (8.0 mg, 16.8 μmol), and Cs₂CO₃ (114.4 mg, 0.351 mmol) in anhydrous MeCN (0.4 mL) at 80° C. for 15 h. The crude was purified by flash chromatography (15-35% EtOAc in heptane) to afford 69b as a slightly yellow oil (65 mg, 95%). R_{f}=0.14 (EtOAc:PE; 3:7); ¹H NMR (600 MHz, CDCl₃) δ 7.49 (d, J=8.4 Hz, 1H), 7.44-7.38 (m, 4H), 7.37-7.33 (m, 1H), 6.48 (dd, J=8.4, 2.4 Hz, 1H), 6.45 (d, J=2.3 Hz, 1H), 6.30 (s, 1H), 5.08 (s, 2H), 4.40 (dd, J=11.6, 3.9 Hz, 1H), 4.35 (dd, J=11.6, 6.0 Hz, 1H), 4.00-3.95 (m, 1H), 3.89-3.83 (m, 8H), 3.79 (td, J=11.3, 2.8 Hz, 1H), 3.72 (dd, J=11.8, 2.6 Hz, 1H), 3.66 (td, J=11.3, 2.9 Hz, 1H), 3.52 (dd, J=11.5, 10.1 Hz, 1H), 2.91 (q, J=7.4 Hz, 2H), 1.20 (t, J=7.4 Hz, 3H); ¹³C NMR (151 MHz, CDCl₃) δ 165.0, 162.4, 161.9, 161.7, 138.9, 136.0, 134.5, 128.7, 128.2, 127.1, 126.3, 104.9, 104.6, 98.4, 93.6, 90.1, 89.2, 74.0, 70.0, 68.2, 66.8, 66.5, 65.8, 55.8, 55.5, 20.3, 13.7.

(S)-5-((6-((1,4-dioxan-2-yl)methoxy)-4-(benzyloxy)-3-ethylpyridin-2-yl)ethynyl)-2-methoxybenzonitrile (69c). The target compound was synthesized as described for compound 40a by Sonogashira Coupling of 68 (52.0 mg, 0.143 mmol) with 5-ethynyl-2-methoxybenzonitrile 44.9 mg, 0.286 mmol) in the presence of PdCl₂(MeCN)₂ (1.5 mg, 5.8 μmol), XPhos (8.2 mg, 17.2 μmol) and Cs₂CO₃ (116.4 mg, 0.357 mmol) in anhydrous MeCN (0.4 mL) at 80° C. for 15 h. The crude was purified by flash chromatography (15-35% EtOAc in heptane) to afford 69c as a white solid (40 mg, 58%). R_{f}=0.10 (EtOAc:PE; 3:7). ¹H NMR (600 MHz, CDCl₃) δ 7.78-7.74 (m, 2H), 7.43-7.38 (m, 4H), 7.38-7.33 (m, 1H), 6.99-6.96 (m, 1H), 6.34 (s, 1H), 5.09 (s, 2H), 4.38-4.32 (m, 2H), 4.00-3.95 (m, 4H), 3.86 (dd, J=11.6, 2.8 Hz, 2H), 3.80 (td, J=11.3, 2.7 Hz, 1H), 3.75-3.71 (m, 1H), 3.66 (td, J=11.3, 2.9 Hz, 1H), 3.52 (dd, J=11.5, 10.1 Hz, 1H), 2.84 (q, J=7.4 Hz, 2H), 1.20 (t, J=7.5 Hz, 3H). ¹³C NMR (151 MHz, CDCl₃) δ 165.1, 162.8, 161.3, 138.0, 137.9, 137.2, 135.8, 128.9, 128.4, 127.3, 127.0, 115.9, 115.6, 111.7, 102.6, 94.5, 89.4, 87.8, 74.0, 70.3, 68.4, 66.9, 66.6, 65.9, 56.5, 20.5, 13.9.

(S)-2-((6-((1,4-dioxan-2-yl)methoxy)-4-(benzyloxy)-3-ethylpyridin-2-yl)ethynyl)-5-methoxybenzonitrile (69d). The target compound was synthesized as described for compound 40a by Sonogashira Coupling of 68 (52.0 mg, 0.143 mmol) with 2-ethynyl-5-methoxybenzonitrile (44.9 mg, 0.286 mmol) in the presence of PdCl₂(MeCN)₂ (1.5 mg, 5.8 μmol), XPhos (8.2 mg, 17.2 μmol) and Cs₂CO₃ (116.4 mg, 0.357 mmol) in anhydrous MeCN (0.4 mL) at 80° C. for 15 h. The crude was purified by flash chromatography (15-35% EtOAc in heptane) to afford 69d as a slightly yellow oil (34 mg, 49%). R_{f}=0.14 (EtOAc:PE; 3:7); ¹H NMR (600 MHz, CDCl₃) δ 7.68 (d, J=8.7 Hz, 1H), 7.43-7.38 (m, 4H), 7.38-7.33 (m, 1H), 7.16 (d, J=2.7 Hz, 1H), 7.11 (dd, J=8.7, 2.7 Hz, 1H), 6.35 (s, 1H), 5.10 (s, 2H), 4.40 (dd, J=11.6, 3.9 Hz, 1H), 4.35 (dd, J=11.6, 6.0 Hz, 1H), 4.01-3.96 (m, 1H), 3.89-3.84 (m, 5H), 3.80 (td, J=11.3, 2.9 Hz, 1H), 3.73 (dd, J=11.6, 2.6 Hz, 1H), 3.66 (td, J=11.4, 2.8 Hz, 1H), 3.52 (dd, J=11.5, 10.1 Hz, 1H), 2.96 (q, J=7.5 Hz, 2H), 1.22 (t, J=7.5 Hz, 3H); ¹³C NMR (151 MHz, CDCl₃) δ 165.3, 162.7, 159.7, 137.4, 135.8, 134.8, 128.9, 128.4, 127.8, 127.2, 119.2, 118.9, 117.7, 117.5, 116.5, 94.7, 91.6, 87.7, 74.0, 70.3, 68.3, 66.9, 66.6, 66.0, 55.9, 20.5, 14.2.

Example 52, (S)-6-((1,4-dioxan-2-yl)methoxy)-2-(3,4-dimethoxyphenethyl)-3-ethylpyridin-4-ol (70a). The target compound was synthesized as described for compound 5 by debenzylation and hydrogenation of 69a (50.0 mg, 0.102 mmol) in MeOH:EtOAc (1:2, 1.2 mL), catalyzed by 10% Pd—C (5.4 mg, 0.005 mmol) under hydrogen atmosphere for 4 h. The crude was purified by flash chromatography (6% MeOH in DCM) to afford 70a as a colorless oil (34 mg, 83%). R_{f}=0.08 (EtOAc:PE; 4:6). ¹H NMR (600 MHz, CDCl₃) δ 6.78 (d, J=7.9 Hz, 1H), 6.75-6.72 (m, 2H), 6.14 (s, 1H), 4.22-4.08 (m, 2H), 3.95-3.90 (m, 1H), 3.85-3.84 (m, 4H), 3.83 (s, 3H), 3.80 (dd, J=11.6, 2.9 Hz, 1H), 3.76-3.68 (m, 2H), 3.60 (td, J=11.3, 3.0 Hz, 1H), 3.47 (dd, J=11.5, 10.1 Hz, 1H), 2.93 (s, 4H), 2.53 (q, J=7.4 Hz, 2H), 1.06 (t, J=7.5 Hz, 3H); ¹³C NMR (151 MHz, CDCl₃) δ 170.2, 160.2, 152.0, 148.9, 147.5, 134.0, 121.3, 120.3, 111.9, 111.4, 93.6, 73.5, 68.2, 66.7, 66.5, 66.4, 56.0, 55.9, 35.1, 34.9, 18.2, 14.1; HPLC: t_{R}=9.51 min, Method C, Purity: 99.99%.

Example 53, (S)-6-((1,4-dioxan-2-yl)methoxy)-2-(2,4-dimethoxyphenethyl)-3-ethylpyridin-4-ol. (70b). The target compound was synthesized as described for compound 5 by debenzylation and hydrogenation of 69b (65.0 mg, 0.132 mmol) in MeOH:EtOAc (1:2, 1.5 mL), catalyzed by 10% Pd—C (7.1 mg, 0.007 mmol) under hydrogen atmosphere for 4 h. The crude was purified by flash chromatography (6% MeOH in DCM) to afford 70b as a colorless oil (41 mg, 77%). R_{f}=0.18 (EtOAc:PE; 5:5); ¹H NMR (600 MHz, CDCl₃) δ 7.04 (d, J=8.2 Hz, 1H), 6.45 (d, J=2.4 Hz, 1H), 6.40 (dd, J=8.2, 2.4 Hz, 1H), 6.12 (s, 1H), 4.13 (dd, J=10.9, 5.6 Hz, 1H), 4.09 (dd, J=10.9, 4.4 Hz, 1H), 3.95-3.91 (m, 1H), 3.84 (dd, J=11.5, 2.7 Hz, 1H), 3.81 (s, 3H), 3.80-3.78 (m, 4H), 3.74 (td, J=11.2, 2.7 Hz, 1H), 3.70 (dd, J=11.9, 2.7 Hz, 1H), 3.60 (td, J=11.4, 3.0 Hz, 1H), 3.47 (dd, J=11.5, 10.1 Hz, 1H), 2.89 (s, 4H), 2.56 (q, J=7.4 Hz, 2H), 1.08 (t, J=7.4 Hz, 3H); ¹³C NMR (151 MHz, CDCl₃) δ 172.1, 159.6, 159.5, 158.2, 151.3, 130.3, 121.8, 104.1, 98.6, 93.4, 73.4, 68.1, 66.9, 66.7, 66.4, 55.4, 55.3, 33.0, 29.5, 18.0, 14.0. HPLC: t_{R}=10.34 min, Method C, Purity: 99.99%.

Example 54, (S)-5-(2-(6-((1,4-dioxan-2-yl)methoxy)-3-ethyl-4-hydroxypyridin-2-yl)ethyl)-2-methoxybenzonitrile (70c). The target compound was synthesized as described for compound 5 by debenzylation and hydrogenation of 69c (40.0 mg, 0.083 mmol) in MeOH:EtOAc (1:2, 1.2 mL), catalyzed by 10% Pd—C (4.4 mg, 0.004 mmol) under hydrogen atmosphere for 6 h. The crude was purified by flash chromatography (6% MeOH in DCM) to afford 70c as a colorless oil (25 mg, 76%). R_{f}=0.15 (EtOAc:PE; 5:5); ¹H NMR (600 MHz, CDCl₃) δ 7.40-7.34 (m, 2H), 6.87 (d, J=8.6 Hz, 1H), 6.15 (s, 1H), 4.19 (dd, J=11.1, 5.6 Hz, 1H), 4.13 (dd, J=11.1, 4.5 Hz, 1H), 3.96-3.92 (m, 1H), 3.89 (s, 3H), 3.84 (dd, J=11.5, 2.7 Hz, 1H), 3.83-3.80 (m, 1H), 3.75 (td, J=11.3, 2.7 Hz, 1H), 3.71 (dd, J=11.9, 2.5 Hz, 1H), 3.61 (td, J=11.3, 2.9 Hz, 1H), 3.49 (dd, J=11.5, 10.0 Hz, 1H), 2.98-2.89 (m, 4H), 2.50 (q, J=7.5 Hz, 2H), 1.05 (t, J=7.4 Hz, 3H); ¹³C NMR (151 MHz, CDCl₃) δ 169.2, 160.6, 159.8, 152.7-151.5 (m), 134.7, 134.2, 133.4, 120.9, 116.6, 111.4, 101.4, 93.8, 73.6, 68.3, 66.7, 66.4, 66.2, 56.1, 34.5, 34.0, 18.2, 14.1. HPLC: t_{R}=9.89 min, Method C, Purity: 99.50%.

Example 55, (S)-2-(2-(6-((1,4-dioxan-2-yl)methoxy)-3-ethyl-4-hydroxypyridin-2-yl)ethyl)-5-methoxybenzonitrile (70d). The target compound was synthesized as described for compound 5 by debenzylation and hydrogenation of 69d (34.0 mg, 0.070 mmol) in MeOH:EtOAc (1:2, 1.2 mL), catalyzed by 10% Pd—C (3.7 mg, 0.003 mmol) under hydrogen atmosphere for 6 h. The crude was purified by flash chromatography (6% MeOH in DCM) to afford 70d as a colorless oil (17 mg, 61%). R_{f}=0.24 (EtOAc:PE; 5:5); ¹H NMR (600 MHz, CDCl₃) δ 7.28 (d, J=8.6 Hz, 1H), 7.08 (d, J=2.8 Hz, 1H), 7.03 (dd, J=8.6, 2.8 Hz, 1H), 6.20 (s, 1H), 4.23-4.11 (m, 2H), 3.96-3.91 (m, 1H), 3.86-3.79 (m, 5H), 3.75 (td, J=11.3, 2.7 Hz, 1H), 3.72-3.69 (m, 1H), 3.62 (td, J=11.3, 2.9 Hz, 1H), 3.49 (dd, J=11.6, 10.0 Hz, 1H), 3.20-3.11 (m, 2H), 3.02-2.93 (m, 2H), 2.52 (q, J=7.5 Hz, 2H), 1.05 (t, J=7.5 Hz, 3H); ¹³C NMR (151 MHz, CDCl₃) δ 169.2, 160.6, 158.0, 151.7, 137.5, 131.2, 121.3, 119.9, 118.1, 117.0, 112.8, 94.0, 73.7, 68.3, 66.8, 66.6, 66.5, 55.7, 34.1, 33.2, 18.2, 14.2; HPLC: t_{R}=9.92 min, Method C, Purity: 99.33%.

(S)-6-((1,4-dioxan-2-yl)methoxy)-4-(benzyloxy)-3-ethyl-2-(4-phenylbut-1-yn-1-yl)pyridine (71). The target compound was synthesized as described for compound 40a by Sonogashira Coupling of 68 (34.2 mg, 0.094 mmol) with but-3-yn-1-ylbenzene (0.02 mL, 0.142 mmol) in the presence of PdCl₂(MeCN)₂ (1.0 mg, 3.9 μmol), XPhos (5.4 mg, 11.3 μmol) and Cs₂CO₃ (76.6 mg, 0.235 mmol) in anhydrous MeCN (0.4 mL) at 80° C. for 19 h. The crude was purified by flash chromatography (5-20% EtOAc in heptane) to afford 71 as a slightly yellow oil (34 mg, 79%). R_{f}=0.47 (EtOAc:PE; 3:7); ¹H NMR (600 MHz, CDCl₃) δ 7.42-7.37 (m, 4H), 7.36-7.32 (m, 1H), 7.32-7.29 (m, 2H), 7.29-7.26 (m, 2H), 7.24-7.20 (m, 1H), 6.27 (s, 1H), 5.05 (s, 2H), 4.33 (dd, J=11.6, 4.0 Hz, 1H), 4.30 (dd, J=11.6, 6.0 Hz, 1H), 3.99-3.92 (m, 1H), 3.84 (td, J=11.1, 2.6 Hz, 2H), 3.79 (td, J=11.3, 2.7 Hz, 1H), 3.72 (dd, J=11.7, 2.5 Hz, 1H), 3.65 (td, J=11.3, 2.9 Hz, 1H), 3.50 (dd, J=11.5, 10.1 Hz, 1H), 2.96 (t, J=7.5 Hz, 2H), 2.79 (t, J=7.5 Hz, 2H), 2.67 (q, J=7.4 Hz, 2H), 1.06 (t, J=7.4 Hz, 3H); ¹³C NMR (151 MHz, CDCl₃) δ 165.0, 162.6, 140.6, 138.8, 136.1, 128.8, 128.6, 128.6, 128.3, 127.2, 126.5, 126.3, 93.8, 92.5, 79.5, 74.1, 70.0, 68.4, 66.9, 66.6, 65.6, 34.9, 21.8, 20.3, 13.8.

Example 56, (S)-6-((1,4-dioxan-2-yl)methoxy)-3-ethyl-2-(4-phenylbutyl)pyridin-4-ol (72). The target compound was synthesized as described for compound 5 by debenzylation and hydrogenation of 71 (34.0 mg, 0.074 mmol) in MeOH:EtOAc (1:2, 1.2 mL), catalyzed by 10% Pd—C (6.0 mg, 0.006 mmol) under hydrogen atmosphere for 5 h. The crude was purified by flash chromatography (5% MeOH in DCM) to afford 72 as a colorless oil (23.3 mg, 85%). R_{f}=0.24 (EtOAc:PE; 5:5); ¹H NMR (600 MHz, CDCl₃) δ 7.29-7.26 (m, 2H), 7.20-7.16 (m, 3H), 6.07 (s, 1H), 4.13 (dd, J=11.0, 5.6 Hz, 1H), 4.08 (dd, J=11.0, 4.4 Hz, 1H), 3.94-3.90 (m, 1H), 3.84-3.68 (m, 4H), 3.60 (td, J=11.3, 3.0 Hz, 1H), 3.46 (dd, J=11.5, 10.0 Hz, 1H), 2.68-2.62 (m, 4H), 2.54 (q, J=7.4 Hz, 2H), 1.74-1.68 (m, 4H), 1.08 (t, J=7.4 Hz, 3H); ¹³C NMR (151 MHz, CDCl₃) δ 171.2, 160.0, 152.3, 142.4, 128.5, 128.4, 125.9, 121.5, 93.5, 73.7, 68.3, 66.8, 66.6, 66.5, 35.8, 32.7, 31.3, 29.0, 18.4, 14.3; HPLC: t_{R}=7.15 min, Method B, Purity: 99.57%.

(S)-6-((1,4-dioxan-2-yl)methoxy)-4-(benzyloxy)-2-(4-butylphenyl)-3-ethylpyridine (73). An argon-flushed microwave vial was charged with 68 (47.0 mg, 0.129 mmol), PdCl₂(MeCN)₂ (1.7 mg, 0.007 mmol), SPhos (5.3 mg, 0.013 mmol), (4-butylphenyl)boronic acid (46.0 mg, 0.258 mmol), and K₃PO₄ (54.8 mg, 0.258 mmol). The vial was sealed, then evacuated and backfilled with argon three times. Degassed toluene (1.0 mL) was added and stirred at 100° C. After 20 h the reaction mixture was cooled to RT, diluted with water (10 mL) and extracted with DCM (3×10 mL). The combined organic phase was dried over anhydrous Na₂SO₄, filtered, evaporated onto Celite and purified by flash chromatography (0-15% EtOAc in heptane) to afford 73 as colorless oil (52 mg, 87%). Rf=0.68 (EtOAc:PE; 3:7); ¹H NMR (600 MHz, CDCl₃) δ 7.45-7.38 (m, 6H), 7.37-7.33 (m, 1H), 7.25-7.22 (m, 2H), 6.35 (s, 1H), 5.12 (s, 2H), 4.37-4.31 (m, 2H), 4.00-3.94 (m, 1H), 3.87-3.82 (m, 2H), 3.79 (td, J=11.3, 2.8 Hz, 1H), 3.72 (dd, J=11.5, 2.2 Hz, 1H), 3.65 (td, J=11.3, 2.9 Hz, 1H), 3.49 (dd, J=11.5, 10.1 Hz, 1H), 2.66 (t, J=7.7 Hz, 2H), 2.60 (q, J=7.3 Hz, 2H), 1.69-1.62 (m, 2H), 1.41 (h, J=7.4 Hz, 2H), 1.14 (t, J=7.3 Hz, 3H), 0.96 (t, J=7.4 Hz, 3H); ¹³C NMR (151 MHz, CDCl₃) δ 166.1, 162.0, 155.3, 142.7, 137.8, 136.3, 128.9, 128.8, 128.3, 128.1, 127.2, 120.8, 92.2, 74.2, 70.1, 68.5, 66.9, 66.6, 65.5, 35.6, 33.7, 22.6, 19.9, 14.8, 14.1.

Example 57, (S)-6-((1,4-dioxan-2-yl)methoxy)-2-(4-butylphenyl)-3-ethylpyridin-4-ol (74). The target compound was synthesized as described for compound 5 by debenzylation of 73 (52.0 mg, 0.113 mmol) in MeOH:EtOAc (1:2, 1.2 mL), catalyzed by 10% Pd—C (6.0 mg, 0.006 mmol) under hydrogen atmosphere for 2 h. The crude was purified by flash chromatography (6% MeOH in DCM) to afford 74 as a white solid (38 mg, 91%). R_{f}=0.15 (EtOAc:PE; 3:7); ¹H NMR (600 MHz, CDCl₃) δ 7.34-7.29 (m, 2H), 7.22 (d, J=8.0 Hz, 2H), 6.29 (s, 1H), 4.21-4.14 (m, 2H), 3.95-3.91 (m, 1H), 3.82-3.78 (m, 2H), 3.73 (td, J=11.2, 2.7 Hz, 1H), 3.70-3.67 (m, 1H), 3.59 (td, J=11.3, 3.0 Hz, 1H), 3.46 (dd, J=11.6, 10.1 Hz, 1H), 2.65 (t, J=7.8 Hz, 2H), 2.49 (q, J=7.3 Hz, 2H), 1.65-1.60 (m, 2H), 1.38 (h, J=7.4 Hz, 2H), 1.08 (t, J=7.4 Hz, 3H), 0.94 (t, J=7.4 Hz, 3H); ¹³C NMR (151 MHz, CDCl₃) δ 171.2, 160.1, 151.2, 143.6, 135.2, 128.7, 128.4, 122.0, 94.6, 73.8, 68.1, 66.7, 66.5, 35.6, 33.6, 22.5, 19.6, 14.4, 14.1; HPLC: t_{R}=8.08 min, Method B, Purity: 99.99%.

Further compounds of the invention can be synthesized by methods analogous to those described above.

### Pharmacological Assays

### Assay I: PRESTO-Tango β-Arrestin Recruitment Assay

PRESTO-Tango β-arrestin recruitment assay (W.K. Kroeze et al., 2015). HTLA cells (a HEK293 cell line stably expressing a tTA-dependent luciferase reporter and a β-arrestin2-TEV fusion gene) were maintained in Dulbecco's Modified Eagle's Medium (DMEM) supplemented with 10% Fetal Bovine Serum (dFBS), 100 U/mL penicillin and 100 μg/mL streptomycin, 2 μg/mL puromycin and 100 μg/mL hygromycin B in a humidified atmosphere at 37° C. in 5% CO₂. For transfection, cells were plated at 9×10⁶ to 10×10⁶ cells per 15-cm cell-culture dish (day 1). The following day (day 2), cells were transfected by adding 5 μg DNA and 15 μg polyethylenimine (PEI) diluted in 1 mL Opti-MEM (Reduced Serum Medium supplied by ThermoFisher). On day 3, transfected cells were transferred at 25000 cells per well in 40 μl of medium into poly-l-lysine-coated and rinsed 384-well white, clear-bottomed cell-culture plates (Greiner Bio-One). On day 4, dilutions of the ligands to be tested were prepared in 1% dFBS DMEM and 10 μL were added to each well. On day 5, 20 μl per well of Bright-Glo solution (Promega) diluted 10-fold in assay buffer (Hanks' Balanced Salt Solution [HBSS], 20 mM HEPES, 1 mM CaCl₂, 1 mM MgCl₂, pH adjusted to 7.4 by 5 M NaOH) was added to each well. After incubation for 20 min at room temperature, luminescence was counted in an Enspire luminescence counter at 384 nm. Results in the form of relative luminescence units (RLU) were exported into Excel spreadsheets, and GraphPad Prism was used for analysis of data. To measure constitutive activity, no ligand was added on day 4.

### GPR84 Antagonistic Activity

The GPR84 antagonistic activity of the compounds was determined using the PRESTO-Tango β-arrestin2 recruitment assay (Assay I, see assays above), from which the concentration of each compound required for 50% inhibition (IC₅₀) of the response from the agonist ZQ-16 (100 nM) was determined. The antagonistic activity for each compound is given in Table 1 as the pIC₅₀, which is the negative log of the IC₅₀ value when converted to molar.

### GPR84 Agonist Activity

The GPR84 antagonistic activity of the compounds was determined using the PRESTO-Tango β-arrestin2 recruitment assay (Assay I, see assays above), from which the concentration of each compound required for 50% of maximal activation (EC₅₀) was determined. Maximal activation is indicated as Emax in % relative to the maximal activation of ZQ16. Thus, Emax values between 0% and 100& indicate partial agonism and negative Emax values indicate inverse agonism. The potency of the agonistic activity for each compound in Table 1 is stated as pEC₅₀, denoting the negative log₁₀ of the molar EC₅₀ value.

Assay II: cAMP

The cAMP assay kit from CisBio (cat. No.: 62AM4PEC) was used for performing the assay. A Flp-In 293 T-REx cell line with a stably integrated gene for GPR84-Gai fusion receptor was grown in 10 cm dishes and induced with doxycycline (1 μL/10 mL growth medium: Dulbecco's Modified Eagle Medium 1×(DMEM): +4.5 g/L glucose, +I-glutamine, −pyruvate, 10% v/v FBS, penicillin/streptomycin (100 units/mL/100 mg/mL respectively), 5 mg/mL blasticidin, 1 μg/mL hygromycin B) when confluency reached ˜60%. The next day, dilutions of the compounds (×2.5) to be tested and agonist EC80 solution (×4) were prepared in the stimulation buffer provided with the kit. Then 4 μL of the dilution solutions were transferred to the final 384 well plate (PerkinElmer, cat. No. 6008289) in triplicate. The growth medium of the cells was discarded, and the dishes were washed with 10 mL 1% PBS. The cells were detached by adding 1 mL Versene to the dishes and they were transferred to a 50 mL falcon tube with the addition of 3 mL stimulation buffer per dish. The cells were counted, and the tube was centrifuged at 1500 rpm for 5 min. The supernatant liquid was decanted, and a precise amount of stimulation buffer was added to result in a concentration of 2*10⁶ cells/mL. A certain amount of cell suspension that it will be added to the final plate was transferred to a vial and a DMSO solution of IBMX was added to a final concentration 0.5 mM. Immediately after, 2.5 μL of cell suspension was added with a repeater to the 384-well plate (˜5000 cells/well). Then 2.5 μL of agonist solution was added and the plates were incubated for 15 min at rt. 1 μL of 10 μM solution of forskolin in stimulation buffer was then added to all the wells with a repeater. After 45 min incubation at rt, 5 μL of cAMP-Cryptate conjugate and 5 μL of Anti-cAMP-d2 conjugate solution in lysis buffer, provided with the kit, were added successively. After incubation for 1 h at rt the plates were counted in an Envision plate reader using a Time Resolved Fluorescence protocol, measuring the light emission at 620 and 665 nm. The results were exported in Excel files and the ratio (signal at 665 nm)/(signal at 620 nm)×10⁴ was calculated and plotted versus the compound concentrations using the GraphPad prism software.

TABLE 1
pIC₅₀ values represent antagonist activity in the Presto-TANGO assay.

| Example | Name | Structure | pIC50 |
|---|---|---|---|
| 1 | 3-Bromo-2-(4- propylphenethyl)-6- ((tetrahydro-2H-pyran-2- yl)methoxy)pyridin-4-ol | 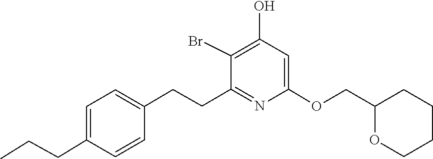 [SMILES: CCCc1ccc(CCc2nc(OCC3CCCCO3)cc(O)c2Br)cc1] | 8.52 |
| 2 | 3-Methyl-2-(4- propylphenethyl)-6- ((tetrahydro-2H-pyran-2- yl)methoxy)pyridin-4-ol | 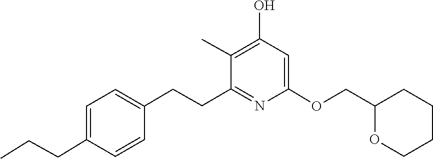 [SMILES: CCCc1ccc(CCc2nc(OCC3CCCCO3)cc(O)c2C)cc1] | 8.88 |
| 3 | 3-Phenyl-2-(4- propylphenethyl)-6- ((tetrahydro-2H-pyran-2- yl)methoxy)pyridin-4-ol | 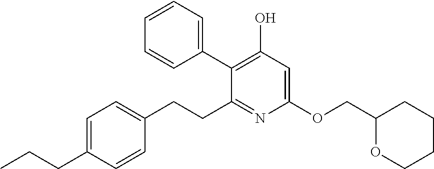 [SMILES: CCCc1ccc(CCc2nc(OCC3CCCCO3)cc(O)c2-c2ccccc2)cc1] | 7.07 |
| 4 | 2-(4-ethoxyphenethyl)-4- hydroxy-6-((tetrahydro- 2H-pyran-2- yl)methoxy)nicotinonitrile | 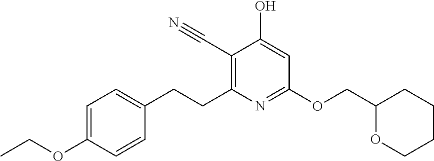 [SMILES: CCOc1ccc(CCc2nc(OCC3CCCCO3)cc(O)c2C#N)cc1] | 6.84 |
| 5 | 7-(4-ethoxyphenyl)-2- ((tetrahydro-2H-pyran-2- yl)methoxy)quinolin-4-ol | 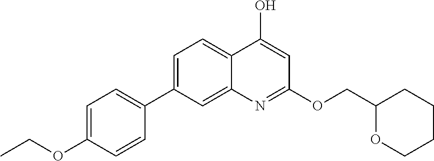 [SMILES: CCOc1ccc(-c2ccc3c(O)cc(OCC4CCCCO4)nc3c2)cc1] | 6.14 |
| 6 | 3-Ethyl-2-(4- propylphenethyl)-6- ((tetrahydro-2H-pyran-2- yl)methoxy)pyridin-4-ol | 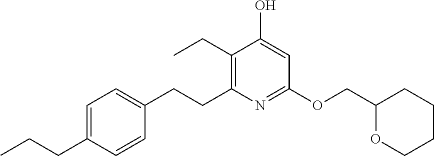 [SMILES: CCCc1ccc(CCc2nc(OCC3CCCCO3)cc(O)c2CC)cc1] | 8.67 |
| 7 | 3-Propyl-2-(4- propylphenethyl)-6- ((tetrahydro-2H-pyran-2- yl)methoxy)pyridin-4-ol | 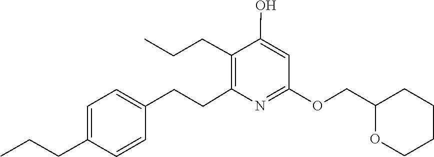 [SMILES: CCCc1ccc(CCc2nc(OCC3CCCCO3)cc(O)c2CCC)cc1] | 8.18 |
| 8 | 3-Isopropyl-2-(4- propylphenethyl)-6- ((tetrahydro-2H-pyran-2- yl)methoxy)pyridin-4-ol | 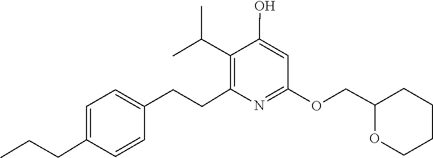 [SMILES: CCCc1ccc(CCc2nc(OCC3CCCCO3)cc(O)c2C(C)C)cc1] | 7.09 |
| 9 | 2-(4-Propylphenethyl)-6- ((tetrahydro-2H-pyran-2- yl)methoxy)-3-(o- tolyl)pyridine-4-ol | 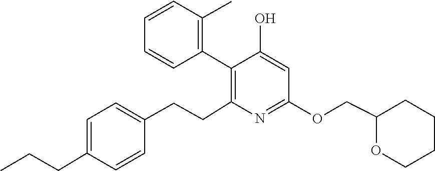 [SMILES: CCCc1ccc(CCc2nc(OCC3CCCCO3)cc(O)c2-c2ccccc2C)cc1] | 6.41 |
| 10 | 2-(4-Propylphenethyl)-6- ((tetrahydro-2H-pyran-2- yl)methoxy)-3-(p- tolyl)pyridine-4-ol | 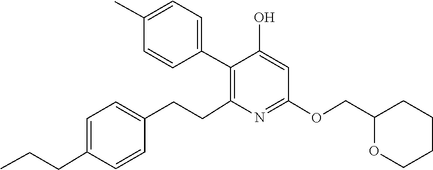 [SMILES: CCCc1ccc(CCc2nc(OCC3CCCCO3)cc(O)c2-c2ccc(C)cc2)cc1] | 7.18 |
| 11 | 3-Phenethyl-2-(4- propylphenethyl)-6- ((tetrahydro-2H-pyran-2- yl)methoxy)pyridin-4-ol | 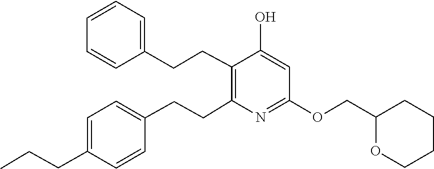 [SMILES: CCCc1ccc(CCc2nc(OCC3CCCCO3)cc(O)c2CCc2ccccc2)cc1] | 6.75 |
| 12 | Ethyl (E)-3-(4-hydroxy-2- (4-propylphenethyl)-6- ((tetrahydro-2H-pyran-2- yl)methoxy)pyridin-3- yl)acrylate | 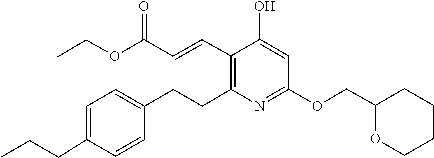 [SMILES: CCCc1ccc(CCc2nc(OCC3CCCCO3)cc(O)c2/C=C/C(=O)OCC)cc1] | 7.66 |
| 13 | Ethyl 3-(4-hydroxy-2-(4- propylphenethyl)-6- ((tetrahydro-2H-pyran-2- yl)methoxy)pyridin-3- yl)propanoate | 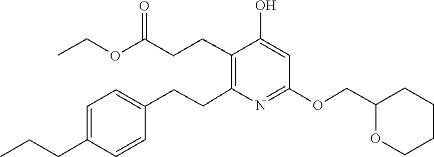 [SMILES: CCCc1ccc(CCc2nc(OCC3CCCCO3)cc(O)c2CCC(=O)OCC)cc1] | 7.88 |
| 14 | Sodium 3-(4-hydroxy-2- (4-propylphenethyl)-6- ((tetrahydro-2H-pyran-2- yl)methoxy)pyridin-3- yl)propanoate | 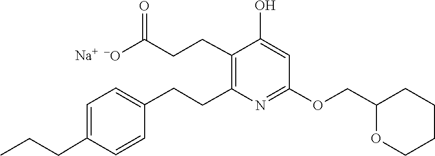 | 8.95 |
| 15 | 3-(3-Hydroxypropyl)-2-(4- propylphenethyl)-6- ((tetrahydro-2H-pyran-2- yl)methoxy)pyridin-4-ol | 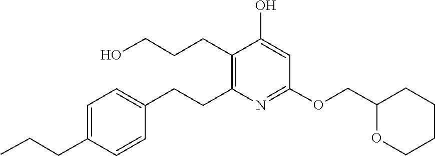 [SMILES: CCCc1ccc(CCc2nc(OCC3CCCCO3)cc(O)c2CCCO)cc1] | 8.77 |
| 16 | 3-(4-Ethylphenyl)-2-(4- propylphenethyl)-6- ((tetrahydro-2H-pyran-2- yl)methoxy)pyridin-4-ol | 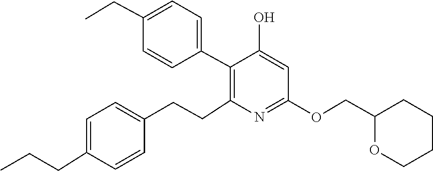 [SMILES: CCCc1ccc(CCc2nc(OCC3CCCCO3)cc(O)c2-c2ccc(CC)cc2)cc1] | 7.15 |
| 17 | 3-(4-Hydroxyphenyl)-2- (4-propylphenethyl)-6- ((tetrahydro-2H-pyran-2- yl)methoxy)pyridin-4-ol | 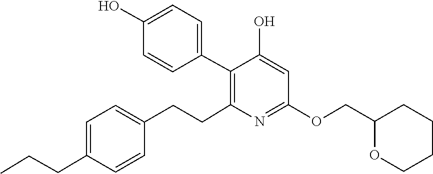 [SMILES: CCCc1ccc(CCc2nc(OCC3CCCCO3)cc(O)c2-c2ccc(O)cc2)cc1] | 7.03 |
| 18 | Methyl 2-(4-(4-hydroxy-2- (4-propylphenethyl)-6- ((tetrahydro-2H-pyran-2- yl)methoxy)pyridin-3- yl)phenoxy)acetate | 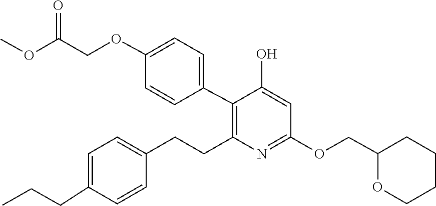 [SMILES: CCCc1ccc(CCc2nc(OCC3CCCCO3)cc(O)c2-c2ccc(OCC(=O)OC)cc2)cc1] | 6.83 |
| 19 | 2-(4-(4-Hydroxy-2-(4- propylphenethyl)-6- ((tetrahydro-2H-pyran-2- yl)methoxy)pyridin-3- yl)phenoxy)acetic acid | 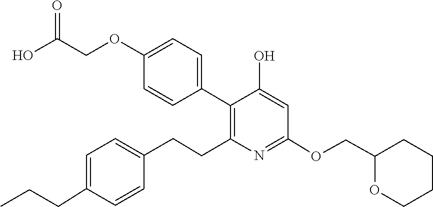 [SMILES: CCCc1ccc(CCc2nc(OCC3CCCCO3)cc(O)c2-c2ccc(OCC(=O)O)cc2)cc1] | 7.03 |
| 20 | Methyl 4-(4-hydroxy-2-(4- propylphenethyl)-6- ((tetrahydro-2H-pyran-2- yl)methoxy)pyridin-3- yl)benzoate | 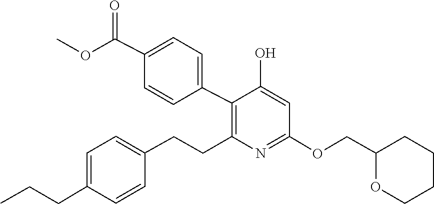 [SMILES: CCCc1ccc(CCc2nc(OCC3CCCCO3)cc(O)c2-c2ccc(C(=O)OC)cc2)cc1] | 6.84 |
| 21 | 4-(4-Hydroxy-2-(4- propylphenethyl)-6- ((tetrahydro-2H-pyran-2- yl)methoxy)pyridin-3- yl)benzoic acid | 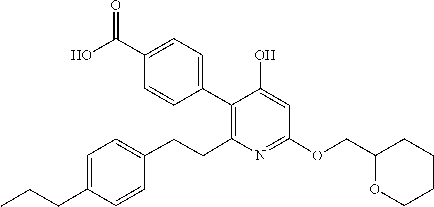 [SMILES: CCCc1ccc(CCc2nc(OCC3CCCCO3)cc(O)c2-c2ccc(C(=O)O)cc2)cc1] | 6.85 |
| 22 | 6-((1,4-Dioxan-2- yl)methoxy)-3-methyl-2- (4- propylphenethyl)pyridin- 4-ol | 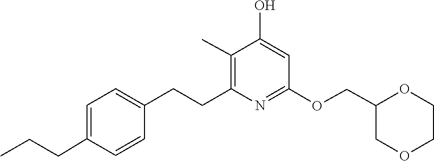 [SMILES: CCCc1ccc(CCc2nc(OCC3COCCO3)cc(O)c2C)cc1] | 8.52 |
| 23 | 6-((1,4-Dioxan-2- yl)methoxy)-2-(4- ethoxyphenethyl)-3- methylpyridin-4-ol | 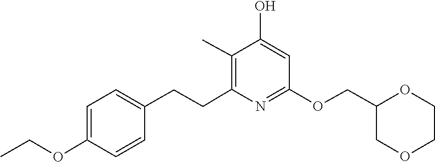 [SMILES: CCOc1ccc(CCc2nc(OCC3COCCO3)cc(O)c2C)cc1] | 8.48 |
| 24 | (S)-6-((1,4-dioxan-2- yl)methoxy)-2-(4- ethoxyphenethyl)-3- methylpyridin-4-ol | 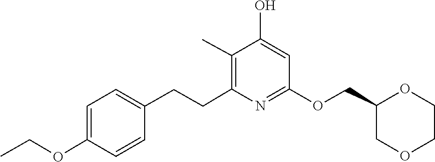 [SMILES: CCOc1ccc(CCc2nc(OC[C@@H]3COCCO3)cc(O)c2C)cc1] | 8.97 |
| 25 | 6-((1,4-Dioxan-2- yl)methoxy)-3-ethyl-2-(4- propylphenethyl)pyridin- 4-ol | 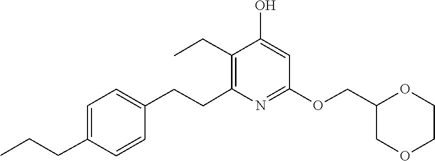 [SMILES: CCCc1ccc(CCc2nc(OCC3COCCO3)cc(O)c2CC)cc1] | 9.11 |
| 26 | 6-((1,4-Dioxan-2- yl)methoxy)-2-(4- ethoxyphenethyl)-3- ethylpyridin-4-ol | 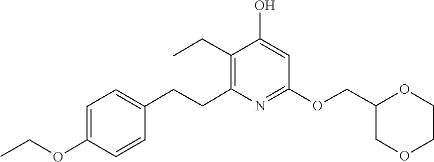 [SMILES: CCOc1ccc(CCc2nc(OCC3COCCO3)cc(O)c2CC)cc1] | 9.41 |
| 27 | tert-Butyl (2-(4-(2-(6- ((1,4-dioxan-2- yl)methoxy)-4-hydroxy-3- methylpyridin-2- yl)ethyl)phenoxy)- ethyl)carbamate | 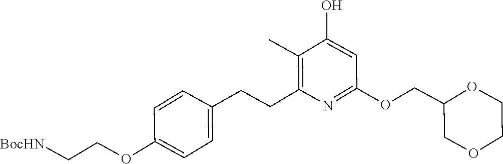 [SMILES: Cc1c(O)cc(OCC2COCCO2)nc1CCc1ccc(OCCNC(=O)OC(C)(C)C)cc1] | 9.55 |
| 28 | Ethyl 2-(4-(2-(6-((1,4- dioxan-2-yl)methoxy)-4- hydroxy-3-methylpyridin- 2-yl)ethyl)phenoxy)- acetate | 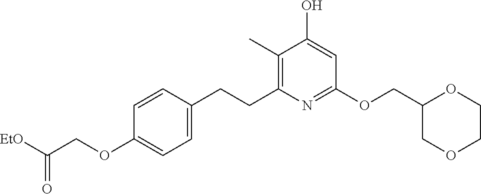 [SMILES: CCOC(=O)COc1ccc(CCc2nc(OCC3COCCO3)cc(O)c2C)cc1] | 7.95 |
| 29 | 2-(4-(2-(6-((1,4-Dioxan-2- yl)methoxy)-4-hydroxy-3- methylpyridin-2- yl)ethyl)phenoxy)ethan- 1-aminium chloride | 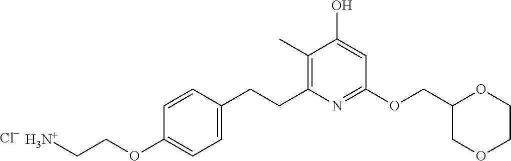 [SMILES: [Cl-] ; Cc1c(O)cc(OCC2COCCO2)nc1CCc1ccc(OCC[NH3+])cc1] | 7.79 |
| 30 | 2-(4-(2-(6-((1,4-Dioxan-2- yl)methoxy)-4-hydroxy-3- methylpyridin-2- yl)ethyl)phenoxy)acetic acid | 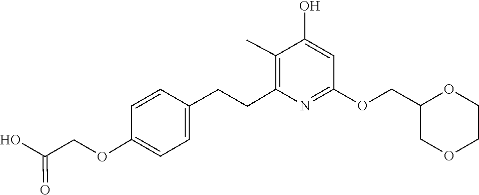 [SMILES: Cc1c(O)cc(OCC2COCCO2)nc1CCc1ccc(OCC(=O)O)cc1] | 5.89 |
| 31 | N-(2-(4-(2-(6-((1,4- dioxan-2-yl)methoxy)-4- hydroxy-3-methylpyridin- 2- yl)ethyl)phenoxy)ethyl)- acetamide | 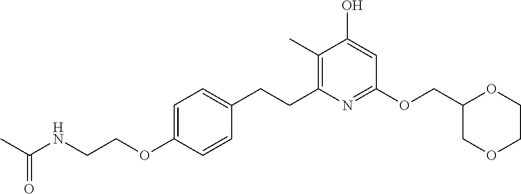 [SMILES: CC(=O)NCCOc1ccc(CCc2nc(OCC3COCCO3)cc(O)c2C)cc1] | 7.75 |
| 32 | tert-Butyl (2-(4-(2-(6- ((1,4-dioxan-2- yl)methoxy)-3-ethyl-4- hydroxypyridin-2- yl)ethyl)phenoxy)ethyl)- carbamate | 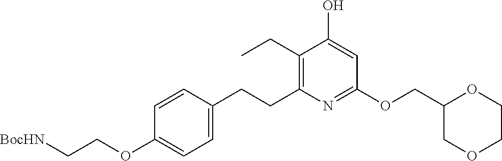 [SMILES: CCc1c(O)cc(OCC2COCCO2)nc1CCc1ccc(OCCNC(=O)OC(C)(C)C)cc1] | 9.68 |
| 33 | tert-Butyl (3-(4-(2-(6- ((1,4-dioxan-2- yl)methoxy)-3-ethyl-4- hydroxypyridin-2- yl)ethyl)phenoxy)- propyl)carbamate | 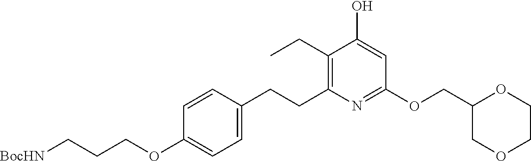 [SMILES: CCc1c(O)cc(OCC2COCCO2)nc1CCc1ccc(OCCCNC(=O)OC(C)(C)C)cc1] | 9.52 |
| 34 | tert-Butyl (2-(2-(4-(2-(6- ((1,4-dioxan-2- yl)methoxy)-3-ethyl-4- hydroxypyridin-2- yl)ethyl)phenoxy)ethoxy)- ethyl)carbamate | 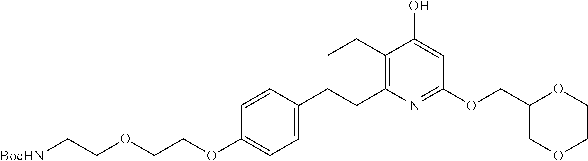 [SMILES: CCc1c(O)cc(OCC2COCCO2)nc1CCc1ccc(OCCOCCNC(=O)OC(C)(C)C)cc1] | 9.48 |
| 35 | 6-((1,4-Dioxan-2- yl)methoxy)-3-ethyl-2-(4- (2-hydroxyethoxy)- phenethyl)pyridin-4-ol | 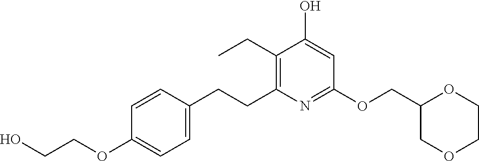 [SMILES: CCc1c(O)cc(OCC2COCCO2)nc1CCc1ccc(OCCO)cc1] | 9.13 |
| 36 | Ethyl 5-(4-(2-(6-((1,4- dioxan-2-yl)methoxy)-3- ethyl-4-hydroxypyridin-2- yl)ethyl)phenoxy)- pentanoate | 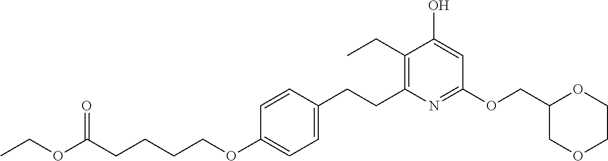 [SMILES: CCOC(=O)CCCCOc1ccc(CCc2nc(OCC3COCCO3)cc(O)c2CC)cc1] | 9.59 |
| 37 | 2-(4-(2-(6-((1,4-Dioxan-2- yl)methoxy)-3-ethyl-4- hydroxypyridin-2- yl)ethyl)phenoxy)- acetamide | 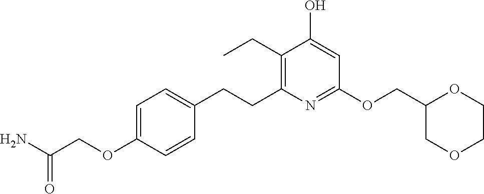 [SMILES: CCc1c(O)cc(OCC2COCCO2)nc1CCc1ccc(OCC(N)=O)cc1] | 8.54 |
| 38 | 6-((1,4-Dioxan-2- yl)methoxy)-3-methyl-2- (4-(2-phenoxyethoxy)- phenethyl)pyridin-4-ol | 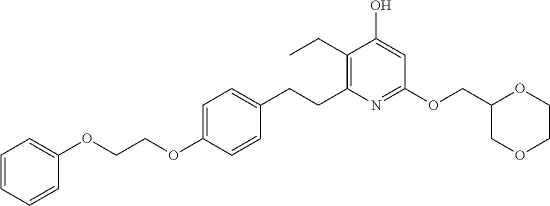 [SMILES: CCc1c(O)cc(OCC2COCCO2)nc1CCc1ccc(OCCOc2ccccc2)cc1] | 9.70 |
| 39 | 6-((1,4-Dioxan-2- yl)methoxy)-3-ethyl-2-(4- (3-(methylsulfonyl)- propoxy)phenethyl)- pyridin-4-ol | 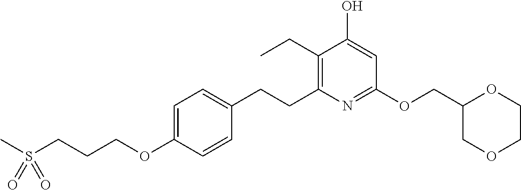 [SMILES: CCc1c(O)cc(OCC2COCCO2)nc1CCc1ccc(OCCCS(C)(=O)=O)cc1] | 9.06 |
| 40 | 6-((1,4-Dioxan-2- yl)methoxy)-3-ethyl-2-(4- ((3-methyl-1,2,4- oxadiazol-5-yl)methoxy)- phenethyl)pyridin-4-ol | 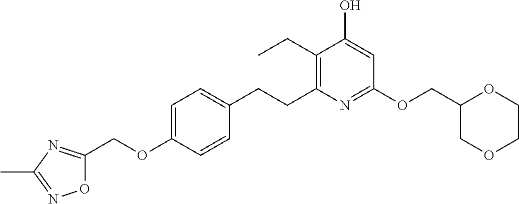 [SMILES: CCc1c(O)cc(OCC2COCCO2)nc1CCc1ccc(OCc2nc(C)no2)cc1] | 8.53 |
| 41 | Methyl 2-((4-(2-(6-((1,4- dioxan-2-yl)methoxy)-3- ethyl-4-hydroxypyridin-2- yl)ethyl)phenoxy)methyl)- oxazole-4-carboxylate | 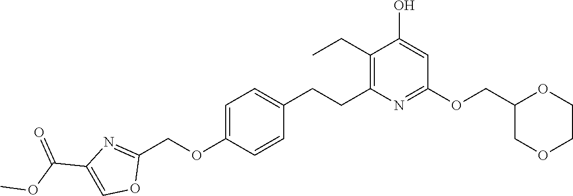 [SMILES: CCc1c(O)cc(OCC2COCCO2)nc1CCc1ccc(OCc2nc(C(=O)OC)co2)cc1] | 9.12 |
| 42 | 2-(2-(4-(2-(6-((1,4-Dioxan- 2-yl)methoxy)-3-ethyl-4- hydroxypyridin-2- yl)ethyl)phenoxy)- ethyl)isoindoline-1,3- dione | 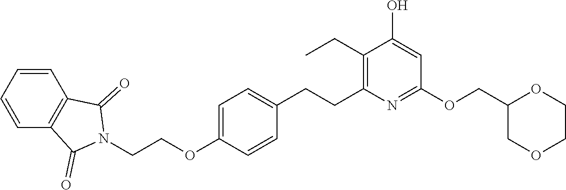 [SMILES: CCc1c(O)cc(OCC2COCCO2)nc1CCc1ccc(OCCN2C(=O)c3ccccc3C2=O)cc1] | 9.01 |
| 43 | 6-((1,4-Dioxan-2- yl)methoxy)-3-ethyl-2-(4- hydroxyphenethyl)- pyridin-4-ol | 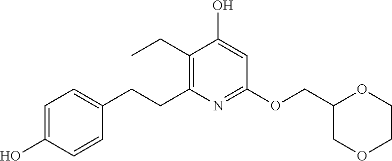 [SMILES: CCc1c(O)cc(OCC2COCCO2)nc1CCc1ccc(O)cc1] | 8.97 |
| 44 | 6-((1,4-Dioxan-2- yl)methoxy)-2-(2-(4- (ethoxy-d5)phenyl)ethyl- 1,1,2,2-d4)-3- ethylpyridin-4-ol | 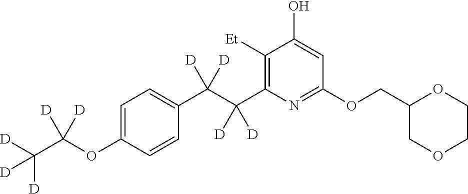 [SMILES: [2H]C([2H])([2H])C([2H])([2H])Oc1ccc(C([2H])([2H])C([2H])([2H])c2nc(OCC3COCCO3)cc(O)c2CC)cc1] | 9.68 |
| 45 | 6-((1,4-Dioxan-2- yl)methoxy)-2-(4-(2- aminoethoxy)phenethyl)- 3-ethylpyridin-4-ol hydrochloride | 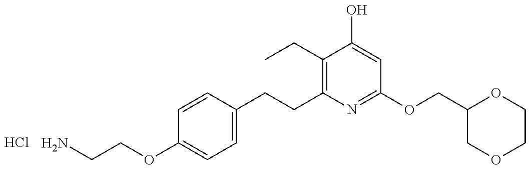 [SMILES: Cl ; CCc1c(O)cc(OCC2COCCO2)nc1CCc1ccc(OCCN)cc1] | 8.63 |
| 46 | 6-((1,4-Dioxan-2- yl)methoxy)-2-(4-(3- aminopropoxy)- phenethyl)-3- ethylpyridin-4-ol hydrochloride | 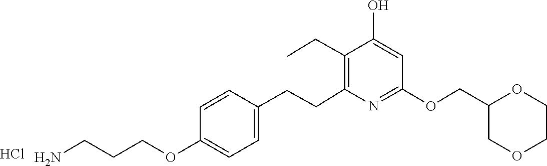 [SMILES: Cl ; CCc1c(O)cc(OCC2COCCO2)nc1CCc1ccc(OCCCN)cc1] | 9.08 |
| 47 | 5-(4-(2-(6-((1,4-Dioxan-2- yl)methoxy)-3-ethyl-4- hydroxypyridin-2- yl)ethyl)phenoxy)- pentanoic acid | 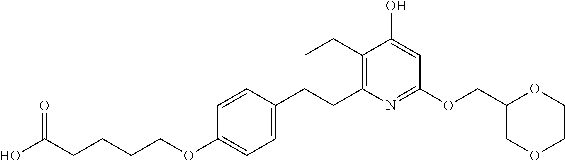 [SMILES: CCc1c(O)cc(OCC2COCCO2)nc1CCc1ccc(OCCCCC(=O)O)cc1] | 8.39 |
| 48 | 2-((4-(2-(6-((1,4-Dioxan-2- yl)methoxy)-3-ethyl-4- hydroxypyridin-2- yl)ethyl)phenoxy)- methyl)oxazole-4- carboxylic acid | 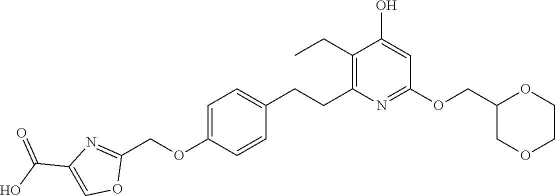 [SMILES: CCc1c(O)cc(OCC2COCCO2)nc1CCc1ccc(OCc2nc(C(=O)O)co2)cc1] | 8.27 |
| 49 | 6-((1,4-Dioxan-2- yl)methoxy)-2-(2-(6- ethoxypyridin-3-yl)ethyl)- 3-ethylpyridin-4-ol | 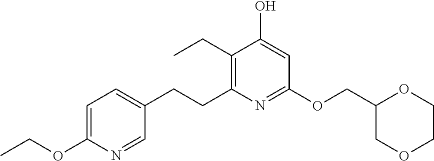 [SMILES: CCOc1ccc(CCc2nc(OCC3COCCO3)cc(O)c2CC)cn1] | 9.30 |
| 50 | 6-((1,4-Dioxan-2- yl)methoxy)-2-(2-(6- ethoxy-5-fluoropyridin-3- yl)ethyl)-3-ethylpyridin-4- ol | 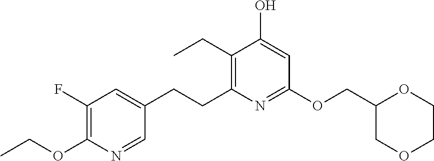 [SMILES: CCOc1ncc(CCc2nc(OCC3COCCO3)cc(O)c2CC)cc1F] | 8.89 |
| 51 | 6-((1,4-Dioxan-2- yl)methoxy)-2-(2-(4- ethoxyphenyl)- cyclopropyl)-3- ethylpyridin-4-ol | 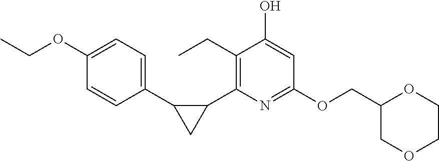 [SMILES: CCOc1ccc(C2CC2c2nc(OCC3COCCO3)cc(O)c2CC)cc1] | 7.06 |
| 52 | (S)-6-((1,4-dioxan-2- yl)methoxy)-2-(3,4- dimethoxyphenethyl)-3- ethylpyridin-4-ol | 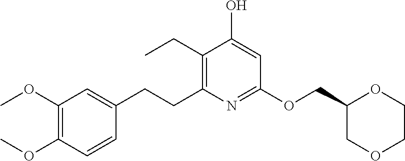 [SMILES: CCc1c(O)cc(OC[C@@H]2COCCO2)nc1CCc1ccc(OC)c(OC)c1] | 8.90 |
| 53 | (S)-6-((1,4-dioxan-2- yl)methoxy)-2-(2,4- dimethoxyphenethyl)-3- ethylpyridin-4-ol | 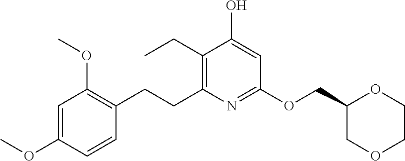 [SMILES: CCc1c(O)cc(OC[C@@H]2COCCO2)nc1CCc1ccc(OC)cc1OC] | 9.45 |
| 54 | (S)-5-(2-(6-((1,4-dioxan-2- yl)methoxy)-3-ethyl-4- hydroxypyridin-2- yl)ethyl)-2- methoxybenzonitrile | 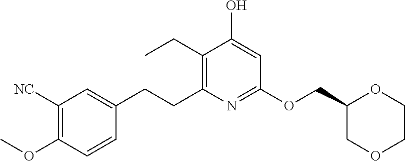 [SMILES: CCc1c(O)cc(OC[C@@H]2COCCO2)nc1CCc1ccc(OC)c(C#N)c1] | 8.81 |
| 55 | (S)-2-(2-(6-((1,4-dioxan-2- yl)methoxy)-3-ethyl-4- hydroxypyridin-2- yl)ethyl)-5- methoxybenzonitrile | 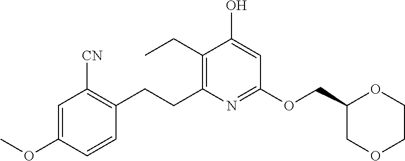 [SMILES: CCc1c(O)cc(OC[C@@H]2COCCO2)nc1CCc1ccc(OC)cc1C#N] | 9.26 |
| 56 | (S)-6-((1,4-dioxan-2- yl)methoxy)-3-ethyl-2-(4- phenylbutyl)pyridin-4-ol | 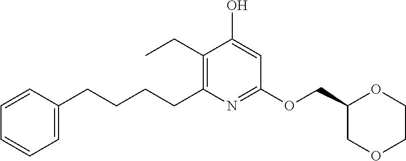 [SMILES: CCc1c(O)cc(OC[C@@H]2COCCO2)nc1CCCCc1ccccc1] | 7.63 |
| 57 | (S)-6-((1,4-dioxan-2- yl)methoxy)-2-(4- butylphenyl)-3- ethylpyridin-4-ol | 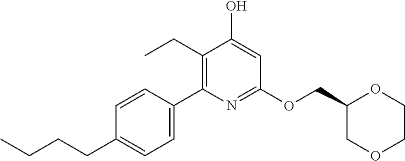 [SMILES: CCCCc1ccc(-c2nc(OC[C@@H]3COCCO3)cc(O)c2CC)cc1] | 6.92 |

All above compounds in the table are examples of embodiments of the invention.

### Pharmacokinetic Evaluation

This study is performed with 6 male BalbC mice (6-7 weeks old, 21-25 g). The first group of 3 mice is dosed intravenous via a bolus in the tail vein (1 mg/kg) and the second group of 3 mice is dosed orally (5 mg/kg) via an oral gavage. The compound solutions are prepared the day before administration and kept at rt. Compounds for iv administration are formulated in PEG 200 and saline (60/40, v/v) and for oral administration in PEG 200 and MQ water (60/40, v/v). The mice are fasted for 12 h before the oral administration and 4 h after, while having free access to water. From each animal and for each time-point, 10 μL of blood is collected by piercing the tail or the submandibular vein and mixed directly with 30 μL of trisodium citrate buffer (0.1 M) in an Eppendorf tube. The samples are kept on ice until further analysis. Samples are collected at seven time-points.

A solution of internal standard (10 μL) in MeCN and additional MeCN (50 μL) is added to the blood-citrate mixture, and the suspension is vortexed and centrifuged at 4000 rpm for 15 min. The supernatant is transferred to HPLC vials and analyzed in LCMS in Single Ion Mode, detecting only the molecular mass of the compound of interest and the internal standards. LCMS mass spectra are obtained with an Agilent 6130 Mass Spectrometer instrument using electron spray ionization (ESI) coupled to an Agilent 1200 HPLC system (ESI-LCMS) with a C18 reverse phase column (Zorbax Eclipse XBD-C18, 4.6 mm×50 mm), autosampler and diode array detector, using a linear gradient of the binary solvent system of buffer A (milliQ water:MeCN:formic acid, 95:5:0.1 v/v %) to buffer B (MeCN:formic acid, 100:0.1 v/v %) with a flow rate of 1 mL/min. Compound blood concentrations are calculated against a 10-level (3.7 log amplitude) calibration curve. PK parameters were calculated from the mean of individual blood concentrations by noncompartmental analysis using PKSolver 2.0 Excel add-in (Zhang, Y., et al. Computer Methods and Programs in Biomedicine 2010, 99, 306-314).

### In Vivo Pharmacological Assays

### CCl4-Induced Liver Fibrosis

Sprague Dawley rats are orally administered with 0.25 μL/g carbon tetrachloride (CCl₄) in olive oil solution, starting from day 0, 3 times per week for 6 weeks. Animals are sacrificed 48 hours after the last CCl₄ administration. The test compound is given by oral gavage after 3 weeks of CCl4 administration and continued throughout the remainder of the study at 3 mg/kg, 10 mg/kg or 30 mg/kg once a day. Alanine aminotransferase (ALT) and aspartate aminotransferase (AST) levels are assessed in the plasma. One lobe of the liver tissue are fixed in 10% formalin, stained for Sirius red and the percentage coverage area is measured.

### NASH-Induced Liver Disease

Non-alcoholic steatohepatitis (NASH) is established in male C57/BL6 mice by a single subcutaneous injection of 200 μg streptozotocin after birth and with a high fat diet ad libitum from 4 until 14 weeks of age. Mice are orally administered with the test compound (3, 10 or 30 mg/kg once daily) from 8 to 14 weeks of age. Plasma ALT levels are determined. One lobe of the liver tissue was fixed in 10% formalin, stained for Sirius red and the percentage coverage area is measured. Hematoxylin and eosin (HE) staining is performed to estimate non-alcoholic fatty liver disease (NAFLD) activity score according to the criteria of Kleiner et al (2005).

### Bleomycin-Induced Mouse Model of Pulmonary Fibrosis

The bleomycin-induced model of pulmonary fibrosis is performed essentially as described by Gagnon et al (2018). Briefly, 10 week old C57BL/6 mice are intratracheally instilled with bleomycin (0.025 U per mouse). Mice are grouped according to their body weight loss and treated with test compound (3, 10 or 30 mg/kg per day) or vehicle from day 7 to day 20 via gastric gavage. On day 21, lungs are prepared for histologic assessment of lesions with Masson's trichrome staining.

### Adenine-Induced Tubulointerstitial Nephritis Mouse Model

The study is performed essentially as described by Tamura et al (2009). Briefly, mice are fed either standard chow with or without supplement of 0.25% adenine ad libitum for 4 weeks. After 1 week of adenine administration, mice were given either vehicle or test compound (3, 10 or 30 mg/kg per day) by gastric gavage for 3 weeks. Kidney sections were stained with Masson's trichrome for histologic evaluation of tubulointerstitial fibrosis and cystic lesions scores.

### Doxorubicin Mouse Model of Nephropathy

The doxorubicin nephropathy mouse model was performed as described by Gagnon et al. (2018).

Briefly, nephrotoxicity was induced in 6-10 weeks old mice by i.v. injection of doxorubicin (10 mg/kg) on day 0. Test compound (3, 10 or 30 mg/kg per day) or vehicle was administered from day −3 to −1 and day 1 to day 10, and mice were sacrificed on the following day. Kidneys were prepared for histologic assessment of glomerular and tubular lesions with hematoxylin and eosin staining.

### REFERENCES

W.K. Kroeze et al., 2015 PRESTO-TANGO: an open-source resource for interrogation of the druggable human GPCR-ome in Nat Struct Mol Biol. 2015 May; 22(5): 362-369.
Mahmud et al. (2017) Three classes of ligands each bind to distinct sites on the orphan G protein-coupled receptor GPR84, Scientific reports, 2017, 7:17953
Gagnon et al. (2018) A Newly Discovered Antifibrotic Pathway Regulated by Two Fatty Acid Receptors: GPR40 and GPR84, Am. J. Pathol. 2018, 188(5):1132-1148
Lynch & Wang (2016) G Protein-Coupled Receptor Signaling in Stem Cells and Cancer. Int J Mol Sci. 17(5): 707
Miyamoto et al (2017) Anti-Inflammatory and Insulin-Sensitizing Effects of Free Fatty Acid Receptors. Handb. Exp. Pharmacol. 236: 221-231.
Milligan et al (2017) Complex pharmacology of free fatty acid receptors. 117(1): 67-110.
Suckow & Briscoe (2017) Key questions for translation of FFA receptors: From pharmacology to medicines. 236: 101-131.
Pillaiyar et al. (2017) Diindolylmethane Derivatives: Potent Agonists of the Immunostimulatory Orphan G Protein-Coupled Receptor GPR84. J. Med. Chem. 60(9): 3636-3655.
Pillaiyar et al. (2018) 6-(Ar)Alkylamino-Substituted Uracil Derivatives: Lipid Mimetics with Potent Activity at the Orphan G Protein-Coupled Receptor 84 (GPR84). ACS Omega 3(3): 3365-3383.
Kôse et al. (2019) An agonist radioligand for the proinflammatory lipid-activated G protein-coupled receptor GPR84 providing structural insights. J. Med. Chem. DIO: 10.1021/acs.jmedchem.9b01339
Kleiner et al. (2005) Design and validation of a histological scoring system for nonalcoholic fatty liver disease. Hepatology 41(6): 1313-1321.
Tamura et al. (2009) Progressive renal dysfunction and macrophage infiltration in interstitial fibrosis in an adenine-induced tubulointerstitial nephritis mouse model. Histochem. Cell Biol. 131(4): 483-490.
Liu et al. (2016) Design and synthesis of 2-alkylpyrimidine-4,6-diol and 6-alkylpyridine-2,4-diol as potent GPR84 agonists. ACS Med. Chem. Lett. 7(6):579-583.

1. A compound of the formula I: 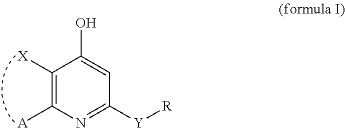
wherein
A is -(A1)ⱼ—(B1)ₖ-(A2)ₗ—(B2)ₘ—H, wherein
A1 is C₁-7 alkylene, C₂₋₇ alkenylene, C₂₋₇ alkynylene, or C₂₋₇ heteroalkylene, optionally substituted with one or two of independently selected U1;
A2 is C₁₋₁₄ alkylene, C₂₋₁₄ alkenylene, C₂₋₁₄ alkynylene, or C₂₋₁₄ heteroalkylene, optionally substituted with one, two, three or four of independently selected U1;
B1 and B2 are independently a C₃-7 aliphatic ring, a mono- or bicyclic aromatic ring or a fused ring, optionally substituted with one, two, three or four of independently selected U2;
H is hydrogen;
j, k, l, and m are independently 0 or 1, wherein at least one of j, k, l and m is 1;
X is C₁₋₇ alkyl, C₂₋₇ heteroalkyl, C₂₋₇ alkenyl, C₂₋₇ heteroalkenyl, C₂₋₇ alkynyl, aryl, arylalkyl, aromatic ring, halogen, —CN, —NO₂, CF₃, —OH or —NH₂, optionally substituted with one, two or three independently selected U3;
------ defines an optional bond between X and A which is formed by substitution of one or two hydrogen radicals at a position on each of A and X such that a single or double bond is formed that results in a 5- or 6-membered aliphatic or aromatic ring fused with the pyridine;
Y is —OCH₂—; and
R is 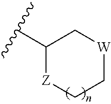
where the ring is optionally substituted with —CF₃, C₁₋₃ alkyl or C₂₋₄ heteroalkyl, and wherein Z is —O—, W is —O— or —CH₂—, and n is 0, 1 or 2, preferably n is 1, or R is a bicyclic fused ring composed of a 1,4-dioxane and an aromatic ring, optionally substituted with one, two or three of independently selected U4;
U1, U2, U3 and U4 are independently hydrogen, C₁₋₃ alkyl, C₁₋₃ haloalkyl, C₂₋₄ heteroalkyl, halogen, hydroxyl, ═O, =NR′=N—OR′—NR′R″, —SR′, —CN, —SO₃H, —CO₂R′, —CONR′, C₂₋₅ alkynyl, C₂₋₅ alkenyl, C₃₋₅ cycloalkyl, C₃₋₅ heterocycloalkyl or —NO₂, where R′, and R″ independently refer to hydrogen, unsubstituted C₁₋₃ alkyl or C₂₋₃ heteroalkyl that can be connected by bonds to form heterocycloalkyl;
or a pharmaceutically acceptable salt thereof, or a solvate thereof, or a solvate of the pharmaceutically acceptable salt thereof.

2. The compound of the formula I 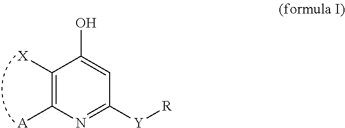
wherein
A is -(A1)ⱼ—(B1)ₖ-(A2)ₗ—(B2)ₘ—H, wherein
A1 and A2 independently are C₁₋₇ alkylene, C₂₋₇ alkenylene, C₂₋₇ alkynylene, or C₁₋₇ heteroalkylene, optionally substituted with one or two of independently selected U1;
B1 and B2 are independently an aliphatic ring, an aromatic ring or a fused ring, optionally substituted with one or two of independently selected U2;
H is hydrogen;
j, k, l, and m are independently 0 or 1, wherein at least one of j, k, l and m is 1;
X is C₁₋₇ alkyl, C₂₋₇ heteroalkyl, C₂₋₇ alkenyl, C₂₋₇ heteroalkenyl, C₂₋₇ alkynyl, halogen, —CN, —NO₂, CF₃, —OH or —NH₂, optionally substituted with one, two or three independently selected U3;
------ defines an optional bond between X and A which is formed by substitution of one or two hydrogen radicals at a position on each of A and X such that a single or double bond is formed that results in a 5- or 6-membered aliphatic or aromatic ring fused with the pyridine;
Y is —OCH₂—; and
R is 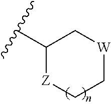
where the ring is optionally substituted with —CF₃, C₁₋₃ alkyl or C₂₋₄ heteroalkyl, and wherein Z is —O—, W is —O— or —CH₂—, and n is 0, 1 or 2, or R is a bicyclic fused ring composed of a 1,4-dioxane and an aromatic ring, optionally substituted with one, two or three of independently selected U4;
U1, U2, U3 and U4 are independently hydrogen, C₁₋₃ alkyl, C₁₋₃ haloalkyl, C₂₋₄ heteroalkyl, halogen, hydroxyl, ═O, =NR′=N—OR′—NR′R″, —SR′, —CN, C₂₋₅ alkynyl, C₂₋₅ alkenyl, C₃₋₅ cycloalkyl, C₃₋₅ heterocycloalkyl or —NO₂, where R′ and R″ independently refer to hydrogen, unsubstituted C₁₋₃ alkyl or C₂₋₃ heteroalkyl that can be connected by bonds to form rings, cycloalkyl or heterocycloalkyl;
or a pharmaceutically acceptable salt thereof, or a solvate thereof, or a solvate of the pharmaceutically acceptable salt thereof.

3. (canceled)

4. (canceled)

5. (canceled)

6. The compound according to claim 1, wherein n=1.

7. (canceled)

8. (canceled)

9. (canceled)

10. The compound according to claim 1, wherein Y is —OCH₂—, and R is 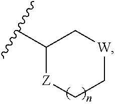
where n=1, Z=—O— and W=—O—.

11. The compound according to claim 1, wherein X is C₁₋₇ alkyl or C₂₋₇ heteroalkyl optionally substituted with one or two of independently selected U3, halogen, CN, or —CF₃.

12. The compound according to claim 1, wherein X is C₁₋₃ alkyl.

13. The compound according to claim 1, wherein X is methyl or ethyl.

14. The compound according to claim 1, wherein
j=1, k=1, l=1, and m=1, resulting in A being -A1-B1-A2-B2-H, or
j=1, k=1, l=1, and m=0, resulting in A being -A1-B1-A2-H.

15. The compound according to claim 1, wherein A1 is C₁₋₇ alkylene or C₁₋₇ heteroalkylene, optionally substituted with one or two of independently selected U1.

16. The compound according to claim 1, wherein A1 is ethylene or -CD₂CD₂-.

17. (canceled)

18. The compound according to claim 1, wherein A1 is ethylene or —CD₂CD₂- substituted by one or two of independently selected U1, and B1 is benzene substituted by one, two, three or four of independently selected U2.

19. The compound according to claim 1, wherein A2 is C₁₋₁₄ alkylene or C₂₋₁₄ heteroalkylene, optionally substituted with one, two, three or four of independently selected U1.

20. The compound according to claim 1, wherein B1 and/or B2 is an aromatic ring, optionally substituted with one or two of independently selected U2.

21. The compound according to claim 1, wherein A is
(i) -A1-B1-A2-H, where A1 is C₁₋₅ alkylene or C₂₋₅ heteroalkylene, B1 is aryl, and A2 is C₁₋₅ alkylene or C₂₋₁₄ heteroalkylene optionally substituted with one, two, three or four of independently selected U1,
(ii) -A1-B1-A2-B2-H, where A1 and A2 are C₁₋₅ alkylene or C₂₋₅ heteroalkylene optionally substituted with one, two, three or four of independently selected U1, and B1 and B2 are aryl optionally substituted with one, two, three or four of independently selected U2, or
(iii) -A1-B1-H, where A1 is C₁₋₅ alkylene or C₂₋₅ heteroalkylene optionally substituted with one, two, three or four of independently selected U1, and B1 is aryl optionally substituted with one or two of independently selected U2.

22. The compound according to claim 1, wherein X is C₁₋₇ alkyl or halogen and A is A1-B1-A2-H, where A1 and A2 are C₁₋₅ alkylene, and B1 is —(C₆H₄)—.

23. (canceled)

24. (canceled)

25. The compound according to claim 1, wherein ------ is absent.

26. (canceled)

27. (canceled)

28. The compound according to claim 1, wherein A is
(i) —(CH₂)₂—(C₆H₄)—(CH₂)₂—CH₃,
(ii) —(CH₂)₂—(C₆H₄)—OCH₂CH₃,
(iii) —(CH₂)₂—(C₆H₄)—O(CH₂)₂₋₄NHCO₂R′, wherein R′ is a branched or straight C₁₋₆ alkyl,
(iv) —(CH₂)₂—(C₆H₄)—O(CH₂)₂₋₄O(CH₂)₂NHCO₂R′, wherein R′ is a branched or straight C₁₋₄ alkyl, or
(v) —(CH₂)₂—(C₆H₄)—O(CH₂)₂₋₄SO₂R′, wherein R′ is a branched or straight C₁₋₄ alkyl.

29. (canceled)

30. (canceled)

31. (canceled)

32. (canceled)

33. (canceled)

34. The compound according to claim 1, wherein X is methyl or ethyl, wherein ------ is absent, and wherein Y is —OCH₂—, and R is 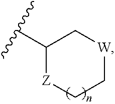
where n=1, Z═O and W=O.

35. The compound according to claim 1, wherein the compound is selected from the group consisting of:
3-methyl-2-(4-propylphenethyl)-6-((tetrahydro-2H-pyran-2-yl)methoxy)pyridin-4-ol,
3-ethyl-2-(4-propylphenethyl)-6-((tetrahydro-2H-pyran-2-yl)methoxy)pyridin-4-ol,
6-((1,4-dioxan-2-yl)methoxy)-3-methyl-2-(4-propylphenethyl)pyridin-4-ol,
(S)-6-((1,4-dioxan-2-yl)methoxy)-2-(4-ethoxyphenethyl)-3-methylpyridin-4-ol,
6-((1,4-dioxan-2-yl)methoxy)-3-ethyl-2-(4-propylphenethyl)pyridin-4-ol,
6-((1,4-dioxan-2-yl)methoxy)-2-(4-ethoxyphenethyl)-3-ethylpyridin-4-ol,
tert-butyl (2-(4-(2-(6-((1,4-dioxan-2-yl)methoxy)-4-hydroxy-3-methylpyridin-2-yl)ethyl)phenoxy)ethyl)carbamate,
2-(4-(2-(6-((1,4-dioxan-2-yl)methoxy)-4-hydroxy-3-methylpyridin-2-yl)ethyl)phenoxy)ethan-1-aminium chloride,
N-(2-(4-(2-(6-((1,4-dioxan-2-yl)methoxy)-4-hydroxy-3-methylpyridin-2-yl)ethyl)phenoxy)ethyl)acetamide,
ethyl 2-(4-(2-(6-((1,4-dioxan-2-yl)methoxy)-4-hydroxy-3-methylpyridin-2-yl)ethyl)phenoxy)acetate,
tert-butyl (2-(4-(2-(6-((1,4-dioxan-2-yl)methoxy)-3-ethyl-4-hydroxypyridin-2-yl)ethyl)phenoxy)ethyl)carbamate,
tert-butyl (3-(4-(2-(6-((1,4-dioxan-2-yl)methoxy)-3-ethyl-4-hydroxypyridin-2-yl)ethyl)phenoxy)propyl)carbamate,
methyl 2-((4-(2-(6-((1,4-dioxan-2-yl)methoxy)-3-ethyl-4-hydroxypyridin-2-yl)ethyl)phenoxy)methyl)oxazole-4-carboxylate,
tert-butyl (2-(2-(4-(2-(6-((1,4-dioxan-2-yl)methoxy)-3-ethyl-4-hydroxypyridin-2-yl)ethyl)phenoxy)ethoxy)ethyl)carbamate,
6-((1,4-dioxan-2-yl)methoxy)-3-ethyl-2-(4-hydroxyphenethyl)pyridin-4-ol,
2-(2-(4-(2-(6-((1,4-dioxan-2-yl)methoxy)-3-ethyl-4-hydroxypyridin-2-yl)ethyl)phenoxy)ethyl)isoindoline-1,3-dione,
6-((1,4-dioxan-2-yl)methoxy)-2-(2-(6-ethoxypyridin-3-yl)ethyl)-3-ethylpyridin-4-ol,
6-((1,4-dioxan-2-yl)methoxy)-2-(2-(6-ethoxy-5-fluoropyridin-3-yl)ethyl)-3-ethylpyridin-4-ol,
6-((1,4-dioxan-2-yl)methoxy)-2-(4-(2-aminoethoxy)phenethyl)-3-ethylpyridin-4-ol hydrochloride,
6-((1,4-dioxan-2-yl)methoxy)-2-(4-(3-aminopropoxy)phenethyl)-3-ethylpyridin-4-ol hydrochloride,
6-((1,4-dioxan-2-yl)methoxy)-3-ethyl-2-(4-(3-(methylsulfonyl)propoxy)phenethyl)pyridin-4-ol,
6-((1,4-dioxan-2-yl)methoxy)-3-ethyl-2-(4-(2-phenoxyethoxy)phenethyl)pyridin-4-ol,
6-((1,4-dioxan-2-yl)methoxy)-3-ethyl-2-(4-((3-methyl-1,2,4-oxadiazol-5-yl)methoxy)phenethyl)pyridin-4-ol,
6-((1,4-dioxan-2-yl)methoxy)-2-(2-(4-(ethoxy-d5)phenyl)ethyl-1,1,2,2-d4)-3-ethylpyridin-4-ol,
(S)-6-((1,4-dioxan-2-yl)methoxy)-2-(4-butylphenyl)-3-ethylpyridin-4-ol,
(S)-6-((1,4-dioxan-2-yl)methoxy)-2-(3,4-dimethoxyphenethyl)-3-ethylpyridin-4-ol,
(S)-6-((1,4-dioxan-2-yl)methoxy)-2-(2,4-dimethoxyphenethyl)-3-ethylpyridin-4-ol,
(S)-5-(2-(6-((1,4-dioxan-2-yl)methoxy)-3-ethyl-4-hydroxypyridin-2-yl)ethyl)-2-methoxybenzonitrile,
(S)-2-(2-(6-((1,4-dioxan-2-yl)methoxy)-3-ethyl-4-hydroxypyridin-2-yl)ethyl)-5-methoxybenzonitrile,
ethyl 5-(4-(2-(6-((1,4-dioxan-2-yl)methoxy)-3-ethyl-4-hydroxypyridin-2-yl)ethyl)phenoxy)pentanoate,
6-((1,4-dioxan-2-yl)methoxy)-3-ethyl-2-(4-(2-hydroxyethoxy)phenethyl)pyridin-4-ol,
2-(4-(2-(6-((1,4-dioxan-2-yl)methoxy)-3-ethyl-4-hydroxypyridin-2-yl)ethyl)phenoxy)acetamide,
sodium 3-(4-hydroxy-2-(4-propylphenethyl)-6-((tetrahydro-2H-pyran-2-yl)methoxy)pyridin-3-yl)propanoate, and
ethyl 3-(4-hydroxy-2-(4-propylphenethyl)-6-((tetrahydro-2H-pyran-2-yl)methoxy)pyridin-3-yl)propanoate.

36. A pharmaceutical composition for use as a medicament, said pharmaceutical composition comprising a compound according to claim 1 and a pharmaceutically acceptable carrier, excipient or diluent.

37. The pharmaceutical composition for use according to claim 36, wherein the composition is for use in the treatment of fibrotic, inflammatory, diabetic or cognitive disease.
